(19) United States
(12) Patent Application Publication
(10) Pub. No.: US 2011/0033389 A1
(43) Pub. Date: Feb. 10, 2011

(54) Modified antibodies for passive immunotherapy

(76) Inventors: Zhifeng Chen, Vista, CA (US); Jehangir Wadia, San Diego, CA (US); Robert Anthony Williamson, La Jolla, CA (US)

Correspondence Address: K&L Gates LLP, 3580 Carmel Mountain Road, Suite 200, San Diego, CA, 92130, US

(21) Appl. No.: 12/799,746

(22) Filed: Apr. 29, 2010

Related U.S. Application Data
(60) Provisional application No. 61/215,020, filed on Apr. 29, 2009.

(51) Int. Cl. A61K 39/42; C07K 19/00; G01N 33/566; C12N 15/63; C07H 21/00; C12N 5/10; C12P 21/02; C07K 7/06; A61P 31/22; A61P 31/12; A61K 49/00

(52) U.S. Cl. 424/9.6; 530/387.3; 435/7.1; 424/134.1; 435/320.1; 536/23.53; 435/328; 435/69.6; 530/327; 530/328; 424/9.1

(57) ABSTRACT

Provided herein are modified therapeutic antibodies containing a protein transduction domain conjugated to a neutralizing antibody. Also provided are methods for generating the modified therapeutic antibodies and methods of using the modified therapeutic antibodies for prevention and treatment of disease.

### RELATED APPLICATIONS

Benefit of priority is claimed to U.S. Provisional Application Ser. No. 61/215,020 to Zhifeng Chen, Joshua Nelson, Jehangir Wadia and Robert Anthony Williamson, entitled “MODIFIED ANTIBODIES FOR PASSIVE IMMUNOTHERAPY,” filed on Apr. 29, 2009.

This application is related to corresponding International Application No. [Attorney Docket No. 3800013-00056/1150PC] to Zhifeng Chen, Joshua Nelson, Jehangir Wadia and Robert Anthony Williamson, entitled “MODIFIED ANTIBODIES FOR PASSIVE IMMUNOTHERAPY,” which also claims priority to U.S. Provisional Application Ser. No. 61/215,020.

The subject matter of each of the above-referenced applications is incorporated by reference in its entirety.

### INCORPORATION BY REFERENCE OF SEQUENCE LISTING PROVIDED ON COMPACT DISCS

An electronic version on compact disc (CD-R) of the Sequence Listing is filed herewith in duplicate (labeled Copy # 1 and Copy # 2), the contents of which are incorporated by reference in their entirety. The computer-readable file on each of the aforementioned compact discs, created on Apr. 29, 2010, is identical, 328 kilobytes in size, and titled 1150SEQ.001.txt.

### FIELD OF INVENTION

Provided herein are modified therapeutic antibodies which bind to and neutralize viruses and other pathogenic microorganisms for the prevention and treatment of diseases.

### BACKGROUND

Multiple pathogenic infections emanate from mucosal surfaces. To combat these pathogens, a mucosal immune system exists that involves secretory glands and lymphoid tissues at mucosal surfaces, including the alimentary system, skin, genital tract, nasal mucosal surface and ocular mucosal surface. The humoral arm of mucosal immunity includes secretory IgA (SIgA) that accounts for the vast majority (roughly 95%) of secreted mucosal antibodies, while IgG subclasses account for the rest (Nesburn et al. (2006) Ocul Surf 4:178-187). Secretory antibodies function both by performing antigen exclusion at mucosal surfaces and by virus and endotoxin neutralization within epithelial cells without causing tissue damage (Brandtzaeg (2007) Vaccine 25:5467-5484). SIgA do not fix complement and, thus, avoid Fc-mediated effector mechanisms contributing to inflammation such as complement (Nesburn et al. (2006) Ocul Surf 4:178-187). SIgA during transcytosis are capable of performing intracellular virus or endotoxin neutralization and clearance (Brandtzaeg (2007) Vaccine 25:5467-5484). Mucosal immunity is somewhat separate from the peripheral systemic immune system (e.g., bone marrow, spleen and lymph nodes) (Toka et al. (2004) Immunol. Rev. 199:100-112). Effective mucosal immunization induces both mucosal and systemic immune responses, while systemic immunizations often do not produce mucosal immunity. To stimulate mucosal immunity, special modes of immunization using molecular adjuvants have been devised (Toka et al. (2004) Immunol. Rev. 199:100-112; Nesburn et al. (2006) Ocul Surf 4:178-187; Ebensen and Guzman (2008) Hum Vaccine 4:13-22). Effective mucosal immunization has proven difficult to elicit reproducibly.

Accordingly, it is among the objects herein, to provide therapeutics and methods for enhancing the efficacy of therapeutic antibodies for passive immunization and therapy, for example, for passive immunization and therapy at mucosal surfaces.

### SUMMARY

Provided herein are modified therapeutic antibodies and methods for enhancing the efficacy of therapeutic antibodies for passive immunization and therapy. Provided herein are modified therapeutic antibodies that contain a protein transduction domain and an antibody, or antigen-binding fragment thereof. Exemplary of such modified antibodies are antibodies, or antigen binding fragments thereof, that contain an antigen binding domain that binds to a surface viral protein. Also provided herein are protein transduction domains. In some examples the protein transduction domains contain a polypeptide having an amino acid sequence selected from among a polypeptide having an amino acid sequence set forth in any of SEQ ID NOS:5-867.

In some examples, the modified therapeutic antibody provided herein contains a protein transduction domain that includes a peptide having the amino acid sequence B₁-X₁-B₂-B₃-B₄-X₂-B₅-B₆-X₃-X₄-B₇, where B₁, B₂, B₃, B₄, B₅, B₆, and B₇ are each independently lysine or arginine and X₁, X₂, X₃, and X₄ are each independently any amino acid except proline. In some examples, X₁, X₂, X₃, and X₄ are each independently any non-basic amino acid except proline. In some examples of such protein transduction domains, the non-basic amino acid is selected from among serine, leucine, alanine, asparagine, aspartic acid and glycine. In some examples of such protein transduction domains, the protein transduction domain contains a peptide having an amino acid sequence selected from among a peptide having an amino acid sequence set forth in any of SEQ ID NOS: 500-855 and 867. In particular examples of such protein transduction domains, the protein transduction domain contains a peptide having an amino acid sequence selected from among RSRRKSRRNGR (SEQ ID NO: 505), RLRRKARRDSR (SEQ ID NO: 741), and KARRKGRRGGK (SEQ ID NO: 723).

In some examples, the modified therapeutic antibody provided herein contains a protein transduction domain that includes a peptide having the amino acid sequence B₁-P₁-X₁-B₂-P₂-B₃-X₂-X₃-X₄-B₄, where P₁ and P₂ are proline, B₁, B₂, B₃ and B₄ are each independently lysine or arginine, and X₁, X₂, X₃ and X₄ are each independently any amino acid. In some examples of such protein transduction domains, X₁, X₂, X₃ and X₄ are each independently any non-basic amino acid. In some examples of such a protein transduction domain, X₁, X₂, X₃ and X₄ are each independently selected from among arginine, lysine, proline and aspartic acid. In some examples of such protein transduction domains, the protein transduction domain is selected from among a peptide having an amino acid sequence set forth in any of SEQ ID NOS: 144-499. In particular examples of such protein transduction domains, the protein transduction domain contains a peptide having an amino acid sequence selected from among RPRRPRPDRR (SEQ ID NO: 160), KPRKPRRPRK (SEQ ID NO: 201), and RPLRPRRKGR (SEQ ID NO: 492).

In some examples, the modified therapeutic antibody provided herein contains a protein transduction domain that includes a peptide having the amino acid sequence B₁-B₂-B₃-B₄-B₅-Q-B₆-B₇-B₈, where Q is glutamine, and B₁, B₂, B₃, B₄, B₅, B₆, B₇, and B₈ are each independently lysine or arginine. In some examples of such protein transduction domains, the protein transduction domain is selected from among a peptide having an amino acid sequence set forth in any of SEQ ID NOS: 5-30. In particular examples of such protein transduction domains, the protein transduction domain contains a peptide having an amino acid sequence selected from among RRKRRQRRRR (SEQ ID NO: 9), RKKRKQKKR (SEQ ID NO: 13), and KRRKRQRRR (SEQ ID NO: 29).

In some examples, the modified therapeutic antibody provided herein contains a protein transduction domain that includes a peptide having the amino acid sequence B₁-B₂-B₃-B₄-B₅-X-B₆-B₇-B₈, where X is any amino acid except glutamine, and B₁, B₂, B₃, B₄, B₅, B₆, B₇, and B₈ are each independently lysine or arginine. In some examples of such protein transduction domains, the amino acid X is selected from among from among arginine, lysine, aspartic acid, glutamic acid and asparagine. In some examples of such protein transduction domains, the protein transduction domain is selected from among a peptide having an amino acid sequence set forth in any of SEQ ID NOS: 31-143 and 860. In particular examples of such protein transduction domains, the protein transduction domain contains a peptide having an amino acid sequence selected from among KKRKKEKKKR (SEQ ID NO: 90), KRRKRNRRRR (SEQ ID NO: 860), and RKRREKRRR (SEQ ID NO: 100).

In some examples, the modified therapeutic antibody provided herein contains a protein transduction domain that is selected from among a peptide having an amino acid sequence set forth in SEQ ID NOS: 868-939. In some examples, the protein transduction domain is an HIV-TAT protein transduction domain selected from among a peptide having an amino acid sequence set forth in SEQ ID NOS: 910-921. In some examples, the protein transduction domain is a polypeptide having the amino acid sequence set forth in SEQ ID NO:915.

Provided herein are modified therapeutic antibodies or antigen-binding fragments thereof that contain an antigen binding domain that binds to a surface viral protein. In some examples, the viral surface protein is a viral envelope protein or a viral capsid protein. In some examples, the viral envelope protein is a glycoprotein.

Provided herein are modified therapeutic antibodies or antigen-binding fragments thereof that contain an antigen binding domain that binds to an enveloped virus or a non-enveloped virus. In some examples, the antibody or antigen-binding fragment thereof binds to a herpes virus, a metapneumovirus or a respiratory syncytial virus. In some examples, the antibody or antigen-binding fragment thereof binds to herpes virus HSV-1 or HSV-2.

Provided herein are modified therapeutic antibodies or antigen-binding fragments thereof that contain an antigen binding domain that binds to a herpes virus envelope protein. In some examples, the virus envelope protein is herpes virus glycoprotein D.

Provided herein are modified therapeutic antibodies that are fusion proteins, where the protein transduction domain is conjugated to the antibody or antigen-binding fragment thereof by recombinant means. Also provided herein are modified therapeutic antibodies where the protein transduction domain is conjugated to the antibody or antigen-binding fragment post-translationally.

Provided herein are modified therapeutic antibodies that are human antibodies. Also provided herein are modified therapeutic antibodies where the antigen binding domain of the antibody or antigen-binding fragment thereof is a humanized antibody or antigen-binding fragment thereof. Provided herein are modified therapeutic antibodies where the antibody or antigen-binding fragment is a single-chain Fv (scFv), Fab, Fab′, F(ab′)₂, Fv, dsFv, diabody, Fd, or Fd′ fragment. In particular examples, the antigen-binding fragment is a single chain antibody. In such examples, the single chain antibody can contain a light chain variable (V_{L}) domain or functional region thereof and a heavy chain variable (V_{H}) domain or functional region thereof. In some examples, the V_{L} domain or functional region thereof contains a complementarity determining region 1 (CDR1), a complementarity determining region 2 (CDR2) and a complementarity determining region 3 (CDR3). In some examples, the V_{H} domain or functional region thereof contains a complementarity determining region 1 (CDR1), a complementarity determining region 2 (CDR2) and a complementarity determining region 3 (CDR3). In some examples, the single chain antibody further contains a peptide liker. In such examples, a peptide linker can be located between the light chain variable domain (V_{L}) and the heavy chain variable domain (V_{H}).

Provided herein are modified therapeutic antibodies the contain the AC8 antibody or antigen binding fragments thereof. In some examples, the antibody contains a heavy chain CDR3 having the amino acid sequence set forth in SEQ ID NO: 1055. In particular examples, the antibody contains a light chain variable domain (V_{L}) having an amino acid sequence set forth in SEQ ID NO: 1052. In particular examples, the antibody contains a heavy chain variable domain (V_{H}) having an amino acid sequence set forth in SEQ ID NO: 1053. In some examples, the antibody contains a light chain having an amino acid sequence set forth in SEQ ID NO:1018. In some examples, the antibody contains a heavy chain having an amino acid sequence set forth in SEQ ID NO:1056. In particular examples, the modified antibody is an AC8 Fab antibody that contains a light chain having an amino acid sequence set forth in SEQ ID NO:1018 and an heavy chain sequence having an amino acid sequence set forth in SEQ ID NO:1016 (AC8FabTAT). In particular examples, the antibody is an AC8 single chain antibody that contains a polypeptide having the amino acid sequence set forth in SEQ ID NO: 1. In particular examples, the modified antibody is an AC8 single chain antibody that contains a polypeptide having the amino acid sequence set forth in SEQ ID NO: 2 (TATscFvAC8).

Provided herein are modified therapeutic antibodies the contain a peptide linker located between the protein transduction sequence and the antibody or antigen-binding fragment thereof. In some examples, the peptide linker contains about 1 to about 50 amino acids.

Provided herein are modified therapeutic antibodies that contain a diagnostic agent. In some examples, the diagnostic agent is selected from among an enzyme, a fluorescent compound, or an electron transfer agent.

Provided herein are combinations containing any modified therapeutic antibody provided herein and an antiviral agent. In some examples, the antiviral agent is selected from among acyclovir, famciclovir, ganciclovir, penciclovir, valacyclovir, valganciclovir, idoxuridine, trifluridine, brivudine, cidofovir, docosanol, fomivirsen, foscarnet and tromantadine.

Provided herein are combinations containing any modified therapeutic antibody provided herein and a viscoelastic agent. In some examples, the viscoelastic agent is hyaluronic acid.

Provided herein are pharmaceutical compositions that contain any modified therapeutic antibody provided herein and a pharmaceutically acceptable carrier or excipient. Provided herein are pharmaceutical compositions that contain any combination provided herein and a pharmaceutically acceptable carrier or excipient. In some examples, the pharmaceutical compositions provided herein are formulated as a gel, ointment, liquid, suspension, aerosol, tablet, pill or powder. In some examples, the pharmaceutical compositions provided herein are formulated as eyedrops or a nasal spray.

Provided herein are methods of treatment or prevention of a viral infection by administering any modified therapeutic antibody provided herein or pharmaceutical composition provided herein. In some examples, the viral infection is a herpes virus infection. In some examples, the antibody or composition is administered topically, parenterally, locally, or systemically. In some examples, the antibody or composition is administered orally, ocularly, intravenously, or directly to a mucosal surface. In some examples, the antibody or composition is administered to a mucosal surface that is selected from among corneal, conjunctival, intravitreal, intra-aqueous, buccal, sublingual, nasal, vaginal, pulmonary, stomachic, intestinal and rectal surfaces.

Provided herein are methods of treatment or prevention of a viral infection by administering any modified therapeutic antibody provided herein or pharmaceutical composition provided herein and an antiviral agent. In some examples, the antiviral agent is selected from among acyclovir, famciclovir, ganciclovir, penciclovir, valacyclovir, valganciclovir, idoxuridine, trifluridine, brivudine, cidofovir, docosanol, fomivirsen, foscarnet and tromantadine.

Provided herein are vectors containing nucleic acid encoding any modified therapeutic antibody provided herein. Provided herein are isolated nucleic acids that encode any modified therapeutic antibody provided herein. Provided herein are isolated cells that contain any modified therapeutic antibody provided herein, any nucleic acid provided herein, or any vector provided herein. The isolated cells provided herein can be a prokaryotic or eukaryotic cell. Also provided herein are methods of expressing a modified therapeutic antibody provided herein by culturing a cell that contains nucleic acid encoding the modified therapeutic antibody under conditions that allow for expression of the encoded modified therapeutic antibody. Also provided herein are transgenic animals that contain the nucleic acid encoding any modified therapeutic antibody provided herein. Also provided herein are methods of producing a modified therapeutic antibody by isolating the modified therapeutic antibody from the transgenic animal. In some examples, the modified therapeutic antibody is isolated from the serum or milk of the transgenic animal.

Provided herein are methods for increasing the therapeutic efficacy of an antiviral antibody or an antigen binding fragment thereof, that include conjugating a protein transduction domain to an antibody or antigen binding fragment thereof that binds a glycoprotein on the surface of a virus, where the antibody or antigen binding fragment can neutralize the virus, whereby conjugation increases the therapeutic efficacy of the antibody or antigen binding fragment compared to the therapeutic efficacy of the antibody or antigen binding fragment in the absence of the protein transduction domain, when administered to a subject.

### DETAILED DESCRIPTION

Outline

| | |
|---|---|
| A. | Definitions |
| B. | Modified Therapeutic Antibodies |
| C. | Structure of Modified Therapeutic Antibodies |

| | |
|---|---|
| 1. | Protein transduction domain |

| | |
|---|---|
| a. | TAT-like transduction domains |

| | |
|---|---|
| i. | TAT-like transduction domains with Gln at position six |
| ii. | TAT-like transduction domains without Gln at position six |

| | |
|---|---|
| b. | Prion-like transduction domains |
| c. | Transduction peptides with basic charges on one face of an alpha-helix |

| | |
|---|---|
| 2. | Antibodies for Modification |

| | |
|---|---|
| a. | General Characteristics of Antibodies |

| | |
|---|---|
| i. | Structural and functional domains of antibodies |
| ii. | Antibody fragments |

| | |
|---|---|
| b. | Selection of antibodies for modification |

| | |
|---|---|
| i. | Neutralizing antibodies |

| | |
|---|---|
| (1) | Herpes virus neutralizing antibodies |

| | |
|---|---|
| (a) | Herpes virus glycoprotein D antibodies |

| | |
|---|---|
| 3. | Attachment of the Protein Transduction Domain |

| | |
|---|---|
| a. | Recombinant methods |

| | |
|---|---|
| i. | Spacer/Linker peptides |

| | |
|---|---|
| b. | Chemical cross-linking |

| | |
|---|---|
| D. | Additional Modifications of Therapeutic Antibodies |

| | |
|---|---|
| 1. | Modifications to reduce immunogenicity |
| 2. | Attachment of a detectable moiety |
| 3. | Modifications to improve binding specificity |

| | |
|---|---|
| E. | Preparation of Modified Therapeutic Antibodies |

| | |
|---|---|
| 1. | Vectors and nucleic acids |
| 2. | Cells and Hosts |

| | |
|---|---|
| a. | Prokaryotic cells |
| b. | Yeast cells |
| c. | Insect cells |
| d. | Mammalian cells |
| e. | Plants |

| | |
|---|---|
| 3. | Purification of antibodies |

| | |
|---|---|
| F. | Therapeutic Methods |

| | |
|---|---|
| 1. | Selection of subjects for therapy |
| 2. | Dosages |
| 3. | Routes of administration |
| 4. | Combination therapies |

| | |
|---|---|
| G. | Diagnostic Methods |

| | |
|---|---|
| 1. | Assays for selection of a protein transduction domain |
| 2. | In vitro assays for analyzing virus neutralization effects of antibodies |
| 3. | In vivo animal models for assessing efficacy of the modified therapeutic |
| | antibodies |
| 4. | In vitro detection of pathogenic infection |
| 5. | In vivo detection of pathogenic infection |
| 6. | Monitoring Infection |

| | |
|---|---|
| H. | Pharmaceutical Compositions, Combinations and articles of |
| | manufacture/Kits |

| | |
|---|---|
| 1. | Pharmaceutical Compositions |
| 2. | Articles of Manufacture/Kits |
| 3. | Combinations |

| | |
|---|---|
| I. | Examples |

### A. DEFINITIONS

Unless defined otherwise, all technical and scientific terms used herein have the same meaning as is commonly understood by one of skill in the art to which the invention(s) belong. All patents, patent applications, published applications and publications, GENBANK sequences, websites and other published materials referred to throughout the entire disclosure herein, unless noted otherwise, are incorporated by reference in their entirety. In the event that there is a plurality of definitions for terms herein, those in this section prevail. Where reference is made to a URL or other such identifier or address, it is understood that such identifiers can change and particular information on the internet can come and go, but equivalent information is known and can be readily accessed, such as by searching the internet and/or appropriate databases. Reference thereto evidences the availability and public dissemination of such information.

As used herein, “antibody” refers to immunoglobulins and immunoglobulin fragments, whether natural or partially or wholly synthetically, such as recombinantly, produced, including any fragment thereof containing at least a portion of the variable region of the immunoglobulin molecule that retains the binding specificity or ability of the full-length immunoglobulin. Hence, an antibody includes any protein having a binding domain that is homologous or substantially homologous to an immunoglobulin antigen binding domain (antibody combining site). For purposes herein, the term antibody includes antibody fragments, such as, but not limited to, Fab, Fab′, F(ab′)₂, single-chain Fvs (scFv), Fv, dsFv, diabody, Fd and Fd′ fragments, Fab fragments, Fd fragments, scFab fragments and scFv fragments. Antibodies include members of any immunoglobulin class, including IgG, IgM, IgD, IgE, IgA and IgY.

As used herein, a “therapeutic antibody” refers to any antibody that is administered for treatment of an animal, including a human. Such antibodies can be prepared by any known method for the production of polypeptides, and hence, include, but are not limited to, recombinantly produced antibodies, synthetically produced antibodies, and therapeutic antibodies extracted from cells or tissues and other sources. As isolated from any source or as produced, therapeutic antibodies can be heterogeneous in length or differ in post-translational modification, such as glycosylation (i.e. carbohydrate content). Heterogeneity of therapeutic antibodies also can differ depending on the source of the therapeutic antibodies. Hence, reference to therapeutic antibodies refers to the heterogeneous population as produced or isolated. When a homogeneous preparation is intended, it will be so-stated. References to therapeutic antibodies herein are to their monomeric, dimeric or other multimeric forms, as appropriate.

As used herein, a “neutralizing antibody” is any antibody that binds to a pathogen and interferes with the ability of the pathogen to infect a cell and/or cause disease in a subject. Exemplary of neutralizing antibodies are neutralizing antibodies that bind to viruses, bacteria, and fungal pathogens. Typically, the neutralizing antibodies provided herein bind to the surface of the pathogen. In examples where the pathogen is a virus, a neutralizing antibody that binds to the virus typically binds to a protein on the surface of the virus. Depending on the class of the virus, the surface protein can be a capsid protein (e.g., a capsid protein of a non-enveloped virus) or a viral envelope protein (e.g., a viral envelope protein of an enveloped virus). In some examples, the protein is a glycoprotein.

As used herein, “specifically bind” or “immunospecifically bind” with respect to an antibody or antigen-binding fragment thereof are used interchangeably herein and refer to the ability of the antibody or antigen-binding fragment to form one or more noncovalent bonds with a cognate antigen, by noncovalent interactions between the antibody combining site(s) of the antibody and the antigen (e.g. human DLL4). Typically, an antibody that immunospecifically binds (or that specifically binds) to an antigen is one that binds to the antigen with an affinity constant Ka of about or 1×10⁷M⁻¹ or 1×10⁸M⁻¹ or greater (or a dissociation constant (IQ) of 1×10⁻⁷M or 1×10⁻⁸M or less). Affinity constants can be determined by standard kinetic methodology for antibody reactions, for example, immunoassays, surface plasmon resonance (SPR) (Rich and Myszka (2000) Curr. Opin. Biotechnol 11:54; Englebienne (1998) Analyst. 123:1599), isothermal titration calorimetry (ITC) or other kinetic interaction assays known in the art (see, e.g., Paul, ed., Fundamental Immunology, 2nd ed., Raven Press, New York, pages 332-336 (1989); see also U.S. Pat. No. 7,229,619 for a description of exemplary SPR and ITC methods for calculating the binding affinity of anti-RSV antibodies). Instrumentation and methods for real time detection and monitoring of binding rates are known and are commercially available (e.g., BiaCore 2000, Biacore AB, Upsala, Sweden and GE Healthcare Life Sciences; Malmqvist (2000) Biochem. Soc. Trans. 27:335).

As used herein, the term “bind selectively” or “selectively binds,” in reference to a polypeptide or an antibody provided herein, means that the polypeptide or antibody binds with a selected epitope without substantially binding to another epitope. Typically, an antibody or fragment thereof that selectively binds to a selected epitope specifically binds to the epitope, such as with an affinity constant Ka of about or 1×10⁷M⁻¹ or 1×10⁸M⁻¹ or greater.

As used herein, an “enveloped virus” is an animal virus which possesses an outer membrane or ‘envelope’, which is a lipid bilayer containing viral proteins, surrounding the virus capsid. The envelope proteins of the virus participate in the assembly of the infectious particle and also are involved in virus entry by binding to receptors present on the host cell and inducing fusion between the viral envelope and a membrane of the host cell. Enveloped viruses can be either spherical or filamentous (rod-shaped). Exemplary enveloped viruses include, but are not limited to, members of the Herpesviridae, Poxyiridae, Hepadnaviridae, Togaviridae, Arenaviridae, Flaviviridae, Orthomyxoviridae, Paramyxoviridae, Bunyaviridae, Rhabdoviridae, Filoviridae, Coronaviridae, and Bornaviridae virus families

As used herein, a “non-enveloped virus” or “naked virus” is a virus that lacks a viral envelope. For infection of a host cell, a non-enveloped virus uses proteins of the viral capsid for attachment to the target cell. Exemplary non-enveloped viruses include, but are not limited to, Adenoviridae, Papillomavirinae, Parvoviridae, Polyomavirinae, Circoviridae, Reoviridae, Picornaviridae, Caliciviridae, and Astroviridae virus families.

As used herein, a “surface protein” of a pathogen is any protein that is located on external surface of the pathogen. The surface protein can be partially or entirely exposed to the external environment (i.e. outer surface). Exemplary of surface proteins are membrane proteins, such as, for example, a protein located on the surface of a viral envelope or bacterial outer membrane (e.g., a membrane glycoprotein). Membrane proteins can be transmembrane proteins (i.e. proteins that traverse the lipid bilayer) or proteins that are non-transmembrane cell surface associated proteins (e.g., anchored or covalently attached to the surface of the membrane, such as attachment to another protein on the surface of the pathogen). Other exemplary surface proteins include viral capsid proteins of non-enveloped enveloped viruses that are at least partially exposed to the external environment.

As used herein, a “modified antibody” or a “modified therapeutic antibody” refers to an antibody that has been modified by attachment of a protein transduction domain.

As used herein, a “protein transduction domain” or “PTD” is a domain that promotes the attachment of the antibody to the surface of a target cell or tissue, for example, at a mucosal surface.

As used herein a “target cell” refers to a cell to which a protein transduction domain can bind or attach. A “target cell” can be any cell, including human cells, that exists either in vivo or in vitro.

As used herein, “monoclonal antibody” refers to a population of identical antibodies, meaning that each individual antibody molecule in a population of monoclonal antibodies is identical to the others. This property is in contrast to that of a polyclonal population of antibodies, which contains antibodies having a plurality of different sequences. Monoclonal antibodies can be produced by a number of well-known methods (Smith et al. (2004) J. Clin. Pathol. 57, 912-917; and Nelson et al., J Clin Pathol (2000), 53, 111-117). For example, monoclonal antibodies can be produced by immortalization of a B cell, for example through fusion with a myeloma cell to generate a hybridoma cell line or by infection of B cells with virus such as EBV. Recombinant technology also can be used to produce monoclonal antibodies in vitro from clonal populations of host cells by transforming the host cells with plasmids carrying artificial sequences of nucleotides encoding the antibodies.

As used herein, a “conventional antibody” refers to an antibody that contains two heavy chains (which can be denoted H and H′) and two light chains (which can be denoted L and L′) and two antibody combining sites, where each heavy chain can be a full-length immunoglobulin heavy chain or any functional region thereof that retains antigen binding capability (e.g. heavy chains include, but are not limited to, V_{H} chains, V_{H}-C_{H}1 chains, V_{H}-C_{H}1-C_{H}2-C_{H}3 chains and V_{H}-C_{H}1-C_{H}2-C_{H}3-C_{H}4), and each light chain can be a full-length light chain or any functional region of (e.g. light chains include, but are not limited to, V_{L} chains and V_{L}-C_{L} chains). Each heavy chain (H and H′) pairs with one light chain (L and L′, respectively)

As used herein, a “full-length antibody” is an antibody having two full-length heavy chains (e.g. V_{H}-C_{H}1-C_{H}2-C_{H}3 or V_{H}-C_{H}1-C_{H}2-C_{H}3-C_{H}4) and two full-length light chains (V_{L}-C_{L}) and hinge regions, such as human antibodies produced naturally by antibody secreting B cells and antibodies with the same domains that are synthetically produced.

As used herein, an “antibody fragment” or “antigen-binding fragment” of an antibody refers to any portion of a full-length antibody that is less than full length but contains at least a portion of the variable region of the antibody that binds antigen (e.g. one or more CDRs and/or one or more antibody combining sites) and thus retains the binding specificity, and at least a portion of the specific binding ability of the full-length antibody; antibody fragments include antibody derivatives produced by enzymatic treatment of full-length antibodies, as well as synthetically, e.g. recombinantly produced derivatives. Examples of antibody fragments include, but are not limited to, Fab, Fab′, F(ab′)₂, single-chain Fvs (scFv), Fv, dsFv, diabody, Fd and Fd′ fragments other fragments, including modified fragments (see, for example, Methods in Molecular Biology, Vol 207: Recombinant Antibodies for Cancer Therapy Methods and Protocols (2003); Chapter 1; p 3-25, Kipriyanov). The fragment can include multiple chains linked together, such as by disulfide bridges and/or by peptide linkers. An antibody fragment generally contains at least about 50 amino acids and typically at least 200 amino acids.

As used herein, an “Fv antibody fragment” or “Fv fragment” is composed of one variable heavy domain (V_{H}) and one variable light (V_{L}) domain linked by noncovalent interactions.

As used herein, a “dsFv” refers to an Fv with an engineered intermolecular disulfide bond, which stabilizes the V_{H}-V_{L} pair.

As used herein, an “Fd fragment” is a fragment of an antibody containing a variable domain (V_{H}) and one constant region domain (C_{H}1) of an antibody heavy chain.

As used herein, a “Fab fragment” is an antibody fragment that results from digestion of a full-length immunoglobulin with papain, or a fragment having the same structure that is produced synthetically, e.g. by recombinant methods. A Fab fragment contains a light chain (containing a V_{L} and C_{L}) and another chain containing a variable domain of a heavy chain (V_{H}) and one constant region domain of the heavy chain (C_{H}1).

As used herein, a “F(ab′)₂ fragment” is an antibody fragment that results from digestion of an immunoglobulin with pepsin at pH 4.0-4.5, or a fragment having the same structure that is produced synthetically, e.g. by recombinant methods. The F(ab′)₂ fragment essentially contains two Fab fragments where each heavy chain portion contains an additional few amino acids, including cysteine residues that form disulfide linkages joining the two fragments.

As used herein, a “Fab′ fragment” is a fragment containing one half (one heavy chain and one light chain) of the F(ab′)₂ fragment.

As used herein, an “Fd′ fragment” is a fragment of an antibody containing one heavy chain portion of a F(ab′)₂ fragment.

As used herein, an “Fv′ fragment” is a fragment containing only the V_{H} and

V_{L} domains of an antibody molecule.

As used herein, an “hsFv” refers to antibody fragments in which the constant domains normally present in a Fab fragment have been substituted with a heterodimeric coiled-coil domain (see, e.g., Arndt et al. (2001) J Mol. Biol. 7:312:221-228).

As used herein, a “scFv fragment” refers to an antibody fragment that contains a variable light chain (V_{L}) and variable heavy chain (V_{H}), covalently connected by a polypeptide linker in any order. The linker is of a length such that the two variable domains are bridged without substantial interference. Exemplary linkers are (Gly-Ser)ₙ residues with some Glu or Lys residues dispersed throughout to increase solubility.

As used herein, the phrase “derived from” when referring to antibody fragments derived from another antibody, such as a monoclonal antibody, refers to the engineering of antibody fragments (e.g., Fab, F(ab′), F(ab′)₂, single-chain Fvs (scFv), Fv, dsFv, diabody, Fd and Fd′ fragments) that retain the binding specificity of the original antibody. Such fragments can be derived by a variety of methods known in the art, including, but not limited to, enzymatic cleavage, chemical crosslinking, recombinant means or combinations thereof. Generally, the derived antibody fragment shares the identical or substantially identical heavy chain variable region (V_{H}) and light chain variable region (V_{L}) of the parent antibody, such that the antibody fragment and the parent antibody bind the same epitope.

As used herein, a “parent antibody” or “source antibody” refers the to an antibody from which an antibody fragment (e.g., Fab, F(ab′), F(ab′)₂, single-chain Fvs (scFv), Fv, dsFv, diabody, Fd and Fd′ fragments) is derived.

As used herein, the term “epitope” means any antigenic determinant on an antigen to which the paratope of an antibody binds. Epitopic determinants typically comprise chemically active surface groupings of molecules such as amino acids or sugar side chains and typically have specific three dimensional structural characteristics, as well as specific charge characteristics.

As used herein, a “fusion protein” is a polypeptide engineered to contain sequences of amino acids corresponding to two distinct polypeptides, which are joined together, such as by expressing the fusion protein from a vector containing two nucleic acids, encoding the two polypeptides, in close proximity, e.g. adjacent, to one another along the length of the vector. Exemplary of a fusion protein is a protein transduction domain (PTD)-antibody fusion.

As used herein, “linker” or “spacer” peptide refers to short sequences of amino acids that join two polypeptide sequences (or nucleic acid encoding such an amino acid sequence). “Peptide linker” refers to the short sequence of amino acids joining the two polypeptide sequences. Exemplary of polypeptide linkers are linkers joining a peptide transduction domain to an antibody or linkers joining two antibody chains in a synthetic antibody fragment such as an scFv fragment. Linkers are well-known and any known linkers can be used in the provided methods. Exemplary of polypeptide linkers are (Gly-Ser)ₙ amino acid sequences, with some Glu or Lys residues dispersed throughout to increase solubility. Other exemplary linkers are described herein; any of these and other known linkers can be used with the provided compositions and methods.

As used herein, “antibody hinge region” or “hinge region” refers to a polypeptide region that exists naturally in the heavy chain of the gamma, delta and alpha antibody isotypes, between the C_{H}1 and C_{H}2 domains that has no homology with the other antibody domains. This region is rich in proline residues and gives the IgG, IgD and IgA antibodies flexibility, allowing the two “arms” (each containing one antibody combining site) of the Fab portion to be mobile, assuming various angles with respect to one another as they bind antigen. This flexibility allows the Fab arms to move in order to align the antibody combining sites to interact with epitopes on cell surfaces or other antigens. Two interchain disulfide bonds within the hinge region stabilize the interaction between the two heavy chains. In some embodiments provided herein, the synthetically produced antibody fragments contain one or more hinge regions, for example, to promote stability via interactions between two antibody chains.

As used herein, “diabodies” are dimeric scFv; diabodies typically have shorter peptide linkers than scFvs, and preferentially dimerize.

As used herein, “humanized antibodies” refer to antibodies that are modified to include “human” sequences of amino acids so that administration to a human does not provoke an immune response. A humanized antibody typically contains complementarily determining regions (CDRs) derived from a non-human species immunoglobulin and the remainder of the antibody molecule derived mainly from a human immunoglobulin. Methods for preparation of such antibodies are known. For example, DNA encoding a monoclonal antibody can be altered by recombinant DNA techniques to encode an antibody in which the amino acid composition of the non-variable regions is based on human antibodies. Computer programs have been designed are available for identifying such regions.

As used herein, “idiotype” refers to a set of one or more antigenic determinants specific to the variable region of an immunoglobulin molecule.

As used herein, an “anti-idiotype antibody” refers to an antibody directed against the antigen-specific part of the sequence of an antibody or T cell receptor. In principle an anti-idiotype antibody inhibits a specific immune response.

As used herein, an “Ig domain” is a domain, recognized as such by those in the art, that is distinguished by a structure, called the Immunoglobulin (Ig) fold, which contains two beta-pleated sheets, each containing anti-parallel beta strands of amino acids connected by loops. The two beta sheets in the Ig fold are sandwiched together by hydrophobic interactions and a conserved intra-chain disulfide bond. Individual immunoglobulin domains within an antibody chain further can be distinguished based on function. For example, a light chain contains one variable region domain (V_{L}) and one constant region domain (C_{L}), while a heavy chain contains one variable region domain (V_{H}) and three or four constant region domains (C_{H}). Each V_{L}, C_{L}, V_{H}, and C_{H} domain is an example of an immunoglobulin domain.

As used herein, a “variable region domain” or “variable region” is a specific Ig domain of an antibody heavy or light chain that contains a sequence of amino acids that varies among different antibodies. Each light chain and each heavy chain has one variable region domain (V_{L} and V_{H}). The variable domains provide antigen specificity, and thus are responsible for antigen recognition. Each variable region contains CDRs that are part of the antigen binding site domain and framework regions (FRs).

As used herein, “antigen binding domain,” “antigen binding site,” “antigen combining site” and “antibody combining site” are used synonymously to refer to a domain within an antibody that recognizes and physically interacts with cognate antigen. A native conventional full-length antibody molecule has two conventional antigen combining sites, each containing portions of a heavy chain variable region and portions of a light chain variable region. A conventional antigen binding site contains the loops that connect the anti-parallel beta strands within the variable region domains. The antigen combining sites can contain other portions of the variable region domains. Each conventional antigen binding site contains three hypervariable regions from the heavy chain and three hypervariable regions from the light chain. The hypervariable regions also are called complementarity-determining regions (CDRs).

As used herein, “hypervariable region,” “HV,” “HVR,” “complementarity-determining region” and “CDR” and “antibody CDR” are used interchangeably to refer to one of a plurality of portions within each variable region that together form an antigen binding site of an antibody. Each variable region domain contains three CDRs, named CDR1, CDR2 and CDR3. The three CDRs are non-contiguous along the linear amino acid sequence, but are proximate in the folded polypeptide. The CDRs are located within the loops that join the parallel strands of the beta sheets of the variable domain.

As used herein, “framework regions” or “FR” are the domains within the antibody variable region domains that are located within the beta sheets; the FR regions are comparatively more conserved, in terms of their amino acid sequences, than the hypervariable regions.

As used herein, a “constant region” domain is a domain in an antibody heavy or light chain that contains a sequence of amino acids that is comparatively more conserved than that of the variable region domain. In conventional full-length antibody molecules, each light chain has a single light chain constant region (C_{L}) domain and each heavy chain contains one or more heavy chain constant region (C_{H}) domains, which include, C_{H}1, C_{H}2, C_{H}3 and C_{H}4. Full-length IgA, IgD and IgG isotypes contain C_{H}1, C_{H}2 C_{H}3 and a hinge region, while IgE and IgM contain C_{H}1, C_{H}2, C_{H}3 and C_{H}4. C_{H}1 and C_{L} domains extend the Fab arm of the antibody molecule, thus contributing to the interaction with antigen and rotation of the antibody arms. Antibody constant regions can serve effector functions, such as, but not limited to, clearance of antigens, pathogens and toxins to which the antibody specifically binds, e.g. through interactions with various cells, biomolecules and tissues.

As used herein, a “functional region” of an antibody is a portion of the antibody that contains at least a V_{H}, V_{L}, C_{H} (e.g. C_{H}1, C_{H}2 or C_{H}3), C_{L} or hinge region domain of the antibody, or at least a functional region thereof.

As used herein, a functional region of a V_{H} domain is at least a portion of the full V_{H} domain that retains at least a portion of the binding specificity of the full V_{H} domain (e.g. by retaining one or more CDRs of the full V_{H} domain), such that the functional region of the V_{H} domain, either alone or in combination with another antibody domain (e.g. V_{L} domain) or region thereof, binds to antigen. Exemplary functional regions of V_{H} domains are regions containing the CDR1, CDR2 and/or CDR3 of the V_{H} domain.

As used herein, a functional region of a V_{L} domain is at least a portion of the full V_{L} domain that retains at least a portion of the binding specificity of the full V_{L} domain (e.g. by retaining one or more CDRs of the full V_{L} domain), such that the function region of the V_{L} domain, either alone or in combination with another antibody domain (e.g. V_{H} domain) or region thereof, binds to antigen. Exemplary functional regions of V_{L} domains are regions containing the CDR1, CDR2 and/or CDR3 of the V_{L} domain.

As used herein, “polypeptide” refers to two or more amino acids covalently joined. The terms “polypeptide” and “protein” are used interchangeably herein.

As used herein, a “peptide” refers to a polypeptide that is from 2 to about or 40 amino acids in length.

As used herein, an “amino acid” is an organic compound containing an amino group and a carboxylic acid group. A polypeptide contains two or more amino acids. For purposes herein, amino acids contained in the modified therapeutic antibodies provided herein include the twenty naturally-occurring amino acids (Table 1), non-natural amino acids, and amino acid analogs (e.g., amino acids wherein the α-carbon has a side chain). As used herein, the amino acids, which occur in the various amino acid sequences of polypeptides appearing herein, are identified according to their well-known, three-letter or one-letter abbreviations (see Table 1). The nucleotides, which occur in the various nucleic acid molecules and fragments, are designated with the standard single-letter designations used routinely in the art.

As used herein, “amino acid residue” refers to an amino acid formed upon chemical digestion (hydrolysis) of a polypeptide at its peptide linkages. The amino acid residues described herein are generally in the “L” isomeric form. Residues in the “D” isomeric form can be substituted for any L-amino acid residue, as long as the desired functional property is retained by the polypeptide. NH₂ refers to the free amino group present at the amino terminus of a polypeptide. COOH refers to the free carboxy group present at the carboxyl terminus of a polypeptide. In keeping with standard polypeptide nomenclature described in J. Biol. Chem., 243:3557-59 (1968) and adopted at 37 C.F.R. §§.1.821-1.822, abbreviations for amino acid residues are shown in Table 1:

TABLE 1
Table of Correspondence

SYMBOL

| 1-Letter | 3-Letter | AMINO ACID |
|---|---|---|
| Y | Tyr | Tyrosine |
| G | Gly | Glycine |
| F | Phe | Phenylalanine |
| M | Met | Methionine |
| A | Ala | Alanine |
| S | Ser | Serine |
| I | Ile | Isoleucine |
| L | Leu | Leucine |
| T | Thr | Threonine |
| V | Val | Valine |
| P | Pro | Proline |
| K | Lys | Lysine |
| H | His | Histidine |
| Q | Gln | Glutamine |
| E | Glu | Glutamic acid |
| Z | Glx | Glutamic Acid and/or Glutamine |
| W | Trp | Tryptophan |
| R | Arg | Arginine |
| D | Asp | Aspartic acid |
| N | Asn | Asparagine |
| B | Asx | Aspartic Acid and/or Asparagine |
| C | Cys | Cysteine |
| X | Xaa | Unknown or other |

All sequences of amino acid residues represented herein by a formula have a left to right orientation in the conventional direction of amino-terminus to carboxyl-terminus. In addition, the phrase “amino acid residue” is defined to include the amino acids listed in the Table of Correspondence (Table 1), modified, non-natural and unusual amino acids. Furthermore, a dash at the beginning or end of an amino acid residue sequence indicates a peptide bond to a further sequence of one or more amino acid residues or to an amino-terminal group such as NH₂ or to a carboxyl-terminal group such as COOH.

In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al., Molecular Biology of the Gene, 4th Edition, 1987, The Benjamin/Cummings Pub. co., p. 224).

Such substitutions can be made in accordance with those set forth in Table 2 as follows:

TABLE 2

| Original residue | Conservative substitution |
|---|---|
| Ala (A) | Gly; Ser |
| Arg (R) | Lys |
| Asn (N) | Gln; His |
| Cys (C) | Ser |
| Gln (Q) | Asn |
| Glu (E) | Asp |
| Gly (G) | Ala; Pro |
| His (H) | Asn; Gln |
| Ile (I) | Leu; Val |
| Leu (L) | Ile; Val |
| Lys (K) | Arg; Gln; Glu |
| Met (M) | Leu; Tyr; Ile |
| Phe (F) | Met; Leu; Tyr |
| Ser (S) | Thr |
| Thr (T) | Ser |
| Trp (W) | Tyr |
| Tyr (Y) | Trp; Phe |
| Val (V) | Ile; Leu |

Other substitutions also are permissible and can be determined empirically or in accord with other known conservative or non-conservative substitutions.

As used herein, “naturally occurring amino acids” refer to the 20 L-amino acids that occur in polypeptides.

As used herein, a “non-basic amino acid” refers to any amino acid except lysine and arginine.

As used herein, the term “non-natural amino acid” refers to an organic compound that has a structure similar to a natural amino acid but has been modified structurally to mimic the structure and reactivity of a natural amino acid. Non-naturally occurring amino acids thus include, for example, amino acids or analogs of amino acids other than the 20 naturally occurring amino acids and include, but are not limited to, the D-isostereomers of amino acids. Exemplary non-natural amino acids are known to those of skill in the art, and include, but are not limited to, 2-Aminoadipic acid (Aad), 3-Aminoadipic acid (Baad), β-alanine/β-Amino-propionic acid (Bala), 2-Aminobutyric acid (Abu), 4-Aminobutyric acid/piperidinic acid (4Abu), 6-Aminocaproic acid (Acp), 2-Aminoheptanoic acid (Ahe), 2-Aminoisobutyric acid (Aib), 3-Aminoisobutyric acid (Baib), 2-Aminopimelic acid (Apm), 2,4-Diaminobutyric acid (Dbu), Desmosine (Des), 2,2′-Diaminopimelic acid (Dpm), 2,3-Diaminopropionic acid (Dpr), N-Ethylglycine (EtGly), N-Ethylasparagine (EtAsn), Hydroxylysine (Hyl), allo-Hydroxylysine (Ahyl), 3-Hydroxyproline (3Hyp), 4-Hydroxyproline (4Hyp), Isodesmosine (Ide), allo-Isoleucine (Aile), N-Methylglycine, sarcosine (MeGly), N-Methylisoleucine (MeIle), 6-N-Methyllysine (MeLys), N-Methylvaline (MeVal), Norvaline (Nva), Norleucine (Nle) and Ornithine (Orn).

As used herein, a “native polypeptide” or a “native nucleic acid” molecule is a polypeptide or nucleic acid molecule, respectively, that can be found in nature. A native polypeptide or nucleic acid molecule can be the wild-type form of a polypeptide or nucleic acid molecule. A native polypeptide or nucleic acid molecule can be the predominant form of the polypeptide, or any allelic or other natural variant thereof. The variant polypeptides and nucleic acid molecules provided herein can have modifications compared to native polypeptides and nucleic acid molecules.

As used herein, the “wild-type” form of a polypeptide or nucleic acid molecule is a form encoded by a gene or by a coding sequence encoded by the gene. Typically, a wild-type form of a gene, or molecule encoded thereby, does not contain mutations or other modifications that alter function or structure. The term wild-type also encompasses forms with allelic variation as occurs among and between species. As used herein, a predominant form of a polypeptide or nucleic acid molecule refers to a form of the molecule that is the major form produced from a gene. A “predominant form” varies from source to source. For example, different cells or tissue types can produce different forms of polypeptides, for example, by alternative splicing and/or by alternative protein processing. In each cell or tissue type, a different polypeptide can be a “predominant form.”

As used herein, an “allelic variant” or “allelic variation” references any of two or more alternative forms of a gene occupying the same chromosomal locus. Allelic variation arises naturally through mutation, and can result in phenotypic polymorphism within populations. Gene mutations can be silent (no change in the encoded polypeptide) or can encode polypeptides having altered amino acid sequence. The term “allelic variant” also is used herein to denote a protein encoded by an allelic variant of a gene. Typically the reference form of the gene encodes a wildtype form and/or predominant form of a polypeptide from a population or single reference member of a species. Typically, allelic variants, which include variants between and among species typically have at least 80%, 90% or greater amino acid identity with a wildtype and/or predominant form from the same species; the degree of identity depends upon the gene and whether comparison is interspecies or intraspecies. Generally, intraspecies allelic variants have at least about 80%, 85%, 90% or 95% identity or greater with a wildtype and/or predominant form, including 96%, 97%, 98%, 99% or greater identity with a wildtype and/or predominant form of a polypeptide. Reference to an allelic variant herein generally refers to variations n proteins among members of the same species.

As used herein, “allele,” which is used interchangeably herein with “allelic variant” refers to alternative forms of a gene or portions thereof. Alleles occupy the same locus or position on homologous chromosomes. When a subject has two identical alleles of a gene, the subject is said to be homozygous for that gene or allele. When a subject has two different alleles of a gene, the subject is said to be heterozygous for the gene. Alleles of a specific gene can differ from each other in a single nucleotide or several nucleotides, and can include substitutions, deletions and insertions of nucleotides. An allele of a gene also can be a form of a gene containing a mutation.

As used herein, “species variants” refer to variants in polypeptides among different species, including different mammalian species, such as mouse and human, and species of microorganisms, such as viruses and bacteria.

As used herein, a polypeptide “domain” is a part of a polypeptide (a sequence of three or more, generally 5 or 7 or more amino acids) that is a structurally and/or functionally distinguishable or definable. Exemplary of a polypeptide domain is a part of the polypeptide that can form an independently folded structure within a polypeptide made up of one or more structural motifs (e.g. combinations of alpha helices and/or beta strands connected by loop regions) and/or that is recognized by a particular functional activity, such as enzymatic activity or antigen binding. A polypeptide can have one or more, typically more than one, distinct domains. For example, the polypeptide can have one or more structural domains and one or more functional domains. A single polypeptide domain can be distinguished based on structure and function. A domain can encompass a contiguous linear sequence of amino acids. Alternatively, a domain can encompass a plurality of non-contiguous amino acid portions, which are non-contiguous along the linear sequence of amino acids of the polypeptide. Typically, a polypeptide contains a plurality of domains. For example, each heavy chain and each light chain of an antibody molecule contains a plurality of immunoglobulin (Ig) domains, each about 110 amino acids in length.

Those of skill in the art are familiar with polypeptide domains and can identify them by virtue of structural and/or functional homology with other such domains. For exemplification herein, definitions are provided, but it is understood that it is well within the skill in the art to recognize particular domains by name. If needed, appropriate software can be employed to identify domains.

As used herein, a “functional region” of a polypeptide is a region of the polypeptide that contains at least one functional domain (which imparts a particular function, such as an ability to interact with a biomolecule, for example, through antigen binding, DNA binding, ligand binding, or dimerization, or by enzymatic activity, for example, kinase activity or proteolytic activity); exemplary of functional regions of polypeptides are antibody domains, such as V_{H}, V_{L}, C_{H}, C_{L}, and portions thereof, such as CDRs, including CDR1, CDR2 and CDR3, and antigen binding portions, such as antibody combining sites.

As used herein, a “structural region” of a polypeptide is a region of the polypeptide that contains at least one structural domain.

As used herein, a “property” of a polypeptide, such as an antibody or other therapeutic polypeptide, refers to any property exhibited by a polypeptide, including, but not limited to, binding specificity, structural configuration or conformation, protein stability, resistance to proteolysis, conformational stability, thermal tolerance, and tolerance to pH conditions. Changes in properties can alter an “activity” of the polypeptide. For example, a change in the binding specificity of the antibody polypeptide can alter the ability to bind an antigen, and/or various binding activities, such as affinity or avidity, or in vivo activities of the therapeutic polypeptide.

As used herein, an “activity” or a “functional activity” of a polypeptide, such as an antibody or other therapeutic polypeptide, refers to any activity exhibited by the polypeptide. Such activities can be empirically determined. Exemplary activities include, but are not limited to, ability to interact with a biomolecule, for example, through antigen binding, DNA binding, ligand binding, or dimerization, enzymatic activity, for example, kinase activity or proteolytic activity. For an antibody (including antibody fragments), activities include, but are not limited to, the ability to specifically bind a particular antigen, affinity of antigen binding (e.g. high or low affinity), avidity of antigen binding (e.g. high or low avidity), on-rate, off-rate, effector functions, such as the ability to promote antigen neutralization or clearance, and in vivo activities, such as the ability to prevent infection or invasion of a pathogen, or to promote clearance, or to penetrate a particular tissue or fluid or cell in the body. Activity can be assessed in vitro or in vivo using recognized assays, such as ELISA, flow cytometry, Surface Plasmon Resonance (SPR), BIAcore or equivalent assays to measure on- or off-rate, immunohistochemistry and immunofluorescence histology and microscopy, cell-based assays, flow cytometry and binding assays (e.g. panning assays). For example, for an antibody polypeptide, activities can be assessed by measuring binding affinities, avidities, and/or binding coefficients (e.g. for on-/off-rates), and other activities in vitro or by measuring various effects in vivo, such as immune effects, e.g. antigen clearance, penetration or localization of the antibody into tissues, protection from disease, e.g. infection, serum or other fluid antibody titers, or other assays that are well known in the art. The results of such assays that indicate that a polypeptide exhibits an activity can be correlated to activity of the polypeptide in vivo, in which in vivo activity can be referred to as therapeutic activity, or biological activity. Activity of a modified polypeptide can be any level or percentage of activity of the unmodified polypeptide, including but not limited to, 1% of the activity, 2%, 3%, 4%, 5%, 10%, 20%, 30%, 40%, 50%, 60%, 70%, 80%, 90%, 91%, 92%, 93%, 94%, 95%, 96%, 97%, 98%, 99%, 100%, 200%, 300%, 400%, 500%, or more of activity compared to the unmodified polypeptide. Assays to determine functionality or activity of modified (e.g. variant) antibodies are well known in the art.

As used herein, “therapeutic activity” refers to the in vivo activity of a therapeutic polypeptide. Generally, the therapeutic activity is the activity that is used to treat a disease or condition. Therapeutic activity of a modified polypeptide can be any level or percentage of therapeutic activity of the unmodified polypeptide, including but not limited to, 1% of the activity, 2%, 3%, 4%, 5%, 10%, 20%, 30%, 40%, 50%, 60%, 70%, 80%, 90%, 91%, 92%, 93%, 94%, 95%, 96%, 97%, 98%, 99%, 100%, 200%, 300%, 400%, 500%, or more of therapeutic activity compared to the unmodified polypeptide.

As used herein, “exhibits at least one activity” or “retains at least one activity” refers to the activity exhibited by a modified polypeptide, such as a variant polypeptide produced according to the provided methods, such as a modified, e.g. variant antibody or other therapeutic polypeptide (e.g. a modified AC8 antibody), compared to the target or unmodified polypeptide, that does not contain the modification. A modified, or variant, polypeptide that retains an activity of a target polypeptide can exhibit improved activity or maintain the activity of the unmodified polypeptide. In some instances, a modified, or variant, polypeptide can retain an activity that is increased compared to an target or unmodified polypeptide. In some cases, a modified, or variant, polypeptide can retain an activity that is decreased compared to an unmodified or target polypeptide. Activity of a modified, or variant, polypeptide can be any level or percentage of activity of the unmodified or target polypeptide, including but not limited to, 1% of the activity, 2%, 3%, 4%, 5%, 10%, 20%, 30%, 40%, 50%, 60%, 70%, 80%, 90%, 91%, 92%, 93%, 94%, 95%, 96%, 97%, 98%, 99%, 100%, 200%, 300%, 400%, 500%, or more activity compared to the unmodified or target polypeptide. In other embodiments, the change in activity is at least about 2 times, 3 times, 4 times, 5 times, 6 times, 7 times, 8 times, 9 times, 10 times, 20 times, 30 times, 40 times, 50 times, 60 times, 70 times, 80 times, 90 times, 100 times, 200 times, 300 times, 400 times, 500 times, 600 times, 700 times, 800 times, 900 times, 1000 times, or more times greater than unmodified or target polypeptide. Assays for retention of an activity depend on the activity to be retained. Such assays can be performed in vitro or in vivo. Activity can be measured, for example, using assays known in the art and described in the Examples below for activities such as but not limited to ELISA and panning assays. Activities of a modified, or variant, polypeptide compared to an unmodified or target polypeptide also can be assessed in terms of an in vivo therapeutic or biological activity or result following administration of the polypeptide.

As used herein, the term “assessing” is intended to include quantitative and qualitative determination in the sense of obtaining an absolute value for the binding of an antibody or portion thereof with a target protein and/or modulation of an activity of a target protein by an antibody or portion thereof, and also of obtaining an index, ratio, percentage, visual or other value indicative of the level of the binding or activity. Assessment can be direct or indirect. For example, binding can be determined by directly labeling of an antibody or portion thereof with a detectable label and/or by using a secondary antibody that itself is labeled. In addition, functional activities can be determined using any of a variety of assays known to one of skill in the art.

As used herein, the term “nucleic acid” refers to at least two linked nucleotides or nucleotide derivatives, including a deoxyribonucleic acid (DNA) and a ribonucleic acid (RNA), joined together, typically by phosphodiester linkages. Also included in the term “nucleic acid” are analogs of nucleic acids such as peptide nucleic acid (PNA), phosphorothioate DNA, and other such analogs and derivatives or combinations thereof. Nucleic acids also include DNA and RNA derivatives containing, for example, a nucleotide analog or a “backbone” bond other than a phosphodiester bond, for example, a phosphotriester bond, a phosphoramidate bond, a phosphorothioate bond, a thioester bond, or a peptide bond (peptide nucleic acid). The term also includes, as equivalents, derivatives, variants and analogs of either RNA or DNA made from nucleotide analogs, single (sense or antisense) and double-stranded nucleic acids. Deoxyribonucleotides include deoxyadenosine, deoxycytidine, deoxyguanosine and deoxythymidine. For RNA, the uracil base is uridine.

Nucleic acids can contain nucleotide analogs, including, for example, mass modified nucleotides, which allow for mass differentiation of nucleic acid molecules; nucleotides containing a detectable label such as a fluorescent, radioactive, luminescent or chemiluminescent label, which allow for detection of a nucleic acid molecule; or nucleotides containing a reactive group such as biotin or a thiol group, which facilitates immobilization of a nucleic acid molecule to a solid support. A nucleic acid also can contain one or more backbone bonds that are selectively cleavable, for example, chemically, enzymatically or photolytically cleavable. For example, a nucleic acid can include one or more deoxyribonucleotides, followed by one or more ribonucleotides, which can be followed by one or more deoxyribonucleotides, such a sequence being cleavable at the ribonucleotide sequence by base hydrolysis. A nucleic acid also can contain one or more bonds that are relatively resistant to cleavage, for example, a chimeric oligonucleotide primer, which can include nucleotides linked by peptide nucleic acid bonds and at least one nucleotide at the 3′ end, which is linked by a phosphodiester bond or other suitable bond, and is capable of being extended by a polymerase. Peptide nucleic acid sequences can be prepared using well-known methods (see, for example, Weiler et al. (1997) Nucleic Acids Res. 25:2792-2799).

As used herein, the terms “polynucleotide” and “nucleic acid molecule” refer to an oligomer or polymer containing at least two linked nucleotides or nucleotide derivatives, including a deoxyribonucleic acid (DNA) and a ribonucleic acid (RNA), joined together, typically by phosphodiester linkages. Polynucleotides also include DNA and RNA derivatives containing, for example, a nucleotide analog or a “backbone” bond other than a phosphodiester bond, for example, a phosphotriester bond, a phosphoramidate bond, a phosphorothioate bond, a thioester bond, or a peptide bond (peptide nucleic acid). Polynucleotides (nucleic acid molecules), include single-stranded and/or double-stranded polynucleotides, such as deoxyribonucleic acid (DNA), and ribonucleic acid (RNA) as well as analogs or derivatives of either RNA or DNA. The term also includes, as equivalents, derivatives, variants and analogs of either RNA or DNA made from nucleotide analogs, single (sense or antisense) and double-stranded polynucleotides. Deoxyribonucleotides include deoxyadenosine, deoxycytidine, deoxyguanosine and deoxythymidine. For RNA, the uracil base is uridine. Polynucleotides can contain nucleotide analogs, including, for example, mass modified nucleotides, which allow for mass differentiation of polynucleotides; nucleotides containing a detectable label such as a fluorescent, radioactive, luminescent or chemiluminescent label, which allow for detection of a polynucleotide; or nucleotides containing a reactive group such as biotin or a thiol group, which facilitates immobilization of a polynucleotide to a solid support. A polynucleotide also can contain one or more backbone bonds that are selectively cleavable, for example, chemically, enzymatically or photolytically cleavable. For example, a polynucleotide can include one or more deoxyribonucleotides, followed by one or more ribonucleotides, which can be followed by one or more deoxyribonucleotides, such a sequence being cleavable at the ribonucleotide sequence by base hydrolysis. A polynucleotide also can contain one or more bonds that are relatively resistant to cleavage, for example, a chimeric oligonucleotide primer, which can include nucleotides linked by peptide nucleic acid bonds and at least one nucleotide at the 3′ end, which is linked by a phosphodiester bond or other suitable bond, and is capable of being extended by a polymerase. Peptide nucleic acid sequences can be prepared using well-known methods (see, for example, Weiler et al. (1997) Nucleic Acids Res. 25:2792-2799).

As used herein, a “DNA construct” is a single or double stranded, linear or circular DNA molecule that contains segments of DNA combined and juxtaposed in a manner not found in nature. DNA constructs exist as a result of human manipulation, and include clones and other copies of manipulated molecules.

As used herein, a “DNA segment” is a portion of a larger DNA molecule having specified attributes. For example, a DNA segment encoding a specified polypeptide is a portion of a longer DNA molecule, such as a plasmid or plasmid fragment, which, when read from the 5′ to 3′ direction, encodes the sequence of amino acids of the specified polypeptide.

As used herein, a “genetic element” refers to a gene, or any region thereof, that encodes a polypeptide or protein or region thereof. In some examples, a genetic element encodes a fusion protein.

As used herein, regulatory region of a nucleic acid molecule means a cis-acting nucleotide sequence that influences expression, positively or negatively, of an operatively linked gene. Regulatory regions include sequences of nucleotides that confer inducible (i.e., require a substance or stimulus for increased transcription) expression of a gene. When an inducer is present or at increased concentration, gene expression can be increased. Regulatory regions also include sequences that confer repression of gene expression (i.e., a substance or stimulus decreases transcription). When a repressor is present or at increased concentration gene expression can be decreased. Regulatory regions are known to influence, modulate or control many in vivo biological activities including cell proliferation, cell growth and death, cell differentiation and immune modulation. Regulatory regions typically bind to one or more trans-acting proteins, which results in either increased or decreased transcription of the gene.

Particular examples of gene regulatory regions are promoters and enhancers. Promoters are sequences located around the transcription or translation start site, typically positioned 5′ of the translation start site. Promoters usually are located within 1 Kb of the translation start site, but can be located further away, for example, 2 Kb, 3 Kb, 4 Kb, 5 Kb or more, up to and including 10 Kb. Enhancers are known to influence gene expression when positioned 5′ or 3′ of the gene, or when positioned in or a part of an exon or an intron. Enhancers also can function at a significant distance from the gene, for example, at a distance from about 3 Kb, 5 Kb, 7 Kb, 10 Kb, 15 Kb or more.

Regulatory regions also include, in addition to promoter regions, sequences that facilitate translation, splicing signals for introns, maintenance of the correct reading frame of the gene to permit in-frame translation of mRNA and, stop codons, leader sequences and fusion partner sequences, internal ribosome binding site (IRES) elements for the creation of multigene, or polycistronic, messages, polyadenylation signals to provide proper polyadenylation of the transcript of a gene of interest and stop codons, and can be optionally included in an expression vector.

As used herein, the term promoter means a portion of a gene containing DNA sequences that provide for the binding of RNA polymerase and initiation of transcription. Promoter sequences are commonly, but not always, found in the 5′ non-coding region of genes.

As used herein, operably or operatively linked when referring to DNA segments means that the segments are arranged so that they function in concert for their intended purposes, e.g., transcription initiates in the promoter and proceeds through the coding segment to the terminator.

As used herein, “synthetic,” with reference to, for example, a synthetic nucleic acid molecule or a synthetic gene or a synthetic peptide refers to a nucleic acid molecule or polypeptide molecule that is produced by recombinant methods and/or by chemical synthesis methods.

As used herein, production by recombinant means by using recombinant DNA methods means the use of the well known methods of molecular biology for expressing proteins encoded by cloned DNA.

As used herein, “expression” refers to the process by which polypeptides are produced by transcription and translation of polynucleotides. The level of expression of a polypeptide can be assessed using any method known in art, including, for example, methods of determining the amount of the polypeptide produced from the host cell. Such methods can include, but are not limited to, quantitation of the polypeptide in the cell lysate by ELISA, Coomassie blue staining following gel electrophoresis, Lowry protein assay and Bradford protein assay.

As used herein, a “host cell” is a cell that is used in to receive, maintain, reproduce and amplify a vector. A host cell also can be used to express the polypeptide encoded by the vector. The nucleic acid contained in the vector is replicated when the host cell divides, thereby amplifying the nucleic acids. In one example, the host cell is a genetic package, which can be induced to express the variant polypeptide on its surface. In another example, the host cell is infected with the genetic package. For example, the host cells can be phage-display compatible host cells, which can be transformed with phage or phagemid vectors and accommodate the packaging of phage expressing fusion proteins containing the variant polypeptides.

As used herein, a “vector” is a replicable nucleic acid from which one or more heterologous proteins can be expressed when the vector is transformed into an appropriate host cell. Reference to a vector includes those vectors into which a nucleic acid encoding a polypeptide or fragment thereof can be introduced, typically by restriction digest and ligation. Reference to a vector also includes those vectors that contain nucleic acid encoding a polypeptide. The vector is used to introduce the nucleic acid encoding the polypeptide into the host cell for amplification of the nucleic acid or for expression/display of the polypeptide encoded by the nucleic acid. The vectors typically remain episomal, but can be designed to effect integration of a gene or portion thereof into a chromosome of the genome. Also contemplated are vectors that are artificial chromosomes, such as yeast artificial chromosomes and mammalian artificial chromosomes. Selection and use of such vehicles are well known to those of skill in the art.

As used herein, a vector also includes “virus vectors” or “viral vectors.” Viral vectors are engineered viruses that are operatively linked to exogenous genes to transfer (as vehicles or shuttles) the exogenous genes into cells.

As used herein, an “expression vector” includes vectors capable of expressing DNA that is operatively linked with regulatory sequences, such as promoter regions, that are capable of effecting expression of such DNA fragments. Such additional segments can include promoter and terminator sequences, and optionally can include one or more origins of replication, one or more selectable markers, an enhancer, a polyadenylation signal, and the like. Expression vectors are generally derived from plasmid or viral DNA, or can contain elements of both. Thus, an expression vector refers to a recombinant DNA or RNA construct, such as a plasmid, a phage, recombinant virus or other vector that, upon introduction into an appropriate host cell, results in expression of the cloned DNA. Appropriate expression vectors are well known to those of skill in the art and include those that are replicable in eukaryotic cells and/or prokaryotic cells and those that remain episomal or those which integrate into the host cell genome.

As used herein, the terms “oligonucleotide” and “oligo” are used synonymously. Oligonucleotides are polynucleotides that contain a limited number of nucleotides in length. Those in the art recognize that oligonucleotides generally are less than at or about two hundred fifty, typically less than at or about two hundred, typically less than at or about one hundred, nucleotides in length. Typically, the oligonucleotides provided herein are synthetic oligonucleotides. The synthetic oligonucleotides contain fewer than at or about 250 or 200 nucleotides in length, for example, fewer than about 20, 30, 40, 50, 60, 70, 80, 90, 100, 110, 120, 130, 140, 150, 160, 170, 180, 190 or 200 nucleotides in length. Typically, the oligonucleotides are single-stranded oligonucleotides. The ending “mer” can be used to denote the length of an oligonucleotide. For example, “100-mer” can be used to refer to an oligonucleotide containing 100 nucleotides in length.

As used herein, “primer” refers to a nucleic acid molecule that can act as a point of initiation of template-directed nucleic acid synthesis under appropriate conditions (for example, in the presence of four different nucleoside triphosphates and a polymerization agent, such as DNA polymerase, RNA polymerase or reverse transcriptase) in an appropriate buffer and at a suitable temperature. It will be appreciated that certain nucleic acid molecules can serve as a “probe” and as a “primer.” A primer, however, has a 3′ hydroxyl group for extension. A primer can be used in a variety of methods, including, for example, polymerase chain reaction (PCR), reverse-transcriptase (RT)-PCR, RNA PCR, LCR, multiplex PCR, panhandle PCR, capture PCR, expression PCR, 3′ and 5′ RACE, in situ PCR, ligation-mediated PCR and other amplification protocols.

As used herein, “primer pair” refers to a set of primers that includes a 5′ (upstream) primer that hybridizes with the 5′ end of a sequence to be amplified (e.g. by PCR) and a 3′ (downstream) primer that hybridizes with the complement of the 3′ end of the sequence to be amplified.

As used herein, “specifically hybridizes” refers to annealing, by complementary base-pairing, of a nucleic acid molecule (e.g. an oligonucleotide) to a target nucleic acid molecule. Those of skill in the art are familiar with in vitro and in vivo parameters that affect specific hybridization, such as length and composition of the particular molecule. Parameters particularly relevant to in vitro hybridization further include annealing and washing temperature, buffer composition and salt concentration. Exemplary washing conditions for removing non-specifically bound nucleic acid molecules at high stringency are 0.1×SSPE, 0.1% SDS, 65° C., and at medium stringency are 0.2×SSPE, 0.1% SDS, 50° C. Equivalent stringency conditions are known in the art. The skilled person can readily adjust these parameters to achieve specific hybridization of a nucleic acid molecule to a target nucleic acid molecule appropriate for a particular application.

As used herein, “primary sequence” refers to the sequence of amino acid residues in a polypeptide or the sequence of nucleotides in a nucleic acid molecule.

As used herein, “similarity” between two proteins or nucleic acids refers to the relatedness between the sequence of amino acids of the proteins or the nucleotide sequences of the nucleic acids. Similarity can be based on the degree of identity of sequences of residues and the residues contained therein. Methods for assessing the degree of similarity between proteins or nucleic acids are known to those of skill in the art. For example, in one method of assessing sequence similarity, two amino acid or nucleotide sequences are aligned in a manner that yields a maximal level of identity between the sequences. “Identity” refers to the extent to which the amino acid or nucleotide sequences are invariant. Alignment of amino acid sequences, and to some extent nucleotide sequences, also can take into account conservative differences and/or frequent substitutions in amino acids (or nucleotides). Conservative differences are those that preserve the physico-chemical properties of the residues involved. Alignments can be global (alignment of the compared sequences over the entire length of the sequences and including all residues) or local (the alignment of a portion of the sequences that includes only the most similar region or regions).

As used herein, when a polypeptide or nucleic acid molecule or region thereof contains or has “identity” or “homology” to another polypeptide or nucleic acid molecule or region, the two molecules and/or regions share greater than or equal to at or about 40% sequence identity, and typically greater than or equal to at or about 50 sequence identity, such as at least at or about 60%, 65%, 70%, 75%, 80%, 85%, 90%, 95%, 96%, 97%, 98%, 99% or 100% sequence identity; the precise percentage of identity can be specified if necessary. A nucleic acid molecule, or region thereof, that is identical or homologous to a second nucleic acid molecule or region can specifically hybridize to a nucleic acid molecule or region that is 100% complementary to the second nucleic acid molecule or region. Identity alternatively can be compared between two theoretical nucleotide or amino acid sequences or between a nucleic acid or polypeptide molecule and a theoretical sequence.

Sequence “identity,” per se, has an art-recognized meaning and the percentage of sequence identity between two nucleic acid or polypeptide molecules or regions can be calculated using published techniques. Sequence identity can be measured along the full length of a polynucleotide or polypeptide or along a region of the molecule. (See, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). While there exist a number of methods to measure identity between two polynucleotide or polypeptides, the term “identity” is well known to skilled artisans (Carrillo, H. & Lipman, D., SIAM J Applied Math 48:1073 (1988)).

Sequence identity compared along the full length of two polynucleotides or polypeptides refers to the percentage of identical nucleotide or amino acid residues along the full-length of the molecule. For example, if a polypeptide A has 100 amino acids and polypeptide B has 95 amino acids, which are identical to amino acids 1-95 of polypeptide A, then polypeptide B has 95% identity when sequence identity is compared along the full length of a polypeptide A compared to full length of polypeptide B. Alternatively, sequence identity between polypeptide A and polypeptide B can be compared along a region, such as a 20 amino acid analogous region, of each polypeptide. In this case, if polypeptide A and B have 20 identical amino acids along that region, the sequence identity for the regions is 100%. Alternatively, sequence identity can be compared along the length of a molecule, compared to a region of another molecule. As discussed below, and known to those of skill in the art, various programs and methods for assessing identity are known to those of skill in the art. High levels of identity, such as 90% or 95% identity, readily can be determined without software.

Whether any two nucleic acid molecules have nucleotide sequences that are at least 60%, 70%, 80%, 85%, 90%, 95%, 96%, 97%, 98% or 99% “identical” can be determined using known computer algorithms such as the “FASTA” program, using for example, the default parameters as in Pearson et al. (1988) Proc. Natl. Acad. Sci. USA 85:2444 (other programs include the GCG program package (Devereux, J. et al. (1984) Nucleic Acids Research 12(I):387), BLASTP, BLASTN, FASTA (Altschul, S. F. et al. (1990) J. Molec. Biol. 215:403; Guide to Huge Computers, Martin J. Bishop, ed., Academic Press, San Diego, 1994, and Carrillo et al. (1988) SIAM J Applied Math 48:1073). For example, the BLAST function of the National Center for Biotechnology Information database can be used to determine identity. Other commercially or publicly available programs include, DNAStar “MegAlign” program (Madison, Wis.) and the University of Wisconsin Genetics Computer Group (UWG) “Gap” program (Madison Wis.)). Percent homology or identity of proteins and/or nucleic acid molecules can be determined, for example, by comparing sequence information using a GAP computer program (e.g., Needleman et al. (1970) J. Mol. Biol. 48:443, as revised by Smith and Waterman ((1981) Adv. Appl. Math. 2:482). Briefly, the GAP program defines similarity as the number of aligned symbols (i.e., nucleotides or amino acids), which are similar, divided by the total number of symbols in the shorter of the two sequences. Default parameters for the GAP program can include: (1) a unary comparison matrix (containing a value of 1 for identities and 0 for non-identities) and the weighted comparison matrix of Gribskov et al. (1986) Nucl. Acids Res. 14:6745, as described by Schwartz and Dayhoff, eds., ATLAS OF PROTEIN SEQUENCE AND STRUCTURE, National Biomedical Research Foundation, pp. 353-358 (1979); (2) a penalty of 3.0 for each gap and an additional 0.10 penalty for each symbol in each gap; and (3) no penalty for end gaps.

In general, for determination of the percentage sequence identity, sequences are aligned so that the highest order match is obtained (see, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; Carrillo et al. (1988) SIAM J Applied Math 48:1073). For sequence identity, the number of conserved amino acids is determined by standard alignment algorithms programs, and can be used with default gap penalties established by each supplier. Substantially homologous nucleic acid molecules specifically hybridize typically at moderate stringency or at high stringency all along the length of the nucleic acid of interest. Also contemplated are nucleic acid molecules that contain degenerate codons in place of codons in the hybridizing nucleic acid molecule.

Therefore, the term “identity,” when associated with a particular number, represents a comparison between the sequences of a first and a second polypeptide or polynucleotide or regions thereof and/or between theoretical nucleotide or amino acid sequences. As used herein, the term at least “90% identical to” refers to percent identities from 90 to 99.99 relative to the first nucleic acid or amino acid sequence of the polypeptide. Identity at a level of 90% or more is indicative of the fact that, assuming for exemplification purposes, a first and second polypeptide length of 100 amino acids are compared, no more than 10% (i.e., 10 out of 100) of the amino acids in the first polypeptide differs from that of the second polypeptide. Similar comparisons can be made between first and second polynucleotides. Such differences among the first and second sequences can be represented as point mutations randomly distributed over the entire length of a polypeptide or they can be clustered in one or more locations of varying length up to the maximum allowable, e.g. 10/100 amino acid difference (approximately 90% identity). Differences are defined as nucleotide or amino acid residue substitutions, insertions, additions or deletions. At the level of homologies or identities above about 85-90%, the result is independent of the program and gap parameters set; such high levels of identity can be assessed readily, often by manual alignment without relying on software.

As used herein, “alignment” of a sequence refers to the use of homology to align two or more sequences of nucleotides or amino acids. Typically, two or more sequences that are related by 50% or more identity are aligned. An aligned set of sequences refers to 2 or more sequences that are aligned at corresponding positions and can include aligning sequences derived from RNAs, such as ESTs and other cDNAs, aligned with genomic DNA sequence.

Related or variant polypeptides or nucleic acid molecules can be aligned by any method known to those of skill in the art. Such methods typically maximize matches, and include methods, such as using manual alignments and by using the numerous alignment programs available (e.g., BLASTP) and others known to those of skill in the art. By aligning the sequences of polypeptides or nucleic acids, one skilled in the art can identify analogous portions or positions, using conserved and identical amino acid residues as guides. Further, one skilled in the art also can employ conserved amino acid or nucleotide residues as guides to find corresponding amino acid or nucleotide residues between and among human and non-human sequences. Corresponding positions also can be based on structural alignments, for example by using computer simulated alignments of protein structure. In other instances, corresponding regions can be identified. One skilled in the art also can employ conserved amino acid residues as guides to find corresponding amino acid residues between and among human and non-human sequences.

As used herein, “analogous” and “corresponding” portions, positions or regions are portions, positions or regions that are aligned with one another upon aligning two or more related polypeptide or nucleic acid sequences (including sequences of molecules, regions of molecules and/or theoretical sequences) so that the highest order match is obtained, using an alignment method known to those of skill in the art to maximize matches. In other words, two analogous positions (or portions or regions) align upon best-fit alignment of two or more polypeptide or nucleic acid sequences. The analogous portions/positions/regions are identified based on position along the linear nucleic acid or amino acid sequence when the two or more sequences are aligned. The analogous portions need not share any sequence similarity with one another. For example, alignment (such that maximizing matches) of the sequences of two homologous nucleic acid molecules, each 100 nucleotides in length, can reveal that 70 of the 100 nucleotides are identical. Portions of these nucleic acid molecules containing some or all of the other non-identical 30 amino acids are analogous portions that do not share sequence identity. Alternatively, the analogous portions can contain some percentage of sequence identity to one another, such as at or about 50%, 55%, 60%, 65%, 70%, 75%, 80%, 85%, 90%, 95%, 96%, 97%, 98%, 99%, or fractions thereof. In one example, the analogous portions are 100% identical.

As used herein, a “modification” is in reference to modification of a sequence of amino acids of a polypeptide or a sequence of nucleotides in a nucleic acid molecule and includes deletions, insertions, and replacements of amino acids and nucleotides, respectively. Methods of modifying a polypeptide are routine to those of skill in the art, such as by using recombinant DNA methodologies.

As used herein, “deletion,” when referring to a nucleic acid or polypeptide sequence, refers to the deletion of one or more nucleotides or amino acids compared to a sequence, such as a target polynucleotide or polypeptide or a native or wild-type sequence.

As used herein, “insertion” when referring to a nucleic acid or amino acid sequence, describes the inclusion of one or more additional nucleotides or amino acids, within a target, native, wild-type or other related sequence. Thus, a nucleic acid molecule that contains one or more insertions compared to a wild-type sequence, contains one or more additional nucleotides within the linear length of the sequence. As used herein, “additions,” to nucleic acid and amino acid sequences describe addition of nucleotides or amino acids onto either termini compared to another sequence.

As used herein, “substitution” refers to the replacing of one or more nucleotides or amino acids in a native, target, wild-type or other nucleic acid or polypeptide sequence with an alternative nucleotide or amino acid, without changing the length (as described in numbers of residues) of the molecule. Thus, one or more substitutions in a molecule does not change the number of amino acid residues or nucleotides of the molecule. Substitution mutations compared to a particular polypeptide can be expressed in terms of the number of the amino acid residue along the length of the polypeptide sequence. For example, a modified polypeptide having a modification in the amino acid at the 19^{th} position of the amino acid sequence that is a substitution of Isoleucine (Ile; I) for cysteine (Cys; C) can be expressed as I19C, Ile19C, or simply C19, to indicate that the amino acid at the modified 19^{th} position is a cysteine. In this example, the molecule having the substitution has a modification at Ile 19 of the unmodified polypeptide.

As used herein, “specifically bind” or “immunospecifically bind” with respect to an antibody refers to the ability of the antibody to form one or more noncovalent bonds with a cognate antigen, by noncovalent interactions between the antibody combining site(s) of the antibody and the antigen.

As used herein, “binding partner” refers to a molecule (such as a polypeptide, lipid, glycolipid, nucleic acid molecule, carbohydrate or other molecule), with which another molecule specifically interacts, for example, through covalent or noncovalent interactions, such as the interaction of an antibody with cognate antigen. The binding partner can be naturally or synthetically produced. In one example, desired variant polypeptides are selected using one or more binding partners, for example, using in vitro or in vivo methods. Exemplary of the in vitro methods include selection using a binding partner coupled to a solid support, such as a bead, plate, column, matrix or other solid support; or a binding partner coupled to another selectable molecule, such as a biotin molecule, followed by subsequent selection by coupling the other selectable molecule to a solid support. Typically, the in vitro methods include wash steps to remove unbound polypeptides, followed by elution of the selected variant polypeptide(s). The process can be repeated one or more times in an iterative process to select variant polypeptides from among the selected polypeptides.

As used herein, a “binding property” is a characteristic of a molecule, e.g. a polypeptide, relating to whether or not, and how, it binds one or more binding partners. Binding properties include ability to bind the binding partner(s), the affinity with which it binds to the binding partner (e.g. high affinity), the avidity with which it binds to the binding partner, the strength of the bond with the binding partner and specificity for binding with the binding partner.

As used herein, affinity describes the strength of the interaction between two or more molecules, such as binding partners, typically the strength of the noncovalent interactions between two binding partners. The affinity of an antibody for an antigen epitope is the measure of the strength of the total noncovalent interactions between a single antibody combining site and the epitope. Low-affinity antibody-antigen interaction is weak, and the molecules tend to dissociate rapidly, while high affinity antibody-antigen binding is strong and the molecules remain bound for a longer amount of time. Methods for calculating affinity are well known, such as methods for determining dissociation constants. Affinity can be estimated empirically or affinities can be determined comparatively, e.g. by comparing the affinity of one antibody and another antibody for a particular antigen.

As used herein, “antibody avidity” refers to the strength of multiple interactions between a multivalent antibody and its cognate antigen, such as with antibodies containing multiple binding sites associated with an antigen with repeating epitopes or an epitope array. A high avidity antibody has a higher strength of such interactions compared with a low avidity antibody.

As used herein, “bind” refers to the participation of a molecule in any attractive interaction with another molecule, resulting in a stable association in which the two molecules are in close proximity to one another. Binding includes, but is not limited to, non-covalent bonds, covalent bonds (such as reversible and irreversible covalent bonds), and includes interactions between molecules such as, but not limited to, proteins, nucleic acids, carbohydrates, lipids, and small molecules, such as chemical compounds including drugs. Exemplary of bonds are antibody-antigen interactions and receptor-ligand interactions. When an antibody “binds” a particular antigen, bind refers to the specific recognition of the antigen by the antibody, through cognate antibody-antigen interaction, at antibody combining sites. Binding also can include association of multiple chains of a polypeptide, such as antibody chains which interact through disulfide bonds.

As used herein, a disulfide bond (also called an S—S bond or a disulfide bridge) is a single covalent bond derived from the coupling of thiol groups. Disulfide bonds in proteins are formed between the thiol groups of cysteine residues, and stabilize interactions between polypeptide domains, such as antibody domains.

As used herein, “coupled” or “conjugated” means attached via a covalent or noncovalent interaction.

As used herein, the phrase “conjugated to an antibody” when referring to the attachment of a protein transduction domain (PTD) to an antibody means that the PTD is attached to the antibody by any known means for linking peptides, such as, for example, by production of fusion protein by recombinant means or post-translationally by chemical means. Conjugation can employ any of a variety of linking agents to effect conjugation, including, but not limited to, peptide or compound linkers or chemical cross-linking agents.

As used herein, “phage display” refers to the expression of polypeptides on the surface of filamentous bacteriophage.

As used herein, a “phage-display compatible cell” or “phage-display compatible host cell” is a host cell, typically a bacterial host cell, that can be infected by phage and thus can support the production of phage displaying fusion proteins containing polypeptides, e.g. variant polypeptides and can thus be used for phage display. Exemplary of phage display compatible cells include, but are not limited to, XL1-blue cells.

As used herein, “panning” refers to an affinity-based selection procedure for the isolation of phage displaying a molecule with a specificity for a binding partner, for example, a capture molecule (e.g. an antigen) or sequence of amino acids or nucleotides or epitope, region, portion or locus therein.

As used herein, “disease” or “disorder” refers to a pathological condition in an organism resulting from cause or condition including, but not limited to, infections, acquired conditions, genetic conditions, and characterized by identifiable symptoms. Diseases and disorders of interest herein are those involving viral infection.

The term “HSV disease” means any disease caused, directly or indirectly, by Herpes Simplex Virus (HSV) as well as diseases which predispose a patient to infection by HSV. Examples of diseases falling into the former category include genital, oral and ocular herpes. Diseases in the latter category (i.e., those which place the patient at risk of severe HSV infection) include, generally, any condition that causes a state of immunosuppression or decreased function of the immune system such as patients who receive organ transplants, AIDS patients, those with hematological or lymphoreticular neoplasms, and infants.

As used herein, “treating” a subject with a disease or condition means that the subject's symptoms are partially or totally alleviated, or remain static following treatment. Hence treatment encompasses prophylaxis, therapy and/or cure. Prophylaxis refers to prevention of a potential disease and/or a prevention of worsening of symptoms or progression of a disease. Treatment also encompasses any pharmaceutical use of a modified therapeutic antibody and compositions provided herein.

As used herein, “prevention” or prophylaxis refers to methods in which the risk of developing disease or condition is reduced.

As used herein, a “pharmaceutically effective agent” includes any therapeutic agent or bioactive agents, including, but not limited to, for example, anesthetics, vasoconstrictors, dispersing agents, conventional therapeutic drugs, including small molecule drugs and therapeutic proteins, such as antibodies.

As used herein, a “therapeutic effect” means an effect resulting from treatment of a subject that alters, typically improves or ameliorates the symptoms of a disease or condition or that cures a disease or condition. Exemplary therapeutic effects include, but are not limited to, decreased viral replication, decreased viral cell-to-cell spread, decreased viralinfection and decreased viral load. A therapeutic effect can be determined by any method known to one of skill in the art, for example, by in vitro or in vivo assays as described in detail elsewhere herein.

As used herein, a “therapeutically effective amount” or a “therapeutically effective dose” refers to the quantity of an agent, compound, material, or composition containing a compound that is at least sufficient to produce a therapeutic effect following administration to a subject. Hence, it is the quantity necessary for preventing, curing, ameliorating, arresting or partially arresting a symptom of a disease or disorder.

As used herein, “therapeutic efficacy” refers to the ability of an agent, compound, material, or composition containing a compound to produce a therapeutic effect in a subject to whom the an agent, compound, material, or composition containing a compound has been administered.

As used herein, increasing the therapeutic efficacy of an antibody or an antigen binding fragment thereof by conjugation to a protein transduction domain means adding a new therapeutic effect and/or improving an existing therapeutic effect compared the antibody that is not conjugated to a PTD. For example, a neutralizing antiviral antibody that is conjugated to a protein transduction domain can prevent viral cell-to-cell spread, compared to a neutralizing antiviral antibody that is not conjugated to a PDT and that does not prevent viral cell-to-cell spread.

As used herein, prevention or prophylaxis refers to methods in which the risk of developing a disease or condition is reduced.

As used herein, the terms “immunotherapeutically” or “immunotherapy” in conjunction with modified antibodies provided denotes both prophylactic as well as therapeutic administration. Thus, the therapeutic antibodies provided can be administered to a subject at risk of contracting a virus infection in order to lessen the likelihood and/or severity of the disease, or administered to subjects already evidencing active virus infection. For example, the therapeutic antibodies provided can be administered to high-risk subjects, such as AIDS patients, in order to lessen the likelihood and/or severity of HSV disease, or administered to subjects already evidencing active HSV infection.

As used herein, “passive immunization” refers to therapeutic treatment of a subject or patient using immunological agents, such as antibodies (e.g., monoclonal antibodies) produced outside a subject or patient, without the purpose of inducing the subject or patient's immune system to produce a specific immune response to the therapeutic agent.

As used herein, “amelioration” of the symptoms of a particular disease or disorder by a treatment, such as by administration of a pharmaceutical composition or other therapeutic, refers to any lessening, whether permanent or temporary, lasting or transient, of the symptoms that can be attributed to or associated with administration of the composition or therapeutic.

As used herein, “mucosal delivery” refers to administration of a therapeutic agents, such as a modified therapeutic antibody provided herein, to a mucous membrane, characterized by a tissue lining of mostly endodermal origin, covered in epithelium, which is involved in absorption and secretion. Exemplary mucosal surfaces include, for example, oral, buccal, gingival, esophageal, bronchial, pulmonary, gastric, intestinal, nasal, rectal, conjunctival, sublingual, urethral, ureteral, uterine, vaginal, uterine, glans penis, glans clitoridis and cystic (bladder) mucosa.

As used herein, the term “diagnostically effective” means that the amount of a detectably labeled modified antibody is administered to a subject in a sufficient amount to enable detection of the site having the HSV antigen for which the antibody is specific. In using the modified antibodies provided herein for the in vivo detection of antigen, the detectably labeled antibody is given in a dose which is diagnostically effective.

As used herein, the term “subject” refers to an animal, including a mammal, such as a human being.

As used herein, a “patient” refers to a human subject.

As used herein, “animal” includes any animal, such as, but are not limited to primates including humans, gorillas and monkeys; rodents, such as mice and rats; fowl, such as chickens; ruminants, such as goats, cows, deer, sheep; pigs and other animals. Non-human animals exclude humans as the contemplated animal. The polypeptides provided herein are from any source, animal, plant, prokaryotic and fungal. Most polypeptides are of animal origin, including mammalian origin.

As used herein, a “unit dose form” refers to physically discrete units suitable for human and animal subjects and packaged individually as is known in the art.

As used herein, a “single dosage formulation” refers to a formulation for direct administration.

As used herein, an “article of manufacture” is a product that is made and sold. As used throughout this application, the term is intended to encompass any of the compositions provided herein contained in articles of packaging.

As used herein, a “fluid” refers to any composition that can flow. Fluids thus encompass compositions that are in the form of semi-solids, pastes, solutions, aqueous mixtures, gels, lotions, creams and other such compositions.

As used herein, an “isolated” or “purified” polypeptide or protein or biologically-active portion thereof is substantially free of cellular material or other contaminating proteins from the cell or tissue from which the protein is derived, or substantially free from chemical precursors or other chemicals when chemically synthesized. Preparations can be determined to be substantially free if they appear free of readily detectable impurities as determined by standard methods of analysis, such as thin layer chromatography (TLC), gel electrophoresis and high performance liquid chromatography (HPLC), used by those of skill in the art to assess such purity, or sufficiently pure such that further purification does not detectably alter the physical and chemical properties, such as enzymatic and biological activities, of the substance. Methods for purification of the compounds to produce substantially chemically pure compounds are known to those of skill in the art. A substantially chemically pure compound, however, can be a mixture of stereoisomers. In such instances, further purification might increase the specific activity of the compound.

As used herein, a “cellular extract” or “lysate” refers to a preparation or fraction which is made from a lysed or disrupted cell.

As used herein, a “control” refers to a sample that is substantially identical to the test sample, except that it is not treated with a test parameter, or, if it is a plasma sample, it can be from a normal volunteer not affected with the condition of interest. A control also can be an internal control.

As used herein, a “composition” refers to any mixture. It can be a solution, suspension, liquid, powder, paste, aqueous, non-aqueous or any combination thereof.

As used herein, a “combination” refers to any association between or among two or more items. The combination can be two or more separate items, such as two compositions or two collections, can be a mixture thereof, such as a single mixture of the two or more items, or any variation thereof. The elements of a combination are generally functionally associated or related.

As used herein, a “kit” is a packaged combination that optionally includes other elements, such as additional reagents and instructions for use of the combination or elements thereof, for a purpose including, but not limited to, activation, administration, diagnosis, and assessment of a biological activity or property.

As used herein, the singular forms “a,” “an” and “the” include plural referents unless the context clearly dictates otherwise. Thus, for example, reference to a polypeptide, comprising “an immunoglobulin domain” includes polypeptides with one or a plurality of immunoglobulin domains.

As used herein, the term “or” is used to mean “and/or” unless explicitly indicated to refer to alternatives only or the alternatives are mutually exclusive.

As used herein, ranges and amounts can be expressed as “about” a particular value or range. About also includes the exact amount. Hence “about 5 amino acids” means “about 5 amino acids” and also “5 amino acids.”

As used herein, “optional” or “optionally” means that the subsequently described event or circumstance does or does not occur and that the description includes instances where said event or circumstance occurs and instances where it does not. For example, an optionally variant portion means that the portion is variant or non-variant.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9):1726-1732).

### B. MODIFIED THERAPEUTIC ANTIBODIES

Provided herein are modified therapeutic antibodies that exhibit improved properties for the prevention and treatment of viral- or other pathogen-mediated diseases compared to the unmodified therapeutic antibody. Such properties include, for example, increased retention of the antibody in the subject, particularly at therapeutic sites, and increased therapeutic efficacy for preventing and/or eliminating infection by a virus or other pathogen.

The modified therapeutic antibodies provided contain a protein transduction domain (PTD) conjugated to an antibody molecule, latter of which can bind to and neutralize viruses or other pathogenic microorganisms. The modified therapeutic antibodies provided herein exhibit increased retention in the mucosa and other target sites for therapy in a subject compared to unmodified therapeutic antibodies that lack the protein transduction domain.

Protein transduction domains (PTDs) previously have been employed to promote the cellular uptake of antibodies, which are immunospecific for intracellular targets, such as oncoproteins, transcription factors and other regulatory proteins (see, e.g., EP 1867661, WO 98/42876). In some cases, PTDs have been conjugated to antibodies that bind to viral regulatory proteins that are released into the cytoplasm of a cell following infection or produced by the virus during viral infection (see, e.g., WO 03/035892). In order to exert a therapeutic effect, such antibodies must translocate across the cell membrane of a target cell to reach their specific intracellular antigens. In contrast, the modified therapeutic antibodies provided herein are derived from neutralizing antibodies that bind to the surfaces of particular viruses or pathogens. Hence, the modified antibodies provided herein are not limited to intracellular interaction with a target antigen. Instead, the modified antibodies provided herein can neutralize a virus or pathogen at multiple sites, both intracellularly and extracellularly. For example, the modified therapeutic antibodies provided can neutralize a virus or pathogen (1) prior to contact of the virus or pathogen with a target cell, such as in the mucosa, blood or lymphatic system; (2) at the target cell surface, through binding of the PTD portion of the modified antibody to the target cell surface and accumulation of the modified antibody at the cell surface resulting in prevention of attachment, fusion or entry of the virus or pathogen into the target cell; and (3) intracellularly, by uptake of the modified antibody into the target cell for prevention of assembly and/or release of newly synthesized virus particles or pathogens.

Modified therapeutic antibodies provided herein can inhibit or prevent one or more events that occur during viral infection of a target cell. For example, the modified therapeutic antibody can inhibit or reduce the attachment of a virus to a target cell surface, the fusion of the viral envelope with a target cell membrane and/or release of the virus contents into the cell. The modified therapeutic antibodies provided herein also can neutralize a virus in the absence of a target cell. In some examples, the modified therapeutic antibody also is internalized into the cell and can inhibit the formation of new viral particles in a virus infected cell and/or inhibit the release of viral particles from the virus infected cell.

The modified antibodies provided herein can be employed, for example, for passive immunization of a subject against a particular virus or for treatment of a subject with a viral infection. In particular examples, the modified therapeutic antibodies provided can be employed for passive immunization against a herpes virus or for treatment of a subject with a herpes virus infection.

Attachment of a protein transduction domain to an antibody molecule promotes the retention of the antibody at a target site(s) in a subject following administration. In particular examples, the conjugation of the antibody molecule to a transduction domain promotes the retention of the antibody at a mucosal surface, such as, for example, ocular, nasal/respiratory, oral or digestive surfaces.

Generally, the modified therapeutic antibodies provided herein are neutralizing antibodies that are conjugated to a protein transduction domain, where the antigen recognition domain of the neutralizing antibody portion of the molecule can immunospecifically bind to an epitope on the surface of a virus or pathogen. In some examples, the neutralizing antibody portion binds to membrane protein of the viral envelope or pathogen or a capsid protein of the viral particle. In some examples, the envelope protein is a glycoprotein.

In particular examples, the neutralizing antibody portion of the modified therapeutic antibody binds to a herpes virus (e.g., herpes simplex virus 1 (HSV-1) and/or herpes simplex virus 2 (HSV-2)). For example, the neutralizing antibody portion of the modified therapeutic antibody can bind to a glycoprotein of a herpes virus. Exemplary herpes virus envelope glycoproteins include, for example, glycoprotein D (gD), glycoprotein H (gH), glycoprotein B (gB), glycoprotein C (gC), glycoprotein G (gG), glycoprotein I (gI), glycoprotein E (gE), glycoprotein J (gJ), glycoprotein K (gK), glycoprotein L (gL), glycoprotein M (gM), and UL32. In a particular example, the neutralizing antibody portion binds to herpes virus glycoprotein D.

The neutralizing antibody portion of the modified therapeutic antibodies provided can be derived from any known antibody or antibody fragment. For example, the neutralizing antibody portion of the modified therapeutic antibody can be an antibody fragment (e.g., Fab, Fab′, F(ab′)₂, single-chain Fvs (scFv), Fv, dsFv, diabody, Fd and Fd′ fragments) that is engineered from a conventional antibody, where the antibody fragment retains the binding specificity of the full length antibody. In some examples, the neutralizing antibody portion of the modified therapeutic antibody is a single chain antibody. In a particular example, the neutralizing antibody portion of the modified therapeutic antibody is an anti-herpes virus AC8 single chain antibody.

To construct the modified therapeutic antibodies provided herein, a protein transduction domain is selected and is conjugated to the therapeutic antibody as described in detail elsewhere herein. Exemplary protein transduction domains for use in the methods are provided herein. Attachment of the protein transduction domain can be effected by any conventional technique. For example, the modified antibody can be produced recombinantly by expression of a nucleic acid encoding a fusion protein containing the therapeutic antibody fused to the transduction domain. In some examples, the transduction domain can be attached to the therapeutic antibody by chemical means as described herein and known in the art. The protein transduction domain can be conjugated to the antibody either directly or indirectly via peptide or other chemical linkers as described herein.

### C. STRUCTURE OF MODIFIED THERAPEUTIC ANTIBODIES

1. Protein Transduction Domain

The modified antibodies provided herein contain an antibody, or antigen binding fragment thereof, conjugated to a protein transduction domain (PTD) that increases the retention of the antibody at a target site for therapy, such as a mucosal site. Any PTD can be employed so long as the PTD promotes the binding of the antibody molecule to target cell surfaces at the therapeutic site (e.g. mucosal site) and/or uptake of the antibody by target cells at the therapeutic site (e.g. mucosal site).

Generally, PTDs include short cationic peptides that can bind to the cell surface through electrostatic attachment to the cell membrane and can be uptaken by the cell by membrane translocation (Kabouridis (2003) TRENDS Biotech 21(11) 498-503). The PTDs provided generally interact with a target cell via binding to glycosaminoglycans (GAGs), such as for example, hyaluronic acid, heparin, heparan sulfate, dermatan sulfate, keratin sulfate or chondroitin sulfate and their derivatives.

The protein transduction domain can be of any length. Generally the length of the PTD ranges from 5 or about 5 to 100 or about 100 amino acids in length. For example, the length of the PTD can range from 5 or about 5 to 25 or about 25 amino acids in length. In some examples, the PTD is 5, 6, 7, 8, 9, 10, 11, 12, 13, 14, 15, 16, 17, 18, 19, 20, 21, 22, 23, 24 or 25 amino acids in length.

A PTD for conjugation to an antibody provided can be selected from, but is not limited to, a PTD described herein or variants thereof. A single PTD can be selected for conjugation to the antibody. Multiple PTDs also can be selected for conjugation to the antibody. For example, multiple copies of the same PTD (e.g., dimer, trimer, tetramer, pentamer, hexamer, heptamer, octamer, nonamer, decamer or larger multimer) or different PTDs can be conjugated to the antibody.

Several proteins and their peptide derivatives have been found to possess cell internalization properties. Exemplary PTDs are known in the art and include, but are not limited to, PTDs listed in Table 3, including, for example, PTDs derived from human immunodeficiency virus 1 (HIV-1) TAT (SEQ ID NOS:910-921; Ruben et al. (1989) J. Virol. 63:1-8), the herpes virus tegument protein VP22 (SEQ ID NO: 926; Elliott and O'Hare (1997) Cell 88:223-233), the homeotic protein of Drosophila melanogaster Antennapedia (Antp) protein (Penetratin PTD; SEQ ID NO: 895; Derossi et al. (1996) J. Biol. Chem. 271:18188-18193), the protegrin 1 (PG-1) anti-microbial peptide SynB (e.g., SynB1 (SEQ ID NO: 907), SynB3 (SEQ ID NO: 908), and Syn B4 (SEQ ID NO: 909); Kokryakov et al. (1993) FEBS Lett. 327:231-236) and the Kaposi fibroblast growth factor (SEQ ID NO: 891; Lin et al., (1995) J. Biol. Chem. 270-14255-14258).

A number of other proteins and their peptide derivatives have been found to possess similar cell internalization properties. The carrier peptides that have been derived from these proteins show little sequence homology with each other, but are all highly cationic and arginine or lysine rich. Indeed, synthetic poly-arginine peptides have been shown to be internalized with a high level of efficiency and can be selected for conjugation to can antibody provided (Futaki et al. (2003) J. Mol. Recognit. 16:260-264; Suzuki et al. (2001) J. Biol. Chem. 276:5836-5840). The PTD also can be selected from among one or more synthetic PTDs, including but not limited to, transportan (SEQ ID NO: 922; Pooga et al. (1988) FASEB J. 12:67-77; Pooga et al. (2001) FASEB J. 15:1451-1453), MAP (SEQ ID NO: 889; Oehlke et al. (1998) Biochim. Biophys. Acta. 1414:127-139), KALA (SEQ ID NO: 887; Wyman et al. (1997) Biochemistry 36:3008-3017) and other cationic peptides, such as, for example, various β-cationic peptides (Akkarawongsa et al. (2008) Antimicrob. Agents and Chemother. 52(6):2120-2129). Additional PTD peptides and variant PTDs also are provided in, for example, U.S. Patent Publication Nos. US 2005/0260756, US 2006/0178297, US 2006/0100134, US 2006/0222657, US 2007/0161595, US 2007/0129305, European Patent Publication No. EP 1867661, PCT Publication Nos. WO 2000/062067, WO 2003/035892, WO 2007/097561, WO 2007/053512 and Table 3 herein. Any such PTDs provided herein or known in the art can be conjugated to a provided therapeutic antibody.

TABLE 3
Known Protein Transduction Domains

| | | SEQ ID |
|---|---|---|
| Protein Transduction Domain (PTD) | Source Protein | NO |
| TRSSRAGLQFPVGRVHRLLRK | Buforin II | 868 |
| RKKRRRESRKKRRRES | DPV3 | 869 |
| GRPRESGKKRKRKRLKP | DPV6 | 870 |
| GKRKKKGKLGKKRDP | DPV7 | 871 |
| GKRKKKGKLGKKRPRSR | DPV7b | 872 |
| RKKRRRESRRARRSPRHL | DPV3/10 | 873 |
| SRRARRSPRESGKKRKRKR | DPV10/6 | 874 |
| VKRGLKLRHVRPRVTRMDV | DPV1047 | 875 |
| VKRGLKLRHVRPRVTRDV | DPV1048 | 876 |
| SRRARRSPRHLGSG | DPV10 | 877 |
| LRRERQSRLRRERQSR | DPV15 | 878 |
| GAYDLRRRERQSRLRRRERQSR | DPV15b | 879 |
| WEAALAEALAEALAEHLAEALAEALEALAA | GALA | 880 |
| KGSWYSMRKMSMKIRPFFPQQ | Fibrinogen beta chain | 881 |
| KTRYYSMKKTTMKIIPFNRL | Fibrinogen gamma | 882 |
| | chain precursor | |
| RGADYSLRAVRMKIRPLVTQ | Fibrinogen alpha chain | 883 |
| LGTYTQDFNKFHTFPQTAIGVGAP | hCT(9-32) | 884 |
| TSLNIHNGQKL | HN-1 | 885 |
| NSAAFEDLRVLS | Influenza virus | 886 |
| | nucleoprotein (NLS) | |
| WEAKLAKALAKALAKHLAKALAKALKACEA | KALA | 887 |
| VPMLKPMLKE | Ku70 | 888 |
| KLALKLALKALKAALKLA | MAP | 889 |
| GALFLGFLGAAGSTMGAWSQPKKKRKV | MPG | 890 |
| AAVALLPAVLLALLAP | Human Fibroblast | 891 |
| | growth factor 4 | |
| | (Kaposi Fibroblast | |
| | growth factor) | |
| VQRKRQKLM | N50 (NLS of NF-kB P50) | 892 |
| KETWWETWWTEWSQPKKKRKV | Pep-1 | 893 |
| SDLWEMMMVSLACQY | Pep-7 | 894 |
| RQIKIWFQNRRMKWKK | Penetratin | 895 |
| GRQIKIWFQNRRMKWKK | Penetratin variant | 896 |
| RRMKWKK | Short Penetratin | 897 |
| ERQIKIWFQNRRMKWKK | Penetratin 42-58 | 898 |
| RRRRRRR | Poly Arginine-R7 | 899 |
| RRRRRRRRR | Poly Arginine-R9 | 900 |
| RVIRVWFQNKRCKDKK | pISL | 901 |
| MANLGYWLLALFVTMWTDVGLCKKRPKP | Prion mouse PrPc1-28 | 902 |
| LLIILRRRIRKQAHAHSK | pVEC | 903 |
| LLIILRRRIRKQAHAH | pVEC variant | 904 |
| VRLPPPVRLPPPVRLPPP | SAP | 905 |
| PKKKRKV | SV-40 (NLS) | 906 |
| RGGRLSYSRRRFSTSTGR | SynBl | 907 |
| RRLSYSRRRF | SynB3 | 908 |
| AWSFRVSYRGISYRRSR | SynB4 | 909 |
| YGRKKRRQRRRPPQ | Tat 47-60 | 910 |
| YGRKKRRQRRR | Tat 47-57 | 911 |
| YGRKKRRQRR | Tat 47-56 | 912 |
| GRKKRRQRR | Tat 48-56 | 913 |
| GRKKRRQRRR | Tat 48-57 | 914 |
| RKKRRQRRR | Tat 49-57 | 915 |
| RKKRRQRR | Tat 49-56 | 916 |
| GRKKRRQRRRPPQ | Tat 48-60 | 917 |
| GRKKR | Tat 48-52 | 918 |
| CFITKALGISYGRKKRRQRRRPPQFSQTHQVSLSKQ | Tat 37-72 | 919 |
| FITKALGISYGRKKRRQRRRPQFSQTHQVSLSKQ | Tat 38-72 | 920 |
| YGRKKRRQRRRPP | Tat 47-59 | 921 |
| GWTLNSAGYLLGKINLKALAALAKKIL | Transportan | 922 |
| AGYLLGKINLKALAALAKKIL | Transportan 10 | 923 |
| GWTLNSAGYLLG | Transportan derivative | 924 |
| INLKALAALAKKIL | Transportan derivative | 925 |
| DAATATRGRSAASRPTERPRAPARSASRPRRPVD | VP22 | 926 |
| DPKGDPKGVTVTVTVTVTGKGDPKPD | VT5 | 927 |
| GALFLGWLGAAGSTMGAWSQPKKKRKV | Signal Sequence-based | 928 |
| | peptide | |
| KLALKLALKALKAALKLA | Amphiphilic | 929 |
| | model peptide | |
| KFFKFFKFFK | Bacterial cell wall | 930 |
| | permeating | |
| LLGDFFRKSKEKIGKEFKRIVQRIKDFLRNLVPRTES | LL-37 | 931 |
| SWLSKTAKKLENSAKKRISEGTAIAIQGGPR | Cecropin P1 | 932 |
| ACYCRIPACIAGERRYGTCIYQGRLWAFCC | alpha defensin | 933 |
| DHYNCVSSGGQCLYSACPIFTKIQGTCYRGKAKCCK | beta defensin | 934 |
| RKCRIWIRVCR | Bactenecin | 935 |
| RRRPRPPYLPRPRPPPFFPPRLPPRIPPGFPPRFPPRFPGKR | PR-39 | 936 |
| ILPWKWPWWPWRR | Indolicidin | 937 |
| GALFLGWLGAAGSTMGAWSQPKKKRKV | MPS | 938 |
| PVIRRVWFQNKRCKDKK | pIs1 | 939 |

The PTD also can be selected from additional PTDs provided herein, which are identified herein and occur in native, or naturally-occurring proteins. Exemplary PTDs from naturally-occurring proteins include, for example, TAT-like transduction domains, prion-like transduction domains (Wadia et al. (2008) PLoS ONE 3(10) e3314: 1-8), and transduction domains with basic charges clustered on one face of the peptide alpha-helix. For example, the PTDs that can be conjugated to a therapeutic antibody provided can be selected from, but not limited to, a peptide provided in Tables 4, 5, 6 and 7 or SEQ ID NOS:5-855, 860 and 867.

While the additional PTDs provided herein (e.g., PTDs Tables 4, 5, 6, and 7 or SEQ ID NOS: 5-855, 860 and 867) are particularly useful for the conjugation to the antibodies provided, such PTDs also can be employed for cell attachment and/or cell membrane transport of other molecules such as, for example, compounds, peptides, oligonucleotides and small particles. In some examples, the PTDs provided in Tables 4, 5, 6, and 7 can be extended by one or more additional amino acids. In particular examples, the added amino acid is selected from a lysine or an arginine. In some examples, the PTDs provided in Tables 4, 5, 6, and 7 can be modified by replacement of a lysine or argine with another basic amino acid. In some examples, the PTDs provided in Tables 4, 5, 6, and 7 can be modified by replacement of a lysine with an arginine or by replacement of an arginine with a lysine.

a. TAT-Like Transduction Domains

### i. TAT-Like Transduction Domains with Gln at Position Six

The modified therapeutic antibodies provided herein can contain a PTD that is a variant of the HIV-TAT PTD, RKKRRQRRR (SEQ ID NO: 915), where position six of the nine amino acid PTD is glutamine. Exemplary PTD variants, which can be conjugated to a therapeutic antibody provided herein, include, but are not limited to variants found in naturally occurring proteins, where the PTD has glutamine at position six of a nine amino acid PTD peptide and either arginine or lysine at each of the other eight positions in the peptide. For example, the modified therapeutic antibodies provided herein can contain a PTD that is a variant of the HIV-TAT PTD, having an amino acid sequence, B₁-B₂-B₃-B₄-B₅-Q-B₆-B₇-B₈ (SEQ ID NO: 1043), where Q is glutamine and B₁, B₂, B₃, B₄, B₅, B₆, B₇ and B₈ are each independently lysine or arginine (the amino acid pattern also can be expressed as [K/R](5)-Q-[K/R](3)).

In some examples, the PTD that is attached to the therapeutic antibody is a peptide having an amino acid sequence B₁-B₂-B₃-B₄-B₅-Q-B₆-B₇-B₈ (where Q is glutamine and B₁, B₂, B₃, B₄, B₅, B₆, B₇ and B₈ are each independently lysine or arginine) and further containing one or more additional amino acids that are located at the N-terminus and/or C-terminus of the peptide, where each additional amino acid is independently arginine or lysine. In some examples, the PTD that is attached to the therapeutic antibody is a peptide having an amino acid sequence, B₁-B₂-B₃-B₄-B₅-Q-B₆-B₇-B₈ (where Q is glutamine and B₁, B₂, B₃, B₄, B₅, B₆, B₇ and B₈ are each independently lysine or arginine) that further contains 1, 2, 3, 4, 5, 6, 7, 8, 9, 10 or more additional amino acids that are located at the N-terminus and/or C-terminus of the peptide, where each additional amino acid is independently arginine or lysine. Typically, the one or more additional amino acids are located at the C-terminus of the PTD peptide (see, for example, SEQ ID NOS:7-9, 15, 18 and 30). When additional amino acids are located at the N-terminus of a modified PTD peptide, it is understood that the glutamine at position 6 of the PTD remains in the analogous position in the modified PTD.

In some examples, the PTD is selected from among variants provided in Table 4 and SEQ ID NOS:5-30. The PTDs identified Table 4 and SEQ ID NOS:5-30 can be conjugated to any antibody or antigen-binding fragment thereof provided herein or known in the art. In particular examples, the PTDs identified Table 4 and SEQ ID NOS:5-30 can be conjugated to a neutralizing antibody (e.g., an antiviral, an antibacterial, or an antifungal neutralizing antibody). In some examples, the PTDs identified Table 4 and SEQ ID NOS:5-30 can be conjugated to an antibody that binds to the surface of a pathogen, such as a viral, bacterial or fungal pathogen. For example, the PTDs identified Table 4 and SEQ ID NOS:5-30 can be conjugated to an antibody that binds to a glycoprotein on the surface of the pathogen.

In some examples, the PTD selected from among the peptides provided in Table 4 and SEQ ID NOS:5-30 is a variant, such as a variant that increases the charge of the peptide. For example, a PTD of Table 4 or SEQ ID NOS:5-30 can be modified to replace one or more lysine residues with an arginine. In some examples, a C-terminal lysine residue is replaced with an arginine.

TABLE 4
TAT-like Protein Transduction Domains with Gln at Position 6
[K/R](5)-Q-[K/R](3)

| Protein | SEQ | | |
|---|---|---|---|
| Transduction | ID | | |
| Domain (PTD) | NO | Source Organism | Source Protein |
| KKRKRQRRK | 5 | Physcomitrella patens | Putative protein |
| | | sub sp. patens | |
| KKRKKQRRK | 6 | Laccaria bicolor (strain S238N- | RNA processing-related |
| | | H82) (Bicoloured deceiver) | protein |
| | | Laccaria laccata var bicolor | |
| RKRRKQRKRK | 7 | Drosophila virilis (Fruit fly) | GJ17789 |
| KKRRKQRKRK | 8 | Drosophila mojavensis (Fruit fly) | GI21820 |
| RRKRRQRRRR | 9 | Feline immunodeficiency virus | Rev |
| KKRRRQKKK | 10 | Culex quinquefasciatus | Polycomb protein esc |
| | | (Southern house mosquito) | |
| KKKRKQKKK | 11 | Ictalurus punctatus | CBF1 interacting corepressor |
| | | (Channe lcatfish) | |
| KRKRKQKKK | 12 | Schizosaccharomyces pombe | C14401 |
| | | (Fission yeast) | |
| RKKRKQKKR | 13 | Homo sapiens (Human) | Ankyrin repeat domain- |
| | | protein 2 | containing |
| KKKRKQKRK | 14 | Nematostella vectensis | Putative protein |
| | | (Starlet sea anemone) | |
| RRRRKQRKKK | 15 | Homo sapiens (Human) | Tumor protein p53-inducible |
| | | protein 13 | |
| KKRRKQKRR | 16 | Danio rerio (Zebrafish) | Nucleolar protein 12 |
| | | Brachydanio rerio | |
| KRRKRQRKR | 17 | Drosophila persimilis (Fruit fly) | GL18495 |
| KKRKRQKRRR | 18 | Drosophila grimshawi (Fruit fly) | GH11647 |
| | | Idiomyia grimshawi | |
| KRRRKQKKR | 19 | Drosophila grimshawi (Fruit fly) | GH12028 |
| | | Idiomyia grimshawi | |
| KRRRKQKKK | 20 | Drosophila virilis (Fruit fly) | GJ19358 |
| KRRRRQRKR | 21 | Ostreococcus lucimarinus | Putative protein |
| | | (strain CCE9901) | |
| KRRRRQRRR | 22 | Tetraodon nigroviridis | Chromosome undetermined |
| | | (Green puffer) | SCAF7487 |
| RKRRRQKKR | 23 | Oryza sativa subsp japonica (Rice) | Os05g0296800 protein |
| KKRKRQKKK | 24 | Entamoeba dispar SAW760 | Actin |
| KKRKRQKRK | 25 | Pichia stipitis (Yeast) | Putative protein |
| RKRKKQRKR | 26 | Trypanosoma cruzi | Protein kinase |
| KKRKKQRKR | 27 | Trypanosoma cruzi | Protein kinase |
| KKKRRQKRR | 28 | Candida albicans (Yeast) | Putative protein kinase |
| KRRKRQRRR | 29 | Drosophila mojavensis (Fruit fly) | GI14201 |
| KKKRKQRRRK | 30 | Homo sapiens (Human) | Fer-1-like protein 4 |

### ii. TAT-Like Transduction Domains Without Gln at Position Six

The modified therapeutic antibodies provided herein can contain a PTD that is a variant of the HIV-TAT PTD, wherein position six of the nine amino acid PTD is an amino acid other than glutamine. In some examples, the amino acid at position six is an amino acid other than a glutamine, lysine or arginine. Exemplary PTD variants, which can be conjugated to a therapeutic antibody provided herein, include, but are not limited to variants found in naturally occurring proteins, where the PTD has an amino acid other than glutamine at position six of a nine amino acid PTD peptide and either arginine or lysine at each of the other eight positions in the peptide. For example, the modified therapeutic antibodies provided herein can contain a PTD that is a variant of the HIV-TAT PTD, having an amino acid sequence, B₁-B₂-B₃-B₄-B₅-X-B₆-B₇-B₈ (SEQ ID NO: 1044), wherein X is any amino acid other than glutamine and B₁, B₂, B₃, B₄, B₅, B₆, B₇ and B₈ are each independently lysine or arginine (amino acid pattern also can be expressed as [K/R](5)-X-[QR](3)). In some examples, X is arginine, lysine, aspartic acid, glutamic acid, or asparagine.

In some examples, the PTD that is attached to the therapeutic antibody is a peptide having an amino acid sequence, B₁-B₂-B₃-B₄-B₅-X-B₆-B₇-B₈ (where X is any amino acid other than glutamine and B₁, B₂, B₃, B₄, B₅, B₆, B₇ and B₈ are each independently lysine or arginine) and further containing one or more additional amino acids that are located at the N-terminus and/or C-terminus of the peptide, where each additional amino acid is independently arginine or lysine. In some examples, the PTD that is attached to the therapeutic antibody is a peptide having an amino acid sequence, B₁-B₂-B₃-B₄-B₅-X-B₆-B₇-B₈ (where X is any amino acid other than glutamine and B₁, B₂, B₃, B₄, B₅, B₆, B₇ and B₈ are each independently lysine or arginine) and further containing 1, 2, 3, 4, 5, 6, 7, 8, 9, 10 or more additional amino acids that are located at the N-terminus and/or C-terminus of the peptide, where each additional amino acid is independently arginine or lysine. Typically, the one or more additional amino acids are located at the C-terminus of the PTD peptide (see, for example, SEQ ID NOS:42, 45-47, 50, 52-53, 63-64, 67, 71, 74-75, 80, 90, 92, 97, 102, 104, 106-107, 114, 123, 125, 127-129, 131-133, 135 and 137). When additional amino acids are located at the N-terminus of a modified PTD peptide, it is understood that the amino acid at position 6 of the PTD remains in the analogous position in the modified PTD.

In some examples, the PTD is selected from among the peptides provided in Table 5 and SEQ ID NOS:31-143 and 860. The PTDs identified Table 5 and SEQ ID NOS:31-143 can be conjugated to any antibody or antigen-binding fragment thereof provided herein or known in the art. In particular examples, the PTDs identified Table 5 and SEQ ID NOS:31-143 and 860 can be conjugated to a neutralizing antibody (e.g. an antiviral, an antibacterial, or an antifungal neutralizing antibody). In some examples, the PTDs identified Table 5 and SEQ ID NOS:31-143 and 860 can be conjugated to an antibody that binds to the surface of a pathogen, such as a viral, bacterial or fungal pathogen. For example, the PTDs identified Table 5 and SEQ ID NOS:31-143 and 860 can be conjugated to an antibody that binds to a glycoprotein on the surface of the pathogen.

In some examples, the PTD selected from among the peptides provided in Table 5 and SEQ ID NOS:31-143 and 860 is a variant, such as a variant that increases the charge of the peptide. For example, a PTD of Table 5 or SEQ ID NOS:31-143 and 860 can be modified to replace one or more lysine residues with an arginine. In some examples, a C-terminal lysine residue is replaced with an arginine. In a particular example, if the PTD selected has the sequence KRRKRNRRRK (SEQ ID NO: 94), the PTD can be modified to replace the C-terminal lysine with an arginine, KRRKRNRRRR (SEQ ID NO: 860).

TABLE 5
TAT-like Transduction Domains without Gln at position 6
[K/R](5)-X-[K/R](3)

| Protein | SEQ | | |
|---|---|---|---|
| Transduction | ID | | |
| Domain (PTD) | NO | Source Organism | Source Protein |
| KKKRKSRKK | 31 | Bacillus subtilis | DNA translocase ftsK |
| RRRRKYKKR | 32 | Hyperthermus butylicus (strain | Conserved crenarchaeal protein-Zn |
| | | DSM5456/JCM 9403) | binding domain |
| RRRKKMKKK | 33 | Alkaliphilus metalliredigens | 30S ribosomal protein S18 |
| | | (strain QYMF) | |
| KKRKKEKKR | 34 | Saccharomyces cerevisiae | Protein YHROO7C-A |
| | | (Baker's yeast) | |
| KRKRKARKK | 35 | Alkaliphilus oremlandii | 30S ribosomal protein S18 |
| | | (strain OhILAs) | |
| RRRRKEKKK | 36 | Neosartorya fischeri (strain ATCC | Mating-type protein |
| | | 1020/DSM 3700/NRRL 181) | |
| KRRRRVRKK | 37 | Pelotomaculum | 50S ribosomal protein L18 |
| | | thermopropionicum (strain DSM | |
| | | 13744/JCM 10971/SI) | |
| KRKKRIRKK | 38 | Syntrophobacter fumaroxidans | LSU ribosomal protein L18P |
| | | (strain DSM 10017/MPOB) | |
| RKRRRIRRR | 39 | Medicago truncatula | Leucine-rich repeat |
| | | (Barrel medic) | |
| KRRRRGRRK | 40 | Sagittula stellata | Biopolymer transport protein, |
| | | | ExbD/TolR family |
| KRKKKHRRR | 41 | Homo sapiens (Human) | Arginine/serine-rich coiled-coil |
| | | | protein 1 |
| KRKKRERKRR | 42 | Trichomonas vaginalis | Polylysine protein |
| KKKRRGKRK | 43 | Platygyra sinensis | Histone H2B |
| KKKRRHKRK | 44 | Chironomus thummi thummi | Histone H2B |
| | | (Midge) | |
| KRKRKAKKRRK | 45 | Homo sapiens (Human) | Transmembrane protein TTMA |
| KKRRRFRRRKK | 46 | Cyanothece (sp CCY 0110) | Penicillin-binding protein lA |
| RKRRRCRRKR | 47 | Burkholderia dolosa (AU0158) | Phosphopantetheinyl-transferase |
| RRRRKGRKR | 48 | Aquifex aeolicus | 50S ribosomal protein L34 |
| RRKRRAKRR | 49 | Southern bean mosaic virus | Capsid protein |
| | | (SBMV) | |
| RRKKKGKKRK | 50 | Drosophila melanogaster | Chromodomain-helicase-DNA- |
| | | (Fruit fly) | binding protein Mi-2 homolog |
| RKKKRVRRR | 51 | Saccharomyces cerevisiae | YLR137W |
| | | (Baker's yeast) | |
| KKKRKHKRKR | 52 | Pichia stipitis (Yeast) | Putative protein |
| RKRRKHKKRKR | 53 | Debaryomyces hansenii (Yeast) | Transcription elongation factor |
| | | (Torulaspora hansenii) | SPT6 |
| KKRKKHKKK | 54 | Candida albicans (Yeast) | Stress response protein NST1 |
| KRRRRGRRR | 55 | Burkholderia mallei (strain | Cyclic di-GMP binding protein |
| | | NCTC10247) | |
| RRRRKHKRR | 56 | Ashbya gossypii (Yeast) | Transcription elongation factor |
| | | (Eremothecium gossypii) | SPT6 |
| RKKKRCRRK | 57 | Eledone cirrhosa (Curled octopus) | Cysteine-rich protamine |
| | | (Ozaena cirrosa) | |
| KKKRRHKRR | 58 | Candida glabrata (Yeast) | Transcription elongation factor |
| | | (Torulopsis glabrata) | SPT6 |
| KKRRKHKRR | 59 | Saccharomyces cerevisiae | Transcription elongation factor |
| | | (Baker's yeast) | SPT6 |
| RRRRKRKRR | 60 | Mus musculus (Mouse) | Tubulin polyglutamylase TTLL13 |
| RRKRRRKRR | 61 | Homo sapiens (Human) | Tubulin polyglutamylase TTLL13 |
| RRRRKKRRR | 62 | Neurospora crassa | Transcription elongation factor |
| | | | spt-6 |
| RRKRKRRRKKK | 63 | Bos taurus (Bovine) | Prokineticin-2 |
| RKRKRSKRKK | 64 | Homo sapiens (Human) | Prokineticin-2 |
| KRKKKGKRK | 65 | Gnetum gnemon (Bago) | RNA polymerase beta chain |
| | | (Gnetum acutatum) | |
| KKKRRGKKK | 66 | Pichia stipitis (Yeast) | Phosphatidylinositol-4-phosphate |
| | | | 5-kinase andrelated FYVE finger- |
| | | | containing proteins Signal |
| | | | transduction mechanisms |
| KRKRRKKKRK | 67 | Adelaide River virus (ARV) | Protein alpha-1 |
| KKRKKEKKK | 68 | Schizosaccharomyces pombe | Meiotically up-regulated gene116 |
| | | (Fission yeast) | protein |
| KKRKRSKKK | 69 | Aspergillus clavatus | Methionine aminopeptidase |
| RRKKRERKK | 70 | Xenopus tropicalis (Western | LOC100036708 protein |
| | | clawed frog) (Silurana tropicalis) | |
| KRKKRTKKKR | 71 | Chlorella vulgaris (Green alga) | 165 kDa protein in psaC atpA |
| | | | intergenic region |
| RKKRRLKKR | 72 | Homo sapiens (Human) | Transient receptor potential cation |
| | | | channel subfamily V member 3 |
| RRKKRRRRK | 73 | Dictyostelium discoideum | Protein DDB 0237901 |
| | | (Slimemold) | |
| KKKRKRRRRK | 74 | Mullus surmuletus (Striped | Protamine-like protein |
| | | redmullet) | |
| RKRRRKRRRR | 75 | Xanthomonas oryzae pv oryzae | Transposase |
| KRKRKRKRR | 76 | Aspergillus niger (strain | Putative protein |
| | | CBS51388/FGSC A1513) | |
| RKRRKRRKK | 77 | Bos taurus (Bovine) | Probetacellulin |
| RKRRKRKKK | 78 | Homo sapiens (Human) | Probetacellulin |
| RKRRKERKK | 79 | Homo sapiens (Human) | Coiled-coil domain-containing |
| | | | protein 140 |
| KKRKKEKKKRK | 80 | Danio rerio (Zebrafish) | Zgc:162339 protein |
| | | (Brachydanio rerio) | |
| KKRRKRRRK | 81 | Homo sapiens (Human) | Lipin-1 |
| RRRKKEKRR | 82 | Ostreococcus lucimarinus | Putative protein |
| | | (strain CCE9901) | |
| RKRKKERKK | 83 | Ostreococcus lucimarinus | Putative protein |
| | | (strain CCE9901) | |
| RRRRKVRRR | 84 | Drosophila melanogaster | Longitudinals lacking protein, |
| | | (Fruit fly) | isoform G |
| KKKRKLKKK | 85 | Bos taurus (Bovine) | Protein C12orf43 homolog |
| RKRKKVRRR | 86 | Anopheles gambiae (African | AGAP005245-PD |
| | | malaria mosquito) | |
| KRKKKSRKK | 87 | Homo sapiens (Human) | Protein C11lorf57 |
| KRRKKLKRK | 88 | Saccharomyces cerevisiae | Protein YEL057C |
| | | (Baker's yeast) | |
| RKRKRRRRK | 89 | Trichomonas vaginalis | Cyclophilin-RNA interacting |
| | | | protein |
| KKRKKEKKKR | 90 | Tobacco mild green mosaic virus | Movement protein |
| | | (TMGMV) (TMV strain U2) | |
| KKKRKIKKR | 91 | Ecotropis obliqua NPV | Late expression factor 5 |
| KKRKKEKKRKK | 92 | Aedes aegypti (Yellowfever | Mediator of RNA polymerase II |
| | | mosquito) | transcription subunit 19 |
| KRKKRNRRRK | 93 | Rattus norvegicus (Rat) | T-cell surface antigen CD2 |
| KRRKRNRRRK | 94 | Mus musculus (Mouse) | T-cell surface antigen CD2 |
| RKRKKKRRK | 95 | Xenopus tropicalis (Western | LOC100124814 protein |
| | | clawed frog) (Silurana tropicalis) | |
| KKRKRSRRKK | 96 | Mus musculus (Mouse) | MKI67 FHA domain-interacting |
| | | | nucleolar phosphoprotein |
| KKRRREKKKRR | 97 | Caenorhabditis elegans | Protein let-756 |
| KKRKKLKKRK | 98 | Homo sapiens (Human) | Ankyrin repeat domain-containing |
| | | | protein 18B |
| KKRKKRKRKK | 99 | Homo sapiens (Human) | G patch domain-containing |
| | | | protein 8 |
| RKRRREKRRR | 100 | Ustilago maydis (Smut fungus) | Vacuolar fusion protein MON1 |
| KKRRKRKKRR | 101 | Microscilla marina (ATCC 23134) | Outer membrane protein OmpA |
| | | | family |
| KRRKRKKRKRK | 102 | Fugu rubripes (Japanese | Homeobox protein Hox-C8a |
| | | pufferfish) (Takifugu rubripes) | |
| RRKRKRRRK | 103 | Mus musculus (Mouse) | Scaffold attachment factor B2 |
| RRKRKTRRRKK | 104 | Homo sapiens (Human) | RING and PHD-finger domain- |
| | | | containing protein KIAA1542 |
| RKKKRRRRK | 105 | Haliotis asinina | BMP2/4 |
| RRRRKEKRRR | 106 | Bos taurus (Bovine) | Target of EGR1 protein 1 |
| RRRRKDKRKR | 107 | Mus musculus (Mouse) | Target of EGR1 protein 1 |
| KKKRKEKKR | 108 | Dictyostelium discoideum | rRNA methyltransferase 3 |
| | | (Slimemold) | homolog |
| KKKRKKRKR | 109 | Mus musculus (Mouse) | Ribosomal RNA processing |
| | | | protein 1 homolog B |
| RRKRREKRR | 110 | Homo sapiens (Human) | Kinesin-like protein KIF3B |
| RRRRKMKRR | 111 | Leishmania braziliensis | RNA-binding protein, |
| RRRRKSRKK | 112 | Bacillus thuringiensis | ATP-dependent RNA helicase, |
| | | (strain A1 Hakam) | DEAD/DEAH box family |
| KKKRKKRKK | 113 | Ajellomyces capsulata | WD repeat-containing protein JIP5 |
| | | (strainNAml/WU24) (Darling's | |
| | | diseasefungus) (Histoplasma | |
| | | capsulatum) | |
| KRKKKSKRRK | 114 | Ostreococcus lucimarinus | Putative protein |
| | | (strain CCE9901) | |
| KRRRRSRKK | 115 | Homo sapiens (Human) | Histone-lysine N- |
| | | | methyltransferase, H3 lysine-9 |
| | | | specific 5 |
| KRRKKRKKK | 116 | Rattus norvegicus (Rat) | Ankyrin repeat and zinc finger |
| | | | domain-containing protein 1 |
| KRRRKHRKR | 117 | Danio rerio (Zebrafish) | LOC798657 protein |
| | | (Brachydanio rerio) | |
| KRKRKPRKK | 118 | Marine gamma proteobacterium | Poly(A) polymerase |
| | | (HTCC2143) | |
| RRKKRVRRK | 119 | Thiobacillus denitrificans | GTP-binding protein engA |
| | | (strain ATCC 25259) | |
| KRRRKERKR | 120 | Pichia stipitis (Yeast) | Kinase of RNA polymerase II |
| | | | carboxy-terminal domain (CTD) |
| KKRKKRRRR | 121 | Paramecium tetraurelia | Chromosome undetermined |
| | | | scaffold 62 |
| RRKRKKRKR | 122 | Human papillomavirus type 41 | Minor capsid protein L2 |
| KKKRRRRKKK | 123 | Homo sapiens (Human) | Probable global transcription |
| | | | activator SNF2L2 |
| RRRKRSRRR | 124 | Ostreococcus lucimarinus | Putative protein |
| | | (strain CCE9901) | |
| RKRRRPRRRK | 125 | Desulfovibrio vulgaris subsp | DEAD/DEAH box helicase |
| | | vulgaris (strain DP4) | domain protein |
| RRRRKNRKK | 126 | Robiginitalea biformata | Signal peptidase I |
| | | (HTCC2501) | |
| KRRRKGKRRKR | 127 | Schizosaccharomyces pombe | Serine/threonine-protein kinase |
| | | (Fission yeast) | ppk4 |
| KKKRRRKRKR | 128 | Geobacter bemidjiensis | Ribonuclease, Rne/Rng family |
| | | (strain Bent) | |
| KKRKRRRKRRR | 129 | Magnetococcus sp (strain MC-1) | Ribonuclease E |
| KRKRRLKKK | 130 | Juncus decipiens | NADH dehydrogenase subunit F |
| RKKKRRKRKK | 131 | Homo sapiens (Human) | Smoothened homolog |
| RRKRRAKKRR | 132 | Aspergillus clavatus | Involucrin repeat protein |
| RRKRRTKRRK | 133 | Caenorhabditis elegans | Similarity to hypothetical protein |
| | | | isof |
| KRRRKARRR | 134 | Janibacter sp (HTCC2649) | Polyphosphate kinase |
| RKKRRKRKKRR | 135 | Microscilla marina (ATCC 23134) | TonB-dependent receptor |
| KKKRKNRKR | 136 | Mus musculus (Mouse) | WD repeat-containing protein 3 |
| KKRRKRRRRK | 137 | Plasmodium falciparum (isolate | DNA-directed RNA polymerase II |
| | | CDC/Honduras) | subunit RPB1 |
| RRRKRLKKK | 138 | Homo sapiens (Human) | AT-rich interactive domain- |
| | | | containing protein 4A |
| KRKRKTKRK | 139 | Pinus thunbergii (Green pine) | Protein ycf2 |
| | | (Japanese black pine) | |
| KRKKRIKRR | 140 | Homo sapiens (Human) | Polycystic kidney disease and |
| | | | receptor for egg jelly-related |
| | | | protein |
| RRKRRLRRR | 141 | Emericella nidulans | Cytokinesis protein sepA |
| | | (Aspergillus nidulans) | |
| KKRRRTRRK | 142 | Ustilago maydis (Smut fungus) | Lysophospholipase NTE1 |
| KKRKRVKRK | 143 | Paramecium tetraurelia | Chromosome undetermined |
| | | scaffold 68 | |

b. Prion-Like Transduction Domains

The modified therapeutic antibodies provided herein can contain a PTD that is a variant of the prion PTD, KPSKPKTLNK (Wadia et al. (2008) PLoS ONE 3(10) e3314: 1-8; SEQ ID NO: 1050), where the PTDs is selected from among PTDs having the amino acid sequence B₁-P₁-X₁-B₂-P₂-B₃-X₂-X₃-X₄-B₄ (SEQ ID NO: 1045), where P₁ and P₂ are proline, B₁, B₂, B₃ and B₄ are each independently lysine or arginine and X₁, X₂, X₃ and X₄ are each independently any amino acid (amino acid pattern also can be expressed as [K/R]-P-X-[K/R]-P-[K/R]-X(3)-[K/R]). In some examples, X₁, X₂, X₃, and X₄ are each independently arginine, lysine, proline or aspartic acid. Exemplary PTD variants, which can be conjugated to a therapeutic antibody provided herein, include, but are not limited to variants found in naturally occurring proteins, where the PTD has the amino acid pattern B₁-P₁-X₁-B₂-P₂-B₃-X₂-X₃-X₄-B₄ described above.

In some examples, the PTD that is attached to the therapeutic antibody is a peptide having an amino acid sequence, B₁-P₁-X₁-B₂-P₂-B₃-X₂-X₃-X₄-B₄ (where P₁ and P₂ are proline, B₁, B₂, B₃ and B₄ are each independently lysine or arginine and X₁, X₂, X₃ and X₄ are each independently any amino acid) and further containing one or more additional amino acids that are located at the N-terminus and/or C-terminus of the peptide, where each additional amino acid is independently arginine or lysine. In some examples, the PTD that is attached to the therapeutic antibody is a peptide having an amino acid sequence, B₁-P₁-X₁-B₂-P₂-B₃-X₂-X₃-X₄-B₄ (where P₁ and P₂ are proline, B₁, B₂, B₃ and B₄ are each independently lysine or arginine and X₁, X₂, X₃ and X₄ are each independently any amino acid) and further containing 1, 2, 3, 4, 5, 6, 7, 8, 9, 10 or more additional amino acids that are located at the N-terminus and/or C-terminus of the peptide, where each additional amino acid is independently arginine or lysine. Typically, the one or more additional amino acids are located a the C-terminus of the peptide (see, for example, SEQ ID NO:203). When additional amino acids are located at the N-terminus of a modified PTD peptide, it is understood that the modified PTD contains amino acids analogous to the unmodified PTD, for example, the modified PTD retains the amino acid sequence B₁-P₁-X₁-B₂-P₂-B₃-X₂-X₃-X₄-B₄ (where P₁ and P₂ are proline, B₁, B₂, B₃ and B₄ are each independently lysine or arginine and X₁, X₂, X₃ and X₄ are each independently any amino acid).

In some examples, the PTD is selected from among the peptides provided herein in Table 6 and SEQ ID NOS:144-499. The PTDs identified Table 6 and SEQ ID NOS:144-499 can be conjugated to any antibody or antigen-binding fragment thereof provided herein or known in the art. In particular examples, the PTDs identified Table 6 and SEQ ID NOS:144-499 can be conjugated to a neutralizing antibody (e.g. an antiviral, an antibacterial, or an antifungal neutralizing antibody). In some examples, the PTDs identified Table 6 and SEQ ID NOS:144-499 can be conjugated to an antibody that binds to the surface of a pathogen, such as a viral, bacterial or fungal pathogen. For example, the PTDs identified Table 6 and SEQ ID NOS:144-499 can be conjugated to an antibody that binds to a glycoprotein on the surface of the pathogen.

In some examples, the PTD selected from among the peptides provided in Table 6 and SEQ ID NOS:144-499 is a variant, such as a variant that increases the charge of the peptide. For example, a PTD of Table 6 or SEQ ID NOS:144-499 can be modified to replace one or more lysine residues with an arginine. In some examples, a C-terminal lysine residue is replaced with an arginine.

TABLE 6
Prion-like Transduction Domains
[K/R]-P-X-[K/R]-P-[K/R]-X(3)-[K/R]

| Protein | SEQ | | |
|---|---|---|---|
| Transduction | ID | | |
| Domain (PTD) | NO | Source Organism | Source Protein |
| KPARPRPPRR | 144 | Burkholderia cenocepacia | TRAP-type transport system |
| | | (strain H12424) | periplasmic component-like |
| | | | protein |
| RPGRPRRPKR | 145 | Salinispora tropica (strain ATCC | Pyruvate |
| | | BAA-916/DSM 44818/CNB-440) | flavodoxin/ferredoxinoxido |
| | | reductase domain protein | |
| KPAKPRKERK | 146 | Roseobacter sp. (SK209-2-6) | Transcriptional regulator |
| RPPKPKRMGR | 147 | Aeropyrum pernix | 30S ribosomal protein Sl4P |
| RPIKPKKLNR | 148 | Bradyrhizobium sp. (strain BTAi1/ | |
| | | Diguanylate ATCC BAA-1182) | cyclase/phosphodiesterase |
| RPTRPRLFSR | 149 | Nocardioides sp. (strain BAA-499/ | Na(+)/H(+) antiporter nhaA 1 |
| | | JS614) | |
| RPERPRPSGK | 150 | Desulfatibacillum alkenivorans | GTP-binding protein LepA |
| | | (AK-01) | |
| KPKKPKSKRR | 151 | Oceanicaulis alexandri | Probable endonuclease III |
| | | (HTCC2633) | protein |
| KPAKPKRWKK | 152 | Kordia algicida (OT-1) | Protein containing StAR- |
| | | | related lipid-transfer |
| | | | (START) domain |
| RPKKPKHRER | 153 | Caenorhabditis briggsae | UPF0493 protein CBG04313 |
| RPPKPKAKPK | 154 | Chlamydomonas reinhardtii | Putative protein |
| KPSRPKGTYR | 155 | Laccaria bicolor (strain S238N- | Putative protein |
| | | H82) (Bicoloured deceiver) | |
| | | (Laccaria laccata va r. bicolor) | |
| KPIKPKKTTK | 156 | Haemophilus aphrophilus | Penicillin-binding protein 3 |
| RPAKPKSQRK | 157 | Synechococcus sp. (strain WH8102) | Translation initiation factor |
| | | | IF-2 |
| RPYRPRPSSR | 158 | Burkholderia pseudomallei | Gp7 |
| | | (Pasteur 52237) | |
| KPGKPKKTIR | 159 | Haemophilus parainfluenzae | Penicillin-binding protein 3 |
| RPRRPRPDRR | 160 | Methylobacterium sp. (strain 4-46) | General secretory system II |
| | | | protein E domain protein |
| KPIRPRHPEK | 161 | Rhodopseudomonas palustris (strain | Lipoyl synthase |
| | | BisA53) | |
| RPTRPRRHVR | 162 | Coprinopsis cinerea (strain | Phosphorylase |
| | | Okayama-7/130/FGSC | |
| | | 9003)(Inky cap fungus) | |
| | | (Hormographiella aspefgillata) | |
| RPVKPRSRCR | 163 | Burkholderia contaminans | Protein recA |
| RPTKPKDKLR | 164 | Nematostella vectensis (Starletsea | Putative protein |
| | | anemone) | |
| RPPRPRVPAR | 165 | Azoarcus sp. (strain EbN1) | Probable chorismate-- |
| | | (Aromatoleum aromaticum (strain | pyruvatelyase |
| | | EbN1)) | |
| RPKKPKSHAR | 166 | Sclerotinia sclerotiorum | Putative protein |
| | | (strainATCC 18683/1980/Ss-1) | |
| | | (White mold) (Whetzelinia | |
| | | sclerotiorum) | |
| RPPKPKLKTR | 167 | Culex quinquefasciatus (Southern | Ribonuclease iii |
| | | house mosquito) | |
| KPCKPKGEKK | 168 | Eurypelma californica (American | Neurotoxin ESTx1 |
| | | tarantula) | |
| RPPRPRRLLR | 169 | Kineococcus radiotolerans (strain | Integral membrane sensor |
| | | ATCC BAA-149/DSM 14245/ | signal transduction histidine |
| | | SRS30216) | kinase |
| KPFKPKSGPR | 170 | Pseudomonas putida (strain GB-1) | Transcriptional regulator, |
| | | | GntR family with |
| | | | aminotransferase domain |
| RPARPRTAAR | 171 | Roseovarius sp. 217 | S-adenosylmethionine: tRNA |
| | | | ribosyltransferase-isomerase |
| KPGRPKKKFR | 172 | Aspergillus clavatus | DUF1665 domain protein |
| RPARPRDSAR | 173 | Novosphingobium aromatici- | S-adenosylmethionine: tRNA |
| | | vorans (strain DSM12444) | ribosyltransferase-isomerase |
| RPARPRSSAR | 174 | Dinoroseobacter shibae | S-adenosylmethionine: tRNA |
| | | (strain DFL 12) | ribosyltransferase-isomerase |
| RPVRPRDAAR | 175 | Erythrobacter sp. NAP1 | S-adenosylmethionine: |
| | | | tRNA-ribosyltransferase |
| RPVRPRPAAR | 176 | Roseovarius nubinhibens ISM | S-adenosylmethionine: tRNA |
| | | | ribosyltransferase-isomerase |
| RPARPRTAAR | 177 | Rhodobacter sphaeroides (strain | S-adenosylmethionine: tRNA |
| | | ATCC 17029/ATH 2.4.9) | ribosyltransferase-isomerase |
| RPARPRDAAR | 178 | Maricaulis maris (strain MCS10) | S-adenosylmethionine: tRNA |
| | | | ribosyltransferase-isomerase |
| RPRRPRRWWR | 179 | Bos taurus (Bovine) | SLC15A3 protein |
| RPRRPRQLTR | 180 | Homo sapiens (Human) | NOX5 variant lacking EF |
| | | | handsHCG2003451, isoform |
| | | | CRAd |
| KPLKPKQGTK | 181 | Chlamydomonas reinhardtii | Putative protein |
| RPVRPRGPSR | 182 | Methylobacterium sp. (strain4-46) | Integrase catalytic region |
| KPYKPKNHIR | 183 | Microscilla marina (ATCC 23134) | Methylmalonyl-CoA mutase |
| RPRKPRPGSR | 184 | Triticum aestivum (Wheat) | MBD3 |
| KPRKPKLERR | 185 | Vitis vinifera (Grape) | Chromosome chr18 scaffold 1 |
| RPGRPRTKPR | 186 | Bat coronavirus HKU5-2 | Nucleocapsid phosphoprotein |
| KPAKPRSFRR | 187 | Mycobacterium vanbaalenii (strain | VanW family protein |
| | | DSM 7251/PYR-1) | |
| KPRKPRAAAK | 188 | Vibrio splendidus 12B01 | Membrane carboxypeptidase |
| KPNKPKFGQR | 189 | Vibrio campbellii AND4 | 3-deoxy-D-manno- |
| | | | octulosonic-acidtransferase |
| KPNKPKFGGR | 190 | Vibrio parahaemolyticus AQ3810 | 3-deoxy-D-manno- |
| | | | octulosonic-acidtransferase |
| KPRKPRASAK | 191 | Vibrio sp. MED222 | Membrane carboxypeptidase |
| KPNKPKFGNR | 192 | Vibrio splendidus 12B01 | 3-deoxy-D-manno- |
| | | | octulosonic-acidtransferase |
| KPNKPKFGSR | 193 | Vibrio sp. Ex25 | 3-deoxy-D-manno- |
| | | | octulosonic-acidtransferase |
| | | | subfamily |
| RPNKPKFGQR | 194 | Vibrio harveyi HY01 | 3-deoxy-D-manno- |
| | | | octulosonic-acidtransferase |
| RPGRPRTYRR | 195 | Orgyia pseudotsugata multicapsid | DNA-binding protein |
| | | polyhedrosis virus (OpMNPV) | |
| RPPKPRWGLR | 196 | Human papillomavirus type 29 | Probable protein E4 |
| RPRKPKYFNR | 197 | Ostreococcus lucimarinus (strain | Putative protein |
| | | CCE9901) | |
| KPARPKPTHR | 198 | Cryptococcus neoformans | rRNA-processing protein |
| | | (Filobasidiella neoformans) | EFG1 |
| KPPKPRKKPK | 199 | Caenorhabditis elegans | Bifunctional heparan sulfate |
| | | | N-deacetylase/N- |
| | | | sulfotransferase 1 |
| KPGKPRKPPK | 200 | Caenorhabditis briggsae | Bifunctional heparan sulfate |
| | | | N-deacetylase/N- |
| | | | sulfotransferase 1 |
| KPRKPRRPRK | 201 | Mus musculus (Mouse) | Zinc finger protein 41 |
| RPIRPRPAMK | 202 | Ralstonia solanacearum UW551 | Beta-hexosaminidase |
| RPGRPKGSTTR | 203 | Coprinopsis cinerea (strain | Putative protein |
| | | Okayama-7/130/FGSC 9003) | |
| | | (Inky cap fungus) (Hormographiella | |
| | | aspergillata) | |
| RPTRPKVRVR | 204 | Azorhizobium caulinodans (strain | Regulatory protein |
| | | ATCC 43989/DSM 5975/ORS | |
| | | 571) | |
| KPGRPKIASK | 205 | Laccaria bicolor (strain S238N- | Putative protein |
| | | H82) (Bicoloured deceiver) | |
| | | (Laccaria laccata var. bicolor) | |
| KPAKPRIAPK | 206 | Vibrio campbellii AND4 | Pseudouridine synthase |
| RPGRPKGSTK | 207 | Laccaria bicolor (strain S238N- | Putative protein |
| | | H82) (Bicoloured deceiver) | |
| | | (Laccaria laccata var. bicolor) | |
| RPEKPRQINR | 208 | Oceanibulbus indolifex HEL-45 | Probable insertion |
| | | | sequencetransposase protein |
| RPGKPRPLLR | 209 | Mus musculus (Mouse) | Gamma-aminobutyric acid |
| | | | (GABA-C) receptor |
| RPRKPRPPRR | 210 | Roseovarius nubinhibens ISM | Penicillin-binding protein, 1 A |
| | | | family protein |
| KPLKPKQAFR | 211 | Idiomarina baltica 0S145 | Apolipoprotein N- |
| | | | acyltransferase |
| KPDKPKGVGK | 212 | Aspergillus niger | Catalytic activity: hydrolysis |
| | | (strain CBS513.88/FGSC A1513) | ofterminal 1 |
| KPARPRQLRK | 213 | Brugia malayi (Filarialnematode | 60S ribosomal protein L34 |
| | | worm) | |
| KPTRPRLRPK | 214 | Synechococcus sp. (strain JA-3- | Translation initiation factor |
| | | 3Ab) (Cyanobacteria bacterium | IF-2 |
| | | Yellowstone A-Prime) | |
| KPGKPRKRKK | 215 | Homo sapiens (Human) | Chromodomain-helicase- |
| | | | DNA-binding protein 3 |
| RPRRPKLNSK | 216 | Pichia guilliermondii (Yeast) | Putative protein |
| | | (Candida guilliermondii) | |
| RPQRPRRQQK | 217 | Chlamydomonas reinhardtii | Putative protein |
| RPNRPRVKKK | 218 | Bartonella tribocorum (strain CIP | TrwJ2 protein |
| | | 105476/IBS 506) | |
| RPHRPKSQTR | 219 | Magnaporthe grisea (Rice | Putative protein |
| | | blastfungus) (Pyricularia grisea) | |
| KPAKPKPAPK | 220 | Magnaporthe grisea (Rice | Putative protein |
| | | blastfungus) (Pyricularia grisea) | |
| RPFRPRTPVR | 221 | Murine cytomegalovirus | M25 protein |
| | | (strainK181) | |
| RPERPRSCYR | 222 | Hoeflea phototrophica DFL-43 | Transcriptional regulator |
| RPKRPRTPQR | 223 | Aeropyrum pernix | 50S ribosomal protein L13P |
| RPSKPRKEKK | 224 | Coprinopsis cinerea (strain | Putative protein |
| | | Okayama-7/130/FGSC 9003) | |
| | | (Inky cap fungus) (Hormographiella | |
| | | aspergillata) | |
| RPPRPRNDFR | 225 | Methanoculleus marisnigri (strain | RNP-1 like RNA-binding |
| | | ATCC 35101/DSM 1498/JR1) | protein |
| KPLKPKQLDR | 226 | Nitratiruptor sp. (strain SB155-2) | Phosphate ABC transporter, |
| | | | substrate-binding protein |
| KPLKPKTLKK | 227 | Sulfurovum sp. (strain NBC37-1) | Phosphate ABC |
| | | | transporter, substrate-binding |
| | | | protein |
| RPQRPKPQRR | 228 | Botryotinia fuckeliana (strain | Putative protein |
| | | B05.10) (Noble rot fungus) (Botrytis | |
| | | cinerea) | |
| KPEKPKKEHK | 229 | Arcobacter butzleri (strain | TonB-dependent receptor |
| | | RM4018) | protein |
| KPKKPKTQEK | 230 | Sindbis virus (STNV) | Structural polyprotein |
| KPPRPRGRGR | 231 | Physcomitrella patens subsp.patens | Putative protein |
| KPKKPKPQEK | 232 | Sindbis virus subtype Ockelbo | Structural polyprotein |
| | | (strain Edsbyn 82-5) (OCKV) | |
| | | (Ockelbo virus) | |
| RPARPRAGGR | 233 | Sphingomonas wittichii | Enoyl-CoA |
| | | (strain RW1/DSM 6014/JCM 10273) | hydratase/isomerase |
| KPRKPRPGRR | 234 | Saimiriine herpesvirus 1 (strain | Glycoprotein B |
| | | MV-5-4-PSL) (SaHV-1) (Marmoset | |
| | | herpesvirus) | |
| KPGKPKGKKK | 235 | Xenopus laevis (African clawed | Transcriptional adapter 1-like |
| | | frog) | protein |
| KPEKPKEKPK | 236 | Actinobacillus succinogenes (strain | TonB family protein |
| | | ATCC 55618/130Z) | |
| RPFKPRIKSR | 237 | Nematostella vectensis (Starletsea | Putative protein |
| | | anemone) | |
| RPARPRETRR | 238 | Ashbya gossypii (Yeast) | Histone H3-like centromeric |
| | | (Eremothecium gossypii) | proteinCSE4 |
| RPWKPRRHHR | 239 | Chlamydomonas reinhardtii | Putative protein |
| KPKKPRTTRR | 240 | Drosophila melanogaster (Fruitfly) | CG14479-PA |
| KPDRPKGLKR | 241 | Homo sapiens (Human) | RNA-binding protein Raly |
| KPYRPKPGSK | 242 | Mus musculus (Mouse) | RNA-binding Raly-like |
| | | | protein |
| KPVKPKTSMK | 243 | Spermophilus tridecemlineatus | Prion protein PrP |
| | | (Thirteen-lined ground squirrel) | |
| KPNRPKGLKR | 244 | Mus musculus (Mouse) | RNA-binding protein Raly |
| KPYRPKLGTK | 245 | Xenopus tropicalis (Western clawed | RNA-binding Raly-like |
| | | frog) (Silurana tropicalis) | protein |
| KPYRPKPGNK | 246 | Bos taurus (Bovine) | RNA-binding Raly-like |
| | | | protein |
| RPVRPRRLRR | 247 | Victivallis vadensis (ATCC BAA- | Binding-protein-dependent |
| | | 548) | transportsystems inner |
| | | | membrane component |
| KPAKPKKTFK | 248 | Mycoplasma pulmonis | 30S ribosomal protein S6 |
| KPMKPKLSNK | 249 | Aspergillus clavatus | DUF726 domain protein |
| RPQKPKSKSR | 250 | Coprinopsis cinerea (strain | Putative protein |
| | | Okayama-7/130/FGSC 9003) | |
| | | (Inky cap fungus) (Hormographiella | |
| | | aspergillata) | |
| KPVKPKTSMK | 251 | Cebus apella (Brown- | Major prion protein |
| | | cappedcapuchin) | |
| KPSKPKTNLK | 252 | Mus musculus (Mouse) | Major prion protein |
| KPSKPKTNMK | 253 | Ateles paniscus (Black | Major prion protein |
| | | spidermonkey) | |
| KPNKPKTSMK | 254 | Cricetulus migratorius (Armenian | Major prion protein |
| | | hamster) | |
| KPIRPRYEVK | 255 | Nematostella vectensis (Starletsea | Putative protein |
| | | anemone) | |
| RPYRPKRRTR | 256 | Clostridium phytofermentans (strain | RNA binding S1 domain |
| | | ATCC 700394/DSM 18823/ISDg) | protein |
| RPFRPKRKTR | 257 | Methanococcus maripaludis (strain | RNA binding S1 domain |
| | | C5/ATCC BAA-1333) | protein |
| RPFKPKKRTK | 258 | Clostridium beijerinckii (strain | RNA binding SI domain |
| | | ATCC 51743/NCIMB 8052) | protein |
| | | (Clostridium acetobutylicum) | |
| RPFRPKRRTR | 259 | Clostridium thermocellum (strain | RNA binding S1 |
| | | ATCC 27405/DSM 1237) | |
| KPEKPKEKLK | 260 | Haemophilus ducreyi | Protein tonB |
| RPYKPKKRTR | 261 | Clostridium kluyveri (strain ATCC | Tex |
| | | 8527/DSM 555/NCIMB10680) | |
| RPFKPKKRTR | 262 | Clostridium novyi (strain NT) | SiRNA binding domain |
| | | | protein |
| KPSKPKTNXK | 263 | Ovis aries (Sheep) | PRNP |
| KPSKPKTNTK | 264 | Ovis aries (Sheep) | Major prion protein |
| KPNKPKTSMK | 265 | Tragelaphus spekii (Sitatunga) | Major prion protein |
| KPKKPRKKIR | 266 | Streptococcus sanguinis (strain | Transcriptional attenuator |
| | | SK36) | LytR |
| KPDKPKTNLK | 267 | Trichosurus vulpecula (Brush-tailed | Major prion protein |
| | | possum) | |
| RPPRPRPDNR | 268 | Shewanella sediminis (strain HAW- | Sensor histidine kinase |
| | | EB3) | |
| RPLRPRARLR | 269 | Anaeromyxobacterde- | NADH dehydrogenase |
| | | halogenans 2CP-1 | (Quinone) |
| RPQKPKKRLR | 270 | Neosartorya fischeri (strain ATCC | Nucleic acid-binding protein |
| | | 1020/DSM 3700/NRRL 181) | |
| | | (Aspergillus fischerianus (strain | |
| | | ATCC 1020 /DSM 3700/NRRL | |
| | | 181)) | |
| RPAKPRSPSR | 271 | Mus musculus (Mouse) | Myelin-associated |
| | | | oligodendrocytebasic protein |
| KPXKPKTNMK | 272 | Kobus ellipsiprymnus (Waterbuck) | Major prion protein |
| KPGRPKARKR | 273 | Porphyromonas gingivalis | 30S ribosomal protein S9 |
| | | (Bacteroides gingivalis) | |
| RPPRPRPDER | 274 | Acidovorax sp. (strain JS42) | Integral membrane sensor |
| | | | signaltransduction histidine |
| | | | kinase |
| KPKKPKKEQR | 275 | Pasteurella multocida | Protein tonB |
| KPGKPRRARR | 276 | Burkholderia pseudomallei (strain | Methyl-accepting chemotaxis |
| | | 668) | protein |
| RPLRPRGSMR | 277 | Bos taurus (Bovine) | G-protein coupled bile acid |
| | | | receptor 1 |
| RPYRPKRTSR | 278 | Planctomyces maris DSM8797 | Ribosomal protein SI-like |
| | | | RNA-binding domain |
| KPKKPKHMKR | 279 | Arabidopsis thaliana (Mouse-ear | Calcium-dependent protein |
| | | cress) | kinaseisoform AK1 |
| RPPRPRKPTK | 280 | Comamonas testosteroni KF-1 | Class I peptide chain release |
| | | | factor |
| KPIKPRQFAK | 281 | Drosophila melanogaster (Fruitfly) | CG18190-PA |
| RPARPRRDPR | 282 | Pseudomonas mendocina (strain | Response regulator receiver |
| | | ymp) | protein |
| RPLRPRPTFR | 283 | Laccaria bicolor (strain S238N- | Putative protein |
| | | H82) (Bicoloured deceiver) | |
| | | (Laccaria laccata var. bicolor) | |
| RPGRPRRKPR | 284 | Saccharopolyspora Erythraea | Transposase |
| | | (strain NRRL 23338) | |
| RPDRPRGDSR | 285 | Culex quinguefasciatus (Southern | Male-specific transcription |
| | | house mosquito) | factorFRU-MB |
| RPSKPRLIPR | 286 | Bos taurus (Bovine) | ZNF689 protein |
| KPPKPRPPER | 287 | Methylobacterium sp. (strain4-46) | TonB family protein |
| KPSKPRLIAR | 288 | Rattus norvegicus (Rat) | Zinc finger protein 689 |
| KPKRPKVTRK | 289 | Cryptococcus neoformans | Histone-lysine N- |
| | | | methyltransferase, H3 lysine- |
| | | | 79 specific |
| RPMRPRLTDR | 290 | Phaeobacter gallaeciensis 2.10 | Glycosyltransferase involved |
| | | | in cellwall biogenesis-like |
| | | | protein |
| KPSKPRLIPR | 291 | Mus musculus (Mouse) | Zinc finger protein 689 |
| KPEKPKLPQR | 292 | Lodderomyces elongisporus (Yeast) | Putative protein |
| | | (Saccharomyces elongisporus) | |
| RPLRPRSRRK | 293 | Mus musculus (Mouse) | Testis-specific H1 histone |
| KPVRPRRLHR | 294 | Mycobacterium ulcerans (strain | Conserved hypothetical |
| | | Agy99) | membrane protein |
| RPKKPRKPRK | 295 | Monosiga brevicollis | Putative protein |
| | | (Choanoflagellate) | |
| RPVKPKDWRR | 296 | Hoeflea phototrophica DFL-43 | Hypothetical cytosolic protein |
| RPQRPRTTSK | 297 | Laccaria bicolor (strain S238N- | Putative protein |
| | | H82) (Bicoloured deceiver) | |
| | | (Laccaria laccata var. bicolor) | |
| KPDKPKEAKK | 298 | Mus musculus (Mouse) | Chorein |
| RPWRPRTFLR | 299 | Mycobacterium bovis | Probable oxidoreductase |
| | | | ephD |
| RPEKPKGKGK | 300 | Mus musculus (Mouse) | Coiled-coil domain- |
| | | | containingprotein 96 |
| KPAKPKGRER | 301 | Acidovorax avenae subsp.citrulli | Phage integrase family |
| | | (strain AAC00-1) | protein |
| RPSKPRPARK | 302 | Anaeromyxobacterde- | TfoX domain protein |
| | | halogenans 2CP-1 | |
| RPGRPRPRRR | 303 | Anaeromyxobacter sp. (strain | LigA |
| | | Fw109-5) | |
| KPVRPKRDFR | 304 | Caenorhabditis elegans | Protein T19H5.4 |
| KPLKPKLLQR | 305 | Ochrobactrum anthropi (strain | Dyp-type peroxidase family |
| | | ATCC 49188/DSM 6882/NCTC | |
| | | 12168) | |
| RPHRPRVKIK | 306 | Rhizobium leguminosarum bv. | NUDIX hydrolase |
| | | trifolii WSM1325 | |
| RPSRPKQIEK | 307 | Brugia malayi (Filarialnematode | Bopl-prov protein |
| | | worm) | |
| RPEKPRDRDR | 308 | Bos taurus (Bovine) | Cdc42 effector protein 1 |
| RPYKPKDKAK | 309 | Shewanella benthica KT99 | Transposase |
| KPKRPKGKGK | 310 | Suberites domuncula (Sponge) | Wiskott-Aldrich syndrome |
| | | | protein |
| KPKKPRAEKR | 311 | Vitis vinifera (Grape) | Chromosome chr9 scaffold 7 |
| RPVKPKYGPK | 312 | Victivallis vadensis ATCC BAA- | Ferrous iron transport protein |
| | | 548 | B |
| KPTKPRPKSK | 313 | Vibrio cholerae | R1pA-like lipoprotein |
| RPLKPKDAIR | 314 | Rhodopseudomonas palustris (strain | UPF0317 protein RPC 1666 |
| | | BisB18) | |
| KPVKPKKEKK | 315 | Streptococcus pneumoniae SP3- | BCR |
| | | BS71 | |
| RPMKPKDAIR | 316 | Rhodopseudomonas palustris (strain | UPF0317 protein RPB 3621 |
| | | HaA2) | |
| KPGRPRKRSR | 317 | Pseudomonas syringae pv. tomato | Insertion sequence |
| KPEKPKTKIR | 318 | Aspergillus niger | Contig An13c0060 |
| | | (strain CBS513.88/FGSC A1513) | |
| RPLRPRRRLR | 319 | Erythrobacter sp. SD-21 | Methyltransferase |
| KPEKPKAEVK | 320 | Comamonas testosteroni KF-1 | Sporulation related |
| RPPKPRFFDR | 321 | Azotobacter vinelandii | USG-1 protein homolog |
| RPDKPRADDR | 322 | Pseudomonas syringae pv. tomato | Translation initiation factor |
| | | | IF-2 |
| KPGKPKADAK | 323 | Homo sapiens (Human) | Zinc finger and homeobox |
| | | | protein 2 |
| RPRKPKVTSR | 324 | Rhodobacter sphaeroides (strain | Plasmid pRiA4b ORF-3 |
| | | ATCC 17025/ATH 2.4.3) | family protein |
| KPEKPKPAAK | 325 | Rhodobacter sphaeroides (strain | Peptidyl-tRNA hydrolase |
| | | ATCC 17029/ATH 2.4.9) | |
| KPEKPKGEAK | 326 | Rhodobacter sphaeroides (strain | Peptidyl-tRNA hydrolase |
| | | ATCC 17025/ATH 2.4.3) | |
| RPHRPRERER | 327 | Mus musculus (Mouse) | Probable protein phosphatase |
| | | | 1B-like |
| RPLRPRERER | 328 | Homo sapiens (Human) | Probable protein phosphatase |
| | | | 1B-like |
| KPKRPRATGR | 329 | Coprinopsis cinerea (strain | Putative protein |
| | | Okayama-7/130/FGSC | |
| | | 9003)(Inky cap | |
| | | fungus)(Honnographiella | |
| | | aspergillata) | |
| RPIKPRIFQK | 330 | Leptospira biflexa serovar Patoc | DNA methylase |
| | | (strain Patoc 1/Ames) | |
| RPTRPKPLQR | 331 | Synechocystis sp. (strain PCC6803) | UPF0026 protein s1r1464 |
| KPNKPKKLLR | 332 | Danio rerio (Zebrafish) | P2X purinoceptor |
| | | (Brachydanio rerio) | |
| KPRKPRQTKK | 333 | Candida glabrata (Yeast) | Mediator of RNA polymerase |
| | | (Torulopsis glabrata) | II transcription subunit 3 |
| KPYRPRGWEK | 334 | Pyrobaculum calidifontis (strain | Radical SAM domain protein |
| | | JCM 11548/VAI) | |
| RPPRPKQSEK | 335 | Lactobacillus brevis (strain ATCC | 50S ribosomal protein L3 |
| | | 367/JCM 1170) | |
| RPRRPRGRSR | 336 | Laccaria bicolor (strain S238N- | Hypothetical magnesium |
| | | H82) (Bicoloured deceiver) | transporter, CorA-like protein |
| RPVRPRPRGR | 337 | Limnobacter sp. MED105 | Conserved transposase-like |
| | | | protein |
| KPAKPPAPKK | 338 | Pseudoalteromonas tunicata D2 | 30S ribosomal subunit protein |
| | | | S3 |
| RPRKPRDKAK | 339 | Bradyrhizobium sp. (strain BTAi1/ | Transposase |
| | | ATCC BAA-1182) | |
| RPARPKTGGR | 340 | Roseobacter sp. AzwK-3b | Transposase, IS256 family |
| | | | protein |
| KPGRPKGRKK | 341 | Desulfococcus oleovorans (strain | Phosphotransferase |
| | | DSM 6200/Hxd3) | KptA/Tpt1 |
| KPDKPKQSQK | 342 | Paramecium tetraurelia | Chromosome undetermined |
| | | | scaffold 68 |
| KPLKPKSFSR | 343 | Desulfatibacillum alkenivorans AK- | Polyribonucleotidenucleotidyl |
| | | 01 | transferase |
| KPKKPRKLKK | 344 | Giardia lamblia ATCC 50803 | Spindle pole protein |
| RPVRPRRSRK | 345 | Halorhodospira halophila (strain | DNA-directed DNA |
| | | DSM 244/SL1) | polymerase |
| | | (Ectothiorhodospira halophila | |
| | | (strain DSM 244/SL1)) | |
| KPLRPRWAHK | 346 | Fervidobacterium nodosum (strain | Carbohydrate kinase, YjeF |
| | | ATCC 35602/DSM 5306/Rt17-B1) | related protein |
| RPARPKDKPK | 347 | Verminephrobacter eiseniae (strain | Integrase |
| | | EF01-2) | |
| RPAKPRDKPK | 348 | Acidovorax sp. (strain JS42) | Integrase |
| RPRRPRDKAK | 349 | Rhizobium meliloti (Sinorhizobium | Transposase TRm23a |
| | | meliloti) | |
| KPMRPRLPRR | 350 | Serratia entomophila | RepA |
| RPERPRPTLR | 351 | Monosiga brevicollis | Putative protein |
| | | (Choanoflagellate) | |
| RPYKPRDKAK | 352 | Rhodobacter sphaeroides (strain | Integrase, catalytic region |
| | | ATCC 17025/ATH 2.4.3) | |
| RPRKPKDKAK | 353 | Escherichia coli O1:K1/APEC | TnpA |
| RPFRPRDKAK | 354 | Acidiphilium cryptum (strain JF-5) | Integrase, catalytic region |
| RPYKPKDKSK | 355 | Marinobacter aquaeolei (strain | Integrase |
| | | ATCC 700491/DSM 11845/VT8) | |
| | | (Marinobacter | |
| | | hydrocarbonoclasticus (strain DSM | |
| | | 11845)) | |
| RPRKPKDKSK | 356 | Shewanella putrefaciens 200 | Integrase |
| KPARPKPQRR | 357 | Roseovarius sp. TM1035 | Pseudouridine synthase |
| KPIKPKYHDR | 358 | Thermoplasma volcanium | CCA-adding enzyme |
| RPPKPRKHPR | 359 | Leptospirillum sp. Group IIUBA | Cytochrome c |
| KPTKPKTCIK | 360 | Nematostella vectensis (Starletsea | Putative protein |
| | | anemone) | |
| RPAKPKRTIK | 361 | Carnobacterium sp. AT7 | Integral membrane protein |
| RPRRPRVGRR | 362 | Saccharopolyspora Erythraea | Ribose transport ATP-binding |
| | | (strain NRRL 23338) | protein |
| KPTKPRASRK | 363 | Caenorhabditis elegans | Protein ZK177.1 |
| KPPRPRPRPR | 364 | Coprinopsis cinerea (strain | Putative protein |
| | | Okayama-7/130/FGSC 9003) | |
| | | (Inky cap fungus) (Hormographiella | |
| | | aspergillata) | |
| KPQKPKVKPK | 365 | Danio rerio (Zebrafish) | Zgc:162349 protein |
| | | (Brachydanio rerio) | |
| RPGRPKNLGK | 366 | Ajellomyces capsulata (strain NAm1 | Putative protein |
| | | /WU24) (Darling's disease fungus) | |
| | | (Histoplasma capsulatum) | |
| RPSRPKGTPR | 367 | Aspergillus niger | Similarity to hypothetical |
| | | (strain CBS513.88/FGSC A1513) | proteinAt2g17590 |
| KPRKPKPPIR | 368 | Roseobacter litoralis Och 149 | Penicillin-insensitive murein |
| | | | endopeptidase |
| RPRRPRRMRR | 369 | Methylobacterium sp. (strain 4-46) | von Willebrand factor type A |
| KPAKPKWFKR | 370 | Oryza sativa subsp. indica (Rice) | Glucose-6-phosphate 1- |
| | | | dehydrogenase |
| RPDRPRGGYR | 371 | Chloroflexus aurantiacus (strain | ATPase associated with |
| | | ATCC 29366/DSM 635/J-10-f1) | various cellular activities |
| | | | AAA 5 |
| RPDRPRGSYR | 372 | Chloroflexus aggregans DSM 9485 | ATPase associated with |
| | | | various cellular activities, |
| | | | AAA 5 |
| KPGKPRPSYK | 373 | Leptospira biflexa serovar Patoc | Zn-dependent peptidase |
| | | (strain Patoc 1/Ames) | |
| KPRRPRKAER | 374 | Halorhodospira halophila (strain | DNA translocase FtsK |
| | | DSM 244/SL1) | |
| | | (Ectothiorhodospira halophila | |
| | | (strain DSM 244/SL1)) | |
| RPDRPKIDIR | 375 | Lactobacillus helveticus | Transcription repressor of |
| | | (strain DPC 4571) | beta-galactosidase gene |
| RPARPKCHKR | 376 | Coprinopsis cinerea (strain | Putative protein |
| | | Okayama-7/130/FGSC 9003) | |
| | | (Inky cap fungus) (Hormographiella | |
| | | aspergillata) | |
| KPGRPKRKRR | 377 | Brugia malayi (Filarialnematode | N terminus of Rad21/Rec8 |
| | | worm) | likeprotein |
| KPRKPRATKK | 378 | Lodderomyces elongisporus (Yeast) | Putative protein |
| | | (Saccharomyces elongisporus) | |
| KPFRPRVAGR | 379 | Rhodobacter capsulatus | Nitrogenase iron- |
| | | (Rhodopseudomonas capsulata) | molybdenumcofactor |
| | | | biosynthesis protein nifE |
| KPVRPKDELR | 380 | Vitis vinifera (Grape) | Chromosome chr4 scaffold 6 |
| KPVRPKDELR | 381 | Cucumis sativus (Cucumber) | Monogalactosyldiacylglycerol |
| | | | synthase, chloroplastic |
| RPEKPKSIGK | 382 | Flavobacterium psychrophilum | Probable ABC-type multidrug |
| | | (strain JIP02/86/ATCC 49511) | transport system, ATPase and |
| | | | permease components |
| KPPKPRTAKK | 383 | Chlorokybus atmophyticus | Pyruvate dehydrogenase |
| | | (Soilalga) | Elcomponent subunit beta |
| RPWRPRFDAR | 384 | Agrobacterium tumefaciens | Orf Bo191 |
| KPVRPKVELR | 385 | Arabidopsis thaliana (Mouse-ear | Monogalactosyldiacylglycerol |
| | | cress) | synthase 1, chloroplastic |
| KPRRPRNKKR | 386 | Desulfatibacillum alkenivorans | PSP1 domain protein |
| | | AK-01 | |
| KPEKPKLMLR | 387 | Hyperthermus butylicus (strain | Phenylalanyl-tRNA |
| | | DSM 5456/JCM 9403) | synthetase beta chain |
| RPPRPRHLVR | 388 | Opitutaceae bacterium TAV2 | Thiamine pyrophosphate |
| | | | proteinTPP binding domain |
| | | | protein |
| KPPRPKEKKR | 389 | Neosartorya fischeri (strain ATCC | WD domain protein |
| | | 1020/DSM 3700/NRRL 181) | |
| | | (Aspergillus fischerianus (strain | |
| | | ATCC 1020/DSM 3700/NRRL 181)) | |
| RPLKPRITNR | 390 | Mus musculus (Mouse) | Transient receptor potential |
| | | | cation channel subfamily V |
| | | | member 6 |
| RPLKPRTNNR | 391 | Homo sapiens (Human) | Transient receptor potential |
| | | | cation channel, subfamily V, |
| | | | member 6 (TRPV6) |
| RPGRPRKLPR | 392 | Homo sapiens (Human) | Zinc finger protein 335 |
| KPWRPRFDAK | 393 | Xanthobacter autotrophicus (strain | Phage integrase family |
| | | ATCC BAA-1158/Py2) | protein |
| KPAKPKPLPK | 394 | Saccharopolyspora erythraea (strain | Endo-1, 4-beta-glucanase |
| | | NRRL 23338) | |
| KPRRPKTGAR | 395 | Bradyrhizobium sp. (strain BTAi1/ | Transposase |
| | | ATCC BAA-1182) | |
| RPPKPKSQTK | 396 | Cyanothece sp. CCY 0110 | Translation initiation factor |
| | | | IF-2 |
| RPRRPRRQEK | 397 | Streptococcus pneumoniae SP3-BS71 | Ribosomal protein S1 |
| RPRRPKRQEK | 398 | Streptococcus gordonii (strain | 30S ribosomal protein Si |
| | | Challis/ATCC 35105/CHI/DL1 | |
| | | /V288) | |
| RPCRPRDPGR | 399 | Acidovorax avenae subsp.citrulli | LigA |
| | | (strain AAC00-1) | |
| KPVRPRGPGR | 400 | Homo sapiens (Human) | Potassium channel subfamily |
| | | | K member 4 |
| RPRRPRSVDR | 401 | Salinispora arenicola | Peptidase M50 |
| | | (strain CNS-205) | |
| RPPRPRHVQR | 402 | Physcomitrella patens subsp.patens | Putative protein |
| KPSRPRGPGR | 403 | Mus musculus (Mouse) | Potassium channel subfamily |
| | | | K member 4 |
| RPSRPKGMPR | 404 | Ajellomyces capsulata (strain | Putative protein |
| | | NAm1/WU24) (Darling's disease | |
| | | fungus) (Histoplasma capsulatum) | |
| RPAKPRPAAR | 405 | Arthrobacter sp. (strain FB24) | Sodium/hydrogen exchanger |
| RPSKPRSHPK | 406 | Paramecium tetraurelia | Chromosome undetermined |
| | | | scaffold 52 |
| RPMRPRCSLR | 407 | Equine herpesvirus 1 (strain Ab4p) | Trans-acting transcriptional |
| | | (EHV-1) (Equine abortion virus) | protein ICPO |
| RPLKPKKPKK | 408 | Vibrio cholerae V52 | ATP-dependent RNA helicase |
| | | | Rh1E |
| RPIKPKKPKK | 409 | Vibrio splendidus 12B01 | ATP-dependent RNA helicase |
| | | | Rh1E |
| KPKKPKKPKK | 410 | Reinekea MED297 | ATP-dependent RNA |
| | | | helicase, DEAD box family |
| | | | Protein |
| RPRRPRPLDR | 411 | Salinispora tropica (strain ATCC | Peptidase M50 |
| | | BAA-916/DSM 44818/CNB-440) | |
| RPIRPKKGVR | 412 | Ajellomyces capsulata (strain | Putative protein |
| | | NAm1/WU24) (Darling's disease | |
| | | fungus) (Histoplasma capsulatum) | |
| RPVKPKPERR | 413 | Leishmania infantum | Endo/exonuclease Mrel 1 |
| RPVKPKLERR | 414 | Leishmania braziliensis | Endo/exonuclease Mrel 1 |
| KPQRPRSAPR | 415 | Mus musculus (Mouse) | FCH domain only protein 1 |
| RPARPKGKAK | 416 | Anaeromyxobacter dehalogenans | Tryptophan synthase, beta |
| | | 2CP-1 | subunit |
| KPKKPRLDKR | 417 | Culex quinquefasciatus (Southern | Peter pan |
| | | house mosquito) | |
| RPSKPRGVER | 418 | Aspergillus clavatus | DnaJ domain protein |
| RPRKPRYFNR | 419 | Aspergillus niger (strain CBS | Contig An07c0220 |
| | | 513.88/FGSC A1513) | |
| RPQKPRADFR | 420 | Oceanibulbus indolifex HEL-45 | DNA primase |
| RPRRPRPPAR | 421 | Frankia sp. (strain EAN1pec) | YibE/F family protein |
| KPTKPRTTTK | 422 | Leishmania infantum | Histone hl-like protein |
| RPGRPRWAGR | 423 | Mycobacterium tuberculosis | PE-PGRS family protein |
| | | (strain F11) | |
| RPAKPRSQQK | 424 | Synechococcus sp. RS9917 | Translation initiation factor |
| RPAKPKSQQR | 425 | Synechococcus sp. (strain CC9605) | Translation initiation factor |
| | | | IF-2 |
| RPRRPKDRIR | 426 | Rhizobium leguminosarum bv. | AAA ATPase central domain |
| | | trifolii WSM1325 | protein |
| RPAKPKSTKK | 427 | Prochlorococcus marinus (strain | Translation initiation factor |
| | | MIT 9211) | IF-2 |
| KPPKPRGIKR | 428 | Monosiga brevicollis | Putative protein |
| | | (Choanoflagellate) | |
| RPAKPKSQKK | 429 | Prochlorococcus marinus (strain | Translation initiation factor |
| | | MIT 9303) | IF-2 |
| RPSKPKSQQK | 430 | Synechococcus sp. (strainWH7803) | Translation initiation factor |
| | | | IF-2 |
| RPRRPRRSSR | 431 | Plesiocystis pacifica SIR-1 | Nuclease SbcCD, C subunit |
| RPSKPRTKHK | 432 | Synechococcus sp. (strainCC9311) | Translation initiation factor |
| | | | IF-2 |
| KPYRPKVRRR | 433 | Saccharomyces cerevisiae (strain | Conserved protein |
| | | YJM789) (Baker's yeast) | |
| RPRKPRKSAR | 434 | Heliobacterium modesticaldum | Transcription-repair coupling |
| | | (strain ATCC 51547/feel) | factor |
| RPGKPKASKK | 435 | Prochlorococcus marinus | Translation initiation factor |
| | | | IF-2 |
| KPKKPKPTDK | 436 | Rhodobacterales bacterium | Exodeoxyribonuclease 7 |
| | | HTCC2150 | largesubunit |
| KPKKPRPKAR | 437 | Plesiocystis pacifica SIR-1 | Heavy metal efflux pump |
| RPAKPRAQQK | 438 | Synechococcus sp. (strain WH7805) | Translation initiation factor |
| | | | IF-2 |
| KPGRPRVGHR | 439 | Acidothermus cellulolyticus (strain | Uroporphyrinogen-III |
| | | ATCC 43068/11B) | synthase /uroporphyrinogen- |
| | | | III C-methyltransferase |
| KPKKPKKPKR | 440 | Clavibacter michiganensis subsp. | Conjugal transfer protein |
| | | michiganensis (strain NCPPB 382) | |
| KPEKPKEKPR | 441 | Sagittula stellata E-37 | ABC transporter |
| RPNRPKKKNK | 442 | Nematostella vectensis (Starletsea | Putative protein |
| | | anemone) | |
| RPHRPRSNTR | 443 | Coprinopsis cinerea (strain | Putative protein |
| | | Okayama-7/130/FGSC 9003) | |
| | | (Inky cap fungus) (Hormographiella | |
| | | aspergillata) | |
| KPHRPKRGGK | 444 | Rattus norvegicus (Rat) | Regulating synaptic |
| | | | membraneexocytosis protein |
| | | | 1 |
| RPGRPRIPRR | 445 | Brugia malayi (Filarialnematode | LF3 |
| | | worm) | |
| RPAKPKAQQR | 446 | Synechococcus sp. (strain CC9902) | Translation initiation factor |
| | | | IF-2 |
| RPSKPKVGKR | 447 | Prochlorococcus marinus (strain | Translation initiation factor |
| | | NATL1A) | IF-2 |
| RPKRPRECTK | 448 | Ostreococcus lucimarinus (strain | Putative protein |
| | | CCE9901) | |
| RPLRPKYLYR | 449 | Laccaria bicolor (strain S238N- | Putative protein |
| | | H82) (Bicoloured deceiver) | |
| | | (Laccaria laccata var. bicolor) | |
| RPLKPRDTEK | 450 | Drosophila melanogaster (Fruitfly) | CG34411-PA |
| KPAKPKSKSK | 451 | Aspergillus fumigatus | Conserved proline-rich |
| | | (Sartoryafumigata) | protein |
| RPDKPKKQEK | 452 | Ostreococcus lucimarinus (strain | Putative protein |
| | | CCE9901) | |
| RPAKPKGKYK | 453 | Bacillus selenitireducens MLS10 | ABC transporter related |
| KPTRPKTKGK | 454 | Laccaria bicolor (strain S238N- | Putative protein |
| | | H82) (Bicoloured deceiver) | |
| | | (Laccaria laccata var. bicolor) | |
| RPRKPRFFNR | 455 | Homo sapiens (Human) | Protein C19orf29 |
| RPPRPKAMDK | 456 | Chlamydomonas reinhardtii | Putative protein |
| KPAKPKSACR | 457 | Giardia lamblia ATCC 50803 | TFIIH basal transcription |
| | | | factorcomplex helicase |
| | | | subunit |
| KPEKPKSKNK | 458 | Neosartorya fischeri (strain ATCC | Conserved proline-rich |
| | | 1020/DSM 3700/NRRL 181) | protein |
| | | (Aspergillus fischerianus (strain | |
| | | ATCC 1020/DSM 3700/NRRL 181)) | |
| KPYKPKKIIK | 459 | Homo sapiens (Human) | Probable E3 ubiquitin-protein |
| | | | ligaseMYCBP2 |
| KPIKPKFKER | 460 | Paramecium tetraurelia | Chromosome undetermined |
| | | | scaffold 21 |
| RPVKPKFIGR | 461 | Burkholderia vietnamiensis (strain | Methionine synthase (B12- |
| | | G4/LMG 22486) (Burkholderia | dependent) |
| | | cepacia (strain R1808)) | |
| KPRRPKHTRK | 462 | Danio rerio (Zebrafish) | Novel protein similar to |
| | | (Brachydanio rerio) | vertebrateasparagine-linked |
| | | | glycoslyation 9homolog |
| | | | (ALG9) (Zgc:63820) |
| KPLRPKLKTR | 463 | Salinispora arenicola (strain CNS- | Thioester reductase domain |
| | | 205) | |
| KPSRPRPKSR | 464 | Homo sapiens (Human) | SET domain-containing |
| | | | protein 5 |
| KPEKPKTQKR | 465 | Bacillus subtilis | MutS2 protein |
| RPHRPKIRSR | 466 | Laccaria bicolor (strain S238N- | Putative protein |
| | | H82) (Bicoloured deceiver) | |
| | | (Laccaria laccata var. bicolor) | |
| RPVRPRRVGK | 467 | Rhodobacterales bacterium | Lipoprotein |
| | | HTCC2654 | |
| RPQRPKVIER | 468 | Homo sapiens (Human) | Protein KIAA0586 |
| RPRRPRREDK | 469 | Aspergillus niger | Contig AnO2c0010 |
| | | (strain CBS513.88/FGSC A1513) | |
| KPRKPRAPRK | 470 | Caenorhabditis briggsae | DNA topoisomerase |
| KPKRPRKKMR | 471 | Danio rerio (Zebrafish) | Protein KRI1 homolog |
| | | (Brachydanio rerio) | |
| KPKKPRAPRK | 472 | Geobacter uraniireducens (strain | Ribonuclease E |
| | | Rf4) (Geobacter uraniumreducens) | |
| KPQRPRKRLR | 473 | Coprinopsis cinerea (strain | Putative protein |
| | | Okayama-7/130/FGSC 9003) | |
| | | (Inky cap fungus) (Hormographiella | |
| | | aspergillata) | |
| KPARPKRQPR | 474 | Shewanella pealeana (strain ATCC | Ribonuclease, Rne/Rng |
| | | 700345/ANG-SQ1) | family |
| KPKRPRGGRK | 475 | Geobacter bemidjiensis (strain Bern | Ribonuclease, Rne/Rng |
| | | /ATCC BAA-1014/DSM 16622) | family |
| RPLRPKAHLK | 476 | Solanum lycopersicum (Tomato) | Beta-galactosidase |
| | | (Lycopersicon esculentum) | |
| KPERPRTAGR | 477 | Homo sapiens (Human) | Protein Shroom2 |
| KPAKPRRSRK | 478 | Sinorhizobium medicae (strain | Ribonuclease, Rne/Rng |
| | | WSM419) | family |
| RPSRPRKHRK | 479 | Sclerotinia sclerotiorum (strain | Putative protein |
| | | ATCC 18683/1980/Ss-1) (White | |
| | | mold) (Whetzelinia sclerotiorum) | |
| KPAKPKMSVK | 480 | Marinomonas (strain MWYL1) | Ribonuclease, Rne/Rng |
| | | | family |
| RPARPRRSSR | 481 | Marinobacter algicola DG893 | Ribonucleases G and E |
| KPRKPRPPSK | 482 | Monosiga brevicollis | Putative protein |
| | | (Choanoflagellate) | |
| RPSKPKKPKK | 483 | Theileria parva | 104 kDa microneme/rhoptry |
| | | | antigen |
| RPSRPKPIGK | 484 | Coprinopsis cinerea (strain | Putative protein |
| | | Okayama-7/130/FGSC 9003) | |
| | | (Inky cap fungus) (Hormographiella | |
| | | aspergillata) | |
| RPGRPRPPDR | 485 | Rhodobacter sphaeroides (strain | Diguanylate |
| | | ATCC 17025/ATH 2.4.3) | phosphodiesterase |
| RPGRPRRERR | 486 | Laccaria bicolor (strain S238N- | Putative protein |
| | | H82) (Bicoloured deceiver) | |
| | | (Laccaria laccata var. bicolor) | |
| KPEKPKPKNK | 487 | Aspergillus clavatus | DNA repair helicase rad5, 16 |
| RPRKPKTIAK | 488 | Physcomitrella patens subsp. patens | Putative protein |
| RPPKPRRPER | 489 | Danio rerio (Zebrafish) | Bone morphogenetic protein |
| | | | receptor type II b |
| | | | (Serine/threonine kinase) |
| RPFKPKANKK | 490 | Paramecium tetraurelia | Chromosome undetermined |
| | | | scaffold 35 |
| RPTRPKVIKK | 491 | Paramecium tetraurelia | Chromosome undetermined |
| | | | scaffold 17 |
| RPLRPRRKGR | 492 | Homo sapiens (Human) | Coiled-coil and C2 domain- |
| | | | containing protein 2A |
| KPTKPKTKKR | 493 | Helicoverpa armigeragranulovirus | DNA polymerase |
| RPLKPKLYEK | 494 | Sphingomonas wittichii | LVIVD repeat protein |
| | | (strain RW1/DSM 6014/JCM 10273) | |
| KPRKPREPKK | 495 | Culex quinquefasciatus (Southern | Chromodomain helicase DNA |
| | | house mosquito) | binding protein |
| KPLKPRLVGR | 496 | Monosiga brevicollis | Putative protein |
| | | (Choanoflagellate) | |
| RPDRPRARDR | 497 | Homo sapiens (Human) | Spectrin beta chain, brain 3 |
| RPRRPRGNAR | 498 | Coprinopsis cinerea (strain | Putative protein |
| | | Okayama-7/130/FGSC 9003) | |
| | | (Inky cap fungus) (Hormographiella | |
| | | aspergillata) | |
| RPKKPKGNFK | 499 | Berne virus (BEV) | Replicase polyprotein lab |

c. Transduction Peptides with Basic Charges on One Face of an Alpha-Helix

The modified therapeutic antibodies provided herein can contain a PTD that has an amino acid sequence according to the prosite pattern B₁-X₁-B₂-B₃-B₄-X₂-B₅-B₆-X₃-X₄-B₇ (SEQ ID NO: 1046), where B₁, B₂, B₃, B₄, B₅, B₆, and B₇ are each independently lysine or arginine and X₁, X₂, X₃ and X₄ are each independently any amino acid (amino acid pattern also can be expressed as [K/R]-X-[K/R](3)-X-[K/R](2)-X(2)-[K/R]). In some examples, X₁, X₂, X₃ and X₄ are each independently any amino acid except proline. In other examples, X₁, X₂, X₃ and X₄ are each independently any non-basic amino acid except proline. In further examples, X₁, X₂, X₃, and X₄ are each independently serine, leucine, alanine, asparagine, aspartic acid and glycine. Exemplary PTDs, which can be conjugated to a therapeutic antibody provided herein, include, but are not limited to, PTDs found in naturally occurring proteins, where the PTD has the amino acid pattern B₁-X₁-B₂-B₃-B₄-X₂-B₅-B₆-X₃-X₄-B₇ described above.

In some examples, the PTD that is attached to the therapeutic antibody is a peptide having an amino acid sequence, B₁-X₁-B₂-B₃-B₄-X₂-B₅-B₆-X₃-X₄-B₇ (where B₁, B₂, B₃, B₄, B₅, B₆, and B₇ are each independently lysine or arginine and X₁, X₂, X₃ and X₄ are each independently any amino acid) and further containing one or more additional amino acids that are located at the N-terminus and/or C-terminus of the peptide, where each additional amino acid is independently arginine or lysine. In some examples, the PTD that is attached to the therapeutic antibody is a peptide having an amino acid sequence, B₁-X₁-B₂-B₃-B₄-X₂-B₅-B₆-X₃-X₄-B₇ (where B₁, B₂, B₃, B₄, B₅, B₆, and B₇ are each independently lysine or arginine and X₁, X₂, X₃, and X₄ are each independently any amino acid) and further containing 1, 2, 3, 4, 5, 6, 7, 8, 9, 10 or more additional amino acids that are located at the N-terminus and/or C-terminus of the peptide, where each additional amino acid is independently arginine or lysine. Typically, the one or more additional amino acids are located at the C-terminus of the peptide. When additional amino acids are located at the N-terminus of a modified PTD peptide, it is understood that the modified PTD contains amino acids analogous to the unmodified PTD, for example, the modified PTD retains the amino acid sequence B₁-X₁-B₂-B₃-B₄-X₂-B₅-B₆-X₃-X₄-B₇ (where B₁, B₂, B₃, B₄, B₅, B₆, and B₇ are each independently lysine or arginine and X₁, X₂, X₃, and X₄ are each independently any amino acid.

In some examples, the PTD is selected from among PTDs provided in Table 7 and SEQ ID NOS:500-855 and 867. The PTDs identified Table 7 and SEQ ID NOS:500-855 and 867 can be conjugated to any antibody or antigen-binding fragment thereof provided herein or known in the art. In particular examples, the PTDs identified Table 7 and SEQ ID NOS:500-855 and 867 can be conjugated to a neutralizing antibody (e.g. an antiviral, an antibacterial, or an antifungal neutralizing antibody). In some examples, the PTDs identified Table 7 and SEQ ID NOS:500-855 and 867 can be conjugated to an antibody that binds to the surface of a pathogen, such as a viral, bacterial or fungal pathogen. For example, the PTDs identified Table 7 and SEQ ID NOS:500-855 and 867 can be conjugated to an antibody that binds to a glycoprotein on the surface of the pathogen.

In some examples, the PTD selected from among the peptides provided in Table 7 and SEQ ID NOS:500-855 and 867 is a variant, such as a variant that increases the charge of the peptide. For example, a PTD of Table 7 or SEQ ID NOS:500-855 and 867 can be modified to replace one or more lysine residues with an arginine. In some examples, a C-terminal lysine residue is replaced with an arginine.

TABLE 7
Protein Transduction Peptides with Display of Basic Charges on One
Face of an Alpha-helix According to Formula:
[K/R]-X-[K/R](3)-X-[K/R](2)-X(2)-[K/R]

| Protein | SEQ | | |
|---|---|---|---|
| Transduction | ID | | |
| Domain (PTD) | NO | Source Organism | Source Protein |
| RHKKRGRRLSR | 500 | Magnetococcus sp. (strain MC-1) | 50S ribosomal protein L17 |
| RHRKKGKKIGR | 501 | Salinibacter ruber (strain | 50S ribosomal protein L17 |
| | | DSM13855) | |
| KDKKKEKKKDK | 502 | Paramecium tetraurelia | Chromosome undetermined |
| | | | scaffold 13 |
| KTRRRPRRSQR | 503 | Human T-cell leukemia virus I | Protein Rex |
| | | (strain Japan ATK-1 subtype A) | |
| | | (HTLV-1) | |
| KKRKKKRKNSK | 504 | Oceanobacillus iheyensis | DNA translocase ftsK |
| RSRRKSRRNGR | 505 | Homo sapiens (Human) | Probable E3 ubiquitin- |
| | | | protein ligase HERC5 |
| KDKRKDKRKDK | 506 | Mus musculus (Mouse) | C14orf45 homolog |
| KNKRKGRKTRR | 507 | Streptococcus mutans | DNA translocase ftsK |
| KNKKRQRRHAR | 508 | Pediococcus pentosaceus | 50S ribosomal protein L18 |
| | | (strainATCC 25745/183-1w) | |
| KARRRIKRWRR | 509 | Pseudotsuga menziesii (Douglas- | Unknown protein 5 |
| | | fir) | |
| RSKKKIRKNVR | 510 | Pinus koraiensis (Korean pine) | 50S ribosomal protein L32 |
| KTRKKLRKHPR | 511 | Pyrococcus abyssi | 50S ribosomal protein L21e |
| RTRKKLRKKPR | 512 | Methanopyrus kandleri | 50S ribosomal protein L21e |
| RERRRTKRRRR | 513 | Arabidopsis thaliana (Mouse- | F-box protein At4g19940 |
| | | earcress) | |
| KRRRRVRKKIR | 514 | Pelotomaculum | 505 ribosomal protein L18 |
| | | thermopropionicum (strain | |
| | | DSM13744/JCM 10971/SI) | |
| KWKRKRKKILR | 515 | Listeria welshimeri serovar 6b | Putative protein |
| | | (strain ATCC 35897/DSM 20650/ | |
| | | SLCC5334) | |
| KGKKRRRRGRK | 516 | Murex brandaris (Purple | Sperm protamine P3 |
| | | dyemurex) | |
| RPRRRCRRRIR | 517 | Orcinus orca (Killer whale) | Sperm protamine-P1 |
| KNKKKLKKKLR | 518 | Danio rerio (Zebrafish) | UPF0384 protein CGI-117 |
| | | (Brachydanio rerio) | homolog |
| KEKKRKKKRLR | 519 | Paramecium tetraurelia | Chromosome undetermined |
| | | | scaffold 13 |
| KRKKKGRKIPK | 520 | Homo sapiens (Human) | Tetratricopeptide repeat |
| | | | protein 26 |
| KSKKKLKKDKK | 521 | Kluyveromyces lactis (Yeast) | ATP-dependent RNA |
| | | (Candida sphaerica) | helicase DBP3 |
| RSKKRCRRCRR | 522 | Eledone cirrhosa (Curledoctopus) | Cysteine-rich protamine |
| | | (Ozaena cirrosa) | |
| KERRRERKKER | 523 | Oryza sativa subsp. Japonica | Zinc finger CCCH domain- |
| | | (Rice) | containing protein 16 |
| KDKKKIKKKSK | 524 | Paramecium tetraurelia | Chromosome undetermined |
| | | | scaffold 15 |
| RCRRKGRRISR | 525 | Scyliorhinus canicula | Spermatid-specific protein |
| | | (Spotteddogfish) (Spotted | S1 |
| | | catshark) | |
| RGRRRGRRRGR | 526 | Octopus vulgaris (Octopus) | Sperm protamine P1 |
| KRKRKAKKRRK | 527 | Homo sapiens (Human) | Transmembrane protein |
| | | | TTMA |
| KYKKRVRRSSR | 528 | Gnetum parvifolium | 30S ribosomal protein S4 |
| KHKKKDKKQKK | 529 | Paramecium tetraurelia | Chromosome undetermined |
| | | | scaffold 24 |
| KRKRRSKKINK | 530 | Saccharomyces cerevisiae (Baker's | Protein INO4 |
| | | yeast) | |
| RQRRRARRRWR | 531 | Simian immunodeficiency virus | Protein Rev |
| | | agm.vervet (isolate AGM3) (SIV- | |
| | | agm.ver) (Simian | |
| | | immunodeficiency virus African | |
| | | green monkey vervet) | |
| RQRRRARRRWK | 532 | Simian immunodeficiency virus | Protein Rev |
| | | agm.vervet (isolate AGM3) (SIV- | |
| | | agm.ver) (Simian | |
| | | immunodeficiency virus African | |
| | | green monkey vervet) | |
| RYRRRQRRSRR | 533 | Lagorchestes hirsutus | Sperm protamine-P1 |
| | | (Rufoushare-wallaby) (Western | |
| | | hare-wallaby) | |
| KSKKRLRRLRK | 534 | Sorangium cellulosum (strain | 50S ribosomal protein L35 |
| | | Soce56) (Polyangium cellulosum | |
| | | (strain So ce56)) | |
| RVKKREKKEEK | 535 | Human adenovirus F serotype | Minor core protein |
| | | 40(HAdV-40) (Human adenovirus | |
| | | 40) | |
| KERKKDKKEKK | 536 | Neurospora crassa | ATP-dependent RNA |
| | | | helicase dbp-3 |
| KTKKKLKKQKK | 537 | Saccharomyces cerevisiae (Baker's | Myb domain-containing |
| | | yeast) | protein YDR026C |
| KPRKKTRKIVK | 538 | Paramecium tetraurelia | Chromosome undetermined |
| | | | scaffold 106 |
| RDRRRGRKCGR | 539 | Mus musculus (Mouse) | 39S ribosomal protein L15 |
| KSRKKTKKGRK | 540 | Ashbya gossypii (Yeast) | ATP-dependent RNA |
| | | (Eremothecium gossypii) | helicase DRS1 |
| RGRRRGRKCGR | 541 | Homo sapiens (Human) | 39S ribosomal protein L15 |
| KDKKKHKKRRR | 542 | Pichia stipitis (Yeast) | Histone H2A.Z-specific |
| | | | chaperone CHZ1 |
| RSKRRGRRSTK | 543 | Drosophila melanogaster | Polycomb protein esc |
| | | (Fruitfly) | |
| RSKRRGRRSHK | 544 | Drosophila virilis (Fruit fly) | Polycomb protein esc |
| RERRRFKRELR | 545 | Aquifex aeolicus | Protein aq 1791 |
| KKRRRIRRSQK | 546 | Archaeoglobus fulgidus | Protein AF 2193 |
| RDRKRRKRDFR | 547 | Prochlorococcus marinus | 50S ribosomal protein L20 |
| RDRRKMKREFR | 548 | Oltmannsiellopsis viridis | 50S ribosomal protein L20 |
| | | (Marineflagellate) | |
| RDRRRKKRDFR | 549 | Campylobacter jejuni subsp.jejuni | 50S ribosomal protein L20 |
| | | serotype 0:6 (strain 81116/NCTC | |
| | | 11828) | |
| RDRRKRKRDFR | 550 | Acaryochloris marina (strain | 50S ribosomal protein L20 |
| | | MBIC 11017) | |
| RWRRRYRRWRR | 551 | Torque teno virus (isolate | Capsid protein |
| | | Human/Finland/He132/2002)(TTV) | |
| | | (Torque teno virus genotype 6) | |
| RDRRRRKRDFR | 552 | Chloroflexus aurantiacus | 50S ribosomal protein L20 |
| | | (strainATCC 29366/DSM 635/J- | |
| | | 10-f1) | |
| RDRKRKKRTFR | 553 | Bradyrhizobium japonicum | 50S ribosomal protein L20 |
| RPRRRGRRAGR | 554 | Homo sapiens (Human) | Homeobox protein HMX1 |
| KHRRKEKRSSR | 555 | Xenopus laevis (African clawed | Arginine/serine-rich coiled- |
| | | frog) | coil protein 2 |
| KEKKKEKKELK | 556 | Dictyostelium discoideum | Exocyst complex |
| | | (Slimemold) | component 6 |
| REKKKSKRRKR | 557 | Homo sapiens (Human) | Zinc finger CCCH domain- |
| | | | containing protein 6 |
| REKKRLRKEER | 558 | Schizosaccharomyces pombe | tRNA (guanine-N(1)-)- |
| | | (Fission yeast) | methyltransferase |
| RKKKRVRRRNK | 559 | Saccharomyces cerevisiae (Baker's | Protein YLR137W |
| | | yeast) | |
| RERKRLKKAKR | 560 | Sclerotinia sclerotiorum | ATP-dependent RNA |
| | | (strainATCC 18683/1980/Ss-1) | helicase dbp3 |
| | | (White mold) (Whetzelinia | |
| | | sclerotiorum) | |
| RERKRQRRSSR | 561 | Danio rerio (Zebrafish) | Probable ATP-dependent |
| | | (Brachydanio rerio) | RNA helicase DDX46 |
| KNKKKSKKKNK | 562 | Neurospora crassa | Glycylpeptide N- |
| | | | tetradecanoyl transferase |
| KERKKAKKAKK | 563 | Gibberella zeae (Fusarium | ATP-dependent RNA |
| | | graminearum) | helicase DBP3 |
| RERKRLKKEKK | 564 | Botryotinia fuckeliana | ATP-dependent RNA |
| | | (strainB05.10) (Noble rot fungus) | helicase dbp3 |
| | | (Botrytis cinerea) | |
| RKRKKRKKGEK | 565 | Gallus gallus (Chicken) | Bromodomain-containing |
| | | | protein 7 |
| KQKRKEKRKRK | 566 | Bos taurus (Bovine) | RNA (guanine-9-)- |
| | | | methyltransferase domain- |
| | | | containing protein 2 |
| KEKRKEKRKRK | 567 | Mus musculus (Mouse) | RNA (guanine-9-)- |
| | | | methyltransferase domain- |
| | | | containing protein 2 |
| RKRRKHKKRKR | 568 | Debaryomyces hansenii (Yeast) | Transcription elongation |
| | | (Torulaspora hansenii) | factor SPT6 |
| RLKKRIRKLER | 569 | Rattus norvegicus (Rat) | 39S ribosomal protein L40 |
| RLKRKIRKLEK | 570 | Homo sapiens (Human) | 39S ribosomal protein L40 |
| RLKKRIRKLEK | 571 | Mus musculus (Mouse) | 39S ribosomal protein L40 |
| RQRKKPRRERR | 572 | Yarrowia lipolytica (Candida | Transcription elongation |
| | | lipolytica) | factor SPT5 |
| KKRKKHKKKSK | 573 | Candida albicans (Yeast) | Stress response protein |
| | | | NST1 |
| KQKRKEKRQKR | 574 | Xenopus tropicalis (Western | RNA (guanine-9-)- |
| | | clawed frog) (Silurana tropicalis) | methyltransferase domain- |
| | | | containing protein 2 |
| RRRKKERRMRR | 575 | Cryptococcus neoformans | Transcription elongation |
| | | (Filobasidiella neoformans) | factor SPT6 |
| RRRRKHKRRPR | 576 | Ashbya gossypii (Yeast) | Transcription elongation |
| | | (Eremothecium gossypii) | factor SPT6 |
| KKKRRHKRRAR | 577 | Candida glabrata (Yeast) | Transcription elongation |
| | | (Torulopsis glabrata) | factor SPT6 |
| RERRRRKKRRR | 578 | Neurospora crassa | Transcription elongation |
| | | | factor spt-6 |
| KKRRKHKRRER | 579 | Saccharomyces cerevisiae (Baker's | Transcription elongation |
| | | yeast) | factor SPT6 |
| RERRKRRREER | 580 | Emericella nidulans (Aspergillus | Transcription elongation |
| | | nidulans) | factor spt6 |
| KEKRKLKKELK | 581 | Saccharomyces cerevisiae (Baker's | rRNA-processing protein |
| | | yeast) | EBP2 |
| RYRRRNRRGCR | 582 | Alouatta seniculus (Red howler | Protamine-2 |
| | | monkey) | |
| KRRKRRKRDPK | 583 | Mus musculus (Mouse) | High mobility group protein |
| | | | B4 |
| REKRKRRREER | 584 | Aspergillus fumigatus (Sartorya | Transcription elongation |
| | | fumigata) | factor spt6 |
| RQRRRHRRGCR | 585 | Hylobates lar (Common gibbon) | Protamine-2 |
| RHRRKHRRGCR | 586 | Pan troglodytes (Chimpanzee) | Protamine-2 |
| RHRRRHRKGCR | 587 | Gorilla gorilla gorilla (Lowland | Protamine-2 |
| | | gorilla) | |
| RRKRKRRRKKK | 588 | Bos taurus (Bovine) | Prokineticin-2 |
| RHRRRHRRGCR | 589 | Erythrocebus patas (Redguenon) | Protamine-2 |
| | | (Cercopithecus patas) | |
| RYRRRPRRGCR | 590 | Callithrix jacchus (Common | Protamine-2 |
| | | marmoset) | |
| REKKKEKKEKK | 591 | Danio rerio (Zebrafish) | ADP-ribosylation factor- |
| | | (Brachydanio rerio) | like protein6-interacting |
| | | | protein 4 |
| RDRKKTKKNKK | 592 | Schizosaccharomyces pombe | Signal recognition particle |
| | | (Fission yeast) | subunit srp14 |
| KPKKRYRRKLK | 593 | Mus musculus (Mouse) | C1orf1 15 homolog |
| KTKKKRKKEKK | 594 | Schizosaccharomyces pombe | Meiotically up-regulated |
| | | (Fission yeast) | gene 116 protein |
| RKRRKKKRKGK | 595 | Adelaide River virus (ARV) | Protein alpha-1 |
| KNKRKLRKIAK | 596 | Methanocaldococcus jannaschii | 50S ribosomal protein L1P |
| | | (Methanococcus jannaschii) | |
| RRKKRERKARK | 597 | Paramecium tetraurelia | Chromosome undetermined |
| | | | scaffold 171 |
| RYRRKLKKYGK | 598 | Homo sapiens (Human) | Protein C1orf1 15 |
| RKKKRKKKSCR | 599 | Homo sapiens (Human) | Protein FAM133A |
| RHRRKDKKTSR | 600 | Danio rerio (Zebrafish) | Arginine/serine-rich coiled- |
| | | (Brachydanio rerio) | coilprotein 2 |
| KEKRRIKKIIR | 601 | Paramecium tetraurelia | Chromosome undetermined |
| | | | scaffold 169 |
| KTRKKMKKAHK | 602 | Mus musculus (Mouse) | Proline-rich protein 13 |
| RDRKREKRKPK | 603 | Bos taurus (Bovine) | U1 small nuclear |
| | | | ribonucleoprotein A |
| RHRRKEKRSSR | 604 | Xenopus tropicalis (Western | Arginine/serine-rich coiled- |
| | | clawed frog) (Silurana tropicalis) | coil protein 2 |
| KPKKKVKKDEK | 605 | Methanocaldococcus jannaschii | Probable RNA-binding |
| | | (Methanococcus jannaschii) | protein MJ0652 |
| KSKRKGKRSSR | 606 | Paramecium tetraurelia | Chromosome undetermined |
| | | | scaffold 178 |
| KVKRRKKKYSR | 607 | Danio rerio (Zebrafish) | Transcription elongation |
| | | (Brachydanio rerio) | factor SPT6 |
| KSRKRAKKMTK | 608 | Saccharomyces cerevisiae (Baker's | Pre-mRNA-splicing factor 8 |
| | | yeast) | |
| RHKRKERKSSR | 609 | Bos taurus (Bovine) | Arginine/serine-rich coiled- |
| | | | coil protein 2 |
| KERKRRKKKSK | 610 | Drosophila melanogaster | CWF19-like protein 2 |
| | | (Fruitfly) | homolog |
| KLRRKQKKVNK | 611 | Schizosaccharomyces pombe | Protein C22F3.11c |
| | | (Fission yeast) | |
| KEKRRSKKRRK | 612 | Homo sapiens (Human) | Zinc finger CCCH domain- |
| | | | containing protein 4 |
| RHKRKVKRHRR | 613 | Paramecium tetraurelia | Chromosome undetermined |
| | | | scaffold 12 |
| KTKKRVRKQAK | 614 | Streptococcus pneumoniae | Transposase for insertion |
| | | | sequence IS1202 |
| RRKKRRRRKHR | 615 | Dictyostelium discoideum | Protein DDB 0237901 |
| | | (Slimemold) | |
| RPRRKRKRQQK | 616 | Cervus albirostris (Thorold's deer) | Sex-determining region Y |
| | | (White-lipped deer) | protein |
| RPRRRAKRPQK | 617 | Bison bison (American bison) | Sex-determining region Y |
| | | | protein |
| RPRRKAKRPQK | 618 | Balaenoptera acutorostrata | Sex-determining region Y |
| | | (Minke whale) (Lesser rorqual) | protein |
| RPRRKPKRPQK | 619 | Kogia simus (Dwarf spermwhale) | Sex-determining region Y |
| | | | protein |
| RPRRKAKRLQK | 620 | Bubalus bubalis (Domestic | Sex-determining region Y |
| | | waterbuffalo) | protein |
| RPRRKAKRSQK | 621 | Balaenoptera physalus | Sex-determining region Y |
| | | (Finbackwhale) (Common rorqual) | protein |
| RPRRKTKRQQK | 622 | Alces alces cameloides | Sex-determining region Y |
| | | (Ussurimoose) (Siberian moose) | protein |
| KKRKRRRRKSK | 623 | Mullus surmuletus (Striped | Protamine-like protein |
| | | redmullet) | |
| KDKRKEKRERK | 624 | Xenopus tropicalis (Western | Protein SFRS12IP1 |
| | | clawed frog) (Silurana tropicalis) | |
| RQRRKGKRMLR | 625 | Paramecium tetraurelia | Chromosome undetermined |
| | | | scaffold 122 |
| KQRRKGKRMLR | 626 | Paramecium tetraurelia | Chromosome undetermined |
| | | | scaffold 114 |
| KEKKKRKKEKR | 627 | Mus musculus (Mouse) | Protein SFRS12IP1 |
| RLRKKTRKRLK | 628 | Sus scrofa (Pig) | Antibacterial peptide |
| | | | PMAP-36 |
| RFRKKFKKLFK | 629 | Bos taurus (Bovine) | Cathelicidin-6 |
| RNKRKLKRLPR | 630 | Paramecium tetraurelia | Chromosome undetermined |
| | | | scaffold 106 |
| KSKKKSKKTKK | 631 | Caenorhabditis elegans | Transcriptional regulator |
| | | | ATRX homolog |
| KKKRRSRKKRK | 632 | Oryza saliva subsp. indica (Rice) | DEAD-box ATP-dependent |
| | | | RNA helicase 13 |
| RMRRRNRKTRR | 633 | Homo sapiens (Human) | Membrane protein |
| | | | FAM174A |
| KIKKRSKRFYK | 634 | Mus musculus (Mouse) | Zinc finger protein 329 |
| RKRRKERKKER | 635 | Homo sapiens (Human) | Coiled-coil domain- |
| | | | containing protein 1 40 |
| RERKKRKRKER | 636 | Mus musculus (Mouse) | Surfeit locus protein 6 |
| RKRKKHKKHFK | 637 | Homo sapiens (Human) | Vacuolar protein sorting- |
| | | | associated protein 13C |
| KDKKKLRRLLK | 638 | Homo sapiens (Human) | Transcription initiation |
| | | | protein SPT3 homolog |
| RVKKRHRRQRR | 639 | Homo sapiens (Human) | Beta-galactoside alpha-2 |
| KEKRKHRKEKK | 640 | Bos taurus (Bovine) | Wiskott-Aldrich syndrome |
| | | | protein family member 2 |
| KARKKEKRRAR | 641 | Ajellomyces capsulata (strain | Protein PXR1 |
| | | NAm1/WU24) (Darling's disease | |
| | | fungus) (Histoplasma capsulatum) | |
| RQRKRERREAR | 642 | Dictyostelium discoideum | Protein SCAR |
| | | (Slimemold) | |
| KDKKKLKRYTK | 643 | Paramecium tetraurelia | Chromosome undetermined |
| | | | scaffold 18 |
| KDKRREKRLLK | 644 | Paramecium tetraurelia | Chromosome undetermined |
| | | | scaffold 110 |
| KSKKRSKKSKK | 645 | Pyrococcus horikoshii | Transcription factor E |
| KKKRKLKKKAK | 646 | Bos taurus (Bovine) | Protein C12orf43 homolog |
| KDKKKDKKDK | 647 | Saccharomyces cerevisiae (Baker's | Protein PXR1 |
| | | yeast) | |
| KDKKKDKKDKK | 648 | Saccharomyces cerevisiae (strain | Protein PXR1 |
| | | YJM789) (Baker's yeast) | |
| KGKRRGRRYVK | 649 | Methanopyrus kandleri | 50S ribosomal protein L4P |
| KSRKKEKKELK | 650 | Rattus norvegicus (Rat) | Cell growth-regulating |
| | | | nucleolar protein |
| KNRKKEKKELK | 651 | Mus musculus (Mouse) | Cell growth-regulating |
| | | | nucleolar protein |
| KKRKREKKELK | 652 | Homo sapiens (Human) | Cell growth-regulating |
| | | | nucleolar protein |
| KERKREKRQMR | 653 | Aspergillus clavatus | Protein pxr1 |
| KTKKKYKKQMK | 654 | Paramecium tetraurelia | Chromosome undetermined |
| | | | scaffold 175 |
| KSKKKNKKDKK | 655 | Dictyostelium discoideum | Cell division control protein |
| | | (Slimemold) | 45 homolog |
| KHKKKEKKSKK | 656 | Paramecium tetraurelia | Chromosome undetermined |
| | | | scaffold 108 |
| RSKKKEKKDKK | 657 | Neurospora crassa | Protein pxr-1 |
| KERKKRKKEKK | 658 | Aedes aegypti (Yellowfever | Mediator of RNA |
| | | mosquito) | polymerase II transcription |
| | | | subunit 19 |
| KNKKKEKKSKK | 659 | Paramecium tetraurelia | Chromosome undetermined |
| | | | scaffold 105 |
| KHKKKEKKSNK | 660 | Paramecium tetraurelia | Chromosome undetermined |
| | | | scaffold 121 |
| KKRRKHKKHSK | 661 | Mus musculus (Mouse) | Protein FAM133B |
| KSRRKLKRGKK | 662 | Mus musculus (Mouse) | Lon protease homolog |
| KSRKRKKKHRK | 663 | Mus musculus (Mouse) | Peptidyl-prolyl cis-trans |
| | | | isomerase G |
| KKRKKHKKHSK | 664 | Gallus gallus (Chicken) | Protein FAM133 |
| KSKKRKKKHRK | 665 | Homo sapiens (Human) | Peptidyl-prolyl cis-trans |
| | | | isomerase G |
| KQKRRKRKLEK | 666 | Ashbya gossypii (Yeast) | Translocation protein |
| | | (Eremothecium gossypii) | SEC62 |
| KPRRKSKRGKK | 667 | Homo sapiens (Human) | Lon protease homolog |
| KLRKKPKRGKK | 668 | Bos taurus (Bovine) | Lon protease homolog |
| REKRKNRKFEK | 669 | Arabidopsis thaliana (Mouse- | Zinc finger protein |
| | | earcress) | CONSTANS-LIKE 3 |
| REKRKDRKFSK | 670 | Arabidopsis thaliana (Mouse- | Protein TIFY 4A |
| | | earcress) | |
| KAKKKPKKGKK | 671 | Gallus gallus (Chicken) | Monocarboxylate |
| | | | transporter 3 |
| KAKKRRRKKVR | 672 | Syntrophobacter fumaroxidans | Translation initiation factor |
| | | (strain DSM 10017/MPOB) | IF-2 |
| KRRKKEKKERK | 673 | Magnaporthe grisea (Rice | Protein PXR1 |
| | | blastfungus) (Pyricularia grisea) | |
| RVKRKSRKEKR | 674 | Arabidopsis thaliana (Mouse- | CAX-interacting protein 4 |
| | | earcress) | |
| KDKKRDKKEKK | 675 | Chaetomium globosum | Protein PXR1 |
| | | (Soilfungus) | |
| KTRRKAKKSDK | 676 | Natranaerobius thermophilus | 50S ribosomal protein L2 |
| | | (strain ATCC BAA-1301/DSM | |
| | | 18059/JW/NM-WN-LF) | |
| KTRKKNKKSNK | 677 | Finegoldia magna (strain ATCC | 50S ribosomal protein L2 |
| | | 29328) (Peptostreptococcus | |
| | | magnus) | |
| RRKKRRRRISK | 678 | Gramella forsetii (strain KT0803) | Translation initiation factor |
| | | | IF-2 |
| KTKRKMRRREK | 679 | Homo sapiens (Human) | Protein Wnt-16 |
| RFKRRNRKARK | 680 | Enterobacteria phage T7 | Protein kinase |
| | | (Bacteriophage T7) | |
| KSRKRGKKRRR | 681 | Idiomarina loihiensis | Translation initiation factor |
| | | | IF-2 |
| KEKRRKKRPPR | 682 | Bos taurus (Bovine) | Transmembrane protein 198 |
| KARKKEKKEKK | 683 | Botryotinia fuckeliana (strain | Protein pxrl |
| | | B05.10) (Noble rot fungus) | |
| | | (Botrytis cinerea) | |
| KSRKRGKRNLK | 684 | Paramecium tetraurelia | Chromosome undetermined |
| | | | scaffold 145 |
| RKRKKGKKTSR | 685 | Bacillus anthracis | Teichoic acids export ATP- |
| | | | binding protein tagH |
| KDKKRKKKRSK | 686 | Saccharomyces cerevisiae (Baker's | Chromosome transmission |
| | | yeast) | fidelityprotein 18 |
| KKRKKRKKLKK | 687 | Homo sapiens (Human) | Ankyrin repeat domain- |
| | | | containing protein 18B |
| KKRRREKKKRR | 688 | Caenorhabditis elegans | Protein let-756 |
| RRKRRERKQRR | 689 | Homo sapiens (Human) | Leucine-rich repeat- |
| | | | containingprotein 47 |
| RKKRRERKQHR | 690 | Mus musculus (Mouse) | Leucine-rich repeat- |
| | | | containing protein 47 |
| KPKKKTRKPSK | 691 | Homo sapiens (Human) | Protein FAM153A |
| KAKKRHKRS PK | 692 | Xylella fastidiosa (strain | Bis(5/-nucleosyl)- |
| | | Temeculal/ATCC 700964) | tetraphosphatase |
| REKKKTRKFDK | 693 | Arabidopsis thaliana (Mouse- | Zinc finger protein |
| | | earcress) | CONSTANS-LIKE 2 |
| KYKRRTKKKVK | 694 | Dictyostelium discoideum | Mitochondrial protein Mp36 |
| | | (Slimemold) | |
| KEKKKEKKKAK | 695 | Dictyostelium discoideum | Oxysterol-binding protein 7 |
| | | (Slimemold) | |
| REKKKMRKFEK | 696 | Arabidopsis thaliana (Mouse- | Zinc finger protein |
| | | earcress) | CONSTANS-LIKE 1 |
| KSKKRKKRKRK | 697 | Homo sapiens (Human) | G patch domain-containing |
| | | | protein 8 |
| KEKKKSRRYEK | 698 | Arabidopsis thaliana (Mouse- | Zinc finger protein |
| | | earcress) | CONSTANS-LIKE 13 |
| RAKRRMKRDLK | 699 | Drosophila melanogaster | Vanin-like protein 2 |
| | | (Fruitfly) | |
| KNKKRNRKAKR | 700 | Pediococcus pentosaceus | Translation initiation factor |
| | | (strainATCC 25745/183-1w) | IF-2 |
| KEKRKKRKKTK | 701 | Rattus norvegicus (Rat) | CBF1-interacting |
| | | | corepressor |
| KGKRRKRKKRK | 702 | Fugu rubripes (Japanese | Homeobox protein Hox-C8a |
| | | pufferfish) (Takifugu rubripes) | |
| KLKKKHKKDKK | 703 | Saccharomyces cerevisiae (Baker's | Protein YRO2 |
| | | yeast) | |
| REKRKTRKFEK | 704 | Arabidopsis thaliana (Mouse- | Zinc finger protein |
| | | earcress) | CONSTANS |
| RQRRRPRRQKR | 705 | Deinococcus radiodurans | Fatty acid/phospholipid |
| | | | synthesisprotein plsX |
| KEKKKDKKKNK | 706 | Dictyostelium discoideum | Multiple RNA-binding |
| | | (Slimemold) | domain-containing protein 1 |
| RERRRNRRQQR | 707 | Arabidopsis thaliana (Mouse- | Probable xyloglucanendo- |
| | | earcress) | transglucosylase/hydrolase |
| | | | protein 30 |
| RFKRRAKRQNR | 708 | Neurospora crassa | Palmitoyltransferase ERF2 |
| KNKKKLRKGSR | 709 | Xenopus laevis (African | Bone morphogenetic protein |
| | | clawedfrog) | 3 |
| RCKKRCRRITR | 710 | Erwinia chrysanthemi | Extracellular phospholipase |
| | | | C |
| RHRRKRKRRRK | 711 | Mus musculus (Mouse) | Scaffold attachment factor |
| | | | B2 |
| KEKKKARKFDK | 712 | Arabidopsis thaliana (Mouse- | Zinc finger protein |
| | | earcress) | CONSTANS-LIKE10 |
| KPKKKFRKIKK | 713 | Soybean chlorotic mottle virus | Capsid protein |
| RRKRKTRRRKK | 714 | Homo sapiens (Human) | RING and PHD-finger |
| | | | domain-containing protein |
| | | | KIAA1542 |
| KQKKRIKKLEK | 715 | Paramecium tetraurelia | Chromosome undetermined |
| | | | scaffold 22 |
| KAKRKRKKKLK | 716 | Danio rerio (Zebrafish) | La-related protein 7 |
| | | (Brachydanio rerio) | |
| KDKKKLKKDDK | 717 | Homo sapiens (Human) | Cylicin-1 |
| RLRRRQKRQNK | 718 | Homo sapiens (Human) | Glycine receptor subunit |
| | | | alpha-2 |
| RVKRRLRRQRK | 719 | Salmonella typhi | Undecaprenyl-phosphate |
| | | | alpha-N-acetylglucosaminyl |
| | | | 1-phosphate transferase |
| RFKRRMRRASK | 720 | Yersinia pestis | Undecaprenyl-phosphate |
| | | | alpha-N-acetylglucosaminyl |
| | | | 1-phosphate transferase |
| KVKRKHKKKHK | 721 | Mus musculus (Mouse) | La-related protein 7 |
| KQKKKGRKRIK | 722 | Drosophila melanogaster | Transcriptional regulator |
| | | (Fruitfly) | ATRX homolog |
| KARRKGRRGGK | 723 | Bos taurus (Bovine) | Early growth response |
| | | | protein 4 |
| KDKRKGRKRSR | 724 | Homo sapiens (Human) | Splicing factor |
| KGKRKHKKKHK | 725 | Rattus norvegicus (Rat) | La-related protein 7 |
| KTKRKHKKKHK | 726 | Homo sapiens (Human) | La-related protein 7 |
| KEKKKTRRYDK | 727 | Arabidopsis thaliana (Mouse- | Zinc finger protein |
| | | earcress) | CONSTANS-LIKE14 |
| KNKKKSKKPSK | 728 | Schizosaccharomyces pombe | Curved DNA-binding |
| | | (Fission yeast) | protein |
| RVKKRNRKEEK | 729 | Saimiriine herpesvirus 2 (strain11) | Thymidine kinase |
| | | (SaHV-2) (Herpesvirus saimiri) | |
| KSKRRSRKRYR | 730 | Mus musculus (Mouse) | Vitronectin |
| RFRKKRRRSQR | 731 | Homo sapiens (Human) | UPF0632 protein B |
| RAKRKHKRLVK | 732 | Arabidopsis thaliana (Mouse- | Phosphatidylinositol-4- |
| | | earcress) | phosphate 5-kinase 9 |
| KSRRRSRKRYR | 733 | Oryctolagus cuniculus (Rabbit) | Vitronectin |
| KDRKRDKKRSR | 734 | Caenorhabditis elegans | Probable splicing factor |
| KVKRKRKKKHK | 735 | Xenopus tropicalis (Western | La-related protein 7 |
| | | clawed frog) (Silurana tropicalis) | |
| RQKRKNRRKNK | 736 | Gallus gallus (Chicken) | Gametogenetin-binding |
| | | | protein 2 |
| KAKKKEKKGKK | 737 | Methanocaldococcus jannaschii | OP5 family protein MJ0694 |
| | | (Methanococcus jannaschii) | |
| KVRKKDKKKEK | 738 | Mus musculus (Mouse) | RNA exonuclease 1 |
| | | | homolog |
| KKKRKKRKKGK | 739 | Ajellomyces capsulata | WD repeat-containing |
| | | (strainNAml/WU24) (Darling's | protein JIP5 |
| | | disease fungus) (Histoplasma | |
| | | capsulatum) | |
| KGKRKKRKRGK | 740 | Coccidioides immitis | WD repeat-containing |
| | | | protein JIP5 |
| RLRRKARRDSR | 741 | Xenopus laevis (African clawed | Midnolin-A |
| | | frog) | |
| KEKRKTRRYDK | 742 | Arabidopsis thaliana (Mouse- | Zinc finger protein |
| | | earcress) | CONSTANS-LIKE15 |
| KQKRKKRRKGK | 743 | Phaeosphaeria nodorum (Septoria | WD repeat-containing |
| | | nodorum) | protein JIP5 |
| RQRKKRKRKSK | 744 | Arabidopsis thaliana (Mouse- | F-box protein At3g19890 |
| | | earcress) | |
| KIKKKNRKIKK | 745 | Buchnera aphidicola subsp. | Arginyl-tRNA synthetase |
| | | Cinara cedri | |
| KTKRRVKKIRK | 746 | Homo sapiens (Human) | U4/U6.U5 tri-snRNP- |
| | | | associatedprotein 1 |
| RLRRKARRDAR | 747 | Homo sapiens (Human) | Midnolin |
| RKRKRIKKSLK | 748 | Coccidioides immitis | Chromosome segregation in |
| | | | meiosisprotein 3 |
| KPRKRRRRPGR | 749 | Neurospora crassa | Mitochondrial Rho GTPase |
| | | | 1 |
| RPRKKRKRPGR | 750 | Aspergillus fumigatus (Sartorya | Mitochondrial Rho GTPase |
| | | fumigata) | 1 |
| RPRKRRKRPGR | 751 | Aspergillus oryzae | Mitochondrial Rho GTPase |
| | | | 1 |
| RWKKRNRRLKK | 752 | Helianthus tuberosus (Jerusalem | Calnexin homolog |
| | | artichoke) (Helianthus tomentosus) | |
| KVRKKAKKAKK | 753 | Homo sapiens (Human) | Bromodomain-containing |
| | | | protein 1 |
| KHRRRERRFGR | 754 | Bos taurus (Bovine) | Calpain-1 catalytic subunit |
| KRRKKRKKKER | 755 | Rattus norvegicus (Rat) | Ankyrin repeat and zinc |
| | | | finger domain-containing |
| | | | protein 1 |
| RERRRDRRQEK | 756 | Koala retrovirus (KoRV) | Gag polyprotein |
| RRRKRNKRERK | 757 | Bos taurus (Bovine) | Ankyrin repeat and zinc |
| | | | finger domain-containing |
| | | | protein 1 |
| KVKKKRKKETK | 758 | Homo sapiens (Human) | Ankyrin repeat domain- |
| KDKRRKRKHHR | 759 | Xenopus tropicalis (Western | Zinc finger matrin-type |
| | | clawed frog) (Silurana tropicalis) | protein 1 |
| KNRKKNKKKIR | 760 | Paramecium tetraurelia | Chromosome undetermined |
| | | | scaffold 171 |
| KRKKKEKKEKK | 761 | Aspergillus fumigatus (Sartorya | Centromere/microtubule- |
| | | fumigata) | binding protein cbf5 |
| KDKKKDKKEKK | 762 | Candida albicans (Yeast) | Nucleolar protein NOP58 |
| KEKKKSKKDKK | 763 | Homo sapiens (Human) | H/ACA ribonucleoprotein |
| | | | complex subunit 4 |
| RKKRKERKDKK | 764 | Paramecium tetraurelia | Chromosome undetermined |
| | | | scaffold 10 |
| KTKKKSKKREK | 765 | Drosophila melanogaster | Pre-mRNA-splicing factor |
| | | (Fruitfly) | Slu7 |
| KGKKRGRRPQK | 766 | Lactococcus lactis subsp. Lactis | RNA methyltransferase |
| | | (Streptococcus lactis) | ywfF |
| RERKKERKLAR | 767 | Podospora anserina | Protein SQS1 |
| RARRRPKRSKK | 768 | Myxococcus xanthus | Transcription-repair- |
| | | | coupling factor |
| RKRKKLRKINK | 769 | Candida glabrata (Yeast) | High-osmolarity- |
| | | (Torulopsis glabrata) | inducedtranscription protein |
| | | | 1 |
| RNRRRSKRRKK | 770 | Saccharomyces cerevisiae (Baker's | Membrane protein |
| | | yeast) | YFLO34W |
| RDKKREKRERK | 771 | Caenorhabditis elegans | Pre-mRNA-splicing factor |
| | | | SLU7 |
| KTKKKRRRGPR | 772 | Xenopus laevis (African clawed | UPF0622 protein |
| | | frog) | |
| KHKKKRRRGPR | 773 | Homo sapiens (Human) | UPF0622 protein |
| KLKRKEKRKKK | 774 | Homo sapiens (Human) | Kinesin-like protein |
| | | | KIF21A |
| KEKKKRRRRKK | 775 | Homo sapiens (Human) | Probable global |
| | | | transcriptionactivator |
| | | | SNF2L2 |
| KQKKRGRRGGK | 776 | Potato leafroll virus (strain 1) | Protein ORF1 |
| | | (PLrV) | |
| KEKRRERKRAK | 777 | Coccidioides immitis | ATP-dependent rRNA |
| | | | helicase SPB4 |
| RVKRKYRRRSK | 778 | Kluyveromyces lactis (Yeast) | SWR1-complex protein 3 |
| | | (Candida phaerica) | |
| KNKKKNKKKNK | 779 | Candida glabrata (Yeast) | KNR4/SMI1 homolog |
| | | (Torulopsis glabrata) | |
| KKKRKIKRLEK | 780 | Saccharomyces cerevisiae (Baker's | Signal recognition particle |
| | | yeast) | subunit SRP72 |
| RSRKRTRRKKK | 781 | Chaetomium globosum | Topoisomerase 1-associated |
| | | (Soilfungus) | factor 1 |
| RSRKRARRKKK | 782 | Neurospora crassa | Topoisomerase 1-associated |
| | | | factor 1 |
| RSRRRARKRKR | 783 | Emericella nidulans (Aspergillus | Topoisomerase 1-associated |
| | | nidulans) | factor 1 |
| RSRRRIKRKAK | 784 | Aspergillus clavatus | Topoisomerase 1-associated |
| | | | factor 1 |
| RSRRRAKRKAK | 785 | Aspergillus fumigatus (Sartorya | Topoisomerase 1-associated |
| | | fumigata) | factor 1 |
| KSKRRARKIKK | 786 | Phaeosphaeria nodorum (Septoria | Topoisomerase 1-associated |
| | | nodorum) | factor 1 |
| RSRRRARRKAK | 787 | Aspergillus niger (strain | Topoisomerase 1-associated |
| | | CBS513.88/FGSC A1513) | factor 1 |
| KRRRKGKRRKR | 788 | Schizosaccharomyces pombe | Serine/threonine-protein |
| | | (Fission yeast) | kinase ppk4 |
| REKKRRKREAK | 789 | Neurospora crassa | ATP-dependent rRNA |
| | | | helicase spb-4 |
| KAKRKVKKFVR | 790 | Caenorhabditis briggsae | Mediator of RNA |
| | | | polymerase II transcription |
| | | | subunit 13 |
| RRKKKRRKLCR | 791 | Mus musculus (Mouse) | Zinc finger protein 804A |
| KGKKKGKKGKK | 792 | Yarrowia lipolytica (Candida | Endopolyphosphatase |
| | | lipolytica) | |
| RARRKARKEKK | 793 | Schizophyllum commune (Bracket | Mating-type protein A-alpha |
| | | fungus) | Y1 |
| KERRRERKKIR | 794 | Ajellomyces capsulata | ATP-dependent rRNA |
| | | (strainNAml/WU24) (Darling's | helicase SPB4 |
| | | disease fungus) (Histoplasma | |
| | | capsulatum) | |
| RAKRRARKEKK | 795 | Schizophyllum commune (Bracket | Mating-type protein A-alpha |
| | | fungus) | Y3 |
| KVKKKLRKKGK | 796 | Homo sapiens (Human) | Protein C18orf34 |
| RARRKERKQRK | 797 | Schizophyllum commune (Bracket | Mating-type protein A-alpha |
| | | fungus) | Y4 |
| KSKRKYKKRPK | 798 | Xenopus laevis (African clawed | Protein KIAA0649 homolog |
| | | frog) | |
| KEKKRLKKKQK | 799 | Mus musculus (Mouse) | ESF1 homolog |
| RS RKKKRRKKK | 800 | Gallus gallus (Chicken) | Smoothened homolog |
| KEKKRLKRKQK | 801 | Homo sapiens (Human) | ESF1 homolog |
| RGKKKRKKFMK | 802 | Bos taurus (Bovine) | Probable ATP-dependent |
| | | | RNAhelicase DDX27 |
| RSKRKFKKRCR | 803 | Mus musculus (Mouse) | Protein KIAA0649 |
| KFKRKEKKKIK | 804 | Paramecium tetraurelia | Chromosome undetermined |
| | | | scaffold 13 |
| KQKRREKREKR | 805 | Gibberella zeae (Fusarium | ATP-dependent RNA |
| | | graminearum) | helicase DBP4 |
| RERKRHRKRSR | 806 | Caenorhabditis elegans | Protein SWAP |
| KQKKREKKLKR | 807 | Magnaporthe grisea (Rice | ATP-dependent RNA |
| | | blastfungus) (Pyricularia grisea) | helicase DBP4 |
| KEKKKDKKEKK | 808 | Gallus gallus (Chicken) | Transcription initiation |
| | | | factor TFIID subunit 3 |
| KRKKKLRKLLK | 809 | Homo sapiens (Human) | Extracellular sulfatase Sulf- |
| | | | 2 |
| RSKRKLKKRCR | 810 | Homo sapiens (Human) | Protein KIAA0649 |
| KSKKKNKKKGK | 811 | Yarrowia lipolytica (Candida | KNR4/SMI1 homolog |
| | | lipolytica) | |
| KEKKKLKKKEK | 812 | Kluyveromyces lactis (Yeast) | Stress response protein |
| | | (Candida sphaerica) | NST1 |
| KDKKREKREKR | 813 | Sclerotinia sclerotiorum | ATP-dependent RNA |
| | | (strainATCC 18683/1980/Ss-1) | helicase dbp4 |
| | | (White mold) (Whetzelinia | |
| | | sclerotiorum) | |
| KQKKREKKEKR | 814 | Aspergillus niger (strain | ATP-dependent RNA |
| | | CBS513.88/FGSC A1513) | helicase dbp4 |
| KKRRKAKRLKK | 815 | Gallus gallus (Chicken) | Tyrosine-protein kinase-like |
| | | | 7 |
| KQKRREKKEKR | 816 | Aspergillus clavatus | ATP-dependent RNA |
| | | | helicase dbp4 |
| RRRKKERKGKK | 817 | Coturnix coturnix (Common quail) | Extracellular sulfatase Sulf- |
| | | | 1 |
| RRRKKERKEKK | 818 | Mus musculus (Mouse) | Extracellular sulfatase Sulf- |
| | | | 1 |
| RRRKKERKEKR | 819 | Homo sapiens (Human) | Extracellular sulfatase Sulf- |
| | | | 1 |
| KDKRRKKRKEK | 820 | Homo sapiens (Human) | AP-3 complex subunit |
| | | | delta-1 |
| RSKKKRRRGRK | 821 | Homo sapiens (Human) | Neuroblastoma breakpoint |
| | | | familymember 8 |
| KEKKKIRRKRR | 822 | Xenopus laevis (African clawed | JmjC domain-containing |
| | | frog) | histonedemethylation |
| | | | protein 1B |
| KKKRKKRKSPK | 823 | Homo sapiens (Human) | Protocadherin-9 |
| RQRKKERKRIK | 824 | Drosophila melanogaster | Extracellular sulfatase |
| | | (Fruitfly) | SULF-1 homolog |
| KYKRRLRREIK | 825 | Paramecium tetraurelia | Chromosome undetermined |
| | | | scaffold 114 |
| KRRRKRKKENK | 826 | Homo sapiens (Human) | Bromodomain and WD |
| | | | repeat-containing protein 1 |
| KRRRKRRKESK | 827 | Mus musculus (Mouse) | Bromodomain and WD |
| | | | repeat-containing protein 1 |
| KARRKVRRDRK | 828 | Yarrowia lipolytica (Candida | Increased rDNA silencing |
| | | lipolytica) | protein 4 |
| RKKKRRRRP PK | 829 | Mus musculus (Mouse) | RNA polymerase-associated |
| | | | protein CTR9 homolog |
| KGKKKCKKLGK | 830 | Drosophila hydei (Fruit fly) | Axoneme-associated |
| | | | proteinmst101(2) |
| KDKKKSKKATK | 831 | Drosophila melanogaster | Nucampholin |
| | | (Fruitfly) | |
| RSKKKDKRKRR | 832 | Saccharomyces cerevisiae (Baker's | Topoisomerase 1-associated |
| | | yeast) | factor 1 |
| KQKKKYKKTKK | 833 | Heterosigma carterae | DNA-directed RNA |
| | | | polymerase subunit beta |
| KLKKRMKRKYK | 834 | Odontella sinensis (Marinecentric | DNA-directed RNA |
| | | diatom) | polymerase subunit beta/ |
| KYRKKIRKLQK | 835 | Paramecium tetraurelia | Chromosome undetermined |
| | | | scaffold 174 |
| KRRKRRRRKNK | 836 | Plasmodium falciparum (isolate | DNA-directed RNA |
| | | CDC/Honduras) | polymerase II subunit RPB1 |
| KLKKKHKKKGK | 837 | Homo sapiens (Human) | WD repeat-containing |
| | | | protein 87 |
| RRRKRLKKKDR | 838 | Homo sapiens (Human) | AT-rich interactive domain- |
| | | | containingprotein 4A |
| KRKRKTKRKTK | 839 | Pinus thunbergii (Green pine) | Protein ycf2 |
| | | (Japanese black pine) | |
| RRRKRLKKKER | 840 | Homo sapiens (Human) | AT-rich interactive domain- |
| | | | containingprotein 4B |
| KGKRKKRRCWR | 841 | Mus musculus (Mouse) | Probable histone-lysine N- |
| | | | methyltransferase NSD2 |
| KRKRRKRKNRK | 842 | Drosophila melanogaster | ATP-dependent helicase |
| | | (Fruitfly) | brm |
| RVRKRNRKSGK | 843 | Homo sapiens (Human) | Methyl-CpG-binding |
| | | | domain protein5 |
| REKRKEKRKKK | 844 | Homo sapiens (Human) | Ankyrin repeat and KH |
| | | | domain-containing protein 1 |
| KGRKKIRKILK | 845 | Homo sapiens (Human) | Transcriptional regulator |
| | | | ATRX |
| REKRKEKRRKK | 846 | Homo sapiens (Human) | Ankyrin repeat domain- |
| | | | containingprotein 17 |
| KDKKKQKKEAK | 847 | Caenorhabditis briggsae | Ankyrin repeat and KH |
| | | | domain-containing protein |
| | | | CBG24701 |
| RKKRKEKRLKR | 848 | Homo sapiens (Human) | Down syndrome cell |
| | | | adhesionmolecule-like |
| | | | protein 1 |
| KDKKKGKKKGK | 849 | Gibberella zeae (Fusarium | Helicase SWR1 |
| | | graminearum) | |
| KDKKKEKRNSK | 850 | Homo sapiens (Human) | Dedicator of cytokinesis |
| | | | protein 1 |
| KIRKKRRRTRR | 851 | Ustilago maydis (Smut fungus) | Lysophospholipase NTE1 |
| KKRKRKRRAGR | 852 | Mus musculus (Mouse) | Fer-l-like protein 4 |
| RPRRRSRKCGK | 853 | Homo sapiens (Human) | Lupus brain antigen 1 |
| | | | homolog |
| RMKRKEKKLLR | 854 | Mus musculus (Mouse) | Zonadhesin |
| KIKKKSRKIKK | 855 | Drosophila melanogaster | Titin |
| | | (Fruitfly) | |
| KARRKGRRGGK | 867 | Homo sapiens (Human) | Early growth response |
| | | | protein 4 |

2. Antibodies for Modification

a. General Characteristics of Antibodies

Antibodies are produced naturally by B cells in membrane-bound and secreted forms and specifically recognize and bind antigen epitopes through cognate interactions. Antibody-antigen binding can initiate multiple effector functions, which cause neutralization and clearance of toxins, pathogens and other infectious agents.

Diversity in antibody specificity arises naturally due to recombination events during B cell development. Through these events, various combinations of multiple antibody V, D and J gene segments, which encode variable regions of antibody molecules, are joined with constant region genes to generate a natural antibody repertoire with large numbers of diverse antibodies. A Human antibody repertoire contains more than 10¹⁰ different antigen specificities and thus theoretically can specifically recognize any foreign antigen. Antibodies include such naturally produced antibodies, as well as synthetically, i.e. recombinantly, produced antibodies, such as antibody fragments, including the modified therapeutic antibodies provided herein.

In folded antibody polypeptides, binding specificity is conferred by antigen binding site domains, which contain portions of heavy and/or light chain variable region domains. Other domains on the antibody molecule serve effector functions by participating in events such as signal transduction and interaction with other cells, polypeptides and biomolecules. These effector functions cause neutralization and/or clearance of the infecting agent recognized by the antibody. Domains of antibody polypeptides can be varied according to the methods herein to alter specific properties.

### i. Structural and Functional Domains of Antibodies

Full-length antibodies contain multiple chains, domains and regions. A full length conventional antibody contains two heavy chains and two light chains, each of which contains a plurality of immunoglobulin (Ig) domains. An Ig domain is characterized by a structure called the Ig fold, which contains two beta-pleated sheets, each containing anti-parallel beta strands connected by loops. The two beta sheets in the Ig fold are sandwiched together by hydrophobic interactions and a conserved intra-chain disulfide bond. The Ig domains in the antibody chains are variable (V) and constant (C) region domains.

Each full-length conventional antibody light chain contains one variable region domain (V₁) and one constant region domain (C_{L}). Each full-length conventional heavy chain contains one variable region domain (V_{H}) and three or four constant region domains (C_{H}) and, in some cases, a hinge region. Owing to recombination events discussed above, nucleic acid sequences encoding the variable region domains differ among antibodies and confer antigen-specificity to a particular antibody. The constant regions, on the other hand, are encoded by sequences that are more conserved among antibodies. These domains confer functional properties to antibodies, for example, the ability to interact with cells of the immune system and serum proteins in order to cause clearance of infectious agents. Different classes of antibodies, for example IgM, IgD, IgG, IgE and IgA, have different constant regions, allowing them to serve distinct effector functions.

Each variable region domain contains three portions called complementarity determining regions (CDRs) or hypervariable (HV) regions, which are encoded by highly variable nucleic acid sequences. The CDRs are located within the loops connecting the beta sheets of the variable region Ig domain. Together, the three heavy chain CDRs (CDR1, CDR2 and CDR3) and three light chain CDRs (CDR1, CDR2 and CDR3) make up a conventional antigen binding site (antibody combining site) of the antibody, which physically interacts with cognate antigen and provides the specificity of the antibody. A whole antibody contains two identical antibody combining sites, each made up of CDRs from one heavy and one light chain. Because they are contained within the loops connecting the beta strands, the three CDRs are non-contiguous along the linear amino acid sequence of the variable region. Upon folding of the antibody polypeptide, the CDR loops are in close proximity, making up the antigen combining site. The beta sheets of the variable region domains form the framework regions (FRs), which contain more conserved sequences that are important for other properties of the antibody, for example, stability.

### ii. Antibody Fragments

Antibodies for modification by the protein transduction domain include antibody fragments, which are derivatives of full-length antibodies that contain less than the full sequence of the full-length antibodies but retain at least a portion specific binding abilities of the full-length antibodies. Examples of antibody fragments include, but are not limited to, Fab, Fab′, F(ab′)₂, single-chain Fvs (scFv), Fv, dsFv, diabody, Fd and Fd′ fragments, and other fragments, including modified fragments (see, for example, Methods in Molecular Biology, Vol 207: Recombinant Antibodies for Cancer Therapy Methods and Protocols (2003); Chapter 1; p 3-25, Kipriyanov). Antibody fragments can include multiple chains linked together, such as by disulfide bridges and can be produced recombinantly. Antibody fragments also can contain synthetic linkers, such as peptide linkers, to link two or more domains.

b. Selection of Antibodies for Modification

Antibodies for modification by a protein transduction domain according to the methods provided herein can be selected from any full-length conventional antibody or antigen-binding fragment thereof, such as, for example, Fab, F(ab′), F(ab′)₂, single-chain Fvs (scFv), Fv, dsFv, diabody, Fd and Fd′ fragments, and other fragments. Exemplary therapeutic antibodies to which a transduction domain can be conjugated thus include, for example, monoclonal antibodies, single chain antibodies (scFv), chimeric antibodies, Fab fragments, F(ab′) fragments, F(ab′)₂ fragments and other antigen binding fragments, containing a V_{H} and/or a V_{L} domain. In specific examples, the antibody selected for modification is a single-chain antibody.

The therapeutic antibodies provided herein can be derived from any antibody isotype, including, but not limited to, IgG, IgM, IgD, IgE, IgA and IgY. Further, the therapeutic antibodies provided herein can be derived from any class (e.g., IgG1, IgG2, IgG3, IgG4, IgA1 and IgA2) or subclass of immunoglobulin molecule.

The modified therapeutic antibodies provided herein can be generated from an antibody of any animal origin including, but not limited to, birds and mammals, for example, Human, murine (e.g. Mouse or rat), donkey, sheep, rabbit, goat, guinea pig, camelid, horse or chicken. The selected antibody for modification can be a chimeric, Humanized or synthetic antibody. The modified therapeutic antibodies provided herein can be monospecific for a particular antigen or can be bispecific, trispecific or of greater multispecificity for two or more antigens. In particular examples, the modified therapeutic antibodies provided herein are Human antibodies or Humanized antibodies.

Single chain antibodies can be recombinantly engineered by joining a heavy chain variable region (V_{H}) and light chain variable region (V_{L}) of a specific antibody. The particular nucleic acid sequences for the variable regions can be cloned by standard molecular biology methods, such as, for example, by polymerase chain reaction (PCR) and other recombinant nucleic acid technologies. Methods for producing sFvs are described, for example, by Whitlow and Filpula (1991) Methods, 2: 97-105; Bird et al. (1988) Science 242:423-426; Pack et al. (1993) Bio/Technology 11:1271-77; and U.S. Pat. Nos. 4,946,778, 5,840,300, 5,667,988, 5,658,727, 5,258,498). Single chain antibodies also can be identified by screening single chain antibody libraries for binding to a target antigen. Methods for the construction and screening of such libraries are well-known in the art.

Antigen-binding antibody fragments, including single chain antibodies, can include the variable regions alone or in combination with one or more of the following: a hinge region, C_{H}1, C_{H}2, C_{H}3 and C_{L} domains.

Generally the modified therapeutic antibodies provided herein immunospecifically bind to a target antigen with a dissociation constant, or Kd, at least or about 1×10⁻² M⁻¹, at least or about 5×10⁻² M⁻¹, at least or about 1×10⁻³ M⁻¹, at least or about 5×10⁻³ M⁻¹, at least or about 1×10⁻⁴ M⁻¹, at least or about 5×10⁴ M⁻¹, at least or about 1×10⁻⁵ M⁻¹, at least or about 5×10⁻⁵ M⁻¹, at least or about 1×10⁻⁶ M⁻¹, at least or about 5×10⁻⁶ M⁻¹, at least or about 1×10⁻⁷ M⁻¹, at least or about 5×10⁻⁷ M⁻¹, at least or about 1×10⁻⁸ M⁻¹, at least or about 5×10⁻⁸ M⁻¹, at least or about 1×10⁻⁹ M⁻¹, at least or about 5×10⁻⁹ M⁻¹, at least or about 1×10⁻¹⁰ M⁻¹, at least or about 5×10⁻¹⁰M⁻¹, at least or about 1×10⁻¹¹ M⁻¹, at least or about 5×10⁻¹¹ M⁻¹, at least or about 1×10⁻¹² M⁻¹, at least or about 5×10⁻¹² M⁻¹, at least or about 1×10⁻¹³ M⁻¹, at least or about 5×10⁻¹³ M⁻¹, at least or about 1×10⁻¹⁴ M⁻¹, at least or about 5×10⁻¹⁴ M⁻¹, at least or about 1×10⁻¹⁵ M⁻¹, or at least or about 5×10⁻¹⁵ M⁻¹.

### i. Neutralizing Antibodies

Generally, the antibodies provided herein for modification are neutralizing antibodies that recognize one or more epitopes on the surface of a pathogen, such as a virus. Such antibodies are employed for the prevention and/or spread of pathogenic disease. Generally, the selected antibody has the ability to inhibit or reduce one or more activities of the pathogen, such as, for example, association with a target cell membrane, fusion with the target cell membrane and cell entry. Exemplary neutralizing antibodies are known in the art and include, for example antibodies that recognize viral, bacterial and fungal pathogens (see, e.g., Zeitlin et al. (1999) Emerg. Infect. Dis. 5(1):54-64). Exemplary neutralizing antibodies include antibodies that recognize pathogenic organisms such as, but not limited to, Staphylococcus aureus, Staphylococcus epidermidis, Streptococus mutans, Streptococus pneumoniae, Chlamydia trachomatis, Clostridium difficile, Escherichia coli, Vibrio cholerae, Porphyromonas gingivalis, Shigella flexneri, Enterococcus faecium, Enterococcus faecalis, Pseudomonas aeruginosa, Klebsiella pneumoniae, Acinetobacter baumannii, Serratia marcescens, Cryptococcus neoformans, Candida albicans, Candida glabrata, Candida krucei, Candida tropicalis, Aspergillus fumigatus, Plasmodium falciparum, and Toxoplasma gondii.

In some examples, the antibody selected for modification can be a neutralizing antibody that recognizes an epitope on the surface of the pathogen. Such epitopes can be found on carbohydrate moieties, lipid moieties, protein or combinations thereof. In particular examples, the selected antibody is an antibody that recognizes an antigen on the membrane surface an enveloped virus. For example, the selected antibody can be an antibody that binds to an epitope on a viral envelope, such as, for example, an epitope on the surface of a viral envelope glycoprotein. In other examples, the antibody can be a neutralizing antibody that binds to an epitope on the surface of a non-enveloped virus, such as for example, an epitope found on a viral capsid protein.

Exemplary antibodies for modification by a protein transduction domain include antibodies that neutralize viruses, including, but not limited to enveloped viruses, such as for example, herpes viruses (e.g., herpes simplex viruses (HSV-1, HSV-2)), paramyxoviruses (e.g., Human metapneumovirus (HMPV), Human respiratory syncytial virus (HRSV), Epstein-Barr Virus (EBV), Varicella Zoster virus (VZV) and cytomegalovirus (CMV)), poxviruses (e.g., vaccinia), rhabdoviruses (e.g. rabies), flaviviruses (e.g., hepatitis C virus (HCV) and West Nile virus (WNV)), coronaviruses (SARS-CoV), orthomyxoviruses (e.g., influenza virus), togaviruses, bunyaviruses, filoviruses, hepatitis delta viruses, hepadnaviruses and retroviruses (e.g., Human immunodeficiency virus (HIV). Exemplary antibodies for modification by a protein transduction domain also can include antibodies that neutralize non-enveloped viruses, including, but not limited to, adenoviruses, papillomaviruses, parvoviruses, polyomaviruses, reoviruses, and picornaviruses (e.g., rhinovirus, hepatovirus, and poliovirus).

In particular examples, the antibody selected for conjugation to a protein transduction domain is selected from among a herpes simplex virus (HSV) neutralizing antibody, a metapneumovirus (MPV) neutralizing antibody, and a respiratory syncytial virus (RSV) neutralizing antibody. Such antibodies are known in the art and can be employed in the methods provided herein (see, e.g., U.S. Pat. Nos. 6,156,313, 5,762,905, 6,685,942, and 7,364,737; U.S. Patent Pub. Nos. US2007/027499, US2006/0228367, US2007/0110757; International PCT Pub. No. WO2008/043052; Burioni et al. (1994) Proc Natl Acad Sci USA 91:355-359; Sanna et al. (1996) Virology 215:101-106; Sanna et al., (1995) Proc Natl Acad Sci USA 92:6439-6443; Cattani et al. (1997) J Clin Microbiol 35:1504-1509; and Ulbrandt et al. (2006) J Virology 80(16):7799-7806).

Non-limiting examples of antibodies that can conjugated to a protein transduction domain include AC8 (anti-HSV-1,2 antibody immunospecific for HSV glycoprotein D; U.S. Pat. No. 6,156,313); Fab 19 (anti-RSV antibody immunospecific for RSV glycoprotein F; U.S. Pat. Nos. 5,762,905, 6,685,942, and 7,364,737); Palivizumab (anti-RSV antibody immunospecific for RSV glycoprotein F; U.S. Pat. Nos. 7,132,100, 7,294,336; MedImmune); Motavizumab (anti-RSV antibody immunospecific for RSV glycoprotein F; MedImmune); CR4098 (anti-rabies virus antibody immunospecific for rabies virus glycoprotein antigenic site III; Crucell); CR57 (anti-rabies virus antibody immunospecific for rabies virus glycoprotein antigenic site I; U.S. Patent Pub. No. US 2003/0157112; Crucell); 17C7 (anti-rabies virus antibody immunospecific for rabies virus glycoprotein antigenic site III or minor site A; U.S. Patent Pub. No. US 2009/0041777; Massachusetts Biologic Laboratories); hE16 (anti-WNV antibody immunospecific for WNV envelope E protein, domain III; U.S. Patent Pub. No. 2006/0057149; Macrogenics); CR4374 (anti-WNV antibody immunospecific for WNV envelope E protein, domain III; U.S. Pat. No. 7,244,430; Crucell); CR3014 and CR3022 (anti-SARS-CoV antibodies immunospecific for SARS-CoV S1-RBD of glycoprotein S; U.S. Patent Pub. No. US 2006/0121580; Crucell); TI-23 (anti-CMV antibody immunospecific for CMV envelope glycoprotein B; U.S. Pat. No. 5,043,281; Teijin Pharma); HCMV37 (anti-CMV antibody immunospecific for CMV envelope glycoprotein B; Scotgen Biopharmaceuticals); TI-57 (anti-VZV antibody immunospecific for VZV envelope glycoprotein III; Teijin Pharma); KD-247 (anti-HIV antibody immunospecific for HIV V3 domain of glycoprotein 120 (gp120); Kaketsuken Chemo-Sero-Therapeutic Research Institute); XTL-6865 (cocktail of AB68 and AB65 anti-HCV antibodies immunospecific for HCV envelope glycoprotein E2; U.S. Patent Pub. No. US 2004/0071710; XTL Biopharmaceuticals); HuMax-HepC (anti-HCV antibody immunospecific for HCV envelope protein E2; U.S. Pat. No. 6,951,646; Genmab) and fragments thereof, including derivative antibodies (e.g., single chain antibodies) having identical or substantially identical complementarity determining regions and/or binding specificities.

Non-limiting examples of antibodies that can conjugated to a protein transduction domain also include preparations of antibodies purified from serum, where the preparation contains one or more antibodies that are specific for a viral envelope glycoprotein.

In some examples, a selected neutralizing antibody is an antibody fragment (e.g., Fab, Fab′, F(ab′)₂, single-chain Fv (scFv), Fv, dsFv, diabody, Fd and Fd′ fragments) or is an antibody fragment derived from a parent neutralizing antibody that retains the binding specificity of the parent antibody. Such antibody fragments can be modified by a selected protein transduction domain provided herein.

The virus neutralizing antibodies for modification by a protein transduction domain include antibodies which immunospecifically bind to one or more antigens on the surface of the virus. Such antibodies include antibodies that have a median effective concentration (EC50) of less than 0.01 nM, less than 0.025 nM, less than 0.05 nM, less than 0.1 nM, less than 0.25 nM, less than 0.5 nM, less than 0.75 nM, less than 1 nM, less than 1.25 nM, less than 1.5 nM, less than 1.75 nM, less than 2 nM, in an in vitro virus neutralization assay. Such assays are known in the art and are provided herein.

### (1) Herpes Virus Neutralizing Antibodies

In some examples, the modified therapeutic antibodies provided herein can immunospecifically bind to and neutralize viruses of the herpes family. For example, the therapeutic antibody selected for modification by a protein transduction domain can be an antibody that binds to herpes viruses, such as, but not limited to herpes simplex viruses type-1 and type-2 (HSV-1 and HSV-2), varicella zoster virus (VZV), cytomegalovirus (CMV), and Epstein Barr virus (EBV). In some examples, the modified therapeutic antibody is an antibody that binds to herpes simplex virus 1 (HSV-1) and/or herpes simplex virus 2 (HSV-2). Thus, in some examples, the antibody selected for modification is an anti-HSV neutralizing antibody. Such antibodies can be conventional antibodies that bind to a herpes virus or can be antibody fragments (e.g., Fab, Fab′, F(ab′)₂, single-chain Fv (scFv), Fv, dsFv, diabody, Fd and Fd′ fragments) that bind to a herpes virus.

In particular examples, the neutralizing antibody portion of the modified therapeutic antibody binds to a glycoprotein of a herpes virus (e.g., herpes simplex virus 1 (HSV-1) and/or herpes simplex virus 2 (HSV-2)). Exemplary herpes virus envelope glycoproteins include, for example, glycoprotein D (gD), glycoprotein H (gH), glycoprotein B (gB), glycoprotein C (gC), glycoprotein G (gG), glycoprotein I (gI), glycoprotein E (gE), glycoprotein J (gJ), glycoprotein K (gK), glycoprotein L (gL), glycoprotein M (gM), and UL32. In a particular example, the neutralizing antibody portion binds to herpes virus glycoprotein D.

An antibody selected for modification by a protein transduction domain can be any antibody that binds to a herpes virus glycoprotein, including antibody fragments that are derived from such antibodies.

(a) Herpes Virus Glycoprotein D Antibodies

In some examples, the antibody selected for modification by a protein transduction domain is an antibody that can immunospecifically bind to glycoprotein D. Glycoprotein D (gD) (SEQ ID NO: 1048) is an envelope glycoprotein required for HSV entry into cells, by interacting with cellular receptors such as nectin-1 (PRR1/HveC/CD111) or the herpes virus entry mediator A (HVEM/HveA) (Spear (2000) Alcohol Res Health 24:115-123). The epitopes of gD have been classified into eight antigenic clusters (Muggeridge et al. (1990) Immunochemistry of viruses, II. The basis for serodiagnosis and vaccines, (van Regenmortel and Neurath eds). New York, N.Y.: Elsevier Science Publishers). Two of these antigenic sites, I and VII, are responsible for eliciting highly protective antibody responses to gD in experimental animals (Eisenberg et al. (1986) Virus attachment and entry into cells (Crowell R, Lonberg-Holm K, eds), Washington, D.C.: Amer. Soc. Microbiol, 74-84; Geerligs et al. (1990) Arch Virol 114:251-258; Geerligs et al., (1990) J Gen Virol 71 (Pt 8):1767-1774). Consistent with the fact that gD is highly conserved between the two serotypes, both of these determinants induce type-common humoral responses. Potent Human recombinant monoclonal antibodies to the major type-common neutralizing determinant, the group I antigenic determinant from a HSV seropositive patient have previously been identified (Burioni et al., (1994) Proc Natl Acad Sci USA 91:355-359; Sanna et al., (1996) Virology 215:101-106). In particular examples, the antibody selected for modification by a protein transduction domain is an antibody that can immunospecifically bind to group I or group VII antigenic sites on glycoprotein D. Exemplary antibodies that can bind to HSV glycoprotein D are known in the art and include, for example, antibodies, including full length conventional antibodies and antibody fragments (e.g., single chain antibodies) described in, for example, U.S. Pat. No. 6,156,313, U.S. Patent Pub. 2007/027499, Sanna et al. (1995) Proc Natl Acad Sci USA 92:6439-6443, and Cattani et al. (1997) J Clin Microbiol 35:1504-1509.

One exemplary anti-HSV antibody that can be employed in the compositions and methods provided is AC8 (U.S. Pat. No. 6,156,313, ATCC Accession No. 69522, variable heavy chain (V_{H}) set forth in SEQ ID NO:1053, variable light chain (V_{L}) set forth in SEQ ID NO:1052). AC8 is a potent neutralizing monoclonal antibody (IgG1) specific for HSV glycoprotein D that acts with a post-attachment neutralization mechanism that can reduce cell-to-cell virus spread and axonal transmission (Burioni et al. (1994) Proc Natl Acad Sci USA 91:355-359; De Logu et al. (1998) J Clin Microbiol 36:3198-3204). AC8 also can interact with infected nerve fibers and terminals in vivo and can prevent axonal spread of the virus to epithelial cells (Mikloska et al. (1999) J Virology 73:5934-5944). In some examples, antibody fragments, such as a Fab or a single chain antibody, can be derived from the AC8 monoclonal antibody, which then can be modified by a selected PTD provided herein or known in the art. For example, the antibody selected for modification can be an AC8 Fab antibody having the heavy chain sequence set forth in SEQ ID NO:1056 and the light chain sequence set forth in SEQ ID NO:1016. An AC8 Fab antibody can thus be modified by any PTD provided herein or known in the art. The PTD can be conjugated to the Fab heavy chain or the Fab light chain. In one example, the PTD is conjugated to the Fab heavy chain. In another example, the PTD is conjugated to the Fab light chain. In a particular example, the AC8 Fab is modified by an HIV-TAT-peptide, RKKRRQRRR (SEQ ID NO: 915) conjugated to the heavy chain. An exemplary TAT-modified AC8 Fab antibody is provided herein having a heavy chain with the sequence of amino acids set forth in SEQ ID NO:1018 and a light chain with the sequence of amino acids set forth in SEQ ID NO:1016. In another example, the antibody selected for modification can be an AC8 single chain antibody having the sequence set forth in SEQ ID NO: 1. An AC8 single chain antibody can thus be modified by any PTD provided herein or known in the art. In a particular example, the AC8 single chain antibody is modified by an HIV-TAT peptide, RKKRRQRRR (SEQ ID NO: 915). An exemplary TAT-modified AC8 single chain antibody is provided herein having the amino acid sequence set forth in SEQ ID NO: 2.

In other particular examples, the AC8 single chain antibody is modified by a PTD peptide, such as any PTD peptide having the amino acid sequence set forth in Tables 3, 4, 5, 6, and 7 or SEQ ID NOS: 5-855, 860 and 867. Examples of such modified AC8 single chain antibodies are provided herein, including, but not limited to, AC8scFvTAT1A (SEQ ID NO: 1031), AC8scFvTAT1B (SEQ ID NO: 1032), AC8scFvTAT1C (SEQ ID NO: 1033), AC8scFvTAT2A (SEQ ID NO: 1034), AC8scFvTAT2B (SEQ ID NO: 1035), AC8scFvTAT2C (SEQ ID NO: 1036), AC8scFvTAT3A (SEQ ID NO: 1037), AC8scFvTAT3B (SEQ ID NO: 1038), AC8scFvTAT3C (SEQ ID NO: 1039), AC8scFvTAT4A (SEQ ID NO: 1040), AC8scFvTAT4B (SEQ ID NO: 1041), and AC8scFvTAT4C (SEQ ID NO: 1042).

In some examples, the antibody selected for modification is an antibody that contains that heavy chain complementarity determining region 3 (CDR3) of the anti-HSV AC8 antibody. For example, the selected antibody contains a CDR3 having the amino acid sequence VAYMLEPTVTAGGLDV (SEQ ID NO: 1051).

3. Attachment of the Protein Transduction Domain

The PTD can be conjugated to the therapeutic antibody in any suitable manner known in the art, such as, for example, conjugation by recombinant means or by chemical coupling. The linkage of the components in the conjugate can be by any method presently known in the art for attaching two moieties, so long as the attachment of the linker moiety to the antibody does not substantially impede binding of the antibody to the target antigen.

Any linker known to those of skill in the art can be used herein. Generally a different set of linkers will be used in conjugates that are fusion proteins from linkers in chemically-produced conjugates. Linkers and linkages that are suitable for chemically linked conjugates include, but are not limited to, disulfide bonds, thioether bonds, hindered disulfide bonds, and covalent bonds between free reactive groups, such as amine and thiol groups. These bonds are produced using heterobifunctional reagents to produce reactive thiol groups on one or both of the polypeptides and then reacting the thiol groups on one polypeptide with reactive thiol groups or amine groups to which reactive maleimido groups or thiol groups can be attached on the other. In some examples, several linkers can be included in order to take advantage of desired properties of each linker. Chemical linkers and peptide linkers can be inserted by covalently coupling the linker to the PTD and the therapeutic antibody. The heterobifunctional agents, described below, can be used to effect such covalent coupling. Peptide linkers also can be linked by expressing DNA encoding the linker and the antibody; the linker and the PTD; or the PTD, linker and antibody as a fusion protein. Flexible linkers and linkers that increase solubility of the conjugates are contemplated for use herein; either alone or with other linkers.

Linkers can be any moiety suitable to associate a PTD and an antibody. Such moieties include, but are not limited to, peptidic linkages; amino acid and peptide linkages, typically containing between one and about 50 amino acids; and chemical linkers, such as heterobifunctional cleavable cross-linkers. Other linkers include, but are not limited to peptides and other moieties that reduce steric hindrance between the PTD and the antibody, linkers that increase the flexibility of the conjugate, linkers that increase the solubility of the conjugate, or linkers that increase the serum stability of the conjugate. In some methods, where cleavage of the PTD is desired, the linkers can include intracellular enzyme substrates, photocleavable linkers and acid cleavable linkers.

a. Recombinant Methods

The modified therapeutic antibody can be produced by genetic engineering as a fusion polypeptide that includes the PTD and the therapeutic antibody which can be expressed in known suitable host cells. Fusion polypeptides, as described herein, can be formed and used in ways analogous to or readily adaptable from standard recombinant DNA techniques. Accordingly, provided herein are nucleic acid molecules and expression vectors comprising a nucleic acid encoding a PTD and the therapeutic antibody. There are an abundance of expression vectors available and one skilled in the art can easily select an appropriate vector. In addition, standard laboratory manuals on genetic engineering provide recombinant DNA methods and methods for making and using expression vectors. If desired, one or more amino acids, i.e. linker peptides, can additionally be inserted between the first peptide domain comprising the PTD and the second polypeptide domain comprising the therapeutic antibody.

Typically, the PTD is conjugated to the antibody in a manner that does not affect the binding specificity of the antibody. The PTD can be coupled directly to the therapeutic antibody on one of the terminal ends (N or C terminus) or on a selected side chain of one of the amino acids of the antibody molecule. The PTD also can be coupled indirectly to the therapeutic antibody by a connecting arm, or spacer, to one of the terminal ends of the peptide or to a side chain of one of the amino acids. In particular examples, where a single chain antibody is employed, the PTD is coupled to the C-terminus of the antibody.

### i. Spacer/Linker Peptides

The modified antibody can contain a peptide spacer, or linker, between the protein transduction domain and the antibody portion of the molecule. Spacer peptides also can be included between one or more domains of the antibody portion of the molecule. For example, where the antibody portion of the modified therapeutic antibody is a single chain antibody, the light chain variable region (V_{L}) of an antibody can be coupled to a heavy chain variable region (V_{H}) via a flexible linker peptide. Various peptide linkers are well-known in the art and can be employed in the provided methods. A peptide linker can include a series of glycine residues (Gly) or Serine (Ser) residues. Exemplary of polypeptide linkers are peptide having the amino acid sequences (Gly-Ser)ₙ, (GlyₘSer)ₙ or (SerₘGly)ₙ, in which m is 1 to 6, generally 1 to 4, and typically 2 to 4, and n is 1 to 30, or 1 to 10, and typically 1 to 4, with some Glu or Lys residues dispersed throughout to increase solubility (see, e.g., International PCT application No. WO 96/06641, which provides exemplary linkers for use in conjugates). In a particular example, the peptide linker is a peptide having the sequence GGSSRSSSSGGGGSGGGG (SEQ ID NO: 1047). Generally, the linker peptides are approximately 1-50 amino acids in length. The linkers used herein can serve merely to link the components of the conjugate, to increase intracellular availability, serum stability, specificity and solubility of the conjugate or provide increased flexibility or relieve steric hindrance in the conjugate.

b. Chemical Cross-Linking

Linkers, such as chemical linkers can be attached to purified antibodies using numerous protocols known in the art (see, e.g., Pierce Chemicals “Solutions, Cross-linking of Proteins: Basic Concepts and Strategies,” Seminar #12, Rockford, Ill.). Generally, for chemical conjugation, selective cross linking to particular sites on the antibody molecule are employed. Many known chemical cross-linking methods are non-specific, i.e., they do not direct the point of coupling to any particular site on PTD or the selected therapeutic antibody. As a result, use of non-specific cross-linking reagents may attack functional sites or sterically block active sites, rendering the conjugated proteins biologically inactive.

The PTD can be coupled directly to the therapeutic antibody on one of the terminal ends (N or C terminus) or on a selected side chain of one of the amino acids of the antibody molecule. The PTD also can be coupled indirectly to the therapeutic antibody by a connecting arm, or spacer, to one of the terminal ends of the peptide or to a side chain of one of the amino acids. In particular examples, the PTD is coupled to the C-terminus of the antibody.

One way to increase the coupling specificity of the PTD to the antibody is to direct chemical coupling to a functional group found only once or a few times in one or both of the polypeptides to be cross-linked. For example, in many proteins, cysteine, which is the only protein amino acid containing a thiol group, can be selected. Also, for example, if a polypeptide contains no lysine residues, a cross-linking reagent specific for primary amines will be selective for the amino terminus of that polypeptide. Successful utilization of this approach to increase coupling specificity requires that the polypeptide have the suitably rare and reactive residues in areas of the molecule that can be altered without loss of the molecule's biological activity.

Cysteine residues can be replaced when they occur in parts of a polypeptide sequence where their participation in a cross-linking reaction is otherwise likely to interfere with biological activity. When a cysteine residue is replaced, it is typically desirable to minimize resulting changes in polypeptide folding. Changes in polypeptide folding are minimized when the replacement is chemically and sterically similar to cysteine. For these reasons, serine typically is used as a replacement for cysteine. A cysteine residue also can be introduced into a polypeptide's amino acid sequence for cross-linking purposes. When a cysteine residue is introduced, introduction at or near the amino or carboxy terminus is typical. Conventional methods are available for such amino acid sequence modifications, whether the polypeptide of interest is produced by chemical synthesis or expression of recombinant DNA.

Coupling of the two constituents can be accomplished via a cross-linking reagent. Intermolecular cross-linking reagents are known in the art and can be utilized (see, e.g., Means and Feeney, Chemical Modification of Proteins, Holden-Day, 1974, pp. 39-43). Exemplary cross-linking reagents include, but are not limited to, N-succinimidyl 3-(2-pyridyldithio) propionate (SPDP) or N,N′-(1,3-phenylene) bismaleimide (both of which are highly specific for sulfhydryl groups and form irreversible linkages); N,N′-ethylene-bis-(iodoacetamide) or other such reagent having 6 to 11 carbon methylene bridges (which relatively specific for sulfhydryl groups); and 1,5-difluoro-2,4-dinitrobenzene (which forms irreversible linkages with amino and tyrosine groups). Other cross-linking reagents useful for this purpose include: p,p′-difluoro-N,N′-dinitrodiphenylsulfone (which forms irreversible cross-linkages with amino and phenolic groups); dimethyl adipimidate (which is specific for amino groups); phenol-1,4-disulfonylchloride (which reacts principally with amino groups); hexamethylenediisocyanate or diisothiocyanate, or azophenyl-p-diisocyanate (which reacts principally with amino groups); glutaraldehyde (which reacts with several different side chains) and bisdiazobenzidine (which reacts primarily with tyrosine and histidine).

Cross-linking reagents can be homobifunctional, i.e., having two functional groups (i.e. reactive groups) that undergo the same reaction. An example of homobifunctional cross-linking reagent is bismaleimidohexane (“BMH”). BMH contains two maleimide functional groups, which react specifically with sulfhydryl-containing compounds under mild conditions (pH 6.5-7.7). The two maleimide groups are connected by a hydrocarbon chain. BMH is useful for irreversible cross-linking of polypeptides that contain cysteine residues.

Cross-linking reagents also can be heterobifunctional. Heterobifunctional cross-linking reagents have two different functional groups, for example an amine-reactive group and a thiol-reactive group, that will cross-link two proteins having free amines and thiols, respectively. Exemplary heterobifunctional cross-linking reagents are succinimidyl 4-(N-maleimidomethyl)cyclohexane-1-carboxylate (“SMCC”), N-maleimidobenzoyl-N-hydroxysuccinimide ester (“MBS”), and succinimide 4-(p-maleimidophenyl) butyrate (“SMPB”), an extended chain analog of MBS. The succinimidyl group of these cross-linking reagents reacts with a primary amine forming an amide bond, and the thiol-reactive maleimide forms a covalent thioether bond with the thiol of a cysteine residue.

Cross-linking reagents often have low solubility in water. A hydrophilic moiety, such as a sulfonate group, can be added to the cross-linking reagent to improve its water solubility. Sulfo-MBS and sulfo-SMCC are examples of crosslinking reagents modified for water solubility. Many cross-linking reagents yield a conjugate that is essentially non-cleavable under cellular conditions. Some cross-linking reagents contain a covalent bond, such as a disulfide, that is cleavable under cellular conditions. For example, Traut's reagent, dithiobis (succinimidylpropionate) (“DSP”), and N-succinimidyl 3-(2-pyridyldithio) propionate (“SPDP”) are well-known cleavable cross-linking reagents. Another example is the hydrazine derivatives such as the 4-(4-N-maleimidophenyl)butyric acid hyrazide (MPBH), 4-(N-maleimidomethyl)cyclohexane-1-carboxyl-hydrazide (M2C2H), or the 3-(2-pyridyldithio) propionyl hydrazide (PDPH). The use of a cleavable cross-linking reagent permits the lysosomal enzyme to separate from the protein transduction domain after delivery into the target cell. Direct disulfide linkage also can be useful.

Numerous cross-linking reagents, including the ones discussed above, are commercially available. Detailed instructions for their use are readily available from the commercial suppliers. A general reference on protein cross-linking and conjugate preparation is: Wong, Chemistry of Protein Conjugation and Cross-linking, CRC Press (1991).

Chemical cross-linking can include the use of spacer arms. Spacer arms provide intramolecular flexibility or adjust intramolecular distances between conjugated domains and thereby can help preserve biological activity. A spacer arm can be in the form of a polypeptide domain that includes spacer amino acids, e.g. proline. A spacer arm can be part of the cross-linking reagent, such as in “long-chain SPDP” (Pierce Chem. Co., Rockford, Ill., cat. No. 21651H).

### D. ADDITIONAL MODIFICATIONS OF THERAPEUTIC ANTIBODIES

The PTD-modified therapeutic antibodies provided herein can be further modified. Exemplary modifications include, but are not limited to, modifications of the primary amino acid sequence of the PTD-modified antibody or alteration of the post-translational modification of the PTD-modified antibody. Exemplary post-translational modifications include, for example, glycosylation, acetylation, pegylation, phosphorylation, amidation, derivatization with protecting/blocking group, proteolytic cleavage, and linkage to a cellular ligand or other protein. Such modifications can be performed prior to or following attachment of the protein transduction domain to the antibody. Generally, the modifications do not result in increased immunogenicity of the antibody.

The modified therapeutic antibodies produced herein can include one or more amino acid substitutions, deletions or additions, either from natural mutation or Human manipulation from the parent antibody from which it was derived. Amino acid modifications can include modifications to alter the post-translational modification of the protein. For example, amino acid modifications can be introduced to alter the glycosylation of the antibody, reduce susceptibility to proteolysis; reduce susceptibility to oxidation; or alter or improve the binding properties of the antibody.

The PTD-modified therapeutic antibodies provided herein can be further modified by the covalent attachment of any type of molecule to the antibody such that covalent attachment does not prevent the antibody from binding to its corresponding epitope. For example, an antibody provided herein that neutralizes a virus can be further modified by covalent attachment of a molecule such that the covalent attachment does not prevent the antibody from binding to the virus. In some examples, the therapeutic antibody is modified prior to conjugation to the PTD. In other examples, the PTD-modified therapeutic antibody is further modified.

1. Modifications to Reduce Immunogenicity

In some examples, the antibodies provided herein can be further modified to reduce the immunogenicity in a subject, such as a Human subject. For example, one or more amino acids in the antibody can be modified to alter potential epitopes for Human T-cells in order to eliminate or reduce the immunogenicity of the antibody when exposed to the immune system of the subject. Exemplary modifications include substitutions, deletions and insertion of one or more amino acids, which eliminate or reduce the immunogenicity of the antibody. The antibodies provided herein can be modified to reduce immunogenicity before, after, or at the same time the antibody is conjugated to the protein transduction domain.

2. Attachment of a Detectable Moiety

In some examples, the antibodies provided herein can be further modified to contain a detectable moiety. The detectable moieties can be detected directly or indirectly. Depending on the detectable moiety selected, the detectable moiety can be detected in vivo and/or in vitro. The detectable moieties can be employed in diagnostic methods for detecting exposure to or localization of a pathogen to which that antibody can bind. The detectable moieties also can be employed in methods of preparation of the modified therapeutic antibodies, such as, for example, purification of the antibody. Typically, detectable moieties are selected such that conjugation of the detectable moiety does not interfere with the binding of the antibody to the target epitope. Methods of labeling antibodies with detectable moieties are known in the art and include, for example, recombinant and chemical methods. Exemplary detectable moieties and methods of use are provided elsewhere herein. The antibodies provided herein can be attached to a detectable moiety before, after, or at the same time the antibody is conjugated to the protein transduction domain.

3. Modifications to Improve Binding Specificity

The binding specificity of antibodies and antibody fragments can be altered or improved by techniques, such as phage display. Methods for phage display generally involve the use of a filamentous phage (phagemid) surface expression vector system for cloning and expressing antibody species of the library. Various phagemid cloning systems to produce combinatorial libraries have been described by others. See, for example the preparation of combinatorial antibody libraries on phagemids as described by Kang, et al., Proc. Natl. Acad. Sci., USA, 88:4363-4366 (1991); Barbas, et al., Proc. Natl. Acad. Sci., USA, 88:7978-7982 (1991); Zebedee, et al., Proc. Natl. Acad. Sci., USA, 89:3175-3179 (1992); Kang, et al., Proc. Natl. Acad. Sci., USA, 88:11120-11123 (1991); Barbas, et al., Proc. Natl. Acad. Sci., USA, 89:4457-4461 (1992); and Gram, et al., Proc. Natl. Acad. Sci., USA, 89:3576-3580 (1992), which references are hereby incorporated by reference.

The resulting phagemid library can be manipulated to increase and/or alter the immunospecificities of the antibodies or antibody fragment of the library to produce and subsequently identify additional antibodies with improved properties, such as increased binding to a target antigen. For example, either or both the H and L chain encoding DNA can be mutagenized in a complementarity determining region (CDR) of the variable region of the immunoglobulin polypeptide, and subsequently screened for desirable immunoreaction and neutralization capabilities. The resulting antibodies can then be screened in one or more of the assays described herein for determining neutralization capacity.

### E. PREPARATION OF MODIFIED THERAPEUTIC ANTIBODIES

The PTD-modified antibodies provided herein can be generated by any suitable method known in the art for the preparation of antibodies. Various combinations of host cells and vectors can be used to receive, maintain, reproduce and amplify nucleic acids (e.g. nucleic acids encoding antibodies such as modified therapeutic antibodies provided herein), and to express polypeptides encoded by the nucleic acids. In general, the choice of host cell and vector depends on whether amplification, polypeptide expression, and/or display on a genetic package, such as a phage, is desired. Methods for transforming host cells are well known. Any known transformation method, for example, electroporation, can be used to transform the host cell with nucleic acids. Procedures for the production of antibodies, such as monoclonal antibodies, and antibody fragments, such as single chain antibodies, are well known in the art.

Monoclonal antibodies can be prepared using a wide variety of techniques known in the art including, but not limited to, the use of hybridoma, recombinant, phage display technologies or a combination thereof. For example, monoclonal antibodies can be produced using hybridoma techniques including those known in the art and taught for example in Harlow et al. Antibodies: A Laboratory Manual (Cold Spring Harbor Laboratory Press, 2nd ed. 1988); Hammerling, Monoclonal Antibodies and T-Cell Hybridomas 5630681 (Elsevier N.Y. 1981).

Polypeptides, such as any set forth herein, including antibodies or fragments thereof, can be produced by any method known to those of skill in the art including in vivo and in vitro methods. Desired polypeptides can be expressed in any organism suitable to produce the required amounts and forms of the proteins, such as for example, needed for analysis, administration and treatment. Expression hosts include prokaryotic and eukaryotic organisms such as E. coli, yeast, plants, insect cells, mammalian cells, including Human cell lines and transgenic animals. Expression hosts can differ in their protein production levels as well as the types of post-translational modifications that are present on the expressed proteins. The choice of expression host can be made based on these and other factors, such as regulatory and safety considerations, production costs and the need and methods for purification.

1. Vectors and Nucleic Acids

Provided herein are nucleic acids encoding the modified antibodies provided herein. Nucleic acid molecules encoding the modified antibodies provided herein can be prepared using well-known recombinant techniques for manipulation of nucleic acid molecules. In one example, the nucleic acid molecule encoding the transduction domain is inserted into a plasmid encoding the antibody where the resulting plasmid encodes a fusion protein containing the transduction domain conjugated to the antibody. Typically the, nucleic acid encoding the protein transduction domain is inserted such that the protein transduction domain is located at the carboxyl terminus of the expressed fusion protein. In some examples the nucleic acid encoding a protein transduction domain is inserted into a plasmid encoding a single chain antibody.

Many expression vectors are available and known to those of skill in the art and can be used for expression of polypeptides. The choice of expression vector will be influenced by the choice of host expression system. In general, expression vectors can include transcriptional promoters and optionally enhancers, translational signals, and transcriptional and translational termination signals. Expression vectors that are used for stable transformation typically have a selectable marker which allows selection and maintenance of the transformed cells. In some cases, an origin of replication can be used to amplify the copy number of the vector.

2. Cells and Hosts

Nucleic acids encoding the modified antibodies provide herein can be expressed in a suitable host. A variety of host cells are known in the art for expressing proteins, such as antibodies, including single chain antibodies. These include but are not limited to mammalian cell systems infected with virus (e.g. vaccinia virus, adenovirus and other viruses); insect cell systems infected with virus (e.g. baculovirus); microorganisms such as yeast containing yeast vectors; or bacteria transformed with bacteriophage, DNA, plasmid DNA, or cosmid DNA. The expression elements of vectors vary in their strengths and specificities. Depending on the host-vector system used, any one of a number of suitable transcription and translation elements can be used. Host cells for the production of the modified antibodies provided herein are known in the art and include plant and animal cells. Exemplary mammalian cells for the production the modified antibodies provided herein include, but are not limited to, COS-1, COS-7, HEK293, BHK21, CHO, BSC-1, HepG2, Ag653, SP2/0, HeLa or any derivative, immortalized or transformed cell thereof. For display of the polypeptides on genetic packages, such as viruses, a host cell is selected that is compatible with such display. Typically, the genetic package is a virus, for example, a bacteriophage, and a host cell is chosen that can be infected with bacteriophage, and accommodate the packaging of phage particles, for example XL1-Blue cells. In another example, the host cell is the genetic package, for example, a bacterial cell genetic package, that expresses the modified antibody on the surface of the host cell.

a. Prokaryotic Cells

Prokaryotes, especially E. coli, provide a system for producing large amounts of proteins and can be used to express the modified therapeutic antibodies provided herein. Typically, E. coli host cells are used for amplification and expression of the provided modified therapeutic antibodies. Transformation of E. coli is simple and rapid technique well known to those of skill in the art. Expression vectors for E. coli can contain inducible promoters, such promoters are useful for inducing high levels of protein expression and for expressing proteins that exhibit some toxicity to the host cells. Examples of inducible promoters include the lac promoter, the trp promoter, the hybrid tac promoter, the T7 and SP6 RNA promoters and the temperature regulated λPL promoter.

Proteins, such as any provided herein, can be expressed in the cytoplasmic environment of E. coli. For some polypeptides, the cytoplasmic environment, can result in the formation of insoluble inclusion bodies containing aggregates of the proteins. Reducing agents such as dithiothreitol and β-mercaptoethanol and denaturants, such as guanidine-HCl and urea can be used to resolubilize the proteins, followed by subsequent refolding of the soluble proteins. An alternative approach is the expression of proteins in the periplasmic space of bacteria which provides an oxidizing environment and chaperonin-like and disulfide isomerases and can lead to the production of soluble protein. For example, for phage display of the proteins, the proteins are exported to the periplasm so that they can be assembled into the phage. Typically, a leader sequence is fused to the protein to be expressed which directs the protein to the periplasm. The leader is then removed by signal peptidases inside the periplasm. Examples of periplasmic-targeting leader sequences include the pelB leader from the pectate lyase gene and the leader derived from the alkaline phosphatase gene. In some cases, periplasmic expression allows leakage of the expressed protein into the culture medium. The secretion of proteins allows quick and simple purification from the culture supernatant. Proteins that are not secreted can be obtained from the periplasm by osmotic lysis. Similar to cytoplasmic expression, in some cases proteins can become insoluble and denaturants and reducing agents can be used to facilitate solubilization and refolding. Temperature of induction and growth also can influence expression levels and solubility, typically temperatures between 25° C. and 37° C. are used. Typically, bacteria produce non-glycosylated proteins. Thus, if proteins require glycosylation for function, glycosylation can be added in vitro after purification from host cells.

b. Yeast Cells

Yeasts such as Saccharomyces cerevisae, Schizosaccharomyces pombe, Yarrowia lipolytica, Kluyveromyces lactis and Pichia pastoris are well known yeast expression hosts that can be used to express the modified therapeutic antibodies provided herein. Yeast can be transformed with episomal replicating vectors or by stable chromosomal integration by homologous recombination. Typically, inducible promoters are used to regulate gene expression. Examples of such promoters include GAL1, GAL7 and GAL5 and metallothionein promoters, such as CUP1, AOX1 or other Pichia or other yeast promoter. Expression vectors often include a selectable marker such as LEU2, TRP1, HIS3 and URA3 for selection and maintenance of the transformed DNA. Proteins expressed in yeast are often soluble. Co-expression with chaperonins such as Bip and protein disulfide isomerase can improve expression levels and solubility. Additionally, proteins expressed in yeast can be directed for secretion using secretion signal peptide fusions such as the yeast mating type alpha-factor secretion signal from Saccharomyces cerevisae and fusions with yeast cell surface proteins such as the Aga2p mating adhesion receptor or the Arxula adeninivorans glucoamylase. A protease cleavage site such as for the Kex-2 protease, can be engineered to remove the fused sequences from the expressed polypeptides as they exit the secretion pathway. Yeast also is capable of glycosylation at Asn-X-Ser/Thr motifs.

c. Insect Cells

Insect cells, particularly using baculovirus expression, can be used to express the modified therapeutic antibodies provided herein. Insect cells express high levels of protein and are capable of most of the post-translational modifications used by higher eukaryotes. Baculovirus have a restrictive host range which improves the safety and reduces regulatory concerns of eukaryotic expression. Typical expression vectors use a promoter for high level expression such as the polyhedrin promoter of baculovirus. Commonly used baculovirus systems include the baculoviruses such as Autographa californica nuclear polyhedrosis virus (AcNPV), and the bombyx mori nuclear polyhedrosis virus (BmNPV) and an insect cell line such as Sf9 derived from Spodoptera frugiperda, Pseudaletia unipuncta (A7S) and Danaus plexippus (DpN 1). For high-level expression, the nucleotide sequence of the molecule to be expressed is fused immediately downstream of the polyhedrin initiation codon of the virus. Mammalian secretion signals are accurately processed in insect cells and can be used to secrete the expressed protein into the culture medium. In addition, the cell lines Pseudaletia unipuncta (A7S) and Danaus plexippus (DpN1) produce proteins with glycosylation patterns similar to mammalian cell systems.

An alternative expression system in insect cells is the use of stably transformed cells. Cell lines such as the Schnieder 2 (S2) and Kc cells (Drosophila melanogaster) and C7 cells (Aedes albopictus) can be used for expression. The Drosophila metallothionein promoter can be used to induce high levels of expression in the presence of heavy metal induction with cadmium or copper. Expression vectors are typically maintained by the use of selectable markers such as neomycin and hygromycin.

d. Mammalian Cells

Mammalian expression systems can be used to express the modified therapeutic antibodies provided herein. Expression constructs can be transferred to mammalian cells by viral infection such as adenovirus or by direct DNA transfer such as liposomes, calcium phosphate, DEAE-dextran and by physical means such as electroporation and microinjection. Expression vectors for mammalian cells typically include an mRNA cap site, a TATA box, a translational initiation sequence (Kozak consensus sequence) and polyadenylation elements. Such vectors often include transcriptional promoter-enhancers for high-level expression, for example the SV40 promoter-enhancer, the Human cytomegalovirus (CMV) promoter and the long terminal repeat of Rous sarcoma virus (RSV). These promoter-enhancers are active in many cell types. Tissue and cell-type promoters and enhancer regions also can be used for expression. Exemplary promoter/enhancer regions include, but are not limited to, those from genes such as elastase I, insulin, immunoglobulin, Mouse mammary tumor virus, albumin, alpha fetoprotein, alpha 1 antitrypsin, beta globin, myelin basic protein, myosin light chain 2, and gonadotropic releasing hormone gene control. Selectable markers can be used to select for and maintain cells with the expression construct. Examples of selectable marker genes include, but are not limited to, hygromycin B phosphotransferase, adenosine deaminase, xanthine-guanine phosphoribosyl transferase, aminoglycoside phosphotransferase, dihydrofolate reductase and thymidine kinase. Fusion with cell surface signaling molecules such as TCR-ζ and Fc_{ε}RI_{γ} can direct expression of the proteins in an active state on the cell surface.

Many cell lines are available for mammalian expression including mouse, rat, human, monkey, chicken and hamster cells. Exemplary cell lines include but are not limited to CHO, Balb/3T3, HeLa, MT2, Mouse NS0 (nonsecreting) and other myeloma cell lines, hybridoma and heterohybridoma cell lines, lymphocytes, fibroblasts, Sp2/0, COS, NIH3T3, HEK293, 293S, 2B8, and HKB cells. Cell lines also are available adapted to serum-free media which facilitates purification of secreted proteins from the cell culture media. One such example is the serum free EBNA-1 cell line (Pham et al., (2003) Biotechnol. Bioeng. 84:332-42.)

e. Plants

Transgenic plant cells and plants can be to express polypeptides such as any described herein. Expression constructs are typically transferred to plants using direct DNA transfer such as microprojectile bombardment and PEG-mediated transfer into protoplasts, and with agrobacterium-mediated transformation. Expression vectors can include promoter and enhancer sequences, transcriptional termination elements and translational control elements. Expression vectors and transformation techniques are usually divided between dicot hosts, such as Arabidopsis and tobacco, and monocot hosts, such as corn and rice. Examples of plant promoters used for expression include the cauliflower mosaic virus promoter, the nopaline syntase promoter, the ribose bisphosphate carboxylase promoter and the ubiquitin and UBQ3 promoters. Selectable markers such as hygromycin, phosphomannose isomerase and neomycin phosphotransferase are often used to facilitate selection and maintenance of transformed cells. Transformed plant cells can be maintained in culture as cells, aggregates (callus tissue) or regenerated into whole plants. Transgenic plant cells also can include algae engineered to produce proteases or modified proteases (see for example, Mayfield et al. (2003) PNAS 100:438-442). Because plants have different glycosylation patterns than mammalian cells, this can influence the choice of protein produced in these hosts.

3. Purification of Antibodies

Methods for purification of polypeptides, including the modified therapeutic antibodies provided, from host cells will depend on the chosen host cells and expression systems. For secreted molecules, proteins generally are purified from the culture media after removing the cells. For intracellular expression, cells can be lysed and the proteins purified from the extract. In one example, polypeptides are isolated from the host cells by centrifugation and cell lysis (e.g. by repeated freeze-thaw in a dry ice/ethanol bath), followed by centrifugation and retention of the supernatant containing the polypeptides. When transgenic organisms such as transgenic plants and animals are used for expression, tissues or organs can be used as starting material to make a lysed cell extract. Additionally, transgenic animal production can include the production of polypeptides in milk or eggs, which can be collected, and if necessary further the proteins can be extracted and further purified using standard methods in the art.

Proteins, such as the modified therapeutic antibodies provided herein, can be purified, for example, from lysed cell extracts, using standard protein purification techniques known in the art including but not limited to, SDS-PAGE, size fraction and size exclusion chromatography, ammonium sulfate precipitation and ionic exchange chromatography, such as anion exchange. Affinity purification techniques also can be utilized to improve the efficiency and purity of the preparations. For example, antibodies, receptors and other molecules that bind proteases can be used in affinity purification. Expression constructs also can be engineered to add an affinity tag to a protein such as a myc epitope, GST fusion or His₆ and affinity purified with myc antibody, glutathione resin and Ni-resin, respectively. Purity can be assessed by any method known in the art including gel electrophoresis and staining and spectrophotometric techniques.

The isolated polypeptides then can be analyzed, for example, by separation on a gel (e.g. SDS-Page gel), size fractionation (e.g. separation on a Sephacryl™ S-200 HiPrep™ 16×60 size exclusion column (Amersham from GE Healthcare Life Sciences, Piscataway, N.J.). Isolated polypeptides also can be analyzed in binding assays, typically binding assays using a binding partner bound to a solid support, for example, to a plate (e.g. ELISA-based binding assays) or a bead, to determine their ability to bind desired binding partners. The binding assays described in the sections below, which are used to assess binding of precipitated phage displaying the polypeptides, also can be used to assess polypeptides isolated directly from host cell lysates. For example, binding assays can be carried out to determine whether antibody polypeptides bind to one or more antigens, for example, by coating the antigen on a solid support, such as a well of an assay plate and incubating the isolated polypeptides on the solid support, followed by washing and detection with secondary reagents, e.g. enzyme-labeled antibodies and substrates.

### F. THERAPEUTIC METHODS

1. Selection of Subjects for Therapy

A patient or subject for therapy with a PTD-modified therapeutic antibody provided herein include, but are not limited to, a patient or subject that has been exposed to a pathogen (e.g., a virus or bacterial pathogen), a patient or subject who exhibits one or more symptoms of a pathogenic infection and patient or subject who is at risk of a pathogenic infection.

In particular examples, the pathogen is a virus, such as herpes virus (e.g., herpes simplex vires, HSV), and the modified therapeutic antibody is effective for neutralization of the virus. In such examples, a patient or subject for therapy with a modified therapeutic antibody provided herein that is specific for neutralizing herpes viruses herein includes, but is not limited to, a patient or subject that has been exposed to a herpes virus, a patient or subject who exhibits one or more symptoms of a herpes virus infection, a patient or subject who is at risk of a herpes virus infection, and a patient or subject that has a herpes virus infection. Exemplary herpes virus infections include those cause by herpes viruses, such as, but not limited to herpes simplex viruses type-1 and type-2 (HSV-1 and HSV-2), varicella zoster virus (VZV), cytomegalovirus (CMV), and Epstein Barr virus (EBV).

The modified therapeutic antibodies provided herein can be administered to a patient or for the treatment of any HSV-mediated disease. For example, the PTD-modified therapeutic antibodies provided herein can be administered to a patient or a subject to alleviate one or more symptoms or conditions associated with a herpes virus infection, including, but not limited to, herpes ocularis, herpes labialis, herpes facialis, dental herpes, herpes gingivostomatitis, herpes pharyngitis, herpes digitalis, herpes genitalis, cutaneous infections, herpes keratitis, eczema herpeticum, encephalitis, herpes gladiatorum, whitlow, herpes stomatitis, ocular infections, neonatal herpes, chicken pox, shingles, zoster ostitis, genital warts, pneumonia and hepatitis. Such diseases and condition are well known and readily diagnosed by physicians or ordinary skill.

The PTD-modified therapeutic antibodies provided herein can be administered to a patient or a subject having a herpes virus infection for the maintenance or suppression therapy of recurring herpes virus disease.

The PTD-modified therapeutic antibodies provided herein can be administered to a patient or a subject at risk of a herpes virus infection, including, but not limited to immunocompromised patients, such as, for example, AIDS patients, transplant recipients, cancer patients, patients with genetically based immunodeficiency, infants, and the elderly.

Tests for various pathogens and pathogenic infection are known in the art and can be employed for the assessing whether a subject is a candidate for therapy with a modified therapeutic antibody provided herein. For example, tests for herpes virus infection, including HSV infection, are well known and include for example, viral culture plaque assays, antigen detection test, polymerase chain reaction (PCR) tests, and various antibody serological tests. Tests for viral infection can be performed on sample obtained from sores in the genital area fluid samples, such as spinal fluid, blood, urine, or tears.

Assessing or determining if a patient or subject is at risk of a herpes virus infection, such as a HSV infection, entails the assessment of various risk factors. Risk factors include multiple sexual partners, increasing age, female gender, low socioeconomic status and Human immunodeficiency virus (HIV) infection. Also, a fetus is at risk of infection during birth if the mother is infected by the virus (e.g. HSV).

2. Dosages

The PTD-modified therapeutic antibody is administered in an amount sufficient to exert a therapeutically useful effect in the absence of undesirable side effects on the patient treated. The therapeutically effective concentration of a modified therapeutic antibody can be determined empirically by testing the polypeptides in known in vitro and in vivo systems such as by using the assays provided herein or known in the art.

An effective amount of antibody to be administered therapeutically will depend, for example, upon the therapeutic objectives, the route of administration, and the condition of the patient. In addition, the attending physician takes into consideration various factors known to modify the action of drugs, including severity and type of disease, patient's health, body weight, sex, diet, time and route of administration, other medications and other relevant clinical factors. Accordingly, it will be necessary for the therapist to titer the dosage and modify the route of administration as required to obtain the optimal therapeutic effect. Typically, the clinician will administer the antibody until a dosage is reached that achieves the desired effect. The progress of this therapy is easily monitored by conventional assays. Exemplary assays for monitoring treatment of a viral infection are know in the art and include for example, viral titer assays.

Generally, the dosage ranges for the administration of the modified therapeutic antibodies provided herein are those large enough to produce the desired effect in which the symptom(s) of the pathogen-mediated disease (e.g. viral disease) are ameliorated or the likelihood of virus infection is decreased. The dosage is not so large as to cause adverse side effects, such as hyperviscosity syndromes, pulmonary edema or congestive heart failure. Generally, the dosage will vary with the age, condition, sex and the extent of the disease in the patient and can be determine by one of skill in the art. The dosage can be adjusted by the individual physician in the event of the appearance of any adverse side effect. Exemplary dosages include, but are not limited to, about or 0.01 mg/kg to about or 300 mg/kg, such as for example, about or 0.01 mg/kg, about or 0.1 mg/kg, about or 0.5 mg/kg, about or 1 mg/kg, about or 5 mg/kg, about or 10 mg/kg, about or 50 mg/kg, about or 100 mg/kg, about or 150 mg/kg, about or 200 mg/kg, about or 250 mg/kg, or about or 300 mg/kg.

For treatment of a viral infection, the dosage of the PTD-modified therapeutic antibody can vary depending on the type and severity of the disease. The modified therapeutic antibodies can be administered in a single dose, in multiple separate administrations, or by continuous infusion. For repeated administrations over several days or longer, depending on the condition, the treatment can be repeated until a desired suppression of disease symptoms occurs or the desired improvement in the patient's condition is achieved. Repeated administrations can include increased or decreased amounts of the modified therapeutic antibody depending on the progress of the treatment. Other dosage regimens also are contemplated.

Therapeutic efficacy of a particular dosage or dosage regimen also can be assessed, for example, by measurement of viral titer in the subject prior to and following administration of one or more doses of the PTD-modified therapeutic antibody. Dosage amounts and/or frequency of administration can be modified depending on the desired rate of clearance of the virus in the subject.

As will be understood by one of skill in the art, the optimal treatment regimen will vary and it is within the scope of the treatment methods to evaluate the status of the disease under treatment and the general health of the patient prior to, and following one or more cycles of therapy in order to determine the optimal therapeutic dosage and frequency of administration. It is to be further understood that for any particular subject, specific dosage regimens can be adjusted over time according to the individual need and the professional judgment of the person administering or supervising the administration of the pharmaceutical formulations, and that the dosages set forth herein are exemplary only and are not intended to limit the scope thereof. The amount of a PTD-modified therapeutic antibody to be administered for the treatment of a disease or condition, for example a viral infection (e.g. a herpes virus infection), can be determined by standard clinical techniques (e.g. viral titer assays). In addition, in vitro assays and animal models can be employed to help identify optimal dosage ranges.

3. Routes of Administration

The PTD-modified therapeutic antibodies provided herein can be administered to a subject by any method known in the art for the administration of polypeptides, including for example systemic or local administration. The modified therapeutic antibodies can be administered by routes, such as parenteral, topical, mucosal, intravenous, intraperitoneal, intramuscular, subcutaneous, or intracavity. The modified therapeutic antibodies can be administered externally to a subject, at the site of the disease for exertion of local or transdermal action. The mode of administration can include topical or any other administration of a composition on, in or around areas of the body that may come on contact with fluid, cells or tissues that are infected, contaminated or have associated therewith a pathogen, such as a virus.

In some examples, the modified therapeutic antibodies provided herein increase the efficacy of mucosal immunization against a virus. Thus, in particular examples the modified therapeutic antibodies are administered to a mucosal surface. For example, the modified antibodies can be delivered via routes such as oral (e.g., buccal, sublingual), ocular (e.g., corneal, conjunctival, intravitreally, intra-aqueous injection), intranasal, genital (e.g., vaginal), rectal, pulmonary, stomachic, or intestinal. The modified therapeutic antibodies provided herein can be administered systemically, such as parenterally, for example, by injection or by gradual infusion over time or enterally (i.e. digestive tract). The modified therapeutic antibodies provided herein also can be administered topically, such as for example, by topical installation (e.g., eyedrops, gels, or ointments), application (e.g. vaginally) of suppositories, liquid solutions, gels, ointments, or inhalation (e.g., nasal sprays, inhalers). Administration can be effected prior to exposure to the virus or subsequent to exposure to the virus.

4. Combination Therapies

The PTD-modified therapeutic antibodies provide herein can be administered in combination with one or more therapeutic agents for the treatment of a disease or condition. For example, the modified therapeutic antibodies provided herein can be administered in combination with one or more antiviral agents for the treatment of a pathogenic infection, such as a viral infection. Such agents can include agents to decrease and/or eliminate the pathogenic infection or agents to alleviate one or more symptoms of a pathogenic infection. In some examples, a plurality of modified therapeutic antibodies also can be administered in combination.

The one or more additional agents can be administered simultaneously, sequentially or intermittently with the modified therapeutic antibody. The agents can be co-administered with the modified therapeutic antibody, for example, as part of the same pharmaceutical composition or same method of delivery, or at the same time as the modified therapeutic antibody but by a different means of delivery. The agents also can be administered at a different time than administration of the modified therapeutic antibody, but close enough in time to the administration of the modified therapeutic antibody to have a combined prophylactic or therapeutic effect.

Exemplary antiviral agents that can be selected for combination therapy with a modified therapeutic antibody provided herein include, but are not limited to, antiviral compounds, antiviral proteins, antiviral peptides, antiviral protein conjugates and antiviral peptide conjugates, including, but not limited to, nucleoside analogs, nucleotide analogs, immunomodulators (e.g. interferons) and immunostimulants.

Exemplary antiviral agents for the treatment of virus infections include, but are not limited to, acyclovir, famciclovir, ganciclovir, penciclovir, valacyclovir, valganciclovir, idoxuridine, trifluridine, brivudine, cidofovir, docosanol, fomivirsen, foscarnet, tromantadine, imiquimod, podophyllotoxin, entecavir, lamivudine, telbivudine, clevudine, adefovir, tenofovir, boceprevir, telaprevir, pleconaril, arbidol, amantadine, rimantadine, oseltamivir, zanamivir, peramivir, inosine, interferon (e.g., Interferon alfa-2b, Peginterferon alfa-2a), ribavirin/taribavirin, abacavir, emtricitabine, lamivudine, didanosine, zidovudine, apricitabine, stampidine, elvucitabine, racivir, amdoxovir, stavudine, zalcitabine, tenofovir, efavirenz, nevirapine, etravirine, rilpivirine, loviride, delavirdine, atazanavir, fosamprenavir, lopinavir, darunavir, nelfinavir, ritonavir, saquinavir, tipranavir, amprenavir, indinavir, enfuvirtide, maraviroc, vicriviroc, PRO 140, ibalizumab, raltegravir, elvitegravir, bevirimat, vivecon, including tautomeric forms, analogs, isomers, polymorphs, solvates, derivatives, or salts thereof.

Exemplary antiviral agents for the treatment of herpes virus infections include, but are not limited to, purine analogs such as, for example, acyclovir, famciclovir, ganciclovir, penciclovir, valacyclovir, valganciclovir; pyrimidine analogs, such as, for example, idoxuridine, trifluridine, brivudine, cidofovir, docosanol, fomivirsen, foscarnet, tromantadine, including tautomeric forms, analogs, isomers, polymorphs, solvates, derivatives, or salts thereof.

The PTD-modified therapeutic antibodies also can be administered in combination with one or more agents capable of stimulating cellular immunity, such as cellular mucosal immunity. Any agent capable of stimulatory cellular immunity can be used. Exemplary immunostimulatory agents include, cytokines, such as, but not limited to, interferons (e.g., IFN-α, β, γ, ω), GMCSF (granulocyte macrophage colony stimulating factor), Interleukin-12 (IL-12), Interleukin-14 (IL-14), and Tumor Necrosis Factor (TNF).

For combination therapies with anti-pathogenic agents, dosages for the administration of such compounds are known in the art or can be determined by one skilled in the art according to known clinical factors (e.g., subject's species, size, body surface area, age, sex, immunocompetence, and general health, duration and route of administration, the kind and stage of the disease, and whether other treatments, such as other anti-pathogenic agents, are being administered concurrently).

The modified therapeutic antibodies provide herein can be administered in combination with one or more agents that increase the availability or exposure of the modified antibody to the site therapy. For example, the modified therapeutic antibodies can be administered with one or more agents that increase the permeability of tissues and/or absorption of the antibody at the site of local administration. In some examples, the modified therapeutic antibodies is administered with one or more agents that increase the permeability and/or absorption of the antibody in ocular tissues. Exemplary of such agents include but are not limited to a viscoelastic agent, such as hyaluronic acid (e.g., Healon®) or hydroxymethylpropylcellulose (Coatel®). Such agents can be administered simultaneously, sequentially or intermittently with the modified therapeutic antibody. In some examples, the agent and the modified therapeutic antibody are administered in a single composition. In some examples, the agent and the modified therapeutic antibody are administered in separate compositions.

### G. DIAGNOSTIC METHODS

1. Assays for Selection of a Protein Transduction Domain

The PTDs provided herein can be selected for modification of a therapeutic antibody based on assays known in the art for measuring the function of a protein transduction domain. For example, known assays which measure the ability of a PTD to bind to a target cell surface and/or are uptaken by the cell can be employed.

The capacity of PTDs to bind to glycosaminoglycans (GAGs), such as glycosaminoglycans present on a target cell surface, can be determined by direct or indirect glycosaminoglycan-binding assays known in the art. Exemplary assays include the affinity co-electrophoresis (ACE) assay for peptide glycosaminoglycan binding described in the International PCT Patent Pub. No. WO 00/45831. Several other methods well known in the art are available for analyzing GAG-peptide interactions, for example, methods described in International PCT Patent Pub. No. WO 01/64738, Weisgraber and Rall (1987) J. Biol. Chem. 262(33):11097-103, or by a modified ELISA test, where the PTD peptide is conjugated to a marker and assayed for binding to multi-well plates, which are coated with a specific GAG (chondroitin sulfate A, B and C, heparin, heparan sulfate, hyaluronic acid, keratan sulfate, syndecan). Binding is determined using specific analysis related to the marker. Such assays can be performed in the presence or absence of conjugation to the therapeutic antibody.

2. In Vitro Assays for Analyzing Virus Neutralization Effects of Antibodies

The PTD-modified antibodies provided herein can be analyzed by any suitable method known in the art for the detection of viral neutralization. Methods for detection of viral neutralization include, but are not limited to, plaque assays and assays for inhibition of syncytium formation. Such assays can be employed to assess, for example, inhibition of viral attachment, viral entry and cell-to-cell spread of the virus (see, e.g. Burioni et al. (1994) Proc. Natl. Acad. Sci. U.S.A. 91:355-359; Sanna et al. (2000) Virology 270:386-3961; De Logu et al. (1998) J Clin Microbiol 36:3198-3204). One of skill in the art can identify any assay capable of measuring viral neutralization.

Standard plaque assays include, for example, plaque reduction assays, plaque size reduction assays, neutralization assays and neutralization kinetic assays. These assays measure the formation of viral plaques (i.e. areas of lysed cells) following infection of target cell monolayers by a virus. Exemplary target cell lines that can be used in plaque reduction assays include, but are not limited to, Vero cells, MRC-5 cells, RC-37 cells, BHK-21/C13 cells and HEp-2 cells. One of skill in the art can identify appropriate target cell lines for use in a plaque assay. Selection of an appropriate cell line for a plaque assay can depend on known factors, such as, for example, cell infectivity and the ability of the virus to propagate in and lyse the target cell.

Plaque reduction assays can be used to measure the ability of the modified therapeutic antibody to effect viral neutralization in solution. In exemplary plaque reduction assays, the antibody and the virus are pre-incubated prior to the addition of target cells. Target cells are then infected with the antibody/virus mixture and a plaque assay is performed following a predetermined infection period. One of skill in the art can determine the incubation times required based on known examples in the art. A reduction in the number of virus plaques produced following infection of the target cells indicates the ability of the antibody to prevent binding of the virus to the target cells independent of antibody attachment to the target cell and/or antibody internalization (see Example 3 below).

Plaque size reduction assays can be used to measure the ability of the modified therapeutic antibody to inhibit viral cell-to-cell spread. In exemplary plaque size reduction assays, the target cells are first infected with the virus for a predetermined infection period and then the antibody is added to the infected cell. One of skill in the art can determine the incubation times required based on known examples in the art. A reduction in the size (i.e. diameter) of the virus plaques indicates that the antibody is capable of preventing viral cell-to-cell spread (see Example 5 below).

Virus neutralization assays can be used to measure the ability of the modified therapeutic antibody to effect viral neutralization at the target cell surface by association of the antibody with the target cell prior to virus exposure. In exemplary virus neutralization assays, the antibody and target cells are pre-incubated for a predetermined period of time to allow for binding of the antibody to the targeted cell. Following the pre-incubation period, the unbound antibody is removed and the target cells are infected with the virus. A reduction in the number of plaques in this assay indicates the ability of the antibody to prevent viral infection dependent upon attachment to the target cell and/or internalization of the antibody. This assay also can be used to measure neutralization kinetics by varying antibody concentrations and pre-incubation times (see Example 4 below).

Exemplary assays for inhibition of syncytium formation can be employed to measure antibody-mediated inhibition of viral cytopathic effects by blocking the formation of syncytia when using a fusogenic viral strain. One of skill in the art can identify an appropriate fusogenic viral strain for use in this assay.

3. In Vivo Animal Models for Assessing Efficacy of the Modified Therapeutic Antibodies

In vivo studies using animal models also can be performed to assess the efficacy of the modified therapeutic antibodies provided herein. The therapeutic effect of modified therapeutic antibodies can be assessed using animal models of the pathogenic infection, including animal models of viral, bacterial or fungal infection. Such animal models are known in the art, and include, but are not limited to, animal models for viral infection, such as but not limited to ocular, flank, and vaginal models of active and latent herpes virus infection (see, e.g., Sanna et al. (1996) Virology 215:101-106; Zeitlin et al. (1996) Virology 225:213-215; Stanberry et al. (1992) J. Infect. Dis. 146:397-404; Bourne et al. (1992) Antimicr. Agents Chemother. 36:2020-2024; Pavann-Langston and Dunkel (1989) Arch. Ophathalmol. 107:1068-1072; Carter et al. (1992) Invest Opthalmol Vis Sci 33:1934-1939; Ritchie et al. (1993) Invest Opthalmol Vis Sci 34:2460-2468).

A variety of assays, such as those employing in vivo animal models, are available to those of skill in the art for evaluating the ability of the modified therapeutic antibodies to inhibit or treat herpes virus infection and/or herpes infection reactivation. Such assays include, but are not limited to, rodent in vitro explant-cocultivation models for HSV1 (Lelb et al. (1989) J. Virol. 63:759-768), and rodent eye and ear models for HSV1 reactivation (see, e.g., Shimeld et al. (1990) J. Gen. Virol. 71:397-404; Hill et al. (1978) J. Gen. Virol. 39:21-28; Gordon et al. (1990) Invest. Opthalmol. Vis. Sci. 31:921-924; Varnell et al. (1987) Curr. Eye Res. 6:277-279; Kaufman et al. (1991) Antiviral Res. 16:227-232).

As described herein, the PTD-modified therapeutic antibodies can be conjugated to a detectable moiety for in vivo detection. Such antibodies can be employed, for example, to evaluate the localization and/or persistence of the modified therapeutic antibody at an in vivo site, such as, for example, a mucosal site. In exemplary assays, the modified therapeutic antibodies can be coupled to a detectable moiety (e.g., a fluorescent dye, such as Cy5) and can be administered to an animal for in vivo detection in tissues, such as the eye or other mucosal surface and in infected nerve fibers and sensory neurons (see, e.g., Sanna et al., (1999) J. Virol. 73:8817-8823). The modified therapeutic antibodies which are coupled to a detectable moiety can be detected in vivo by any suitable method known in the art. The PTD-modified therapeutic antibodies which are coupled to a detectable moiety also can be detected in samples, such as tissue or fluid samples obtained from the subject following administration of the antibody. In an exemplary assay, a modified therapeutic antibody can be detected by scanning confocal microscopy (LSCM) of corneal samples obtained from a subject that has been administered the antibody (e.g., by topical instillation of the eye or sub-conjunctival injection).

4. In Vitro Detection of Pathogenic Infection

In general, pathogens, such as HSV, can be detected in a subject or patient based on the presence of one or more HSV proteins and/or polynucleotides encoding such proteins in a biological sample (e.g., blood, sera, sputum urine and/or other appropriate cells or tissues) obtained from a subject or patient. Such proteins can be used as markers to indicate the presence or absence of HSV in a subject or patient. The modified antibodies provided herein can be employed for detection of the level of antigen and/or epitope that binds to the agent in the biological sample.

A variety of assay formats are known to those of ordinary skill in the art for using a PTD-modified antibody to detect polypeptide markers in a sample (see, e.g., Harlow and Lane, Antibodies: A Laboratory Manual, Cold Spring Harbor Laboratory, 1988). In general, the presence or absence of HSV in a subject or patient can be determined by contacting a biological sample obtained from a subject or patient with a modified antibody provided herein and detecting in the sample a level of polypeptide that binds to the modified antibody.

In some examples, the assay involves the use of modified antibody provided herein immobilized on a solid support to bind to and remove the target polypeptide from the remainder of the sample. The bound polypeptide can then be detected using a detection reagent that contains a reporter group and specifically binds to the antibody/polypeptide complex. Such detection reagents can comprise, for example, a binding agent that specifically binds to the polypeptide or an antibody or other agent that specifically binds to the binding agent.

In some examples, a competitive assay can be utilized, in which a polypeptide is labeled with a reporter group and allowed to bind to the immobilized modified antibody after incubation of the modified antibody with the sample. The extent to which components of the sample inhibit the binding of the labeled polypeptide to the modified antibody is indicative of the reactivity of the sample with the immobilized modified antibody. Suitable polypeptides for use within such assays include full length HSV proteins and portions thereof, including HSV glycoprotein D, to which a modified antibody binds, as described above.

The solid support can be any material known to those of ordinary skill in the art to which the protein can be attached. For example, the solid support can be a test well in a microtiter plate or a nitrocellulose or other suitable membrane. The support also can be a bead or disc, such as glass, fiberglass, latex or a plastic material such as polystyrene or polyvinylchloride. The support also can be a magnetic particle or a fiber optic sensor, such as those disclosed, for example, in U.S. Pat. No. 5,359,681. The modified antibody can be immobilized on the solid support using a variety of techniques known to those of skill in the art. The modified antibody can be immobilized by adsorption to a well in a microtiter plate or to a membrane. In such cases, adsorption can be achieved by contacting the modified antibody, in a suitable buffer, with the solid support for a suitable amount of time. The contact time varies with temperature, but is typically between about 1 hour and about 1 day. In general, contacting a well of a plastic microtiter plate (such as polystyrene or polyvinylchloride) with an amount of modified antibody ranging from about 10 ng to about 10 μg, and typically about 100 ng to about 1 μg, is sufficient to immobilize an adequate amount of modified antibody.

Covalent attachment of modified antibody to a solid support can generally be achieved by first reacting the support with a bifunctional reagent that will react with both the support and a functional group, such as a hydroxyl or amino group, on the modified antibody. For example, the modified antibody can be covalently attached to supports having an appropriate polymer coating using benzoquinone or by condensation of an aldehyde group on the support with an amine and an active hydrogen on the binding partner (see, e.g., Pierce Immunotechnology Catalog and Handbook, 1991, at A12-A13).

In some examples, the assay is performed in a flow-through or strip test format, wherein the modified antibody is immobilized on a membrane, such as nitrocellulose. In the flow-through test, polypeptides within the sample bind to the immobilized modified antibody as the sample passes through the membrane. A second, labeled binding agent then binds to the modified antibody-polypeptide complex as a solution containing the second binding agent flows through the membrane.

Additional assay protocols exist in the art that are suitable for use with the HSV proteins or PTD-modified antibodies provided herein. The above descriptions are intended to be exemplary only. For example, it will be apparent to those of ordinary skill in the art that the above protocols can be readily modified to use HSV polypeptides to detect antibodies that bind to such polypeptides in a biological sample. The detection of such protein-specific antibodies can allow for the identification of HSV infection.

To improve sensitivity, multiple HSV protein markers can be assayed within a given sample. It will be apparent that modified antibodies specific for different HSV polypeptides can be combined within a single assay. Further, multiple primers or probes can be used concurrently. The selection of HSV protein markers can be based on routine experiments to determine combinations that results in optimal sensitivity. In addition, or alternatively, assays for HSV proteins provided herein can be combined with assays for other known HSV antigens.

5. In Vivo Detection of Pathogenic Infection

The modified therapeutic antibodies provided herein can be employed as an in vivo diagnostic agent. For example, the modified therapeutic antibodies can provide an image of infected tissues (e.g., HSV infection in brain and neurons) using detection methods such as, for example, magnetic resonance imaging, X-ray imaging, computerized emission tomography and other imaging technologies. For the imaging of HSV infected tissues, for example, the antibody portion of the modified therapeutic antibody generally will bind to HSV (e.g., binding a HSV gD antigen or epitope), and the imaging agent will be an agent detectable upon imaging, such as a paramagnetic, radioactive or fluorescent agent that is coupled to the modified antibody. Generally, for use as a diagnostic agent, the modified therapeutic antibody is coupled directly or indirectly to the imaging agent.

Many appropriate imaging agents are known in the art, as are methods for their attachment to modified therapeutic antibodies (see, e.g., U.S. Pat. Nos. 5,021,236 and 4,472,509). Exemplary attachment methods involve the use of a metal chelate complex employing, for example, an organic chelating agent such a DTPA attached to the antibody (U.S. Pat. No. 4,472,509). The antibodies also can be reacted with an enzyme in the presence of a coupling agent such as glutaraldehyde or periodate. Conjugates with fluorescein markers are prepared in the presence of such coupling agents or by reaction with an isothiocyanate.

For in vivo diagnostic imaging, the type of detection instrument available is considered when selecting a given radioisotope. The radioisotope selected has a type of decay which is detectable for a given type of instrument. Another factor in selecting a radioisotope for in vivo diagnosis is that the half-life of the radioisotope be long enough so that it is still detectable at the time of maximum uptake by the target, but short enough so that deleterious radiation with respect to the host is minimized. Typically, a radioisotope used for in vivo imaging will lack a particle emission, but produce a large number of photons in the 140-250 keV range, which can be readily detected by conventional gamma cameras.

For in vivo diagnosis, radioisotopes can be bound to the antibodies provided herein either directly or indirectly by using an intermediate functional group. Exemplary intermediate functional groups which can be used to bind radioisotopes, which exist as metallic ions, to antibodies include bifunctional chelating agents, such as diethylene-triaminepentaacetic acid (DTPA) and ethylenediaminetetraacetic acid (EDTA) and similar molecules. Examples of metallic ions which can be bound to the modified therapeutic antibodies provided herein include, but are not limited to, ⁷²Arsenic, ²¹¹Astatine, ¹⁴Carbon, ⁵¹Chromium, ³⁶Chlorine, ⁵⁷Cobalt, ⁵⁸Cobalt, ⁶⁷Copper, ¹⁵²Europium, ⁶⁷Gallium, ⁶⁸Gallium, ³Hydrogen, ¹²³Iodine, ¹²⁵Iodine, ¹³¹Iodine, ¹¹¹Indium, ⁵⁹Iron, ³²Phosphorus, 186Rhenium, 188Rhenium, ⁹⁷Ruthenium, ⁷⁵Selenium, ³⁵Sulphur, ^{99m} Technicium, ²⁰¹Thallium, ⁹⁰Yttrium and ⁸⁹Zirconium.

The modified antibodies provided herein can be labeled with a paramagnetic isotope for purposes of in vivo diagnosis, as in magnetic resonance imaging (MRI) or electron spin resonance (ESR). In general, any conventional method for visualizing diagnostic imaging can be utilized. Generally, gamma and positron emitting radioisotopes are used for camera imaging and paramagnetic isotopes for MRI. Elements which are particularly useful in such techniques include, but are not limited to, ¹⁵⁷Gd, ⁵⁵Mn, 162Dy ⁵²Cr, and ⁵⁶Fe.

Exemplary paramagnetic ions include, but are not limited to, chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and erbium (III). Ions useful, for example, in X-ray imaging, include but are not limited to lanthanum (III), gold (III), lead (II), and bismuth (III).

The concentration of detectably labeled modified antibody which is administered is sufficient such that the binding to HSVis detectable compared to the background. Further, it is desirable that the detectably labeled modified antibody be rapidly cleared from the circulatory system in order to give the best target-to-background signal ratio.

The dosage of detectably labeled modified antibody for in vivo diagnosis will vary depending on such factors as age, sex, and extent of disease of the individual. The dosage of Human monoclonal antibody can vary, for example, from about 0.01 mg/m² to about 500 mg/m², 0.1 mg/m² to about 200 mg/m², or about 0.1 mg/m² to about 10 mg/m². Such dosages can vary, for example, depending on whether multiple injections are given, tissue, and other factors known to those of skill in the art.

6. Monitoring Infection

The modified antibodies provided herein can be used in vitro and in vivo to monitor the course of pathogenic disease therapy. Thus, for example, the increase or decrease in the number of cells infected with a pathogen (e.g. HSV) or changes in the concentration of the pathogen present in the body or in various body fluids can be measured. Thus, the modified therapeutic antibodies can be employed to determine whether a particular therapeutic regimen aimed at ameliorating the pathogenic disease is effective.

### H. PHARMACEUTICAL COMPOSITIONS, COMBINATIONS AND ARTICLES OF MANUFACTURE/KITS

1. Pharmaceutical Compositions

Provided herein are pharmaceutical compositions containing a modified therapeutic antibody provided herein. The pharmaceutical composition can be used for therapeutic, prophylactic, and/or diagnostic applications. The modified antibodies provided herein can be formulated with a pharmaceutically acceptable carrier or diluent. Generally, such pharmaceutical compositions utilize components which will not significantly impair the biological properties of the antibody, such as the binding to its specific epitope. Each component is both pharmaceutically and physiologically acceptable in the sense of being compatible with the other ingredients and not injurious to the patient. The formulations can conveniently be presented in unit dosage form and can be prepared by methods well known in the art of pharmacy, including but not limited to, tablets, pills, powders, liquid solutions or suspensions (e.g., including injectable, ingestible) and topical formulations (e.g., eyedrops, gels or ointments), aerosols (e.g., nasal sprays), liposomes, suppositories, injectable and infusible solution and sustained release forms. See, e.g., Gilman, et al. (eds. 1990) Goodman and Gilman's: The Pharmacological Bases of Therapeutics, 8th Ed., Pergamon Press; and Remington's Pharmaceutical Sciences, 17th ed. (1990), Mack Publishing Co., Easton, Pa.; Avis, et al. (eds. 1993) Pharmaceutical Dosage Forms: Parenteral Medications Dekker, NY; Lieberman, et al. (eds. 1990) Pharmaceutical Dosage Forms Tablets Dekker, NY; and Lieberman, et al. (eds. 1990) Pharmaceutical Dosage Forms: Disperse Systems Dekker, NY. When administered systematically, the therapeutic composition is sterile, pyrogen-free and in a parenterally acceptable solution having due regard for pH, isotonicity, and stability. These conditions are known to those skilled in the art.

Pharmaceutical compositions provided herein can be in various forms, e.g., in solid, semi-solid, liquid, powder, aqueous, or lyophilized form. Examples of suitable pharmaceutical carriers are known in the art and include but are not limited to water, buffers, saline solutions, phosphate buffered saline solutions, various types of wetting agents, sterile solutions, alcohols, gum arabic, vegetable oils, benzyl alcohols, gelatin, glycerin, carbohydrates such as lactose, sucrose, amylose or starch, magnesium stearate, talc, silicic acid, viscous paraffin, perfume oil, fatty acid monoglycerides and diglycerides, pentaerythritol fatty acid esters, hydroxy methylcellulose, powders, among others. Pharmaceutical compositions provided herein can contain other additives including, for example, antioxidants, preservatives, antimicrobial agents, analgesic agents, binders, disintegrants, coloring, diluents, excipients, extenders, glidants, solubilizers, stabilizers, tonicity agents, vehicles, viscosity agents, flavoring agents, emulsions, such as oil/water emulsions, emulsifying and suspending agents, such as acacia, agar, alginic acid, sodium alginate, bentonite, carbomer, carrageenan, carboxymethylcellulose, cellulose, cholesterol, gelatin, hydroxyethyl cellulose, hydroxypropyl cellulose, hydroxypropyl methylcellulose, methylcellulose, octoxynol 9, oleyl alcohol, povidone, propylene glycol monostearate, sodium lauryl sulfate, sorbitan esters, stearyl alcohol, tragacanth, xanthan gum, and derivatives thereof, solvents, and miscellaneous ingredients such as crystalline cellulose, microcrystalline cellulose, citric acid, dextrin, dextrose, liquid glucose, lactic acid, lactose, magnesium chloride, potassium metaphosphate, starch, among others (see, generally, Alfonso R. Gennaro (2000) Remington: The Science and Practice of Pharmacy, 20th Edition. Baltimore, Md.: Lippincott Williams & Wilkins). Such carriers and/or additives can be formulated by conventional methods and can be administered to the subject at a suitable dose. Stabilizing agents such as lipids, nuclease inhibitors, polymers, and chelating agents can preserve the compositions from degradation within the body

Pharmaceutical compositions suitable for use include compositions wherein one or more modified therapeutic antibodies are contained in an amount effective to achieve their intended purpose. Determination of a therapeutically effective amount is well within the capability of those skilled in the art. Therapeutically effective dosages can be determined by using in vitro and in vivo methods as described herein.

2. Articles of Manufacture/Kits

Pharmaceutical compositions of selected modified therapeutic antibodies or nucleic acids encoding selected antibodies, or a derivative or a biologically active portion thereof can be packaged as articles of manufacture containing packaging material, a pharmaceutical composition which is effective for vaccination and/or treating the disease or disorder, and a label that indicates that selected antibody or nucleic acid molecule is to be used for vaccination and/or treating the disease or disorder.

The articles of manufacture provided herein contain packaging materials. Packaging materials for use in packaging pharmaceutical products are well known to those of skill in the art. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. The pharmaceutical composition also can be incorporated in, applied to or coated on a barrier or other protective device that is used for contraception from infection.

The modified therapeutic antibodies, nucleic acid molecules encoding the antibodies thereof, pharmaceutical compositions or combinations provided herein also can be provided as kits. Kits can optionally include one or more components such as instructions for use, devices and additional reagents, and components, such as tubes, containers and syringes for practice of the methods. Exemplary kits can include the modified therapeutic antibodies provided herein, and can optionally include instructions for use, a device for administering the modified therapeutic antibodies to a subject, a device for detecting the modified therapeutic antibodies in a subject, a device for detecting the modified therapeutic antibodies in samples obtained from a subject, and a device for administering an additional therapeutic agent to a subject.

The kit can, optionally, include instructions. Instructions typically include a tangible expression describing the modified therapeutic antibodies and, optionally, other components included in the kit, and methods for administration, including methods for determining the proper state of the subject, the proper dosage amount, dosing regimens, and the proper administration method for administering the modified therapeutic antibodies. Instructions also can include guidance for monitoring the subject over the duration of the treatment time

Kits also can include a pharmaceutical composition described herein and an item for diagnosis. For example, such kits can include an item for measuring the concentration, amount or activity of the selected modified therapeutic antibody in a subject.

Kits provided herein also can include a device for administering the modified therapeutic antibodies to a subject. Any of a variety of devices known in the art for administering medications to a subject can be included in the kits provided herein. Exemplary devices include a hypodermic needle, an intravenous needle, a catheter, a needle-less injection, but are not limited to, a hypodermic needle, an intravenous needle, a catheter, a needle-less injection device, an inhaler and a liquid dispenser such as an eyedropper. Typically the device for administering the modified therapeutic antibodies of the kit will be compatible with the desired method of administration of the modified therapeutic antibodies. For example, a modified therapeutic antibody to be delivered subcutaneously can be included in a kit with a hypodermic needle and syringe.

3. Combinations

Provided herein are combinations of the modified therapeutic antibodies provided herein and a second agent, such as a second modified therapeutic antibody or other therapeutic or diagnostic agent. A combination can include any modified therapeutic antibody or reagent for effecting therapy thereof in accord with the methods provided herein. For example, a combination can include any modified therapeutic antibody and an antiviral agent. Combinations also can include any modified therapeutic antibody and an agent that increases the permeability and/or absorption of the antibody the site of therapy, such as, for example, a viscoelastic agent, such as hyaluronic acid. Combinations also can include a modified therapeutic antibody provided herein with one or more additional therapeutic antibodies. Combinations of the modified therapeutic antibodies provided herein also can contain pharmaceutical compositions containing the modified therapeutic antibodies or host cells containing nucleic acids encoding the modified therapeutic antibodies as described herein. The combinations provided herein can be formulated as a single composition or in separate compositions.

### I. EXAMPLES

The following examples are included for illustrative purposes only and are not intended to limit the scope of the invention.

### Example 1

### TAT-Modified Single Chain Antibodies

In this example, wildtype and modified AC8 single chain antibodies were generated, cloned and expressed. The modified AC8 single chain antibodies (AC8scFv) include AC8scFvTat, in which an HIV-Tat peptide protein transduction domain (PTD) was conjugated to the carboxy terminus (C-terminus) of the AC8 scFv antibody; AC8scFvscrambled, a control antibody in which the amino acid residues of the HCDR3 region of AC8 were modified, or scrambled (the modification inhibits binding of the antibody to HSV); and AC8scFvTATscrambled, a second control antibody in which the scrambled AC8 antibody was conjugated to the HIV-Tat peptide.

### A. Generation of Wildtype and Tat-Modified AC8 Single Chain Antibodies

A selected protein transduction domain (PTD), an HIV-TAT peptide (SEQ ID NO: 915), was conjugated to a neutralizing anti-HSV single chain antibody to assess effects of the PTD on the antiviral properties of the anti-HSV antibody. The neutralizing antibody employed in the study was a single chain antibody which immunoreacts with glycoprotein D of Herpes simplex virus. This single chain antibody (scFv) was derived from a Human monoclonal anti-HSV Fab antibody (AC8) (U.S. Pat. No. 6,156,313), which is specific for Herpes simplex virus Type-1 and Type-2.

1. Wildtype AC8scFv

The AC8 single chain antibody (AC8scFv) generated for the study contains a 22 amino acid leader sequence (amino acids 1-22 of SEQ ID NO: 1), the Ig light chain variable region of the AC8 Fab antibody (amino acids 23-129 of SEQ ID NO: 1), an 18 amino acid linker sequence (amino acids 130-147 of SEQ ID NO: 1), the Ig heavy chain variable region of the AC8 Fab antibody (amino acids 148-277 of SEQ ID NO: 1), a histidine purification sequence (amino acids 283-288 of SEQ ID NO: 1) and a hemagglutinin (HA) tag (amino acids 291-300 of SEQ ID NO: 1). The Ig light chain variable region of the AC8 Fab antibody contains three light chain complementarity determining regions, LCDR1, LCDR2 and LCDR3. The Ig heavy chain variable region of the AC8 Fab antibody contains three heavy chain complementarity determining regions, HCDR1, HCDR2 and HCDR3. The amino acid sequence of the AC8scFv antibody is as follows (the HCDR3 region is shown in bold):

(SEQ ID NO: 1)
MKKTAIAIAVALAGFATVAQAAEIVLTQSPGTLSLSPGERATLSCRASQS
VSSAYLAWYQQKPGQAPRLLIYGASSRATGIPDRFSGSGSGTDFTLTISR
LEPEDFAVYYCQQYGRSPTFGGGTKVEIKGGSSRSSSSGGGGSGGGGQVQ
LVQSGAEVKKPGSSVKVSCKASGGSFSSYAINWVRQAPGQGLEWMGGLMP
IFGTTNYAQKFQDRLTITADVSTSTAYMQLSGLTYEDTAMYYCARVAYML
EPTVTAGGLDVWGQGTTVTVSSASTKGGQAGQHHHHHHGAYPYDVPDYAS

2. AC8scFvTAT

The TAT-modified version of the AC8 single chain antibody (AC8scFvTAT) contains the AC8scFv single chain antibody conjugated to a 9 amino acid long TAT PTD sequence (SEQ ID NO: 915) at the carboxy terminus of the AC8scFv single chain antibody. The amino acid sequence of the AC8scFvTAT modified antibody is as follows (the HCDR3 region is shown in bold and the TAT PTD sequence is underlined):

(SEQ ID NO: 2)
MKKTAIAIAVALAGFATVAQAAEIVLTQSPGTLSLSPGERATLSCRASQS
VSSAYLAWYQQKPGQAPRLLIYGASSRATGIPDRFSGSGSGTDFTLTISR
LEPEDFAVYYCQQYGRSPTFGGGTKVEIKGGSSRSSSSGGGGSGGGGQVQ
LVQSGAEVKKPGSSVKVSCKASGGSFSSYAINWVRQAPGQGLEWMGGLMP
IFGTTNYAQKFQDRLTITADVSTSTAYMQLSGLTYEDTAMYYCARVAYML
EPTVTAGGLDVWGQGTTVTVSSASTKGGQAGQHHHHHHGAYPYDVPDYAS
RGRKKRRQRRR

### B. Generation of Scrambled AC8 Single Chain Antibodies

For comparison, two control single chain antibodies were generated. Each control antibody contains a variant AC8 single chain antibody where the AC8 single chain antibody has a modified heavy chain complementarity determining region 3 (HCDR3). The modified HCDR3 abolishes binding of the AC8 single chain antibody to HSV.

1. AC8scFvscrambled

Control antibody, AC8scFvscrambled, contains the HCDR3-modified AC8 single chain antibody. The amino acid sequence of the AC8scFvscrambled antibody is as follows (the modified HCDR3 region is shown in bold):

(SEQ ID NO: 3)
MKKTAIAIAVALAGFATVAQAAEIVLTQSPGTLSLSPGERATLSCRASQS
VSSAYLAWYQQKPGQAPRLLIYGASSRATGIPDRFSGSGSGTDFTLTISR
LEPEDFAVYYCQQYGRSPTFGGGTKVEIKGGSSRSSSSGGGGSGGGGQVQ
LVQSGAEVKKPGSSVKVSCKASGGSFSSYAINWVRQAPGQGLEWMGGLMP
IFGTTNYAQKFQDRLTITADVSTSTAYMQLSGLTYEDTAMYYCARVVADY
LMGLGEAPTTVWGQGTTVTVSSASTKGGQAGQHHHHHHGAYPYDVPDYAS

2. AC8scFvTATscrambled

Control antibody, AC8scFvTATscrambled, contains the HCDR3-modified AC8 single chain antibody conjugated to a 9 amino acid long TAT PTD sequence at the carboxy terminus of the antibody. The amino acid sequence of the AC8scFvTATscrambled antibody is as follows (the modified HCDR3 region is shown in bold and the TAT PTD sequence is underlined):

(SEQ ID NO: 4)
MKKTAIAIAVALAGFATVAQAAEIVLTQSPGTLSLSPGERATLSCRASQS
VSSAYLAWYQQKPGQAPRLLIYGASSRATGIPDRFSGSGSGTDFTLTISR
LEPEDFAVYYCQQYGRSPTFGGGTKVEIKGGSSRSSSSGGGGSGGGGQVQ
LVQSGAEVKKPGSSVKVSCKASGGSFSSYAINWVRQAPGQGLEWMGGLMP
IFGTTNYAQKFQDRLTITADVSTSTAYMQLSGLTYEDTAMYYCARVVADY
LMGLGEAPTTVWGQGTTVTVSSASTKGGQAGQHHHHHHGAYPYDVPDYAS
RGRKKRRQRRR

### C. Cloning and Expression of AC8 Single Chain Antibodies

The AC8 single chain antibodies described above were constructed by standard recombinant methods using nucleic acid encoding the Human AC8 anti-HSV Fab antibody (U.S. Pat. No. 6,156,313). The nucleic acid encoding the AC81 g heavy and Ig light chains was derived from the pDR12 vector encoding the AC8 Fab. The nucleotide sequences encoding the AC8scFv (SEQ ID NO: 1009), AC8scFvTAT (SEQ ID NO: 1010), AC8scFvscrambled (SEQ ID NO: 1011), AC8scFvTATscrambled (SEQ ID NO: 1012) were subcloned into the prokaryotic expression vector pET28 (Novagen, SEQ ID NO: 1007) by standard polymerase chain reaction (PCR) techniques using the following DNA oligonucleotide primers:

Forward primer (127mer):
(SEQ ID NO: 1013)
5′GCCTGACATATGAAGACACGGCCATGTATTACTGTGCGAGAGTTGTCG
CCGACTATTTGATGGGTTTGGGGGAAGCACCTACTACCGTCTGGGGCCAA
GGGACCACGGTCACCGTGAGCTCAGCTTC 3′
Reverse Primer (127mer):
(SEQ ID NO: 1014)
5′GAAGCTGAGCTCACGGTGACCGTGGTCCCTTGGCCCCAGACGGTAGTA
GGTGCTTCCCCCAAACCCATCAAATAGTCGGCGACAACTCTCGCACAGTA
ATACATGGCCGTGTCTTCATATGTCAGGC 3′

The nucleotide sequence of pET28 AC8scFv is provided (SEQ ID NO: 1008).

The recombinant pET28 vectors encoding the various single chain antibodies were transformed into E. coli strain BL21(DE3)pLysS cells for expression of the single chain antibody. The host cell was transformed with the pET28 vectors containing AC8scFv antibody genes. Single bacterial colonies were selected for analysis. The identity of the contained vectors was confirmed by sequencing.

For protein production, the transformed bacterial cells were inoculated into 4 liters of SB medium containing 70 mg/ml Kanamycin and incubated at 37° C. with shaking (250 rpm). When the OD 600 nm of the bacterial culture reached 0.8-1.0, 1M IPTG was added to the cultures (final IPTG concentration 1 ug/ml), and the cultures were incubated overnight at 30° C.

The overnight culture was spun at 4000 rpm for 30 min. The bacterial pellet was harvested and resuspended in 1×PBS (Phosphate Buffered Saline) containing Protease Inhibitor Cocktail (Santa Cruz Biotechnology, sc-29131). The resuspension was sonicated on wet ice and then centrifuged at 11,000 rpm for 25 min.

The supernatant was collected and applied to a Ni-NTA agarose column (Qiagen, Cat No: 1018244). The column was washed and the antibodies were eluted with elution buffer containing 250 mM imidazole. The eluted antibodies were dialyzed against PBS (pH 7.0) and then concentrated with centrifugal filters (Millipore, UFC801024). The antibody solution was filtered sterilized with 0.22 gm syringe filters (Millipore, SLGP033RB). The antibodies were then analyzed, aliquotted into Eppendorf tubes and stored at −80° C.

The AC8scFv, AC8scFvTAT, AC8scFvscrambled, AC8scFvTATscrambled antibodies exhibited similar expression levels in bacteria. Approximately 2-4 mg per antibody was purified from 1 liter of overnight bacterial culture.

### Example 2

### Cloning and Expression of AC8FabTAT

In this example, a selected protein transduction domain (PTD), an HIV-TAT peptide (SEQ ID NO: 915), was conjugated to a neutralizing anti-HSV Fab antibody to assess effects of the PTD on the antiviral properties of the anti-HSV antibody. The neutralizing antibody employed in the study was anti-HSV Fab antibody AC8 (see U.S. Pat. No. 6,156,313), which immunoreacts with glycoprotein D of Herpes simplex virus and is specific for Herpes simplex virus Type-1 and Type-2.

The HIV-Tat peptide was conjugated to the carboxy terminus of the AC81 g heavy chain. The amino acid sequence of the Ig Heavy chain containing the HIV-Tat peptide (encoded by the nucleotides set forth in SEQ ID NO: 1015) is as follows (the TAT PTD sequence is underlined):

(SEQ ID NO: 1016)
MEWSWVFLFFLSVTTGVHSQVQLVQSGAEVKKPGSSVKVSCKASGGSFSS
YAINWVRQAPGQGLEWMGGLMPIFGTTNYAQKFQDRLTITADVSTSTAYM
QLSGLTYEDTAMYYCARVAYMLEPTVTAGGLDVWGQGTTVTVASASTKGP
SVFPLAPSSKSTSGGTAALGCLVKDYFPEPVTVSWNSGALTSGVHTFPAV
LQSSGLYSLSSVVTVPSSSLGTQTYICNVNHKPSNTKVDKKVEPKSCDKT
RGGRKKRRQRRR

The Ig Heavy chain contains a signal sequence (amino acids 1-19 of SEQ ID NO: 1016), the AC8 heavy chain (amino acids 20-149 of SEQ ID NO: 1016), the heavy chain constant region CH1 (amino acids 150-253 of SEQ ID NO: 1016) and the HIV-Tat peptide (amino acids 254-262 of SEQ ID NO: 1016).

The amino acid sequence of the Ig Light chain (encoded by the nucleotides set forth in SEQ ID NO: 1017) is as follows:

(SEQ ID NO: 1018)
MGVPTQVLGLLLLWLTDARCEIVLTQSPGTLSLSPGERATLSCRASQSVS
SAYLAWYQQKPGQAPRLLIYGASSRATGIPDRFSGSGSGTDFTLTISRLE
PEDFAVYYCQQYGRSPTFGGGTKVEIKRTVAAPSVFIFPPSDEQLKSGTA
SVVCLLNNFYPREAKVQWKVDNALQSGNSQESVTEQDSKDSTYSLSNTLT
LSKADYEKHKVYACEVTHQGLRSPVTKSFNRGEC

The Ig Light chain contains a signal sequence (amino acids 1-20 of SEQ ID NO: 1018), the AC8 light chain (amino acids 21-127 of SEQ ID NO: 1018) and the light chain constant region (amino acids 128-234 of SEQ ID NO: 1018).

Fab antibody AC8FabTAT was constructed by standard recombinant methods, cloned into vector pDR12Fab and soluble Fab was expressed.

### Example 3

### HSV-1 Neutralization in Solution

In this example, the ability of the TAT-modified anti-HSV single chain antibody to bind to and neutralize HSV-1 virus in solution was assessed by a plaque reduction assay. In this experiment, the HSV-1 virus and the AC8 antibodies are pre-incubated in the absence of target cells. The mixture is then added to the cells and virus infection is measured by a standard plaque assay described herein. The assay thus measures the anti-viral protective effects of the AC8 antibody that are not dependent on attachment of the antibody to the target cell and/or internalization of the antibody.

Methods

Vero cells (ATCC, cat no: CCL-81; Manassas, Va.) were employed for host cell infection. Vero cells were grown in DMEM (HyClone, cat no: ATH32679) with 10% FBS (HyClone, cat no: SV30014.03), supplemented with 1% L-Glutamine (HyClone, cat no: SH30034.01) and 1% Penicillin-Streptomycin solution (HyClone, cat no: SV30010). The Vero cells were maintained in a 37° C. incubator with 5% CO2 and passaged twice per week.

On Day 1 of the experiment, Vero cells were prepared in 6-well cell culture plates. The cells were plated in DMEM at a density (approximately 1×10⁶ cells per well) which allows formation of a cell monolayer (>90% confluence) by Day 2. On Day 2, each antibody (AC8scFvTAT, AC8scFv or AC8 Fab) was serially diluted in plain DMEM (final antibody concentrations tested: 3 μg/ml, 1 μg/ml, 0.33 μg/ml, 0.11 μg/ml and 0.037 μg/ml). The HSV virus (ATCC, cat no: VR-733; Manassas, Va.) also was diluted in plain DMEM (800 PFU/ml). 250 ul of the diluted virus was added to 250 ul of each diluted antibody solution and mixed by pipetting. For the virus control sample, 250 ul virus was added to 250 μl plain DMEM. Each test and control sample was prepared in duplicate. The antibody-virus or virus control mixture was incubated at 37° C. for one hour. Following incubation, the culture media was aspirated from the 6-well cell culture plates containing the Vero host cells. 500 μl of the virus-antibody or virus control mixture was then transferred to each well. The cells were then incubated at 37° C. for one hour with a gentle mixing every 15 minutes.

Following the incubation period, the culture media containing the antibody-virus or virus control mixture was aspirated and 2.5 ml of overlay medium was added to each well (overlay medium was prepared by mixing equal volumes of 2×DMEM/4% FBS (37° C.) and 1% agarose (56° C.)). The 6 well cell culture plates were then incubated at 37° C. for approximately 72 hours. Plaque formation was checked under microscope periodically. Once the plaques matured, the plates were fixed with 10% Formalin for 30 minutes at room temperature. The formalin was then discarded and the plates were stained with 1% crystal violet solution in 70% methanol. The stained monolayers were then washed with ddH₂O and the plaques were counted. The plaque reduction rate for virus alone control samples was set at 0% neutralization, and the plaque reduction rates for all antibody dilutions were calculated accordingly. The ED50 and ED90 of each antibody was calculated using Prism software.

Results

No difference was observed between AC8Fab, AC8scFv and AC8scFvTAT for neutralizing HSV1 virus (Table 8). This result indicates that the AC8 antibodies were able to bind to and neutralize the HSV-1 virus in solution. The ED50 for each antibody was around 0.2 μg/ml.

TABLE 8

| | Plaque Reduction Rate Percentage | | | | | |
|---|---|---|---|---|---|---|
| Antibody | (Average plaque number per plate) | | | | | |
| Tested | 3 μg/ml | 1 μg/ml | 0.33 μg/ml | 0.11 μg/ml | 0.037 μg/ml | Virus control |
| AC8scFvTAT | 99 (4.3) | 98 (7.3) | 76 (78) | 35 (210) | 8 (295) | 0 (322) |
| AC8scFv | 98 (5) | 98 (7) | 74 (83.3) | 32 (219) | 10 (291) | 0 (322) |
| AC8Fab | 98 (4.7) | 99 (3.7) | 73 (87) | 30 (225) | 6 (302) | 0 (322) |

In a separate independent experiment the ED50 of the AC8scFvTAT, AC8scFv and AC8 Fab antibodies was compared to the ED50 of the AC8scFvscrambled and AC8scFvTATscrambled control antibodies. In this experiment, the AC8scFvTAT, AC8scFv and AC8Fab antibodies exhibited an ED50 similar to the first experiment (all three antibodies had an ED50 of approximately 0.15 μg/ml). The AC8scFvscrambled and AC8scFvTAT scrambled antibodies did not exhibit any virus neutralization activity.

### Example 4

### Virus Neutralization by Association with the Target Cell

In this example, the ability of the TAT-modified anti-HSV single chain antibody to attach to target cells and/or become internalized by the target cells in order to inhibit subsequent viral infection and/or virus production was assessed by a virus prevention assay. In this experiment, target cells are first incubated with the AC8 antibody alone. Following the pre-incubation, the free antibody is removed and the HSV-1 virus is added to the cells for a second incubation period. Following the virus incubation, the virus is removed from the cells and plaque formation is measured after several days incubation. The assay thus measures the anti-viral protective effects of the AC8 antibody that are dependent on attachment of the antibody to the target cell and/or internalization of the antibody.

Methods

Vero cells (ATCC, cat no: CCL-81; Manassas, Va.) were employed for host cell infection. Vero cells were grown in DMEM (HyClone, cat no: ATH32679) with 10% FBS (HyClone, cat no: SV30014.03), supplemented with 1% L-Glutamine (HyClone, cat no: SH30034.01) and 1% Penicillin-Streptomycin solution (HyClone, cat no: SV30010). The Vero cells were maintained in a 37° C. incubator with 5% CO₂ and passaged twice per week.

On Day 1 of the virus neutralization experiment, Vero cells were prepared in 6-well cell culture plates. The cells were plated in DMEM at a density (approximately 1×10⁶ cells per well) which allows formation of a cell monolayer (>90% confluence) by Day 2. On Day 2, each antibody (AC8scFvTAT, AC8scFv, AC8scFvTATscrambled or AC8scFvscrambled) was serially diluted in plain DMEM (Antibody concentrations tested: 5 μg/ml, 50 μg/ml and 500 μg/ml). 500 ul of diluted antibody was added to each well and incubated at 37° C. for one hour. The cells were then washed three times with 1×PBS to remove free antibody. 1 ml of plain DMEM was then added to each well and the cells were incubated at 37° C. for 0, 8, or 24 hours.

The HSV virus (ATCC, cat no: VR-733; Manassas, Va.) was diluted in plain DMEM (400 PFU/ml). After the second incubation period, the media was replaced with 500 ul diluted virus and incubated at 37° C. for one hour. Following the virus incubation period, the culture media containing the virus was aspirated and 2.5 ml of overlay medium was added to each well (overlay medium was prepared by mixing equal volumes of 2×DMEM/4% FBS (37° C.) and 1% agarose (56° C.)). The 6 well cell culture plates were then incubated at 37° C. for approximately 72 hours. Plaque formation was checked under microscope periodically. Once the plaques matured, the plates were fixed with 10% Formalin for 1 hour at room temperature. The formalin was then discarded and the plates were stained with 1% crystal violet solution in 70% methanol. The stained monolayers were then washed with ddH₂O and the plaques were counted. The plaque numbers for each of the test samples was compared to the control plaque number. The ED50 and ED90 of each antibody was calculated using Prism software. The percentage neutralization rate was set at 0% neutralization for the virus alone control samples, and the percentage neutralization for the antibody dilutions were calculated accordingly.

Results

Cells treated with the TAT-modified AC8 single chain antibody exhibited statistically significant (p<0.01) protection from HSV-1 virus infection up to 24 hours after antibody treatment at concentrations greater than 50 μg/mL. The unmodified anti-HSV single chain antibody and both CDR3 scrambled antibodies failed to demonstrate any neutralization of the virus.

### Example 5

### Inhibition of Cell to Cell Virus Spread

In this example, the ability of the TAT-modified anti-HSV single chain antibody to inhibit cell to cell virus spread was assessed by a plaque size reduction assay. In this experiment, target cells are first incubated with the virus alone. Following the pre-incubation, the free virus is removed and the AC8 antibody is added to the cells. The size of plaques formed is measured after several days incubation. The assay thus measures the inhibition of viral spread following initial virus infection.

Methods

Vero cells (ATCC, cat no: CCL-81; Manassas, Va.) were employed for host cell infection. Vero cells were grown in DMEM (HyClone, cat no: ATH32679) with 10% FBS (HyClone, cat no: SV30014.03), supplemented with 1% L-Glutamine (HyClone, cat no: SH30034.01) and 1% Penicillin-Streptomycin solution (HyClone, cat no: SV30010). The Vero cells were maintained in a 37° C. incubator with 5% CO₂ and passaged twice per week.

On Day 1 of the virus neutralization experiment, Vero cells were prepared in 6-well cell culture plates. The cells were plated in DMEM at a density (approximately 1×10⁶ cells per well) which allows formation of a cell monolayer (>90% confluence) by Day 2. On Day 2, HSV-1 virus was diluted in plain DMEM (400 PFU/ml). Each antibody (AC8scFvTAT or AC8scFv) was serially diluted in 1×DMEM with 2% FBS and 0.75% methylcellulose (Antibody concentrations tested: 0 μg/ml no antibody control, 0.08 μg/ml, 0.4 μg/ml, 2 μg/ml, 10 μg/ml and 50 μg/ml).

500 ul of the HSV-1 virus dilution was first added to each well of Vero cells and incubated at 37° C. for two hours with gentle shaking every 15 minutes. Following virus incubation the cells were washed once with PBS to remove free virus. Then 1 ml of each antibody dilution was added to each well and incubated at 37° C. for three days.

Following the three day incubation, the plates were fixed with 10% Formalin for 1 hour at room temperature. The formalin was then discarded and the plates were stained with 1% crystal violet solution in 70% methanol. The stained monolayers were then washed with ddH₂O. The size of plaques were measured using ImageTool software.

Results

The TAT-modified AC8 scFv was over 15-fold better at reducing cell-to-cell virus spread than AC8 scFv alone. The TAT-modified AC8 scFv had an EC50 equal to 0.6 μg/mL and the AC8 scFv had an EC50 equal to 10 μg/mL.

### Example 6

### Cloning and Expression of Various PTD Antibodies

In this example, various protein transduction domains (PTDs) were conjugated to the carboxy terminus (C-terminus) of a neutralizing anti-HSV single chain antibody. The neutralizing antibody employed in the study was a single chain antibody which immunoreacts with glycoprotein D of Herpes simplex virus. This single chain antibody (scFv) was derived from a Human monoclonal anti-HSV Fab antibody (AC8) (U.S. Pat. No. 6,156,313), which is specific for Herpes simplex virus Type-1 and Type-2.

A. Tat-Like Transduction Domains with Gln at Position 6 Modified AC8 scFv Antibodies

Single chain antibodies AC8scFvTAT1A, AC8scFvTAT1B, and AC8scFvTAT1C each contain a Tat-like transduction domain with Gln at position 6 conjugated to the C-terminus of the AC8 scFv antibody.

1. AC8scFvTAT1A

Single chain antibody AC8scFvTAT1A contains the AC8scFv single chain antibody conjugated to a 9 amino acid long PTD (Feline Immunodeficiency Virus Rev peptide, SEQ ID NO: 9). The amino acid sequence of the AC8scFvTAT1A modified antibody (encoded by the nucleotides set forth in SEQ ID NO: 1019) is as follows (the HCDR3 region is shown in bold and the Feline Immunodeficiency Virus Rev peptide sequence is underlined):

(SEQ ID NO: 1031)
MKKTAIAIAVALAGFATVAQAAEIVLTQSPGTLSLSPGERATLSCRASQS
VSSAYLAWYQQKPGQAPRLLIYGASSRATGIPDRFSGSGSGTDFTLTISR
LEPEDFAVYYCQQYGRSPTFGGGTKVEIKGGSSRSSSSGGGGSGGGGQVQ
LVQSGAEVKKPGSSVKVSCKASGGSFSSYAINWVRQAPGQGLEWMGGLMP
IFGTTNYAQKFQDRLTITADVSTSTAYMQLSGLTYEDTAMYYCARVAYML
EPTVTAGGLDVWGQGTTVTVSSASTKGGQAGQHHHHHHGAYPYDVPDYAS
GGRRKRRQRRRR

2. AC8scFvTAT1B

Single chain antibody AC8scFvTAT1B contains the AC8scFv single chain antibody conjugated to a 9 amino acid long PTD sequence (Human ankyrin repeat domain containing protein 2, SEQ ID NO: 13). The amino acid sequence of the AC8scFvTAT1B modified antibody (encoded by the nucleotides set forth in SEQ ID NO: 1020) is as follows (the HCDR3 region is shown in bold and the Human ankyrin repeat domain containing protein 2 sequence is underlined):

(SEQ ID NO: 1032)
MKKTAIAIAVALAGFATVAQAAEIVLTQSPGTLSLSPGERATLSCRASQS
VSSAYLAWYQQKPGQAPRLLIYGASSRATGIPDRFSGSGSGTDFTLTISR
LEPEDFAVYYCQQYGRSPTFGGGTKVEIKGGSSRSSSSGGGGSGGGGQVQ
LVQSGAEVKKPGSSVKVSCKASGGSFSSYAINWVRQAPGQGLEWMGGLMP
IFGTTNYAQKFQDRLTITADVSTSTAYMQLSGLTYEDTAMYYCARVAYML
EPTVTAGGLDVWGQGTTVIVSSASTKGGQAGQHHHHHHGAYPYDVPDYAS
GGRKKRKQKKR

3. AC8scFvTAT1C

Single chain antibody AC8scFvTAT1C contains the AC8scFv single chain antibody conjugated to a 9 amino acid long PTD sequence (Fruit fly protein GI14201, SEQ ID NO: 29). The amino acid sequence of the AC8scFvTAT1C modified antibody (encoded by the nucleotides set forth in SEQ ID NO: 1021) is as follows (the HCDR3 region is shown in bold and the Fruit fly protein GI14201 sequence is underlined):

(SEQ ID NO: 1033)
MKKTAIAIAVALAGFATVAQAAEIVLTQSPGTLSLSPGERATLSCRASQS
VSSAYLAWYQQKPGQAPRLLIYGASSRATGIPDRFSGSGSGTDFTLTISR
LEPEDFAVYYCQQYGRSPTFGGGTKVEIKGGSSRSSSSGGGGSGGGGQVQ
LVQSGAEVKKPGSSVKVSCKASGGSFSSYAINWVRQAPGQGLEWMGGLMP
IFGTTNYAQKFQDRLTITADVSTSTAYMQLSGLTYEDTAMYYCARVAYML
EPTVTAGGLDVWGQGTTVTVSSASTKGGQAGQHHHHHHGAYPYDVPDYAS
GGKRRKRQRRR

B. Tat-Like Transduction Domains without Gln at Position 6 Modified AC8 scFv Antibodies

Single chain antibodies AC8scFvTAT2A, AC8scFvTAT2B, and AC8scFvTAT2C each contain a Tat-like transduction domain without Gln at position 6 conjugated to the C-terminus of the AC8 scFv antibody.

1. AC8scFvTAT2A

Single chain antibody AC8scFvTAT2A contains the AC8scFv single chain antibody conjugated to a 10 amino acid long PTD sequence (Tobacco Mild Green Mosiac Virus Movement protein, SEQ ID NO: 90). The amino acid sequence of the AC8scFvTAT2A modified antibody (encoded by the nucleotides set forth in SEQ ID NO: 1022) is as follows (the HCDR3 region is shown in bold and the Tobacco Mild Green Mosiac Virus Movement protein sequence is underlined):

(SEQ ID NO: 1034)
MKKTAIAIAVALAGFATVAQAAEIVLTQSPGTLSLSPGERATLSCRASQS
VSSAYLAWYQQKPGQAPRLLIYGASSRATGIPDRFSGSGSGTDFTLTISR
LEPEDFAVYYCQQYGRSPTFGGGTKVEIKGGSSRSSSSGGGGSGGGGQVQ
LVQSGAEVKKPGSSVKVSCKASGGSFSSYAINWVRQAPGQGLEWMGGLMP
IFGTTNYAQKFQDRLTITADVSTSTAYMQLSGLTYEDTAMYYCARVAYML
EPTVTAGGLDVWGQGTTVTVSSASTKGGQAGQHHHHHHGAYPYDVPDYAS
GGKKRKKEKKKR

2. AC8scFvTAT2B

Single chain antibody AC8scFvTAT2B contains the AC8scFv single chain antibody conjugated to a 10 amino acid long PTD sequence (Mouse T-cell surface antigen CD2, SEQ ID NO: 860). The amino acid sequence of the AC8scFvTAT2B modified antibody (encoded by the nucleotides set forth in SEQ ID NO: 1023) is as follows (the HCDR3 region is shown in bold and the Mouse T-cell surface antigen CD2 sequence is underlined):

(SEQ ID NO: 1035)
MKKTAIAIAVALAGFATVAQAAEIVLTQSPGTLSLSPGERATLSCRASQS
VSSAYLAWYQQKPGQAPRLLIYGASSRATGIPDRFSGSGSGTDFTLTISR
LEPEDFAVYYCQQYGRSPTEGGGTKVEIKGGSSRSSSSGGGGSGGGGQVQ
LVQSGAEVKKPGSSVKVSCKASGGSFSSYAINWVRQAPGQGLEWMGGLMP
IFGTTNYAQKFQDRLTITADVSTSTAYMQLSGLTYEDTAMYYCARVAYML
EPTVTAGGLDVWGQGTTVTVSSASTKGGQAGQHHHHHHGAYPYDVPDYAS
GGKRRKRNRRRR

3. AC8scFvTAT2C

Single chain antibody AC8scFvTAT2C contains the AC8scFv single chain antibody conjugated to a 10 amino acid long PTD sequence (Smut fungus vacuolar fusion protein MON1, SEQ ID NO: 100). The amino acid sequence of the AC8scFvTAT2C modified antibody (encoded by the nucleotides set forth in SEQ ID NO: 1024) is as follows (the HCDR3 region is shown in bold and the Smut fungus vacuolar fusion protein MON1 sequence is underlined):

(SEQ ID NO: 1036)
MKKTAIAIAVALAGFATVAQAAEIVLTQSPGTLSLSPGERATLSCRASQS
VSSAYLAWYQQKPGQAPRLLIYGASSRATGIPDRFSGSGSGTDFTLTISR
LEPEDFAVYYCQQYGRSPTFGGGTKVEIKGGSSRSSSSGGGGSGGGGQVQ
LVQSGAEVKKPGSSVKVSCKASGGSFSSYAINWVRQAPGQGLEWMGGLMP
IFGTTNYAQKFQDRLTITADVSTSTAYMQLSGLTYEDTAMYYCARVAYML
EPTVTAGGLDVWGQGTTVTVSSASTKGGQAGQHHHHHHGAYPYDVPDYAS
GGRKRRREKRRR

C. Prion-Like Transduction Domain Modified AC8 scFv Antibodies

Single chain antibodies AC8scFvTAT3A, AC8scFvTAT3B, and AC8scFvTAT3C each contain a prion-like transduction domain conjugated to the C-terminus of the AC8 scFv antibody.

1. AC8scFvTAT3A

Single chain antibody AC8scFvTAT3A contains the AC8scFv single chain antibody conjugated to a 10 amino acid long PTD sequence (Methylobacterium (strain 4-46) general secretary system II protein E domain protein, SEQ ID NO: 160). The amino acid sequence of the AC8scFvTAT3A modified antibody (encoded by the nucleotides set forth in SEQ ID NO: 1025) is as follows (the HCDR3 region is shown in bold and the Methylobacterium (strain 4-46) general secretary system II protein E domain protein sequence is underlined):

(SEQ ID NO: 1037)
MKKTAIAIAVALAGFATVAQAAEIVLTQSPGTLSLSPGERATLSCRASQS
VSSAYLAWYQQKPGQAPRLLIYGASSRATGIPDRFSGSGSGTDFTLTISR
LEPEDFAVYYCQQYGRSPTFGGGTKVEIKGGSSRSSSSGGGGSGGGGQVQ
LVQSGAEVKKPGSSVKVSCKASGGSFSSYAINWVRQAPGQGLEWMGGLMP
IFGTTNYAQKFQDRLTITADVSTSTAYMQLSGLTYEDTAMYYCARVAYML
EPTVTAGGLDVWGQGTTVTVSSASTKGGQAGQHHHHHHGAYPYDVPDYAS
GGRPRRPRPDRR

2. AC8scFvTAT3B

Single chain antibody AC8scFvTAT3B contains the AC8scFv single chain antibody conjugated to a 10 amino acid long PTD sequence (Mouse zinc finger protein 41, SEQ ID NO: 201). The amino acid sequence of the AC8scFvTAT3B modified antibody (encoded by the nucleotides set forth in SEQ ID NO: 1026) is as follows (the HCDR3 region is shown in bold and the Mouse zinc finger protein 41 sequence is underlined):

(SEQ ID NO: 1038)
MKKTAIAIAVALAGFATVAQAAEIVLTQSPGTLSLSPGERATLSCRASQS
VSSAYLAWYQQKPGQAPRLLIYGASSRATGIPDRFSGSGSGTDFTLTISR
LEPEDFAVYYCQQYGRSPTFGGGTKVEIKGGSSRSSSSGGGGSGGGGQVQ
LVQSGAEVKKPGSSVKVSCKASGGSFSSYAINWVRQAPGQGLEWMGGLMP
IFGTTNYAQKFQDRLTITADVSTSTAYMQLSGLTYEDTAMYYCARVAYML
EPTVTAGGLDVWGQGTTVTVSSASTKGGQAGQHHHHHHGAYPYDVPDYAS
GGKPRKPRRPRK

3. AC8scFvTAT3C

Single chain antibody AC8scFvTAT3C contains the AC8scFv single chain antibody conjugated to a 10 amino acid long PTD sequence (Human coiled-coil and C2 domain containing protein 2A, SEQ ID NO: 492). The amino acid sequence of the AC8scFvTAT3C modified antibody (encoded by the nucleotides set forth in SEQ ID NO: 1027) is as follows (the HCDR3 region is shown in bold and the Human coiled-coil and C2 domain containing protein 2A sequence is underlined):

(SEQ ID NO: 1039)
MKKTAIAIAVALAGFATVAQAAEIVLTQSPGTLSLSPGERATLSCRASQS
VSSAYLAWYQQKPGQAPRLLIYGASSRATGIPDRFSGSGSGTDFTLTISR
LEPEDFAVYYCQQYGRSPTFGGGTKVEIKGGSSRSSSSGGGGSGGGGQVQ
LVQSGAEVKKPGSSVKVSCKASGGSFSSYAINWVRQAPGQGLEWMGGLMP
IFGTTNYAQKFQDRLTITADVSTSTAYMQLSGLTYEDTAMYYCARVAYML
EPTVTAGGLDVWGQGTTVTVSSASTKGGQAGQHHHHHHGAYPYDVPDYAS
GGRPLRPRRKGR

D. Protein Transduction Peptides with Display of Basic Charges on One Face of an Alpha-Helix Modified AC8 scFv Antibodies

Single chain antibodies AC8scFvTAT4A, AC8scFvTAT4B, and AC8scFvTAT4C each contain a protein transduction peptide with display of basic charges on one face of an alpha-helix conjugated to the C-terminus of the AC8 scFv antibody.

1. AC8scFvTAT4A

Single chain antibody AC8scFvTAT4A contains the AC8scFv single chain antibody conjugated to an 11 amino acid long PTD sequence (Human Putative E3 ubiquitin-protein ligase HERC5, SEQ ID NO: 505). The amino acid sequence of the AC8scFvTAT4A modified antibody (encoded by the nucleotides set forth in SEQ ID NO: 1028) is as follows (the HCDR3 region is shown in bold and the Human Putative E3 ubiquitin-protein ligase HERC5 sequence is underlined):

(SEQ ID NO: 1040)
MKKTAIAIAVALAGFATVAQAAEIVLTQSPGTLSLSPGERATLSCRASQS
VSSAYLAWYQQKPGQAPRLLIYGASSRATGIPDRFSGSGSGTDFTLTISR
LEPEDFAVYYCQQYGRSPTFGGGTKVEIKGGSSRSSSSGGGGSGGGGQVQ
LVQSGAEVKKPGSSVKVSCKASGGSFSSYAINWVRQAPGQGLEWMGGLMP
IFGTTNYAQKFQDRLTITADVSTSTAYMQLSGLTYEDTAMYYCARVAYML
EPTVTAGGLDVWGQGTTVTVSSASTKGGQAGQHHHHHHGAYPYDVPDYAS
GGRSRRKSRRNGR

2. AC8svFvTAT4B

Single chain antibody AC8scFvTAT4B contains the AC8scFv single chain antibody conjugated to an 11 amino acid long PTD sequence (African clawed frog Midnolin-A peptide, SEQ ID NO: 741). The amino acid sequence of the AC8scFvTAT4B modified antibody (encoded by the nucleotides set forth in SEQ ID NO: 1′029) is as follows (the HCDR3 region is shown in bold and the African clawed frog Midnolin-A peptide sequence is underlined):

(SEQ ID NO: 1041)
MKKTAIAIAVALAGFATVAQAAEIVLTQSPGTLSLSPGERATLSCRASQS
VSSAYLAWYQQKPGQAPRLLIYGASSRATGIPDRFSGSGSGTDFTLTISR
LEPEDFAVYYCQQYGRSPTFGGGTKVEIKGGSSRSSSSGGGGSGGGGQVQ
LVQSGAEVKKPGSSVKVSCKASGGSFSSYAINWVRQAPGQGLEWMGGLMP
IFGTTNYAQKFQDRLTITADVSTSTAYMQLSGLTYEDTAMYYCARVAYML
EPTVTAGGLDVWGQGTTVTVSSASTKGGQAGQHHHHHHGAYPYDVPDYAS
GGRLRRKARRDSR

3. AC8scFvTAT4C

Single chain antibody AC8scFvTAT4C contains the AC8scFv single chain antibody conjugated to an 11 amino acid long PTD sequence (Bovine early growth response protein 4, SEQ ID NO: 723). The amino acid sequence of the AC8scFvTAT4C modified antibody (encoded by the nucleotides set forth in SEQ ID NO: 1030) is as follows (the HCDR3 region is shown in bold and the Bovine early growth response protein 4 sequence is underlined):

(SEQ ID NO: 1042)
MKKTAIAIAVALAGFATVAQAAEIVLTQSPGTLSLSPGERATLSCRASQS
VSSAYLAWYQQKPGQAPRLLIYGASSRATGIPDRFSGSGSGTDFTLTISR
LEPEDFAVYYCQQYGRSPTFGGGTKVEIKGGSSRSSSSGGGGSGGGGQVQ
LVQSGAEVKKPGSSVKVSCKASGGSFSSYAINWVRQAPGQGLEWMGGLMP
IFGTTNYAQKFQDRLTITADVSTSTAYMQLSGLTYEDTAMYYCARVAYML
EPTVTAGGLDVWGQGTTVTVSSASTKGGQAGQHHHHHHGAYPYDVPDYAS
GGKARRKGRRGGK

### E. Cloning and Expression of AC8 Single Chain Antibodies

The AC8 single chain antibodies described in Example 6A-6D above were constructed by standard recombinant methods using nucleic acid encoding the Human AC8 anti-HSV Fab antibody (U.S. Pat. No. 6,156,313). The nucleic acid encoding the AC81 g heavy and Ig light chains was derived from the pDR12 vector encoding the AC8 Fab. The nucleotide sequences encoding the AC8scFvTAT1A (SEQ ID NO: 1019), AC8scFvTAT1B (SEQ ID NO: 1020), AC8scFvTAT1C (SEQ ID NO: 1021), AC8scFvTAT2A (SEQ ID NO: 1022), AC8scFvTAT2B (SEQ ID NO: 1023), AC8scFvTAT2C (SEQ ID NO: 1024), AC8scFvTAT3A (SEQ ID NO: 1025), AC8scFvTAT3B (SEQ ID NO: 1026), AC8scFvTAT3C (SEQ ID NO: 1027), AC8scFvTAT4A (SEQ ID NO: 1028), AC8scFvTAT4B (SEQ ID NO: 1029), and AC8scFvTAT4C (SEQ ID NO: 1030), were subcloned into the prokaryotic expression vector pET28 (Novagen, SEQ ID NO: 1007) by standard polymerase chain reaction (PCR) techniques using the following DNA oligonucleotide primers:

Forward primer (127 mer):
(SEQ ID NO: 1013)
5′GCCTGACATATGAAGACACGGCCATGTATTACTGTGCGAGAGTTGTCG
CCGACTATTTGATGGGTTTGGGGGAAGCACCTACTACCGTCTGGGGCCAA
GGGACCACGGTCACCGTGAGCTCAGCTTC 3′
Reverse Primer (127 mer):
(SEQ ID NO: 1014)
5′GAAGCTGAGCTCACGGTGACCGTGGTCCCTTGGCCCCAGACGGTAGTA
GGTGCTTCCCCCAAACCCATCAAATAGTCGGCGACAACTCTCGCACAGTA
ATACATGGCCGTGTCTTCATATGTCAGGC 3′

The nucleotide sequence of pET28 AC8scFv is provided (SEQ ID NO: 1008).

The recombinant pET28 vectors encoding the various single chain antibodies were transformed into E. coli strain BL21(DE3)pLysS cells for expression of the single chain antibodies. The host cell was transformed with the pET28 vectors containing AC8scFv antibody genes. Single bacterial colonies were selected for analysis. The identity of the contained vectors was confirmed by sequencing.

For protein production, the transformed bacterial cells were inoculated into 4 liters of SB medium containing 70 mg/ml Kanamycin and incubated at 37° C. with shaking (250 rpm). When the OD 600 nm of the bacterial culture reached 0.8-1.0, 1M IPTG was added to the cultures (final IPTG concentration 1 μg/ml), and the cultures were incubated overnight at 30° C.

The overnight culture was spun at 4000 rpm for 30 min. The bacterial pellet was harvested and resuspended in 1×PBS (Phosphate Buffered Saline) containing Protease Inhibitor Cocktail (Santa Cruz Biotechnology, sc-29131). The resuspension was sonicated on wet ice and then centrifuged at 11,000 rpm for 25 min.

The supernatant was collected and applied to a Ni-NTA agarose column (Qiagen, Cat No: 1018244). The column was washed and the antibodies were eluted with elution buffer containing 250 mM imidazole. The eluted antibodies were dialyzed against PBS (pH 7.0) and then concentrated with centrifugal filters (Millipore, UFC801024). The antibody solution was filtered sterilized with 0.22 um syringe filters (Millipore, SLGP033RB). The antibodies were then analyzed, aliquotted into Eppendorf tubes and stored at −80° C.

### Example 7

### HSV-1 Neutralization by Various PTD Antibodies in Solution

In this example, the ability of the various PTD-modified anti-HSV single chain antibodies to bind to and neutralize HSV-1 virus in solution was assessed by a plaque reduction assay as described in Example 3. In this experiment, the HSV-1 virus and the AC8 antibodies are pre-incubated in the absence of target cells. The mixture is then added to the cells and virus infection is measured by a standard plaque assay described herein. As described above, the assay measures the anti-viral protective effects of the AC8 antibody that are not dependent on attachment of the antibody to the target cell and/or internalization of the antibody.

Methods

The experiment was performed using the methods essentially as described in Example 3 for assaying HSV-1 neutralization in solution with minor changes. For example, 24 well plates instead of 6 well plates were employed for the assay. The amounts of VERO cells plated into each well the total volumes employed were adjusted accordingly to produce the desired number of plaques. Two sets of experiments were performed.

In the first experiment, the AC8scFvTAT, AC8scFvTAT1A, AC8scFvTAT1B, and AC8scFvTAT1C antibodies (produced in BL21 cells; Example 6) were assayed (Table 9). The antibody AC8FabTAT (produced in 293F cells) was employed as positive control for virus neutralization in this experiment. The no-antibody virus control number in this experiment was approximately 44 plaques per well.

In the second experiment, the AC8scFvTAT, AC8scFvTAT2A, AC8scFvTAT2B, and AC8scFvTAT2C antibodies (produced in BL21 cells; Example 6) were assayed (Table 10). The no-antibody virus control number in this experiment was approximately 74 plaques per well.

Results

In the first set of antibodies assayed, all four AC8 scFv antibodies (AC8scFvTAT, AC8scFvTAT1A, AC8scFvTAT1B, and AC8scFvTAT1C) were able to bind to and neutralize the HSV-1 virus in solution as well as the AC8FabTAT antibody (Table 9). The ED50 for each antibody was around 0.1 μg/ml to 0.15 μg/ml.

TABLE 9

| | Plaque Reduction Rate Percentage | | | | | | |
|---|---|---|---|---|---|---|---|
| | (Average plaque number per plate) | | | | | | |
| | | | | | | | Virus |
| Antibody Tested | 3 μg/ml | 1 μg/ml | 0.33 μg/ml | 0.11 μg/ml | 0.037 μg/ml | 0.012 μg/ml | control |
| AC8Fab | 100 (0) | 100 (0) | 77 (10) | 33 (29.5) | 14 (38) | 44.5 (0) | 0 (44) |
| AC8scFvTAT | 98 (1) | 99 (0.5) | 98 (1) | 57 (19) | 10 (39.5) | 44 (0) | 0 (44) |
| AC8scFvTAT1A | 100 (0) | 100 (0) | 96 (1.5) | 57 (19) | 14 (38) | 43 (2) | 0 (44) |
| AC8scFvTAT1B | 100 (0) | 100 (0) | 98 (1) | 40 (26.5) | 9 (40) | 43 (2) | 0 (44) |
| AC8scFvTAT1C | 100 (0) | 100 (0) | 100 (0) | 42 (26.5) | 1 (43.5) | 44.5 (0) | 0 (44) |

In the second set of antibodies assayed, all four AC8 scFv antibodies (AC8scFvTAT, AC8scFvTAT2A, AC8scFvTAT2B, and AC8scFvTAT2C) also were able to bind to and neutralize the HSV-1 virus in solution (Table 10). The ED50 for AC8scFvTAT2A, AC8scFvTAT2B, and AC8scFvTAT2C antibodies was approximately 0.2 μg/ml.

TABLE 10

| | Plaque Reduction Rate Percentage | | | | | |
|---|---|---|---|---|---|---|
| | (Average plaque number per plate) | | | | | |
| | | | | | | Virus |
| Antibody Tested | 5 μg/ml | 1 μg/ml | 0.2 μg/ml | 0.04 μg/ml | 0.008 μg/ml | control |
| AC8scFvTAT | 97 (2) | 99 (1) | 47 (38.7) | 22 (57.7) | 0 (73.3) | 0 (73.7) |
| AC8scFvTAT2A | 98 (1.3) | 97 (2.3) | 36 (47) | 1 (73) | 0 (75.3) | 0 (73.7) |
| AC8scFvTAT2B | 100 (0) | 95 (3.7) | 42 (42.7) | 0 (76) | 0 (73.3) | 0 (73.7) |
| AC8scFvTAT2C | 99 (1) | 99 (1) | 44 (41) | 1 (73) | 1 (73) | 0 (73.7) |

### Example 8

### Virus Neutralization by Association with the Target Cell

In this example, the ability of the various PTD-modified anti-HSV single chain antibodies to attach to target cells and/or become internalized by the target cells in order to inhibit subsequent viral infection and/or virus production was assessed by a virus prevention assay as described in Example 4. In this experiment, target cells are first incubated with the AC8 antibody alone. Following the pre-incubation, the free antibody is removed and the HSV-1 virus is added to the cells for a second incubation period. Following the virus incubation, the virus is removed from the cells and plaque formation is measured after several days incubation. As described above, the assay measures the anti-viral protective effects of the AC8 antibody that are dependent on attachment of the antibody to the target cell and/or internalization of the antibody.

Methods

The experiment was performed using the methods essentially as described in Example 3 for assaying HSV-1 neutralization that is dependent on association of the antibody with the target cell. Briefly, on Day 1 of the virus neutralization experiment, Vero cells (ATCC, cat no: CCL-81; Manassas, Va.) were prepared in 24-well cell culture plates at a cell density which allows formation of a cell monolayer (>90% confluence) by Day 2. On Day 2, each antibody was serially diluted in plain DMEM (Antibody concentrations tested in triplicate: 0.2 μg/ml, 0.6 μg/ml, 1.9 μg/ml, 5.6 μg/ml, 16.7 μg/ml, and 50 μg/ml). 100 ul of diluted antibody was added to each well and incubated at 37° C. for one hour. The cells were then washed three times with 1×PBS to remove free antibody. The HSV virus (ATCC, cat no: VR-733; Manassas, Va.) was diluted in plain DMEM (80 PFU/100 ul). 100 ul of diluted virus was added to the cells and incubated at 37° C. for one hour. Following the virus incubation period, the culture media containing the virus was aspirated and 1 ml of overlay medium was added to each well (overlay medium was prepared by mixing equal volumes of 2×DMEM/4% FBS (37° C.) and 1% agarose (56° C.)). The 24-well cell culture plates were then incubated at 37° C. for approximately 72 hours. Plaque formation was checked under microscope periodically. Once the plaques matured, the plates were fixed with 10% Formalin for 1 hour at room temperature. The formalin was then discarded and the plates were stained with 1% crystal violet solution in 70% methanol. The stained monolayers were then washed with ddH₂O and the plaques were counted. The plaque numbers for each of the test samples was compared to the control plaque number. The ED50 and ED90 of each antibody was calculated using Prism software. The percentage neutralization rate was set at 0% neutralization for the virus alone control samples, and the percentage neutralization for the antibody dilutions were calculated accordingly.

Two sets of experiments were performed. In the first experiment, the AC8scFvTAT, AC8scFvTAT1A, AC8scFvTAT1B, AC8scFvTAT1C, and AC8FabTAT antibodies were assayed (Table 11). In this experiment, the AC8Fab, AC8scFv, and AC8scFvTATscrambled antibodies were employed as negative controls. The AC8Fab and AC8scFv antibodies do not contain a PTD that provides for attachment to the target cells and the AC8scFvTATscrambled antibodies do not bind HSV-1.

In the second experiment, the AC8scFvTAT, AC8scFvTAT2A, AC8scFvTAT2B, and AC8scFvTAT2C antibodies were assayed (Table 12). The AC8scFv and AC8scFvTATscrambled antibodies were employed as negative controls in this experiment.

Results

Cells treated with the TAT-modified AC8 single chain antibodies exhibited statistically significant protection from HSV-1 virus infection in both sets of experiments. In the first experiment, the ED50 for the tested antibodies against HSV-1 infection were: 13 μg/ml for AC8scFvTAT, 10.1 μg/ml for AC8scFvTAT1A, 6.5 μg/ml for AC8scFvTAT1B, 9.9 μg/ml for AC8scFvTAT1C, and 27 μg/ml for AC8FabTAT. The single chain antibodies that contained TAT variant PTDs, 1A, 1B, and 1C, were as effective as TAT wild-type PTD in preventing HSV-1 infection. Further, the single chain antibodies were more effective than the AC8FabTAT antibody in preventing HSV-1 infection. The AC8Fab, AC8scFv, and AC8scFvTATscrambled negative control antibodies did not inhibit HSV-1 infection as expected.

TABLE 11

| | Plaque Reduction Rate Percentage | | | | | | |
|---|---|---|---|---|---|---|---|
| | (Average plaque number per plate) | | | | | | |
| | | | | | | | Virus |
| Antibody Tested | 50 μg/ml | 16.7 μg/ml | 5.6 μg/ml | 1.9 μg/ml | 0.6 μg/ml | 0.2 μg/ml | control |
| AC8scFvTAT | 98 (1.7) | 74 (19.3) | 19 (60) | 0 (75.7) | 0 (76.7) | 0 (77.7) | 0 (74) |
| AC8scFvTAT1A | 98 (1.3) | 89 (8) | 44 (41.3) | 4 (71.3) | 0 (73.7) | 0 (75.7) | 0 (74) |
| AC8scFvTAT1B | 100 (0.3) | 80 (15) | 16 (62.3) | 0 (75.3) | 0 (76.7) | 0 (74.3) | 0 (74) |
| AC8scFvTAT1C | 90 (7.7) | 60 (30) | 6 (69.3) | 0 (77.3) | 0 (75.3) | 0 (73.7) | 0 (74) |
| AC8FabTAT | 68 (23.7) | 37 (46.7) | 13 (64.3) | 0 (73.3) | 0 (77) | 0 (76.7) | 0 (74) |
| AC8Fab | 0 (73.3) | 0 (73.3) | 0 (75.3) | 0 (78) | 4 (70.7) | 0 (74.7) | 0 (74) |
| AC8scFv | 1 (73) | 3 (72) | 0 (73.3) | 0 (75.3) | 3 (71.7) | 0 (76.7) | 0 (74) |
| AC8scFvTAT | 3 (71.7) | 0 (73.3) | 0 (74.7) | 0 (74.7) | 0 (73.3) | 3 (71.3) | 0 (74) |
| scrambled | | | | | | | |

In the second experiment, the ED50 for the tested antibodies against HSV-1 infection were: 3 μg/ml for AC8scFvTAT2A, 5.2 μg/ml for AC8scFvTAT2B, 25 μg/ml for AC8scFvTAT2C, 7.1 μg/ml for AC8scFvTAT. The single chain antibodies that contained TAT variant PTDs, 2A, 2B, and 2C, exhibited inhibition of HSV-1 infection. In addition, AC8scFvTAT2A appeared more effective than AC8scFvTAT in inhibiting HSV-1 infection. The AC8scFv and AC8scFvTATscrambled negative control antibodies did not inhibit HSV-1 infection as expected.

TABLE 12

| | Plaque Reduction Rate Percentage | | | | | | |
|---|---|---|---|---|---|---|---|
| | (Average plaque number per plate) | | | | | | |
| | | | | | | | Virus |
| Antibody Tested | 50 μg/ml | 16.7 μg/ml | 5.6 μg/ml | 1.9 μg/ml | 0.6 μg/ml | 0.2 μg/ml | control |
| AC8scFvTAT | 97 (2.3) | 91 (6) | 41 (39) | 20 (52.7) | 0 (67.3) | 0 (66.7) | 0 (66) |
| AC8scFvTAT2A | 98 (1) | 95 (3) | 69 (20.7) | 37 (41.3) | 9 (60.3) | 4 (63) | 0 (66) |
| AC8scFvTAT2B | 99 (0.7) | 93 (4.7) | 55 (29.7) | 4 (63.3) | 0 (73.7) | 0 (76) | 0 (66) |
| AC8scFvTAT2C | 76 (16) | 36 (42.3) | 21 (52.3) | 5 (62.7) | 0 (67) | 0 (71.3) | 0 (66) |
| AC8scFv | 0 (71.7) | ND | ND | ND | ND | ND | ND |
| AC8scFvTAT | 0 (73) | ND | ND | ND | ND | ND | ND |
| scrambled | | | | | | | |

Since modifications will be apparent to those of skill in this art, it is intended that this invention be limited only by the scope of the appended claims.

1. A modified therapeutic antibody comprising:
a protein transduction domain; and
an antibody or antigen-binding fragment thereof, wherein the antigen binding domain of the antibody or antigen-binding fragment thereof specifically binds to a viral surface protein.

2. The modified therapeutic antibody of claim 1, wherein the protein transduction domain is selected from among a protein transduction domain comprising:
a) a polypeptide having an amino acid sequence B₁-X₁-B₂-B₃-B₄-X₂-B₅-B₆-X₃-X₄-B₇, wherein:
B₁, B₂, B₃, B₄, B₅, B₆, and B₇ are each independently lysine or arginine; and
X₁, X₂, X₃, and X₄ are each independently any amino acid except proline;
b) a polypeptide having an amino acid sequence B₁-P₁-X₁-B₂-P₂-B₃-X₂-X₃-X₄-B₄, wherein:
P₁ and P₂ are proline;
B₁, B₂, B₃ and B₄ are each independently lysine or arginine; and
X₁, X₂, X₃ and X₄ are each independently any amino acid;
c) a polypeptide having an amino acid sequence B₁-B₂-B₃-B₄-B₅-Q-B₆-B₇-B₈, wherein:
Q is glutamine; and
B₁, B₂, B₃, B₄, B₅, B₆, B₇, and B₈ are each independently lysine or arginine; and
d) a polypeptide having an amino acid sequence B₁-B₂-B₃-B₄-B₅-X-B₆-B₇-B₈, wherein:
X is any amino acid except glutamine; and
B₁, B₂, B₃, B₄, B₅, B₆, B₇, and B₈ are each independently lysine or arginine.

3. The modified therapeutic antibody of claim 2, wherein the protein transduction domain comprises a polypeptide having an amino acid sequence B₁-X₁-B₂-B₃-B₄-X₂-B₅-B₆-X₃-X₄-B₇, wherein each of X₁, X₂, X₃, and X₄ is independently selected from among any non-basic amino acid except proline.

4. The modified therapeutic antibody of claim 3, wherein each of X₁, X₂, X₃, and X₄ is independently selected from among serine, leucine, alanine, asparagine, aspartic acid and glycine.

5. The modified therapeutic antibody of claim 2, wherein:
the protein transduction domain comprises a polypeptide having an amino acid sequence B₁-X₁-B₂-B₃-B₄-X₂-B₅-B₆-X₃-X₄-B₇; and
the domain is selected from among a polypeptide having an amino acid sequence set forth in any of SEQ ID NOS:500-855 and 867.

6. The modified therapeutic antibody of claim 2, wherein:
the protein transduction domain comprises a polypeptide having an amino acid sequence B₁-X₁-B₂-B₃-B₄-X₂-B₅-B₆-X₃-X₄-B₇; and
the protein transduction domain comprises a polypeptide having an amino acid sequence selected from among RSRRKSRRNGR (SEQ ID NO: 505), RLRRKARRDSR (SEQ ID NO: 741) and KARRKGRRGGK (SEQ ID NO: 723).

7. The modified therapeutic antibody of claim 2, wherein the protein transduction domain comprises a polypeptide having an amino acid sequence B₁-P₁—X₁-B₂-P₂-B₃-X₂-X₃-X₄-B₄.

8. The modified therapeutic antibody of claim 7, wherein X₁, X₂, X₃, and X₄ are each independently selected from among arginine, lysine, proline and aspartic acid.

9. The modified therapeutic antibody of claim 7, wherein the protein transduction domain is selected from among polypeptides having an amino acid sequence set forth in any of SEQ ID NOS:144-499.

10. The modified therapeutic antibody of claim 7, wherein the protein transduction domain comprises a polypeptide having an amino acid sequence selected from among RPRRPRPDRR (SEQ ID NO: 160), KPRKPRRPRK (SEQ ID NO: 201), and RPLRPRRKGR (SEQ ID NO: 492).

11. The modified therapeutic antibody of claim 2, wherein the protein transduction domain comprises a polypeptide having an amino acid sequence B₁-B₂-B₃-B₄-B₅-Q-B₆-B₇-B₈.

12. The modified therapeutic antibody of claim 11, wherein the protein transduction domain is selected from among a polypeptide having an amino acid sequence set forth in any of SEQ ID NOS:5-30.

13. The modified therapeutic antibody of claim 11, wherein the protein transduction domain comprises a polypeptide having an amino acid sequence selected from among RRKRRQRRRR (SEQ ID NO: 9), RKKRKQKKR (SEQ ID NO: 13), and KRRKRQRRR (SEQ ID NO: 29).

14. The modified therapeutic antibody of claim 2, wherein the protein transduction domain comprises a polypeptide having an amino acid sequence B₁-B₂-B₃-B₄-B₅-X-B₆-B₇-B₈.

15. The modified therapeutic antibody of claim 14, wherein X is selected from among arginine, lysine, aspartic acid, glutamic acid and asparagine.

16. The modified therapeutic antibody of claim 14, wherein the protein transduction domain is selected from among polypeptides having an amino acid sequence set forth in any of SEQ ID NOS:31-143 and 860.

17. The modified therapeutic antibody of claim 14, wherein the protein transduction domain comprises a polypeptide having an amino acid sequence selected from among KKRKKEKKKR (SEQ ID NO: 90), KRRKRNRRRR (SEQ ID NO: 860) and RKRREKRRR (SEQ ID NO: 100).

18. The modified therapeutic antibody of claim 1, wherein the protein transduction domain is selected from among polypeptides having an amino acid sequence set forth in SEQ ID NOS: 868-939.

19. The modified therapeutic antibody claim 1, wherein the viral surface protein is an envelope protein or a capsid protein.

20. The modified therapeutic antibody of claim 19, wherein the viral envelope protein is a glycoprotein.

21. The modified therapeutic antibody of claim 1, wherein the virus is selected from a herpes virus (HSV), a metapneumovirus or a respiratory syncytial virus.

22. The modified therapeutic antibody of claim 1, wherein the virus is a HSV-1 or HSV-2.

23. The modified therapeutic antibody of claim 22, wherein the viral envelope protein is an herpes virus glycoprotein D.

24. The modified therapeutic antibody of claim 23, wherein the protein transduction domain is an HIV-TAT domain.

25. The modified therapeutic antibody of claim 1 that is a human or humanized antibody or antigen-binding fragment thereof.

26. The modified therapeutic antibody of claim 1, wherein the antibody or antigen-binding fragment thereof is a single-chain Fv (scFv), Fab, Fab′, F(ab′)₂, Fv, dsFv, diabody, Fd, or Fd′ fragment.

27. The modified therapeutic antibody of claim 26, wherein the antibody is a single-chain Fv and the single chain antibody comprises a light chain variable (V_{L}) domain or a functional region thereof that contains a complementarity determining region 1 (CDR1), a complementarity determining region 2 (CDR2) and a complementarity determining region 3 (CDR3) and/or the heavy chain variable (V_{H}) domain or functional region thereof that contains a complementarity determining region 1 (CDR1), a complementarity determining region 2 (CDR2) and a complementarity determining region 3 (CDR3).

28. The modified therapeutic antibody of 1, wherein the antibody portion is AC8 (ATCC Accession No. 69522) or an antigen binding fragment thereof.

29. The modified therapeutic antibody of claim 28, wherein the antibody comprises a light chain variable domain (V_{L}) having an amino acid sequence set forth in SEQ ID NO: 1052 and a heavy chain variable domain (V_{H}) having an amino acid sequence set forth in SEQ ID NO: 1053.

30. The modified therapeutic antibody of claim 1, wherein the antibody or antigen-binding fragment thereof comprises a light chain having an amino acid sequence set forth in SEQ ID NO: 1018 and a heavy chain having an amino acid sequence set forth in SEQ ID NO: 1056.

31. The modified therapeutic antibody of claim 1, wherein the antibody or antigen-binding fragment thereof comprises the amino acid sequence set forth in SEQ ID NO: 1.

32. The modified therapeutic antibody of claim 1, comprising a polypeptide having the amino acid sequence set forth in SEQ ID NO: 2.

33. The modified therapeutic antibody of claim 1, comprising a light chain having the amino acid sequence set forth in SEQ ID NO:1018 and an heavy chain having the amino acid sequence set forth in SEQ ID NO:1016.

34. The modified therapeutic antibody of claim 1, comprising a peptide linker located between the protein transduction domain and the antibody or antigen-binding fragment thereof.

35. The modified therapeutic antibody of claim 1, further comprising a diagnostic agent.

36. The modified therapeutic antibody of claim 35, wherein the diagnostic agent is selected from among an enzyme, a fluorescent compound, or an electron transfer agent.

37. A pharmaceutical composition comprising:
a modified therapeutic antibody of claim 1; and
a pharmaceutically acceptable carrier or excipient.

38. The pharmaceutical composition of claim 37 that is formulated as a gel, ointment, liquid, suspension, aerosol, tablet, pill or powder.

39. The pharmaceutical composition of claim 37 that is formulated as eyedrops or a nasal spray.

40. A method of treatment or prevention of a viral infection, comprising administering the pharmaceutical composition of claim 37 to a subject.

41. The method of claim 40, wherein the viral infection is a herpes virus infection.

42. The method of claim 40, wherein the composition is administered topically, parenterally, locally, or systemically.

43. The method of claim 40, wherein the composition is administered orally, ocularly, intravenously, or directly to a mucosal surface.

44. A vector, comprising nucleic acid encoding the modified therapeutic antibody of claim 1.

45. A nucleic acid molecule, comprising a sequence of nucleotides that encodes a modified therapeutic antibody of claim 1 or a heavy or light chain portion thereof comprising the protein transduction domain.

46. An isolated cell or cell culture, comprising a nucleic acid molecule of claim 44.

47. A method for producing a modified therapeutic antibody, comprising:
culturing a cell of claim 46 under conditions, whereby the encoded modified therapeutic antibody is expressed.

48. A method for increasing the therapeutic efficacy of an antiviral antibody or an antigen binding fragment thereof, comprising:
conjugating a protein transduction domain to an antibody or antigen-binding fragment thereof wherein the antigen binding domain of the antibody or antigen-binding fragment thereof binds a glycoprotein on the surface of a virus thereby increasing the therapeutic efficacy of the antibody or antigen-binding fragment thereof compared to the therapeutic efficacy of the antibody or antigen-binding fragment thereof in the absence of the protein transduction domain,
wherein the antibody or antigen-binding fragment is a neutralizing antibody or fragment thereof.

49. A protein transduction domain, wherein the protein transduction domain comprises a polypeptide selected from among:
a) a polypeptide having an amino acid sequence B₁-X₁-B₂-B₃-B₄-X₂-B₅-B₆-X₃-X₄-B₇, wherein:
B₁, B₂, B₃, B₄, B₅, B₆, and B₇ are each independently lysine or arginine; and
X₁, X₂, X₃, and X₄ are each independently any amino acid except proline;
b) a polypeptide having an amino acid sequence B₁-P₁-X₁-B₂-P₂-B₃-X₂-X₃-X₄-B₄, wherein:
P₁ and P₂ are proline;
B₁, B₂, B₃ and B₄ are each independently lysine or arginine; and
X₁, X₂, X₃ and X₄ are each independently any amino acid;
c) a polypeptide having an amino acid sequence B₁-B₂-B₃-B₄-B₅-Q-B₆-B₇-Bg, wherein:
Q is glutamine; and
B₁, B₂, B₃, B₄, B₅, B₆, B₇, and B₈ are each independently lysine or arginine provided that the protein transduction domain does not have the sequence RKKRRQRRR; or
d) a polypeptide having an amino acid sequence B₁-B₂-B₃-B₄-B₅-X-B₆-B₇-B₈, wherein:
X is any amino acid except glutamine; and
B₁, B₂, B₃, B₄, B₅, B₆, B₇, and B₈ are each independently lysine or arginine.

50. The protein transduction domain of claim 49 selected from among polypeptides having an amino acid sequence set forth in any of SEQ ID NOS:5-855, 860 and 867.

51. A modified therapeutic antibody comprising:
a protein transduction domain of claim 50; and
an antibody or antigen-binding fragment thereof.

52. The modified therapeutic antibody of claim 51, wherein the antibody or antigen-binding fragment thereof binds to a viral protein.

53. The modified therapeutic antibody of claim 52, wherein the viral protein is from a herpes virus, a metapneumovirus or a respiratory syncytial virus.


SEQUENCE LISTING (1056 sequences)

SEQ ID NO: 1 - protein, 300 aa, Artificial Sequence
  AC8scFv
MKKTAIAIAVALAGFATVAQAAEIVLTQSPGTLSLSPGERATLSCRASQSVSSAYLAWYQQKPGQAPRLLIYGASSRATGIPDRFSGSGSGTDFTLTISRLEPEDFAVYYCQQYGRSPTFGGGTKVEIKGGSSRSSSSGGGGSGGGGQVQLVQSGAEVKKPGSSVKVSCKASGGSFSSYAINWVRQAPGQGLEWMGGLMPIFGTTNYAQKFQDRLTITADVSTSTAYMQLSGLTYEDTAMYYCARVAYMLEPTVTAGGLDVWGQGTTVTVSSASTKGGQAGQHHHHHHGAYPYDVPDYAS

SEQ ID NO: 2 - protein, 311 aa, Artificial Sequence
  AC8scFvTAT
MKKTAIAIAVALAGFATVAQAAEIVLTQSPGTLSLSPGERATLSCRASQSVSSAYLAWYQQKPGQAPRLLIYGASSRATGIPDRFSGSGSGTDFTLTISRLEPEDFAVYYCQQYGRSPTFGGGTKVEIKGGSSRSSSSGGGGSGGGGQVQLVQSGAEVKKPGSSVKVSCKASGGSFSSYAINWVRQAPGQGLEWMGGLMPIFGTTNYAQKFQDRLTITADVSTSTAYMQLSGLTYEDTAMYYCARVAYMLEPTVTAGGLDVWGQGTTVTVSSASTKGGQAGQHHHHHHGAYPYDVPDYASRGRKKRRQRRR

SEQ ID NO: 3 - protein, 300 aa, Artificial Sequence
  AC8scFvscrambled
MKKTAIAIAVALAGFATVAQAAEIVLTQSPGTLSLSPGERATLSCRASQSVSSAYLAWYQQKPGQAPRLLIYGASSRATGIPDRFSGSGSGTDFTLTISRLEPEDFAVYYCQQYGRSPTFGGGTKVEIKGGSSRSSSSGGGGSGGGGQVQLVQSGAEVKKPGSSVKVSCKASGGSFSSYAINWVRQAPGQGLEWMGGLMPIFGTTNYAQKFQDRLTITADVSTSTAYMQLSGLTYEDTAMYYCARVVADYLMGLGEAPTTVWGQGTTVTVSSASTKGGQAGQHHHHHHGAYPYDVPDYAS

SEQ ID NO: 4 - protein, 311 aa, Artificial Sequence
  AC8scFvTATscrambled
MKKTAIAIAVALAGFATVAQAAEIVLTQSPGTLSLSPGERATLSCRASQSVSSAYLAWYQQKPGQAPRLLIYGASSRATGIPDRFSGSGSGTDFTLTISRLEPEDFAVYYCQQYGRSPTFGGGTKVEIKGGSSRSSSSGGGGSGGGGQVQLVQSGAEVKKPGSSVKVSCKASGGSFSSYAINWVRQAPGQGLEWMGGLMPIFGTTNYAQKFQDRLTITADVSTSTAYMQLSGLTYEDTAMYYCARVVADYLMGLGEAPTTVWGQGTTVTVSSASTKGGQAGQHHHHHHGAYPYDVPDYASRGRKKRRQRRR

SEQ ID NO: 5 - protein, 9 aa, Physcomitrella patens
  Physcomitrella Predicted Transduction Domain
KKRKRQRRK

SEQ ID NO: 6 - protein, 9 aa, Laccaria bicolor
  Bicoloured deceiver (strain S238N-H82) RNA processing-related protein
KKRKKQRRK

SEQ ID NO: 7 - protein, 10 aa, Drosophila virilis
  Fruit fly protein GJ17789
RKRRKQRKRK

SEQ ID NO: 8 - protein, 10 aa, Drosophila mojavensis
  Fruit fly protein GI21820
KKRRKQRKRK

SEQ ID NO: 9 - protein, 10 aa, Artificial Sequence
  FIV Rev
RRKRRQRRRR

SEQ ID NO: 10 - protein, 9 aa, Culex quinquefasciatus
  Southern house mosquito Polycomb protein esc
KKRRRQKKK

SEQ ID NO: 11 - protein, 9 aa, Ictalurus punctatus
  Channel catfish CBF1 interacting corepressor
KKKRKQKKK

SEQ ID NO: 12 - protein, 9 aa, Schizosaccharomyces pombe
  Fission yeast uncharacterized protein C14401
KRKRKQKKK

SEQ ID NO: 13 - protein, 9 aa, Homo sapiens
  Human ankyrin repeat domain containing protein 2
RKKRKQKKR

SEQ ID NO: 14 - protein, 9 aa, Nematostella vectensis
  Starlet sea anemone Putative protein
KKKRKQKRK

SEQ ID NO: 15 - protein, 10 aa, Homo sapiens
  Human tumor protein p53-inducible protein 13
RRRRKQRKKK

SEQ ID NO: 16 - protein, 9 aa, Danio rerio
  Zebrafish nucleolar protein 12
KKRRKQKRR

SEQ ID NO: 17 - protein, 9 aa, Drosophila persimilis
  Fruit fly protein GL18495
KRRKRQRKR

SEQ ID NO: 18 - protein, 10 aa, Drosophila grimshawi
  Fruit fly protein GH11647
KKRKRQKRRR

SEQ ID NO: 19 - protein, 9 aa, Drosophila grimshawi
  Fruit fly protein GH12028
KRRRKQKKR

SEQ ID NO: 20 - protein, 9 aa, Drosophila virilis
  Fruit fly protein GJ19358
KRRRKQKKK

SEQ ID NO: 21 - protein, 9 aa, Ostreococcus lucimarinus strain CCE9901
  Ostreococcus lucimarinus Putative protein
KRRRRQRKR

SEQ ID NO: 22 - protein, 9 aa, Tetraodon nigroviridis
  Green puffer chromosome undetermined SCAF7487
KRRRRQRRR

SEQ ID NO: 23 - protein, 9 aa, Oryza sativa
  Japonica rice Os05g0296800 protein
RKRRRQKKR

SEQ ID NO: 24 - protein, 9 aa, Entamoeba dispar
  Actin from Entamoeba dispar
KKRKRQKKK

SEQ ID NO: 25 - protein, 9 aa, Pichia stipitis
  Yeast  Putative protein
KKRKRQKRK

SEQ ID NO: 26 - protein, 9 aa, Trypanosoma cruzi
  Trypanosoma cruzi protein kinase
RKRKKQRKR

SEQ ID NO: 27 - protein, 9 aa, Trypanosoma cruzi
  Trypanosoma cruzi protein kinase
KKRKKQRKR

SEQ ID NO: 28 - protein, 9 aa, Candida albicans
  Yeast  Putative protein
KKKRRQKRR

SEQ ID NO: 29 - protein, 9 aa, Drosophila mojavensis
  Fruit fly protein GI14201
KRRKRQRRR

SEQ ID NO: 30 - protein, 10 aa, Homo sapiens
  Human Fer-1-like protein 4
KKKRKQRRRK

SEQ ID NO: 31 - protein, 9 aa, Bacillus subtilis
  Bacillus subtilis DNA translocase ftsK
KKKRKSRKK

SEQ ID NO: 32 - protein, 9 aa, Hyperthermus butylicus
  Hyperthermus butylicus (strain DSM 5456/JCM 9403) Conserved crenarchaeal protein-Zn binding domain
RRRRKYKKR

SEQ ID NO: 33 - protein, 9 aa, Alkaliphilus metalliredigens (strain QYMF)
  Alkaliphilus metalliredigens (strain QYMF) 30S ribosomal protein S18
RRRKKMKKK

SEQ ID NO: 34 - protein, 9 aa, Saccharomyces cerevisiae
  Baker′s yeast uncharacterized protein YHR007C-A
KKRKKEKKR

SEQ ID NO: 35 - protein, 9 aa, Alkaliphilus oremlandii
  Alkaliphilus oremlandii 30S ribosomal protein S18
KRKRKARKK

SEQ ID NO: 36 - protein, 9 aa, Neosartorya fischeri
  Neosartorya fischeri (strain ATCC 1020 / DSM 3700 / NRRL 181) Maiting-type protein
RRRRKEKKK

SEQ ID NO: 37 - protein, 9 aa, Pelotomaculum thermopropionicum
  Pelotomaculum thermopropionicum (strain DSM 13744 / JCM 10971 /SI) 50S ribosomal protein L18
KRRRRVRKK

SEQ ID NO: 38 - protein, 9 aa, Syntrophobacter fumaroxidans
  Syntrophobacter fumaroxidans (strain DSM 10017 / MPOB) LSU ribosomal protein L18P
KRKKRIRKK

SEQ ID NO: 39 - protein, 9 aa, Medicago truncatula
  Barrel medic Leucine-rich repeat
RKRRRIRRR

SEQ ID NO: 40 - protein, 9 aa, Sagittula stellata
  Sagittula stellata Biopolymer transport protein, ExbD/TolR family
KRRRRGRRK

SEQ ID NO: 41 - protein, 9 aa, Homo sapiens
  Human Arginine/serine-rich coiled-coil protein 1
KRKKKHRRR

SEQ ID NO: 42 - protein, 10 aa, Trichomonas vaginalis
  Trichomonas vaginalis Polylysine protein
KRKKRERKRR

SEQ ID NO: 43 - protein, 9 aa, Platygyra sinensis
  Platygyra sinensis Histone H2B
KKKRRGKRK

SEQ ID NO: 44 - protein, 9 aa, Chironomus thummi thummi
  Midge Histone H2B
KKKRRHKRK

SEQ ID NO: 45 - protein, 11 aa, Homo sapiens
  Human Transmembrane protein TTMA
KRKRKAKKRRK

SEQ ID NO: 46 - protein, 11 aa, Cyanothece
  Cyanothece CCY 0110 Penicillin-binding protein 1A
KKRRRFRRRKK

SEQ ID NO: 47 - protein, 10 aa, Burkholderia dolosa
  Burkholderia dolosa AUO158 Phosphopantetheinyl transferase
RKRRRCRRKR

SEQ ID NO: 48 - protein, 9 aa, Aquifex aeolicus
  Aquifex aeolicus 50S ribosomal protein L34
RRRRKGRKR

SEQ ID NO: 49 - protein, 9 aa, Southern bean mosaic virus
  Southern bean mosaic virus Capsid protein
RRKRRAKRR

SEQ ID NO: 50 - protein, 10 aa, Drosophila melanogaster
  Fruit fly Chromodomain-helicase- DNA-binding protein Mi-2 homolog
RRKKKGKKRK

SEQ ID NO: 51 - protein, 9 aa, Saccharomyces cerevisiae
  Baker′s yeast Uncharacterized protein YLR137W
RKKKRVRRR

SEQ ID NO: 52 - protein, 10 aa, Pichia stipitis
  Yeast  Putative protein
KKKRKHKRKR

SEQ ID NO: 53 - protein, 11 aa, Debaryomyces hansenii
  Yeast Transcription elongation factor SPT6
RKRRKHKKRKR

SEQ ID NO: 54 - protein, 9 aa, Candida albicans
  Yeast Stress response protein NST1
KKRKKHKKK

SEQ ID NO: 55 - protein, 9 aa, Burkholderia mallei
  Burkholderia mallei (strain NCTC 10247) Cyclic di-GMP binding protein
KRRRRGRRR

SEQ ID NO: 56 - protein, 9 aa, Ashbya gossypii
  Yeast Transcription elongation factor SPT6
RRRRKHKRR

SEQ ID NO: 57 - protein, 9 aa, Eledone cirrhosa
  Curled octopus Cysteine-rich protamine
RKKKRCRRK

SEQ ID NO: 58 - protein, 9 aa, Candida glabrata
  Yeast Transcription elongation factor SPT6
KKKRRHKRR

SEQ ID NO: 59 - protein, 9 aa, Saccharomyces cerevisiae
  Baker′s yeast Transcription elongation factor SPT6
KKRRKHKRR

SEQ ID NO: 60 - protein, 9 aa, Mus musculus
  Mouse Tubulin polyglutamylase TTLL13
RRRRKRKRR

SEQ ID NO: 61 - protein, 9 aa, Homo sapiens
  Human Tubulin polyglutamylase TTLL13
RRKRRRKRR

SEQ ID NO: 62 - protein, 9 aa, Neurospora crassa
  Neurospora crassa Transcription elongation factor spt-6
RRRRKKRRR

SEQ ID NO: 63 - protein, 11 aa, Bos taurus
  Bovine Prokineticin-2
RRKRKRRRKKK

SEQ ID NO: 64 - protein, 10 aa, Homo sapiens
  Human Prokineticin-2
RKRKRSKRKK

SEQ ID NO: 65 - protein, 9 aa, Gnetum gnemon
  Bago RNA polymerase beta chain
KRKKKGKRK

SEQ ID NO: 66 - protein, 9 aa, Pichia stipitis
  Yeast PIP5K and related FYVE finger-containing proteins Signal transduction mechanisms
KKKRRGKKK

SEQ ID NO: 67 - protein, 10 aa, Adelaide River virus
  Adelaide River virus Protein alpha-1
KRKRRKKKRK

SEQ ID NO: 68 - protein, 9 aa, Schizosaccharomyces pombe
  Fission yeast Meiotically up-regulated gene 116 protein
KKRKKEKKK

SEQ ID NO: 69 - protein, 9 aa, Aspergillus clavatus
  Aspergillus clavatus Methionine aminopeptidase
KKRKRSKKK

SEQ ID NO: 70 - protein, 9 aa, Xenopus tropicalis
  Western clawed frog LOC100036708 protein
RRKKRERKK

SEQ ID NO: 71 - protein, 10 aa, Chlorella vulgaris
  Green alga Uncharacterized 165 kDa protein in psaC-atpA intergenic region
KRKKRTKKKR

SEQ ID NO: 72 - protein, 9 aa, Homo sapiens
  Human Transient receptor potential cation channel subfamily V member 3
RKKRRLKKR

SEQ ID NO: 73 - protein, 9 aa, Dictyostelium discoideum
  Slime mold Uncharacterized protein DDB_0237901
RRKKRRRRK

SEQ ID NO: 74 - protein, 10 aa, Mullus surmuletus
  Striped red mullet Protamine-like protein
KKKRKRRRRK

SEQ ID NO: 75 - protein, 10 aa, Xanthomonas oryzae
  Xanthomonas oryzae Transposase
RKRRRKRRRR

SEQ ID NO: 76 - protein, 9 aa, Aspergillus niger
  Aspergillus niger (strain CBS 51388 / FGSC A1513) Putative protein
KRKRKRKRR

SEQ ID NO: 77 - protein, 9 aa, Bos taurus
  Bovine Probetacellulin
RKRRKRRKK

SEQ ID NO: 78 - protein, 9 aa, Homo sapiens
  Human Probetacellulin
RKRRKRKKK

SEQ ID NO: 79 - protein, 9 aa, Homo sapiens
  Human Coiled-coil domain-containing protein 140
RKRRKERKK

SEQ ID NO: 80 - protein, 11 aa, Danio rerio
  Zebrafish Zgc162339 protein
KKRKKEKKKRK

SEQ ID NO: 81 - protein, 9 aa, Homo sapiens
  Human Lipin-1
KKRRKRRRK

SEQ ID NO: 82 - protein, 9 aa, Ostreococcus lucimarinus
  Ostreococcus lucimarinus (strain CCE9901) Putative protein
RRRKKEKRR

SEQ ID NO: 83 - protein, 9 aa, Ostreococcus lucimarinus
  Ostreococcus lucimarinus (strain CCE9901) Putative protein
RKRKKERKK

SEQ ID NO: 84 - protein, 9 aa, Drosophila melanogaster
  Fruit fly Longitudinals lacking protein, isoform G
RRRRKVRRR

SEQ ID NO: 85 - protein, 9 aa, Bos taurus
  Bovine Uncharacterized protein C12orf43 homolog
KKKRKLKKK

SEQ ID NO: 86 - protein, 9 aa, Anopheles gambiae
  African malaria mosquito protein AGAP005245-PD
RKRKKVRRR

SEQ ID NO: 87 - protein, 9 aa, Homo sapiens
  Human Uncharacterized protein C11orf57
KRKKKSRKK

SEQ ID NO: 88 - protein, 9 aa, Saccharomyces cerevisiae
  Baker′s yeast Uncharacterized protein YEL057C
KRRKKLKRK

SEQ ID NO: 89 - protein, 9 aa, Trichomonas vaginalis
  Trichomonas vaginalis Cyclophilin-RNA interacting protein
RKRKRRRRK

SEQ ID NO: 90 - protein, 10 aa, Tobacco mild green mosaic virus
  TMGMV Movement protein
KKRKKEKKKR

SEQ ID NO: 91 - protein, 9 aa, Ecotropis obliqua NPV
  Ecotropis obliqua NPV Late expression factor 5
KKKRKIKKR

SEQ ID NO: 92 - protein, 11 aa, Aedes aegypti
  Yellowfever mosquito Mediator of RNA polymerase II transcription subunit 19
KKRKKEKKRKK

SEQ ID NO: 93 - protein, 10 aa, Rattus norvegicus
  Rat T-cell surface antigen CD2
KRKKRNRRRK

SEQ ID NO: 94 - protein, 10 aa, Mus musculus
  Mouse T-cell surface antigen CD2
KRRKRNRRRK

SEQ ID NO: 95 - protein, 9 aa, Xenopus tropicalis
  Western clawed frog LOC100124814 protein
RKRKKKRRK

SEQ ID NO: 96 - protein, 10 aa, Mus musculus
  Mouse MKI67 FHA domain interacting nucleolar phosphoprotein
KKRKRSRRKK

SEQ ID NO: 97 - protein, 11 aa, Caenorhabditis elegans
  C. elegans Protein let-756
KKRRREKKKRR

SEQ ID NO: 98 - protein, 10 aa, Homo sapiens
  Human Ankyrin repeat domain containing protein 18B
KKRKKLKKRK

SEQ ID NO: 99 - protein, 10 aa, Homo sapiens
  Human G patch domain-containing protein 8
KKRKKRKRKK

SEQ ID NO: 100 - protein, 10 aa, Ustilago maydis
  Smut fungus Vacuolar fusion protein MON1
RKRRREKRRR

SEQ ID NO: 101 - protein, 10 aa, Microscilla marina
  Microscilla marina (ATCC 23134) Outer membrane protein OmpA family
KKRRKRKKRR

SEQ ID NO: 102 - protein, 11 aa, Fugu rubripes
  Japanese pufferfish Homeobox protein Hox-C8a
KRRKRKKRKRK

SEQ ID NO: 103 - protein, 9 aa, Mus musculus
  Mouse Scaffold attachment factor B2
RRKRKRRRK

SEQ ID NO: 104 - protein, 11 aa, Homo sapiens
  Human RING and PHD-finger domain-containing protein KIAA1542
RRKRKTRRRKK

SEQ ID NO: 105 - protein, 9 aa, Haliotis asinina
  Haliotis asinina protein BMP2/4
RKKKRRRRK

SEQ ID NO: 106 - protein, 10 aa, Bos taurus
  Bovine Target of EGR1 protein 1
RRRRKEKRRR

SEQ ID NO: 107 - protein, 10 aa, Mus musculus
  Mouse Target of EGR1 protein 1
RRRRKDKRKR

SEQ ID NO: 108 - protein, 9 aa, Dictyostelium discoideum
  Slime mold rRNA methyltransferase 3 homolog
KKKRKEKKR

SEQ ID NO: 109 - protein, 9 aa, Mus musculus
  Mouse Ribosomal RNA processing protein 1 homolog B
KKKRKKRKR

SEQ ID NO: 110 - protein, 9 aa, Homo sapiens
  Human Kinesin-like protein KIF3B
RRKRREKRR

SEQ ID NO: 111 - protein, 9 aa, Leishmania braziliensis
  Leishmania braziliensis RNA-binding protein
RRRRKMKRR

SEQ ID NO: 112 - protein, 9 aa, Bacillus thuringiensis
  Bacillus thuringiensis (strain Al Hakam) ATP-dependent RNA helicase, DEAD/DEAH box family
RRRRKSRKK

SEQ ID NO: 113 - protein, 9 aa, Ajellomyces capsulata
  Darling′s disease fungus (strain NAm1 / WU24) WD repeat-containing protein JIP5
KKKRKKRKK

SEQ ID NO: 114 - protein, 10 aa, Ostreococcus lucimarinus
  Ostreococcus lucimarinus (strain CCE9901) Putative protein
KRKKKSKRRK

SEQ ID NO: 115 - protein, 9 aa, Homo sapiens
  Human Histone-lysine Nmethyltransferase, H3 lysine-9 specific 5
KRRRRSRKK

SEQ ID NO: 116 - protein, 9 aa, Rattus norvegicus
  Rat Ankyrin repeat and zinc finger domain-containing protein 1
KRRKKRKKK

SEQ ID NO: 117 - protein, 9 aa, Danio rerio
  Zebrafish LOC798657 protein
KRRRKHRKR

SEQ ID NO: 118 - protein, 9 aa, Marine gamma proteobacterium HTCC2143
  Marine gamma proteobacterium HTCC2143 Poly(A) polymerase
KRKRKPRKK

SEQ ID NO: 119 - protein, 9 aa, Thiobacillus denitrificans
  Thiobacillus denitrificans (strain ATCC 25259) GTP-binding protein engA
RRKKRVRRK

SEQ ID NO: 120 - protein, 9 aa, Pichia stipitis
  Yeast Kinase of RNA polymerase II carboxy-terminal domain (CTD), alpha subunit
KRRRKERKR

SEQ ID NO: 121 - protein, 9 aa, Paramecium tetraurelia
  Paramecium tetraurelia Chromosome undetermined scaffold_62
KKRKKRRRR

SEQ ID NO: 122 - protein, 9 aa, Human papillomavirus
  HPV type 41 Minor capsid protein L2
RRKRKKRKR

SEQ ID NO: 123 - protein, 10 aa, Homo sapiens
  Human Probable global transcription activator SNF2L2
KKKRRRRKKK

SEQ ID NO: 124 - protein, 9 aa, Ostreococcus lucimarinus
  Ostreococcus lucimarinus (strain CCE9901) Putative protein
RRRKRSRRR

SEQ ID NO: 125 - protein, 10 aa, Desulfovibrio vulgaris
  Desulfovibrio vulgaris (strain DP4) DEAD/DEAH box helicase domain protein
RKRRRPRRRK

SEQ ID NO: 126 - protein, 9 aa, Robiginitalea biformata
  Robiginitalea biformata HTCC2501 Signal peptidase I
RRRRKNRKK

SEQ ID NO: 127 - protein, 11 aa, Schizosaccharomyces pombe
  Fission yeast Serine/threonine-protein kinase ppk4
KRRRKGKRRKR

SEQ ID NO: 128 - protein, 10 aa, Geobacter bemidjiensis
  Geobacter bemidjiensis (strain Bem) Ribonuclease, Rne/Rng family
KKKRRRKRKR

SEQ ID NO: 129 - protein, 11 aa, Magnetococcus
  Magnetococcus (strain MC-1) Ribonuclease E
KKRKRRRKRRR

SEQ ID NO: 130 - protein, 9 aa, Juncus decipiens
  Juncus decipiens NADH dehydrogenase subunit F
KRKRRLKKK

SEQ ID NO: 131 - protein, 10 aa, Homo sapiens
  Human Smoothened homolog
RKKKRRKRKK

SEQ ID NO: 132 - protein, 10 aa, Aspergillus clavatus
  Aspergillus clavatus, Involucrin repeat protein
RRKRRAKKRR

SEQ ID NO: 133 - protein, 10 aa, Caenorhabditis elegans
  C. elegans Similarity to hypothetical protein isof
RRKRRTKRRK

SEQ ID NO: 134 - protein, 9 aa, Janibacter
  Janibacter HTCC2649 Polyphosphate kinase
KRRRKARRR

SEQ ID NO: 135 - protein, 11 aa, Microscilla marina
  Microscilla marina ATCC 23134 TonB-dependent receptor
RKKRRKRKKRR

SEQ ID NO: 136 - protein, 9 aa, Mus musculus
  Mouse WD repeat-containing protein 3
KKKRKNRKR

SEQ ID NO: 137 - protein, 10 aa, Plasmodium falciparum
  Plasmodium falciparum (isolate CDC / Honduras) DNA-directed RNA polymerase II subunit RPB1
KKRRKRRRRK

SEQ ID NO: 138 - protein, 9 aa, Homo sapiens
  Human AT-rich interactive domain containing protein 4A
RRRKRLKKK

SEQ ID NO: 139 - protein, 9 aa, Pinus thunbergii
  Green pine Protein ycf2
KRKRKTKRK

SEQ ID NO: 140 - protein, 9 aa, Homo sapiens
  Human Polycystic kidney disease and receptor for egg jelly related protein
KRKKRIKRR

SEQ ID NO: 141 - protein, 9 aa, Emericella nidulans
  Emericella nidulans Cytokinesis protein sepA
RRKRRLRRR

SEQ ID NO: 142 - protein, 9 aa, Ustilago maydis
  Smut fungus Lysophospholipase NTE1
KKRRRTRRK

SEQ ID NO: 143 - protein, 9 aa, Paramecium tetraurelia
  Paramecium tetraurelia Chromosome undetermined scaffold_68
KKRKRVKRK

SEQ ID NO: 144 - protein, 10 aa, Burkholderia cenocepacia
  Burkholderia cenocepacia (strain HI2424) TRAP-type uncharacterized transport system periplasmic component-like protein
KPARPRPPRR

SEQ ID NO: 145 - protein, 10 aa, Salinispora tropica
  Salinispora tropica (strain ATCC BAA-916 / DSM 44818 / CNB-440) Pyruvate flavodoxin/ferredoxin oxidoreductase domain protein
RPGRPRRPKR

SEQ ID NO: 146 - protein, 10 aa, Roseobacter
  Roseobacter SK209-2-6 Transcriptional regulator
KPAKPRKERK

SEQ ID NO: 147 - protein, 10 aa, Aeropyrum pernix
  Aeropyrum pernix Transcriptional regulator
RPPKPKRMGR

SEQ ID NO: 148 - protein, 10 aa, Bradyrhizobium
  Bradyrhizobium (strain BTAi1 / ATCC BAA-1182) Diguanylate cyclase/ phosphodiesterase
RPIKPKKLNR

SEQ ID NO: 149 - protein, 10 aa, Nocardioides
  Nocardioides (strain BAA-499 / JS614) Na(+)/H(+) antiporter nhaA 1
RPTRPRLFSR

SEQ ID NO: 150 - protein, 10 aa, Desulfatibacillum alkenivorans
  Desulfatibacillum alkenivorans AK-01 GTP-binding protein LepA
RPERPRPSGK

SEQ ID NO: 151 - protein, 10 aa, Oceanicaulis alexandrii
  Oceanicaulis alexandrii HTCC2633 Probable endonuclease III protein
KPKKPKSKRR

SEQ ID NO: 152 - protein, 10 aa, Kordia algicida
  Kordia algicida OT-1 Protein containing StAR-related lipid-transfer (START) domain
KPAKPKRWKK

SEQ ID NO: 153 - protein, 10 aa, Caenorhabditis briggsae
  Caenorhabditis briggsae UPF0493 protein CBG04313
RPKKPKHRER

SEQ ID NO: 154 - protein, 10 aa, Chlamydomonas reinhardtii
  Chlamydomonas reinhardtii  Putative protein
RPPKPKAKPK

SEQ ID NO: 155 - protein, 10 aa, Laccaria bicolor
  Bicoloured deceiver (strain S238NH82)  Putative protein
KPSRPKGTYR

SEQ ID NO: 156 - protein, 10 aa, Haemophilus aphrophilus
  Haemophilus aphrophilus Penicillin-binding protein 3
KPIKPKKTTK

SEQ ID NO: 157 - protein, 10 aa, Synechococcus
  Synechococcus (strain WH8102) Translation initiation factor IF-2
RPAKPKSQRK

SEQ ID NO: 158 - protein, 10 aa, Burkholderia pseudomallei
  Burkholderia pseudomallei Pasteur 52237, protein Gp7
RPYRPRPSSR

SEQ ID NO: 159 - protein, 10 aa, Haemophilus parainfluenzae
  Haemophilus parainfluenzae Penicillin-binding protein 3
KPGKPKKTIR

SEQ ID NO: 160 - protein, 10 aa, Methylobacterium
  Methylobacterium (strain 4-46) General secretory system II protein E domain protein
RPRRPRPDRR

SEQ ID NO: 161 - protein, 10 aa, Rhodopseudomonas palustris
  Rhodopseudomonas palustris (strain BisA53) Lipoyl synthase
KPIRPRHPEK

SEQ ID NO: 162 - protein, 10 aa, Coprinopsis cinerea
  Inky cap fungus (strain Okayama-7 / 130 / FGSC 9003) Phosphorylase
RPTRPRRHVR

SEQ ID NO: 163 - protein, 10 aa, Burkholderia contaminans
  Burkholderia contaminans Protein recA
RPVKPRSRCR

SEQ ID NO: 164 - protein, 10 aa, Nematostella vectensis
  Starlet sea anemone  Putative protein
RPTKPKDKLR

SEQ ID NO: 165 - protein, 10 aa, Aromatoleum aromaticum
  Aromatoleum aromaticum (strain EbN1) Probable chorismate--pyruvate lyase
RPPRPRVPAR

SEQ ID NO: 166 - protein, 10 aa, Sclerotinia sclerotiorum
  White mold (strain ATCC 18683 / 1980 / Ss-1) Putative protein
RPKKPKSHAR

SEQ ID NO: 167 - protein, 10 aa, Culex quinquefasciatus
  Southern house mosquito Ribonuclease iii
RPPKPKLKTR

SEQ ID NO: 168 - protein, 10 aa, Eurypelma californica
  American tarantula Neurotoxin ESTx1
KPCKPKGEKK

SEQ ID NO: 169 - protein, 10 aa, Kineococcus radiotolerans
  Kineococcus radiotolerans (strain ATCC BAA-149 / DSM 14245 / SRS30216) Integral membrane sensor signal transduction histidine kinase
RPPRPRRLLR

SEQ ID NO: 170 - protein, 10 aa, Pseudomonas putida
  Pseudomonas putida (strain GB-1) Transcriptional regulator from GntR family with aminotransferase domain
KPFKPKSGPR

SEQ ID NO: 171 - protein, 10 aa, Roseovarius
  Roseovarius sp. 217 S-adenosylmethioninetRNA ribosyltransferase-isomerase
RPARPRRAAR

SEQ ID NO: 172 - protein, 10 aa, Aspergillus clavatus
  Aspergillus clavatus DUF1665 domain protein
KPGRPKKKFR

SEQ ID NO: 173 - protein, 10 aa, Novosphingobium aromaticivorans
  Novosphingobium aromaticivorans (strain DSM 12444) S-adenosylmethioninetRNA ribosyltransferase-isomerase
RPARPRDSAR

SEQ ID NO: 174 - protein, 10 aa, Dinoroseobacter shibae
  Dinoroseobacter shibae (strain DFL 12) S-adenosylmethioninetRNA ribosyltransferase-isomerase
RPARPRSSAR

SEQ ID NO: 175 - protein, 10 aa, Erythrobacter
  Erythrobacter sp. NAP1 S-adenosylmethioninetRNAribosyltransferase
RPVRPRDAAR

SEQ ID NO: 176 - protein, 10 aa, Roseovarius nubinhibens
  Roseovarius nubinhibens ISM S-adenosylmethioninetRNA ribosyltransferase-isomerase
RPVRPRPAAR

SEQ ID NO: 177 - protein, 10 aa, Rhodobacter sphaeroides
  Rhodobacter sphaeroides (strain ATCC 17029 / ATH 2.4.9) S-adenosylmethioninetRNA ribosyltransferase-isomerase
RPARPRTAAR

SEQ ID NO: 178 - protein, 10 aa, Maricaulis maris
  Maricaulis maris (strain MCS10) S-adenosylmethioninetRNA ribosyltransferase-isomerase
RPARPRDAAR

SEQ ID NO: 179 - protein, 10 aa, Bos taurus
  Bovine SLC15A3 protein
RPRRPRRWWR

SEQ ID NO: 180 - protein, 10 aa, Homo sapiens
  Human NOX5 variant lacking EF hands (HCG2003451), isoform CRA_d
RPRRPRQLTR

SEQ ID NO: 181 - protein, 10 aa, Chlamydomonas reinhardtii
  Chlamydomonas reinhardtii  Putative protein
KPLKPKQGTK

SEQ ID NO: 182 - protein, 10 aa, Methylobacterium
  Methylobacterium (strain 4-46) Integrase catalytic region
RPVRPRGPSR

SEQ ID NO: 183 - protein, 10 aa, Microscilla marina
  Microscilla marina ATCC 23134 Methylmalonyl-CoA mutase
KPYKPKNHIR

SEQ ID NO: 184 - protein, 10 aa, Triticum aestivum
  Wheat MBD3 protein
RPRKPRPGSR

SEQ ID NO: 185 - protein, 10 aa, Vitis vinifera
  Grape Chromosome chr18 scaffold_1
KPRKPKLERR

SEQ ID NO: 186 - protein, 10 aa, Bat coronavirus
  Bat coronavirus HKU5-2 Nucleocapsid phosphoprotein
RPGRPRTKPR

SEQ ID NO: 187 - protein, 10 aa, Mycobacterium vanbaalenii
  Mycobacterium vanbaalenii (strain DSM 7251 / PYR-1) VanW family protein
KPAKPRSFRR

SEQ ID NO: 188 - protein, 10 aa, Vibrio splendidus
  Vibrio splendidus 12B01 Membrane carboxypeptidase
KPRKPRAAAK

SEQ ID NO: 189 - protein, 10 aa, Vibrio campbellii
  Vibrio campbellii AND4 3-deoxy-D-manno-octulosonic-acid transferase
KPNKPKFGQR

SEQ ID NO: 190 - protein, 10 aa, Vibrio parahaemolyticus
  Vibrio parahaemolyticus AQ3810 3-deoxy-D-manno-octulosonic-acid transferase
KPNKPKFGGR

SEQ ID NO: 191 - protein, 10 aa, Vibrio
  Vibrio MED222 Membrane carboxypeptidase
KPRKPRASAK

SEQ ID NO: 192 - protein, 10 aa, Vibrio splendidus
  Vibrio splendidus 12B01 3-deoxy-D-manno-octulosonic-acid transferase
KPNKPKFGNR

SEQ ID NO: 193 - protein, 10 aa, Vibrio
  Vibrio Ex25 3-deoxy-D-manno-octulosonic-acid transferase subfamily
KPNKPKFGSR

SEQ ID NO: 194 - protein, 10 aa, Vibrio harveyi
  Vibrio harveyi HY01 3-deoxy-D-manno-octulosonic-acid transferase
RPNKPKFGQR

SEQ ID NO: 195 - protein, 10 aa, Orgyia pseudotsugata
  OpMNPV DNA-binding protein
RPGRPRTYRR

SEQ ID NO: 196 - protein, 10 aa, Human papillomavirus
  HPV type 29 Probable protein E4
RPPKPRWGLR

SEQ ID NO: 197 - protein, 10 aa, Ostreococcus lucimarinus
  Ostreococcus lucimarinus (strain CCE9901) Putative protein
RPRKPKYFNR

SEQ ID NO: 198 - protein, 10 aa, Cryptococcus neoformans
  Cryptococcus neoformans rRNA-processing protein EFG1
KPARPKPTHR

SEQ ID NO: 199 - protein, 10 aa, Caenorhabditis elegans
  C. elegans Bifunctional heparan sulfate Ndeacetylase/ N-sulfotransferase 1
KPPKPRKKPK

SEQ ID NO: 200 - protein, 10 aa, Caenorhabditis briggsae
  C. briggsae Bifunctional heparan sulfate Ndeacetylase/ N-sulfotransferase 1
KPGKPRKPPK

SEQ ID NO: 201 - protein, 10 aa, Mus musculus
  Mouse Zinc finger protein 41
KPRKPRRPRK

SEQ ID NO: 202 - protein, 10 aa, Ralstonia solanacearum
  Ralstonia solanacearum UW551 Beta-hexosaminidase
RPIRPRPAMK

SEQ ID NO: 203 - protein, 11 aa, Coprinopsis cinerea
  Inky cap fungus (strain Okayama-7 / 130 / FGSC 9003) Putative protein
RPGRPKGSTTR

SEQ ID NO: 204 - protein, 10 aa, Azorhizobium caulinodans
  Azorhizobium caulinodans (strain ATCC 43989 / DSM 5975 / ORS 571) Regulatory protein
RPTRPKVRVR

SEQ ID NO: 205 - protein, 10 aa, Laccaria bicolor
  Bicoloured deceiver (strain S238NH82) Putative protein
KPGRPKIASK

SEQ ID NO: 206 - protein, 10 aa, Vibrio campbellii
  Vibrio campbellii AND4 Pseudouridine synthase
KPAKPRIAPK

SEQ ID NO: 207 - protein, 10 aa, Laccaria bicolor
  Bicoloured deceiver (strain S238NH82) Putative protein
RPGRPKGSTK

SEQ ID NO: 208 - protein, 10 aa, Oceanibulbus indolifex
  Oceanibulbus indolifex HEL-45 Probable insertion sequence transposase protein
RPEKPRQINR

SEQ ID NO: 209 - protein, 10 aa, Mus musculus
  Mouse Gamma-aminobutyric acid (GABAC) receptor
RPGKPRPLLR

SEQ ID NO: 210 - protein, 10 aa, Roseovarius nubinhibens
  Roseovarius nubinhibens  ISM Penicillin-binding protein, 1A family protein
RPRKPRPPRR

SEQ ID NO: 211 - protein, 10 aa, Idiomarina baltica
  Idiomarina baltica OS145 Apolipoprotein N-acyltransferase
KPLKPKQAFR

SEQ ID NO: 212 - protein, 10 aa, Aspergillus niger
  Aspergillus niger (strain CBS 513.88 / FGSC A1513) Catalytic activity hydrolysis of terminal 1
KPDKPKGVGK

SEQ ID NO: 213 - protein, 10 aa, Brugia malayi
  Filarial nematode worm 60S ribosomal protein L34
KPARPRQLRK

SEQ ID NO: 214 - protein, 10 aa, Synechococcus
  Cyanobacteria  bacterium Yellowstone A-Prime Translation initiation factor IF-2
KPTRPRLRPK

SEQ ID NO: 215 - protein, 10 aa, Homo sapiens
  Human Chromodomain-helicase-DNA binding protein 3
KPGKPRKRKK

SEQ ID NO: 216 - protein, 10 aa, Pichia guilliermondii
  Yeast Putative protein
RPRRPKLNSK

SEQ ID NO: 217 - protein, 10 aa, Chlamydomonas reinhardtii
  Chlamydomonas reinhardtii  Putative protein
RPQRPRRQQK

SEQ ID NO: 218 - protein, 10 aa, Bartonella tribocorum
  Bartonella tribocorum (strain CIP 105476 / IBS 506) TrwJ2 protein
RPNRPRVKKK

SEQ ID NO: 219 - protein, 10 aa, Magnaporthe grisea
  Rice blast fungus Putative protein
RPHRPKSQTR

SEQ ID NO: 220 - protein, 10 aa, Magnaporthe grisea
  Rice blast fungus Putative protein
KPAKPKPAPK

SEQ ID NO: 221 - protein, 10 aa, Murine cytomegalovirus
  Murine cytomegalovirus (strain K181) M25 protein
RPFRPRTPVR

SEQ ID NO: 222 - protein, 10 aa, Hoeflea phototrophica
  Hoeflea phototrophica DFL-43 Transcriptional regulator
RPERPRSCYR

SEQ ID NO: 223 - protein, 10 aa, Aeropyrum pernix
  Aeropyrum pernix 50S ribosomal protein L13P
RPKRPRTPQR

SEQ ID NO: 224 - protein, 10 aa, Coprinopsis cinerea
  Inky cap fungus (strain Okayama-7 / 130 / FGSC 9003)  Putative protein
RPSKPRKEKK

SEQ ID NO: 225 - protein, 10 aa, Methanoculleus marisnigri
  Methanoculleus marisnigri (strain ATCC 35101 / DSM 1498 / JR1) RNP-1 like RNA-binding protein
RPPRPRNDFR

SEQ ID NO: 226 - protein, 10 aa, Nitratiruptor
  Nitratiruptor strain SB155-2 Phosphate ABC transporter, substrate-binding protein
KPLKPKQLDR

SEQ ID NO: 227 - protein, 10 aa, Sulfurovum
  Sulfurovum (strain NBC37-1) Phosphate ABC transporter, substrate-binding protein
KPLKPKTLKK

SEQ ID NO: 228 - protein, 10 aa, Botryotinia fuckeliana
  Noble rot fungus (strain B05.10)  Putative protein
RPQRPKPQRR

SEQ ID NO: 229 - protein, 10 aa, Arcobacter butzleri
  Arcobacter butzleri (strain RM4018) TonB-dependent receptor protein
KPEKPKKEHK

SEQ ID NO: 230 - protein, 10 aa, Sindbis virus
  SINV Structural polyprotein
KPKKPKTQEK

SEQ ID NO: 231 - protein, 10 aa, Physcomitrella paten
  Physcomitrella patens  Putative protein
KPPRPRGRGR

SEQ ID NO: 232 - protein, 10 aa, Sindbis virus
  Sindbis virus (strain Edsbyn 82-5)
KPKKPKPQEK

SEQ ID NO: 233 - protein, 10 aa, Sphingomonas wittichii
  Sphingomonas wittichii (strain RW1 / DSM 6014 / JCM 10273) Enoyl-CoA hydratase/isomerase
RPARPRAGGR

SEQ ID NO: 234 - protein, 10 aa, Saimiriine herpesvirus
  Marmoset herpesvirus (strain MV-5-4-PSL) Glycoprotein B
KPRKPRPGRR

SEQ ID NO: 235 - protein, 10 aa, Xenopus laevis
  African clawed frog Transcriptional adapter 1-like protein
KPGKPKGKKK

SEQ ID NO: 236 - protein, 10 aa, Actinobacillus succinogenes
  Actinobacillus succinogenes (strain ATCC 55618 / 130Z) TonB family protein
KPEKPKEKPK

SEQ ID NO: 237 - protein, 10 aa, Nematostella vectensis
  Starlet sea anemone Putative protein
RPFKPRIKSR

SEQ ID NO: 238 - protein, 10 aa, Ashbya gossypii
  Yeast Histone H3-like centromeric protein CSE4
RPARPRETRR

SEQ ID NO: 239 - protein, 10 aa, Chlamydomonas reinhardtii
  Chlamydomonas reinhardtii  Putative protein
RPWKPRRHHR

SEQ ID NO: 240 - protein, 10 aa, Drosophila melanogaster
  Fruit fly protein CG14479-PA
KPKKPRTTRR

SEQ ID NO: 241 - protein, 10 aa, Homo sapiens
  Human RNA-binding protein Raly
KPDRPKGLKR

SEQ ID NO: 242 - protein, 10 aa, Mus musculus
  Mouse RNA-binding Raly-like protein
KPYRPKPGSK

SEQ ID NO: 243 - protein, 10 aa, Spermophilus tridecemlineatus
  Thirteen-lined ground squirrel Prion protein PrP
KPVKPKTSMK

SEQ ID NO: 244 - protein, 10 aa, Mus musculus
  Mouse RNA-binding protein Raly
KPNRPKGLKR

SEQ ID NO: 245 - protein, 10 aa, Xenopus tropicalis
  Western clawed frog RNA-binding Raly-like protein
KPYRPKLGTK

SEQ ID NO: 246 - protein, 10 aa, Bos taurus
  Bovine RNA-binding Raly-like protein
KPYRPKPGNK

SEQ ID NO: 247 - protein, 10 aa, Victivallis vadensis
  Victivallis vadensis (ATCC BAA-548) Binding-protein-dependent transport systems inner membrane component
RPVRPRRLRR

SEQ ID NO: 248 - protein, 10 aa, Mycoplasma pulmonis
KPAKPKKTFK

SEQ ID NO: 249 - protein, 10 aa, Aspergillus clavatus
  Aspergillus clavatus DUF726 domain protein
KPMKPKLSNK

SEQ ID NO: 250 - protein, 10 aa, Coprinopsis cinerea
  Inky cap fungus (strain Okayama-7 / 130 / FGSC 9003) Putative protein
RPQKPKSKSR

SEQ ID NO: 251 - protein, 10 aa, Cebus apella
  Brown-capped capuchin Major prion protein
KPSKPKTSMK

SEQ ID NO: 252 - protein, 10 aa, Mus musculus
  Mouse Major prion protein
KPSKPKTNLK

SEQ ID NO: 253 - protein, 10 aa, Ateles paniscus
  Black spider monkey Major prion protein
KPSKPKTNMK

SEQ ID NO: 254 - protein, 10 aa, Cricetulus migratorius
  Armenian hamster Major prion protein
KPNKPKTSMK

SEQ ID NO: 255 - protein, 10 aa, Nematostella vectensis
  Starlet sea anemone Putative protein
KPIRPRYEVK

SEQ ID NO: 256 - protein, 10 aa, Clostridium phytofermentans
  Clostridium phytofermentans (strain ATCC 700394 / DSM 18823 / ISDg) RNA binding S1 domain protein
RPYRPKRRTR

SEQ ID NO: 257 - protein, 10 aa, Methanococcus maripaludis
  Methanococcus maripaludis (strain C5 / ATCC BAA-1333) RNA binding S1 domain protein
RPFRPKRKTR

SEQ ID NO: 258 - protein, 10 aa, Clostridium beijerinckii
  Clostridium beijerinckii (strain ATCC 51743 / NCIMB 8052) RNA binding S1 domain protein
RPFKPKKRTK

SEQ ID NO: 259 - protein, 10 aa, Clostridium thermocellum
  Clostridium thermocellum (strain ATCC 27405 / DSM 1237) RNA binding S1
RPFRPKRRTR

SEQ ID NO: 260 - protein, 10 aa, Haemophilus ducreyi
  Haemophilus ducreyi Protein tonB
KPEKPKEKLK

SEQ ID NO: 261 - protein, 10 aa, Clostridium kluyveri
  Clostridium kluyveri (strain ATCC 8527 / DSM 555 / NCIMB 10680) Tex
RPYKPKKRTR

SEQ ID NO: 262 - protein, 10 aa, Clostridium novyi
  Clostridium novyi (strain NT) S1 RNA binding domain protein
RPFKPKKRTR

SEQ ID NO: 263 - protein, 10 aa, Ovis aries
  Sheep PRNP
KPSKPKTNXK

SEQ ID NO: 264 - protein, 10 aa, Ovis aries
  Sheep Major prion protein
KPSKPKTNTK

SEQ ID NO: 265 - protein, 10 aa, Tragelaphus spekii
  Sitatunga Major prion protein
KPNKPKTNMK

SEQ ID NO: 266 - protein, 10 aa, Streptococcus sanguinis
  Streptococcus sanguinis (strain SK36) Transcriptional attenuator LytR
KPKKPRKKIR

SEQ ID NO: 267 - protein, 10 aa, Trichosurus vulpecula
  Brushtailed possum Major prion protein
KPDKPKTNLK

SEQ ID NO: 268 - protein, 10 aa, Shewanella sediminis
  Shewanella sediminis (strain HAW-EB3) Sensor histidine kinase
RPPRPRPDNR

SEQ ID NO: 269 - protein, 10 aa, Anaeromyxobacter dehalogenans
  Anaeromyxobacter dehalogenans 2CP-1 NADH dehydrogenase (Quinone)
RPLRPRARLR

SEQ ID NO: 270 - protein, 10 aa, Neosartorya fischeri
  Neosartorya fischeri (strain ATCC 1020 / DSM 3700 / NRRL 181) Nucleic acid-binding protein
RPQKPKKRLR

SEQ ID NO: 271 - protein, 10 aa, Mus musculus
  Mouse Myelin-associated oligodendrocyte basic protein
RPAKPRSPSR

SEQ ID NO: 272 - protein, 10 aa, Kobus ellipsiprymnus
  Waterbuck Major prion protein
KPXKPKTNMK

SEQ ID NO: 273 - protein, 10 aa, Porphyromonas gingivalis
  Porphyromonas gingivalis 30S ribosomal protein S9
KPGRPKARKR

SEQ ID NO: 274 - protein, 10 aa, Acidovorax
  Acidovorax (strain JS42) Integral membrane sensor signal transduction histidine kinase
RPPRPRPDER

SEQ ID NO: 275 - protein, 10 aa, Pasteurella multocida
  Pasteurella multocida Protein tonB
KPKKPKKEQR

SEQ ID NO: 276 - protein, 10 aa, Burkholderia pseudomallei
  Burkholderia pseudomallei (strain 668) Methyl-accepting chemotaxis protein
KPGKPRRARR

SEQ ID NO: 277 - protein, 10 aa, Bos taurus
  Bovine G-protein coupled bile acid receptor 1
RPLRPRGSMR

SEQ ID NO: 278 - protein, 10 aa, Planctomyces maris
  Planctomyces maris DSM 8797 Ribosomal protein S1-like RNAbinding domain
RPYRPKRTSR

SEQ ID NO: 279 - protein, 10 aa, Arabidopsis thaliana
  Mouseear cress Calcium-dependent protein kinase isoform AK1
KPKKPKHMKR

SEQ ID NO: 280 - protein, 10 aa, Comamonas testosteroni
  Comamonas testosteroni KF-1 Class I peptide chain release factor
RPPRPRKPTK

SEQ ID NO: 281 - protein, 10 aa, Drosophila melanogaster
  Fruit fly protein CG18190-PA
KPIKPRQFAK

SEQ ID NO: 282 - protein, 10 aa, Pseudomonas mendocina
  Pseudomonas mendocina (strain ymp) Response regulator receiver protein
RPARPRRDPR

SEQ ID NO: 283 - protein, 10 aa, Laccaria bicolor
  Bicoloured deceiver (strain S238NH82) Putative protein
RPLRPRPTFR

SEQ ID NO: 284 - protein, 10 aa, Saccharopolyspora erythraea
  Saccharopolyspora erythraea (strain NRRL 23338) Transposase
RPGRPRRKPR

SEQ ID NO: 285 - protein, 10 aa, Culex quinquefasciatus
  Southern house mosquito Male-specific transcription factor FRU-MB
RPDRPRGDSR

SEQ ID NO: 286 - protein, 10 aa, Bos taurus
  Bovine ZNF689 protein
RPSKPRLIPR

SEQ ID NO: 287 - protein, 10 aa, Methylobacterium
  Methylobacterium (strain 4-46) TonB family protein
KPPKPRPPER

SEQ ID NO: 288 - protein, 10 aa, Rattus norvegicus
  Rat Zinc finger protein 689
KPSKPRLIAR

SEQ ID NO: 289 - protein, 10 aa, Cryptococcus neoformans
  Cryptococcus neoformans Histone-lysine N-methyltransferase, H3 lysine-79 specific
KPKRPKVTRK

SEQ ID NO: 290 - protein, 10 aa, Phaeobacter gallaeciensis
  Phaeobacter gallaeciensis 2.1 Glycosyltransferase involved in cell wall biogenesis-like protein
RPMRPRLTDR

SEQ ID NO: 291 - protein, 10 aa, Mus musculus
  Mouse Zinc finger protein 689
KPSKPRLIPR

SEQ ID NO: 292 - protein, 10 aa, Lodderomyces elongisporus
  Yeast Putative protein
KPEKPKLPQR

SEQ ID NO: 293 - protein, 10 aa, Mus musculus
  Mouse Testis-specific H1 histone
RPLRPRSRRK

SEQ ID NO: 294 - protein, 10 aa, Mycobacterium ulcerans
  Mycobacterium ulcerans (strain Agy99) Conserved hypothetical membrane protein
KPVRPRRLHR

SEQ ID NO: 295 - protein, 10 aa, Monosiga brevicollis
  Monosiga brevicollis Putative protein
RPKKPRKPRK

SEQ ID NO: 296 - protein, 10 aa, Hoeflea phototrophica
  Hoeflea phototrophica DFL-43 Hypothetical cytosolic protein
RPVKPKDWRR

SEQ ID NO: 297 - protein, 10 aa, Laccaria bicolor
  Bicoloured deceiver (strain S238NH82) Putative protein
RPQRPRTTSK

SEQ ID NO: 298 - protein, 10 aa, Mus musculus
  Mouse Chorein
KPDKPKEAKK

SEQ ID NO: 299 - protein, 10 aa, Mycobacterium bovis
  Mycobacterium bovis Probable oxidoreductase ephD
RPWRPRTFLR

SEQ ID NO: 300 - protein, 10 aa, Mus musculus
  Mouse Coiled-coil domain-containing protein 96
RPEKPKGKGK

SEQ ID NO: 301 - protein, 10 aa, Acidovorax avenae
  Acidovorax avenae (strain AAC00-1) Phage integrase family protein
KPAKPKGRER

SEQ ID NO: 302 - protein, 10 aa, Anaeromyxobacter dehalogenans
  Anaeromyxobacter dehalogenans 2CP-1 TfoX domain protein
RPSKPRPARK

SEQ ID NO: 303 - protein, 10 aa, Anaeromyxobacter
  Anaeromyxobacter (strain Fw109-5) LigA
RPGRPRPRRR

SEQ ID NO: 304 - protein, 10 aa, Caenorhabditis elegans
  C. elegans Uncharacterized protein T19H5.4
KPVRPKRDFR

SEQ ID NO: 305 - protein, 10 aa, Ochrobactrum anthropi
  Ochrobactrum anthropi (strain ATCC 49188 / DSM 6882 / NCTC 12168) Dyp-type peroxidase family
KPLKPKLLQR

SEQ ID NO: 306 - protein, 10 aa, Rhizobium leguminosarum
  Rhizobium leguminosarum (WSM1325) NUDIX hydrolase
RPHRPRVKIK

SEQ ID NO: 307 - protein, 10 aa, Brugia malayi
  Filarial nematode worm Bop1-prov protein
RPSRPKQIEK

SEQ ID NO: 308 - protein, 10 aa, Bos taurus
  Bovine Cdc42 effector protein 1
RPEKPRDRDR

SEQ ID NO: 309 - protein, 10 aa, Shewanella benthica
  Shewanella benthica KT99 Transposase
RPYKPKDKAK

SEQ ID NO: 310 - protein, 10 aa, Suberites domuncula
  Sponge Wiskott-Aldrich syndrome protein
KPKRPKGKGK

SEQ ID NO: 311 - protein, 10 aa, Vitis vinifera
  Grape Chromosome chr9 scaffold_7
KPKKPRAEKR

SEQ ID NO: 312 - protein, 10 aa, Victivallis vadensis
  Victivallis vadensis ATCC BAA-548 Ferrous iron transport protein B
RPVKPKYGPK

SEQ ID NO: 313 - protein, 10 aa, Vibrio cholerae
  Vibrio cholerae RlpA-like lipoprotein
KPTKPRPKSK

SEQ ID NO: 314 - protein, 10 aa, Rhodopseudomonas palustris
  Rhodopseudomonas palustris (strain BisB18) UPF0317 protein RPC_1666
RPLKPKDAIR

SEQ ID NO: 315 - protein, 10 aa, Streptococcus pneumoniae
  Streptococcus pneumoniae SP3-BS71 BCR
KPVKPKKEKK

SEQ ID NO: 316 - protein, 10 aa, Rhodopseudomonas palustris
  Rhodopseudomonas palustris (strain HaA2) UPF0317 protein RPB_3621
RPMKPKDAIR

SEQ ID NO: 317 - protein, 10 aa, Pseudomonas syringae
  Pseudomonas syringae Insertion sequence
KPGRPRKRSR

SEQ ID NO: 318 - protein, 10 aa, Aspergillus niger
  Aspergillus niger (strain CBS 513.88 / FGSC A1513) Contig An13c0060
KPEKPKTKIR

SEQ ID NO: 319 - protein, 10 aa, Erythrobacter
  Erythrobacter SD-21 Methyltransferase
RPLRPRRRLR

SEQ ID NO: 320 - protein, 10 aa, Comamonas testosteroni
  Comamonas testosteroni KF-1 Sporulation related
KPEKPKAEVK

SEQ ID NO: 321 - protein, 10 aa, Azotobacter vinelandii
  Azotobacter vinelandii USG-1 protein homolog
RPPKPRFFDR

SEQ ID NO: 322 - protein, 10 aa, Pseudomonas syringae
  Pseudomonas syringae Translation initiation factor IF-2
RPDKPRADDR

SEQ ID NO: 323 - protein, 10 aa, Homo sapiens
  Human Zinc fingers and homeoboxes protein 2
KPGKPKADAK

SEQ ID NO: 324 - protein, 10 aa, Rhodobacter sphaeroides
  Rhodobacter sphaeroides (strain ATCC 17025 / ATH 2.4.3) Plasmid pRiA4b ORF-3 family protein
RPRKPKVTSR

SEQ ID NO: 325 - protein, 10 aa, Rhodobacter sphaeroides
  Rhodobacter sphaeroides (strain ATCC 17029 / ATH 2.4.9) Peptidyl-tRNA hydrolase
KPEKPKPAAK

SEQ ID NO: 326 - protein, 10 aa, Rhodobacter sphaeroides
  Rhodobacter sphaeroides (strain ATCC 17025 / ATH 2.4.3) Peptidyl-tRNA hydrolase
KPEKPKGEAK

SEQ ID NO: 327 - protein, 10 aa, Mus musculus
  Mouse Probable protein phosphatase 1Blike
RPHRPRERER

SEQ ID NO: 328 - protein, 10 aa, Homo sapiens
  Human Probable protein phosphatase 1Blike
RPLRPRERER

SEQ ID NO: 329 - protein, 10 aa, Coprinopsis cinerea
  Inky cap fungus (strain Okayama-7 / 130 / FGSC 9003)  Putative protein
KPKRPRATGR

SEQ ID NO: 330 - protein, 10 aa, Leptospira biflexa
  Leptospira biflexa (strain Patoc 1 / Ames) DNA methylase
RPIKPRIFQK

SEQ ID NO: 331 - protein, 10 aa, Synechocystis
  Synechocystis (strain PCC 6803) UPF0026 protein slr1464
RPTRPKPLQR

SEQ ID NO: 332 - protein, 10 aa, Danio rerio
  Zebrafish P2X purinoceptor
KPNKPKKLLR

SEQ ID NO: 333 - protein, 10 aa, Candida glabrata
  Yeast Mediator of RNA polymerase II transcription subunit 3
KPRKPRQTKK

SEQ ID NO: 334 - protein, 10 aa, Pyrobaculum calidifontis
  Pyrobaculum calidifontis (strain JCM 11548 / VA1) Radical SAM domain protein
KPYRPRGWEK

SEQ ID NO: 335 - protein, 10 aa, Lactobacillus brevis
  Lactobacillus brevis (strain ATCC 367 / JCM 1170) 50S ribosomal protein L3
RPPRPKQSEK

SEQ ID NO: 336 - protein, 10 aa, Laccaria bicolor
  Bicoloured deceiver (strain S238N-H82) Hypothetical magnesium transporter, CorA-like protein
RPRRPRGRSR

SEQ ID NO: 337 - protein, 10 aa, Limnobacter
  Limnobacter MED105 Conserved transposase-like protein
RPVRPRPRGR

SEQ ID NO: 338 - protein, 10 aa, Pseudoalteromonas tunicata
  Pseudoalteromonas tunicata D2 30S ribosomal subunit protein S3
KPAKPRAPKK

SEQ ID NO: 339 - protein, 10 aa, Bradyrhizobium
  Bradyrhizobium (strain BTAi1 / ATCC BAA-1182) Transposase
RPRKPRDKAK

SEQ ID NO: 340 - protein, 10 aa, Roseobacter
  Roseobacter AzwK-3b Transposase, IS256 family protein
RPARPKTGGR

SEQ ID NO: 341 - protein, 10 aa, Desulfococcus oleovorans
  Desulfococcus oleovorans (strain DSM 6200 / Hxd3) Phosphotransferase KptA/Tpt1
KPGRPKGRKK

SEQ ID NO: 342 - protein, 10 aa, Paramecium tetraurelia
  Paramecium tetraurelia Chromosome undetermined scaffold_68
KPDKPKQSQK

SEQ ID NO: 343 - protein, 10 aa, Desulfatibacillum alkenivorans
  Desulfatibacillum alkenivorans AK-01 Polyribonucleotide nucleotidyltransferase
KPLKPKSFSR

SEQ ID NO: 344 - protein, 10 aa, Giardia lamblia
  Giardia lamblia (ATCC 50803) Spindle pole protein
KPKKPRKLKK

SEQ ID NO: 345 - protein, 10 aa, Halorhodospira halophila
  Halorhodospira halophila (strain DSM 244 / SL1) DNA-directed DNA polymerase
RPVRPRRSRK

SEQ ID NO: 346 - protein, 10 aa, Fervidobacterium nodosum
  Fervidobacterium nodosum  (strain ATCC 35602 / DSM 5306 / Rt17-B1) Carbohydrate kinase, YjeF related protein
KPLRPRWAHK

SEQ ID NO: 347 - protein, 10 aa, Verminephrobacter eiseniae
  Verminephrobacter eiseniae (strain EF01-2) Integrase
RPARPKDKPK

SEQ ID NO: 348 - protein, 10 aa, Acidovorax
  Acidovorax (strain JS42) Integrase
RPAKPRDKPK

SEQ ID NO: 349 - protein, 10 aa, Rhizobium meliloti
  Rhizobium meliloti Transposase TRm23a
RPRRPRDKAK

SEQ ID NO: 350 - protein, 10 aa, Serratia entomophila
  Serratia entomophila RepA
KPMRPRLPRR

SEQ ID NO: 351 - protein, 10 aa, Monosiga brevicollis
  Monosiga brevicollis Putative protein
RPERPRPTLR

SEQ ID NO: 352 - protein, 10 aa, Rhodobacter sphaeroides
  Rhodobacter sphaeroides (strain ATCC 17025 / ATH 2.4.3) Integrase, catalytic region
RPYKPRDKAK

SEQ ID NO: 353 - protein, 10 aa, Escherichia coli
  E. coli O1K1 / APEC protein TnpA
RPRKPKDKAK

SEQ ID NO: 354 - protein, 10 aa, Acidiphilium cryptum
  Acidiphilium cryptum (strain JF-5) Integrase, catalytic region
RPFRPRDKAK

SEQ ID NO: 355 - protein, 10 aa, Marinobacter aquaeolei
  Marinobacter aquaeolei (strain ATCC 700491 / DSM 11845 / VT8) Integrase
RPYKPKDKSK

SEQ ID NO: 356 - protein, 10 aa, Shewanella putrefaciens
  Shewanella putrefaciens 200 Integrase
RPRKPKDKSK

SEQ ID NO: 357 - protein, 10 aa, Roseovarius
  Roseovarius (TM1035) Pseudouridine synthase
KPARPKPQRR

SEQ ID NO: 358 - protein, 10 aa, Thermoplasma volcanium
  Thermoplasma volcanium CCA-adding enzyme
KPIKPKYHDR

SEQ ID NO: 359 - protein, 10 aa, Leptospirillum
  Leptospirillum (Group II UBA) Cytochrome c
RPPKPRKHPR

SEQ ID NO: 360 - protein, 10 aa, Nematostella vectensis
  Starlet sea anemone Putative protein
KPTKPKTCIK

SEQ ID NO: 361 - protein, 10 aa, Carnobacterium
  Carnobacterium AT7 Integral membrane protein
RPAKPKRTIK

SEQ ID NO: 362 - protein, 10 aa, Saccharopolyspora erythraea
  Saccharopolyspora erythraea (strain NRRL 23338) Ribose transport ATP-binding protein
RPRRPRVGRR

SEQ ID NO: 363 - protein, 10 aa, Caenorhabditis elegans
  C. elegans Uncharacterized protein ZK177.1
KPTKPRASRK

SEQ ID NO: 364 - protein, 10 aa, Coprinopsis cinerea
  Inky cap fungus (strain Okayama-7 / 130 / FGSC 9003) Putative protein
KPPRPRPRPR

SEQ ID NO: 365 - protein, 10 aa, Danio rerio
  Zebrafish Zgc162349 protein
KPQKPKVKPK

SEQ ID NO: 366 - protein, 10 aa, Ajellomyces capsulata
  Darling′s disease fungus (strain NAm1 / WU24) Putative protein
RPGRPKNLGK

SEQ ID NO: 367 - protein, 10 aa, Aspergillus niger
  Aspergillus niger (strain CBS 513.88 / FGSC A1513) Similarity to hypothetical protein At2g17590
RPSRPKGTPR

SEQ ID NO: 368 - protein, 10 aa, Roseobacter litoralis
  Roseobacter litoralis Och 149 Penicillin-insensitive murein endopeptidase
KPRKPKPPIR

SEQ ID NO: 369 - protein, 10 aa, Methylobacterium
  Methylobacterium (strain 4-46) von Willebrand factor type A
RPRRPRRMRR

SEQ ID NO: 370 - protein, 10 aa, Oryza sativa
  Rice Glucose-6-phosphate 1- dehydrogenase
KPAKPKWFKR

SEQ ID NO: 371 - protein, 10 aa, Chloroflexus aurantiacus
  Chloroflexus aurantiacus (strain ATCC 29366 / DSM 635/J-10-fl) ATPase associated with various cellular activities AAA_5
RPDRPRGGYR

SEQ ID NO: 372 - protein, 10 aa, Chloroflexus aggregans
  Chloroflexus aggregans DSM 9485 ATPase associated with various cellular activities, AAA_5
RPDRPRGSYR

SEQ ID NO: 373 - protein, 10 aa, Leptospira biflexa
  Leptospira biflexa (strain Patoc 1 / Ames) Zn-dependent peptidase
KPGKPRPSYK

SEQ ID NO: 374 - protein, 10 aa, Halorhodospira halophila
  Halorhodospira halophila (strain DSM 244 / SL1) DNA translocase FtsK
KPRRPRKAER

SEQ ID NO: 375 - protein, 10 aa, Lactobacillus helveticus
  Lactobacillus helveticus (strain DPC 4571) Transcription repressor of betagalactosidase gene
RPDRPKIDIR

SEQ ID NO: 376 - protein, 10 aa, Coprinopsis cinerea
  Inky cap fungus (strain Okayama-7 / 130 / FGSC 9003) Putative protein
RPARPKCHKR

SEQ ID NO: 377 - protein, 10 aa, Brugia malayi
  Filarial nematode worm N terminus of Rad21 / Rec8 like protein
KPGRPKRKRR

SEQ ID NO: 378 - protein, 10 aa, Lodderomyces elongisporus
  Yeast Putative protein
KPRKPRATKK

SEQ ID NO: 379 - protein, 10 aa, Rhodobacter capsulatus
  Rhodobacter capsulatus Nitrogenase iron-molybdenum cofactor biosynthesis protein nifE
KPFRPRVAGR

SEQ ID NO: 380 - protein, 10 aa, Vitis vinifera
  Grape Chromosome chr4 scaffold_6
KPVRPKDELR

SEQ ID NO: 381 - protein, 10 aa, Cucumis sativus
  Cucumber Monogalactosyldiacylglycerol synthase, chloroplastic
KPIRPKIELR

SEQ ID NO: 382 - protein, 10 aa, Flavobacterium psychrophilum
  Flavobacterium psychrophilum (strain JIP02/86 / ATCC 49511) Putative  ABC-type multidrug transport system, ATPase and permease components
RPEKPKSIGK

SEQ ID NO: 383 - protein, 10 aa, Chlorokybus atmophyticus
  Soil alga Pyruvate dehydrogenase E1 component subunit beta
KPPKPRTAKK

SEQ ID NO: 384 - protein, 10 aa, Agrobacterium tumefaciens
  Agrobacterium tumefaciens protein Orf_Bo191
RPWRPRFDAR

SEQ ID NO: 385 - protein, 10 aa, Arabidopsis thaliana
  Mouse-ear cress  Monogalactosyldiacylglycerol synthase 1, chloroplastic
KPVRPKVELR

SEQ ID NO: 386 - protein, 10 aa, Desulfatibacillum alkenivorans
  Desulfatibacillum alkenivorans (AK-01) PSP1 domain protein
KPRRPRNKKR

SEQ ID NO: 387 - protein, 10 aa, Hyperthermus butylicus
  Hyperthermus butylicus (strain DSM 5456 / JCM 9403) Phenylalanyl-tRNA synthetase beta chain
KPEKPKLMLR

SEQ ID NO: 388 - protein, 10 aa, Opitutaceae bacterium
  Opitutaceae bacterium (TAV2) Thiamine pyrophosphate protein TPP binding domain protein
RPPRPRHLVR

SEQ ID NO: 389 - protein, 10 aa, Neosartorya fischeri
  Neosartorya fischeri (strain ATCC 1020 / DSM 3700 / NRRL 181) WD domain protein
KPPRPKEKKR

SEQ ID NO: 390 - protein, 10 aa, Mus musculus
  Mouse Transient receptor potential cation channel subfamily V member 6
RPLKPRITNR

SEQ ID NO: 391 - protein, 10 aa, Homo sapiens
  Human Transient receptor potential cation channel, subfamily V, member 6 (TRPV6)
RPLKPRTNNR

SEQ ID NO: 392 - protein, 10 aa, Homo sapiens
  Human Zinc finger protein 335
RPGRPRKLPR

SEQ ID NO: 393 - protein, 10 aa, Xanthobacter autotrophicus
  Xanthobacter autotrophicus (strain ATCC BAA-1158 / Py2) Phage integrase family protein
KPWRPRFDAK

SEQ ID NO: 394 - protein, 10 aa, Saccharopolyspora erythraea
  Saccharopolyspora erythraea (strain NRRL 23338) Endo-1, 4-beta-glucanase
KPAKPKPLPK

SEQ ID NO: 395 - protein, 10 aa, Bradyrhizobium
  Bradyrhizobium (strain BTAi1 / ATCC BAA-1182) Transposase
KPRRPKTGAR

SEQ ID NO: 396 - protein, 10 aa, Cyanothece
  Cyanothece (CCY 0110) Translation initiation factor IF-2
RPPKPKSQTK

SEQ ID NO: 397 - protein, 10 aa, Streptococcus pneumoniae
  Streptococcus pneumoniae SP3-BS71 Ribosomal protein S1
RPRRPRRQEK

SEQ ID NO: 398 - protein, 10 aa, Streptococcus gordonii
  Streptococcus gordonii (strain Challis / ATCC 35105 / CH1 / DL1 / V288) 30S ribosomal protein S1
RPRRPKRQEK

SEQ ID NO: 399 - protein, 10 aa, Acidovorax avenae
  Acidovorax avenae (strain AAC00-1) LigA
RPCRPRDPGR

SEQ ID NO: 400 - protein, 10 aa, Homo sapiens
  Human Potassium channel subfamily K member  4
KPVRPRGPGR

SEQ ID NO: 401 - protein, 10 aa, Salinispora arenicola
  Salinispora arenicola (strain CNS-205) Peptidase M50
RPRRPRSVDR

SEQ ID NO: 402 - protein, 10 aa, Physcomitrella patens
  Physcomitrella patens  Putative protein
RPPRPRHVQR

SEQ ID NO: 403 - protein, 10 aa, Mus musculus
  Mouse Potassium channel subfamily K member 4
KPSRPRGPGR

SEQ ID NO: 404 - protein, 10 aa, Ajellomyces capsulata
  Darling′s disease fungus (strain NAm1 / WU24) Putative protein
RPSRPKGMPR

SEQ ID NO: 405 - protein, 10 aa, Arthrobacter
  Arthrobacter (strain FB24) Sodium/hydrogen exchanger
RPAKPRPAAR

SEQ ID NO: 406 - protein, 10 aa, Paramecium tetraurelia
  Paramecium tetraurelia Chromosome undetermined scaffold_52
RPSKPRSHPK

SEQ ID NO: 407 - protein, 10 aa, Equine herpesvirus
  Equine herpesvirus 1 (strain Ab4p) (EHV-1) Trans-acting transcriptional protein ICP0
RPMRPRCSLR

SEQ ID NO: 408 - protein, 10 aa, Vibrio cholerae
  Vibrio cholerae V52 ATP-dependent RNA helicase RhlE
RPLKPKKPKK

SEQ ID NO: 409 - protein, 10 aa, Vibrio splendidus
  Vibrio splendidus 12B01 ATP-dependent RNA helicase RhlE
RPIKPKKPKK

SEQ ID NO: 410 - protein, 10 aa, Reinekea
  Reinekea MED297 ATP-dependent RNA helicase, DEAD box family protein
KPKKPKKPKK

SEQ ID NO: 411 - protein, 10 aa, Salinispora tropica
  Salinispora tropica (strain ATCC BAA-916 / DSM 44818 / CNB-440) Peptidase M50
RPRRPRPLDR

SEQ ID NO: 412 - protein, 10 aa, Ajellomyces capsulata
  Darling′s disease fungus  Putative protein
RPIRPKKGVR

SEQ ID NO: 413 - protein, 10 aa, Leishmania infantum
  Leishmania infantum Endo/exonuclease Mre11
RPVKPKPERR

SEQ ID NO: 414 - protein, 10 aa, Leishmania braziliensis
  Leishmania braziliensis Endo/exonuclease Mre11
RPVKPKLERR

SEQ ID NO: 415 - protein, 10 aa, Mus musculus
  Mouse FCH domain only protein 1
KPQRPRSAPR

SEQ ID NO: 416 - protein, 10 aa, Anaeromyxobacter dehalogenans
  Anaeromyxobacter dehalogenans 2CP-1 Tryptophan synthase, beta subunit
RPARPKGKAK

SEQ ID NO: 417 - protein, 10 aa, Culex quinquefasciatus
  Southern house mosquito Peter pan
KPKKPRLDKR

SEQ ID NO: 418 - protein, 10 aa, Aspergillus clavatus
  Aspergillus clavatus DnaJ domain protein
RPSKPRGVER

SEQ ID NO: 419 - protein, 10 aa, Aspergillus niger
  Aspergillus niger (strain CBS 513.88 / FGSC A1513) Contig An07c0220
RPRKPRYFNR

SEQ ID NO: 420 - protein, 10 aa, Oceanibulbus indolifex
  Oceanibulbus indolifex HEL-45 DNA primase
RPQKPRADFR

SEQ ID NO: 421 - protein, 10 aa, Frankia
  Frankia (strain EAN1pec) YibE/F family protein
RPRRPRPPAR

SEQ ID NO: 422 - protein, 10 aa, Leishmania infantum
  Leishmania infantum Histone h1-like protein
KPTKPRTTTK

SEQ ID NO: 423 - protein, 10 aa, Mycobacterium tuberculosis
  Mycobacterium tuberculosis (strain F11) PE-PGRS family protein
RPGRPRWAGR

SEQ ID NO: 424 - protein, 10 aa, Synechococcus
  Synechococcus (RS9917) Translation initiation factor
RPAKPRSQQK

SEQ ID NO: 425 - protein, 10 aa, Synechococcus
  Synechococcus (strain CC9605) Translation initiation factor IF-2
RPAKPKSQQR

SEQ ID NO: 426 - protein, 10 aa, Rhizobium leguminosarum
  Rhizobium leguminosarum (WSM1325) AAA ATPase central domain protein
RPRRPKDRIR

SEQ ID NO: 427 - protein, 10 aa, Prochlorococcus marinus
  Prochlorococcus marinus (strain MIT 9211) Translation initiation factor IF-2
RPAKPKSTKK

SEQ ID NO: 428 - protein, 10 aa, Monosiga brevicollis
  Monosiga brevicollis  Putative protein
KPPKPRGIKR

SEQ ID NO: 429 - protein, 10 aa, Prochlorococcus marinus
  Prochlorococcus marinus (strain MIT 9303) Translation initiation factor IF-2
RPAKPKSQKK

SEQ ID NO: 430 - protein, 10 aa, Synechococcus
  Synechococcus (strain WH7803) Translation initiation factor IF-2
RPSKPKSQQK

SEQ ID NO: 431 - protein, 10 aa, Plesiocystis pacifica
  Plesiocystis pacifica SIR-1 Nuclease SbcCD, C subunit
RPRRPRRSSR

SEQ ID NO: 432 - protein, 10 aa, Synechococcus
  Synechococcus (strain CC9311) Translation initiation factor IF-2
RPSKPRTKHK

SEQ ID NO: 433 - protein, 10 aa, Saccharomyces cerevisiae
  Baker′s yeast (strain YJM789) Conserved protein
KPYRPKVRRR

SEQ ID NO: 434 - protein, 10 aa, Heliobacterium modesticaldum
  Heliobacterium modesticaldum (strain ATCC 51547 / Ice1) Transcription-repair coupling factor
RPRKPRKSAR

SEQ ID NO: 435 - protein, 10 aa, Prochlorococcus marinus
  Prochlorococcus marinus Translation initiation factor IF-2
RPGKPKASKK

SEQ ID NO: 436 - protein, 10 aa, Rhodobacterales bacterium
  Rhodobacterales bacterium (HTCC2150) Exodeoxyribonuclease 7 large subunit
KPKKPKPTDK

SEQ ID NO: 437 - protein, 10 aa, Plesiocystis pacifica
  Plesiocystis pacifica SIR-1 Heavy metal efflux pump
KPKKPRPKAR

SEQ ID NO: 438 - protein, 10 aa, Synechococcus
  Synechococcus (strain WH7805) Translation initiation factor IF-2
RPAKPRAQQK

SEQ ID NO: 439 - protein, 10 aa, Acidothermus cellulolyticus
  Acidothermus cellulolyticus (strain ATCC 43068 / 11B) Uroporphyrinogen-III synthase /uroporphyrinogen-III Cmethyltransferase
KPGRPRVGHR

SEQ ID NO: 440 - protein, 10 aa, Clavibacter michiganensis
  Clavibacter michiganensis (strain NCPPB 382) Conjugal transfer protein
KPKKPKKPKR

SEQ ID NO: 441 - protein, 10 aa, Sagittula stellata
  Sagittula stellata (E-37) ABC transporter
KPEKPKEKPR

SEQ ID NO: 442 - protein, 10 aa, Nematostella vectensis
  Starlet sea anemone Putative protein
RPNRPKKKNK

SEQ ID NO: 443 - protein, 10 aa, Coprinopsis cinerea
  Inky cap fungus Putative protein
RPHRPRSNTR

SEQ ID NO: 444 - protein, 10 aa, Rattus norvegicus
  Rat Regulating synaptic membrane exocytosis protein 1
KPHRPKRGGK

SEQ ID NO: 445 - protein, 10 aa, Brugia malayi
  Filarial nematode worm LF3 protein
RPGRPRIPRR

SEQ ID NO: 446 - protein, 10 aa, Synechococcus
  Synechococcus (strain CC9902) Translation initiation factor IF-2
RPAKPKAQQR

SEQ ID NO: 447 - protein, 10 aa, Prochlorococcus marinus
  Prochlorococcus marinus (strain NATL1A) Translation initiation factor IF-2
RPSKPKVGKR

SEQ ID NO: 448 - protein, 10 aa, Ostreococcus lucimarinus
  Ostreococcus lucimarinus (strain CCE9901) Putative protein
RPKRPRECTK

SEQ ID NO: 449 - protein, 10 aa, Laccaria bicolor
  Bicoloured deceiver (strain S238NH82) Putative protein
RPLRPKYLYR

SEQ ID NO: 450 - protein, 10 aa, Drosophila melanogaster
  Fruit fly protein CG34411-PA
RPLKPRDTEK

SEQ ID NO: 451 - protein, 10 aa, Aspergillus fumigatus
  Aspergillus fumigatus Conserved proline-rich protein
KPAKPKSKSK

SEQ ID NO: 452 - protein, 10 aa, Ostreococcus lucimarinus
  Ostreococcus lucimarinus (strain CCE9901) Putative protein
RPDKPKKQEK

SEQ ID NO: 453 - protein, 10 aa, Bacillus selenitireducens
  Bacillus selenitireducens (MLS10) ABC transporter related
RPAKPKGKYK

SEQ ID NO: 454 - protein, 10 aa, Laccaria bicolor
  Bicoloured deceiver (strain S238NH82) Putative protein
KPTRPKTKGK

SEQ ID NO: 455 - protein, 10 aa, Homo sapiens
  Human Uncharacterized protein C19orf29
RPRKPRFFNR

SEQ ID NO: 456 - protein, 10 aa, Chlamydomonas reinhardtii
  Chlamydomonas reinhardtii  Putative protein
RPPRPKAMDK

SEQ ID NO: 457 - protein, 10 aa, Giardia lamblia
  Giardia lamblia (ATCC 50803) TFIIH basal transcription factor complex helicase subunit
KPAKPKSACR

SEQ ID NO: 458 - protein, 10 aa, Neosartorya fischeri
  Neosartorya fischeri (strain ATCC 1020 / DSM 3700 / NRRL 181) Conserved proline-rich protein
KPEKPKSKNK

SEQ ID NO: 459 - protein, 10 aa, Homo sapiens
  Human Probable E3 ubiquitin-protein ligase MYCBP2
KPYKPKKIIK

SEQ ID NO: 460 - protein, 10 aa, Paramecium tetraurelia
  Paramecium tetraurelia Chromosome undetermined scaffold_21
KPIKPKFKER

SEQ ID NO: 461 - protein, 10 aa, Burkholderia vietnamiensis
  Burkholderia vietnamiensis (strain G4 / LMG 22486) Methionine synthase (B12- dependent)
RPVKPKFIGR

SEQ ID NO: 462 - protein, 10 aa, Danio rerio
  Zebrafish Novel protein similar to vertebrate asparagine-linked glycoslyation 9 homolog (ALG9) (Zgc63820)
KPRRPKHTRK

SEQ ID NO: 463 - protein, 10 aa, Salinispora arenicola
  Salinispora arenicola (strain CNS-205) Thioester reductase domain
KPLRPKLKTR

SEQ ID NO: 464 - protein, 10 aa, Homo sapiens
  Human SET domain-containing protein 5
KPSRPRPKSR

SEQ ID NO: 465 - protein, 10 aa, Bacillus subtilis
  Bacillus subtilis MutS2 protein
KPEKPKTQKR

SEQ ID NO: 466 - protein, 10 aa, Laccaria bicolor
  Bicoloured deceiver (strain S238NH82) Putative protein
RPHRPKIRSR

SEQ ID NO: 467 - protein, 10 aa, Rhodobacterales bacterium
  Rhodobacterales bacterium HTCC2654 Lipoprotein
RPVRPRRVGK

SEQ ID NO: 468 - protein, 10 aa, Homo sapiens
  Human Uncharacterized protein KIAA0586
RPQRPKVIER

SEQ ID NO: 469 - protein, 10 aa, Aspergillus niger
  Aspergillus niger (strain CBS 513.88 / FGSC A1513) Contig An02c0010
RPRRPRREDK

SEQ ID NO: 470 - protein, 10 aa, Caenorhabditis briggsae
  Caenorhabditis briggsae DNA topoisomerase
KPRKPRAPRK

SEQ ID NO: 471 - protein, 10 aa, Danio rerio
  Zebra Protein KRI1 homolog
KPKRPRKKMR

SEQ ID NO: 472 - protein, 10 aa, Geobacter uraniireducens
  Geobacter uraniireducens (strain Rf4) Ribonuclease E
KPKKPRAPRK

SEQ ID NO: 473 - protein, 10 aa, Coprinopsis cinerea
  Inky cap fungus (strain Okayama-7 / 130 / FGSC 9003)  Putative protein
KPQRPRKRLR

SEQ ID NO: 474 - protein, 10 aa, Shewanella pealeana
  Shewanella pealeana (strain ATCC 700345 / ANG-SQ1) Ribonuclease, Rne/Rng family
KPARPKRQPR

SEQ ID NO: 475 - protein, 10 aa, Geobacter bemidjiensis
  Geobacter bemidjiensis (strain Bem / ATCC BAA-1014 / DSM 16622) Ribonuclease, Rne/Rng family
KPKRPRGGRK

SEQ ID NO: 476 - protein, 10 aa, Solanum lycopersicum
  Tomato Beta-galactosidase
RPLRPKAHLK

SEQ ID NO: 477 - protein, 10 aa, Homo sapiens
  Human Protein Shroom2
KPERPRTAGR

SEQ ID NO: 478 - protein, 10 aa, Sinorhizobium medicae
  Sinorhizobium medicae (strain WSM419) Ribonuclease, Rne/Rng family
KPAKPRRSRK

SEQ ID NO: 479 - protein, 10 aa, Sclerotinia sclerotiorum
  White mold  Putative protein
RPSRPRKHRK

SEQ ID NO: 480 - protein, 10 aa, Marinomonas
  Marinomonas (strain MWYL1) Ribonuclease, Rne/Rng family
KPAKPKMSVK

SEQ ID NO: 481 - protein, 10 aa, Marinobacter algicola
  Marinobacter algicola (DG893) Ribonucleases G and E
RPARPRRSSR

SEQ ID NO: 482 - protein, 10 aa, Monosiga brevicollis
  Monosiga brevicollis  Putative protein
KPRKPRPPSK

SEQ ID NO: 483 - protein, 10 aa, Theileria parva
  Theileria parva 104 kDa microneme/rhoptry antigen
RPSKPKKPKK

SEQ ID NO: 484 - protein, 10 aa, Coprinopsis cinerea
  Inky cap fungus (strain Okayama-7 / 130 / FGSC 9003) Putative protein
RPSRPKPIGK

SEQ ID NO: 485 - protein, 10 aa, Rhodobacter sphaeroides
  Rhodobacter sphaeroides (strain ATCC 17025 / ATH 2.4.3) Diguanylate phosphodiesterase
RPGRPRPPDR

SEQ ID NO: 486 - protein, 10 aa, Laccaria bicolor
  Bicoloured deceiver (strain S238NH82) Putative protein
RPGRPRRERR

SEQ ID NO: 487 - protein, 10 aa, Aspergillus clavatus
  Aspergillus clavatus DNA repair helicase rad5, 16
KPEKPKPKNK

SEQ ID NO: 488 - protein, 10 aa, Physcomitrella patens
  Physcomitrella patens  Putative protein
RPRKPKTIAK

SEQ ID NO: 489 - protein, 10 aa, Danio rerio
  Zebrafish Bone morphogenetic protein receptor type II b (Serine/threoninekinase)
RPPKPRRPER

SEQ ID NO: 490 - protein, 10 aa, Paramecium tetraurelia
  Paramecium tetraurelia Chromosome undetermined scaffold_35
RPFKPKANKK

SEQ ID NO: 491 - protein, 10 aa, Paramecium tetraurelia
  Paramecium tetraurelia Chromosome undetermined scaffold_17
RPTRPKVIKK

SEQ ID NO: 492 - protein, 10 aa, Homo sapiens
  Human Coiled-coil and C2 domain containing protein 2A
RPLRPRRKGR

SEQ ID NO: 493 - protein, 10 aa, Helicoverpa armigera granulovirus
  Helicoverpa armigera granulovirus DNA polymerase
KPTKPKTKKR

SEQ ID NO: 494 - protein, 10 aa, Sphingomonas wittichii
  Sphingomonas wittichii (strain RW1 / DSM 6014 / JCM 10273) LVIVD repeat protein
RPLKPKLYEK

SEQ ID NO: 495 - protein, 10 aa, Culex quinquefasciatus
  Southern house mosquito Chromodomain helicase DNA binding protein
KPRKPREPKK

SEQ ID NO: 496 - protein, 10 aa, Monosiga brevicollis
  Monosiga brevicollis  Putative protein
KPLKPRLVGR

SEQ ID NO: 497 - protein, 10 aa, Homo sapiens
  Human Spectrin beta chain, brain 3
RPDRPRARDR

SEQ ID NO: 498 - protein, 10 aa, Coprinopsis cinerea
  Inky cap fungus (strain Okayama-7 / 130 / FGSC 9003)  Putative protein
RPRRPRGNAR

SEQ ID NO: 499 - protein, 10 aa, Berne virus
  BEV Replicase polyprotein 1ab
RPKKPKGNFK

SEQ ID NO: 500 - protein, 11 aa, Magnetococcus
  Magnetococcus (strain MC-1) 50S ribosomal protein L17
RHKKRGRRLSR

SEQ ID NO: 501 - protein, 11 aa, Salinibacter ruber
  Salinibacter ruber (strain DSM 13855) 50S ribosomal protein L17
RHRKKGKKIGR

SEQ ID NO: 502 - protein, 11 aa, Paramecium tetraurelia
  Paramecium tetraurelia Chromosome undetermined scaffold_13
KDKKKEKKKDK

SEQ ID NO: 503 - protein, 11 aa, Human T-cell leukemia virus 1
  HTLV-1 (strain Japan ATK-1 subtype A) Protein Rex
KTRRRPRRSQR

SEQ ID NO: 504 - protein, 11 aa, Oceanobacillus iheyensis
  Oceanobacillus iheyensis DNA translocase ftsK
KKRKKKRKNSK

SEQ ID NO: 505 - protein, 11 aa, Homo sapiens
  Human Putative  E3 ubiquitin-protein ligase HERC5
RSRRKSRRNGR

SEQ ID NO: 506 - protein, 11 aa, Mus musculus
  Mouse Uncharacterized protein C14orf45 homolog
KDKRKDKRKDK

SEQ ID NO: 507 - protein, 11 aa, Streptococcus mutans
  Streptococcus mutans DNA translocase ftsK
KNKRKGRKTRR

SEQ ID NO: 508 - protein, 11 aa, Pediococcus pentosaceus
  Pediococcus pentosaceus (strain ATCC 25745 / 183-1w) 50S ribosomal protein L18
KNKKRQRRHAR

SEQ ID NO: 509 - protein, 11 aa, Pseudotsuga menziesii
  Douglas-fir Unknown protein 5
KARRRIKRWRR

SEQ ID NO: 510 - protein, 11 aa, Pinus koraiensis
  Korean pine 50S ribosomal protein L32
RSKKKIRKNVR

SEQ ID NO: 511 - protein, 11 aa, Pyrococcus abyssi
  Pyrococcus abyssi 50S ribosomal protein L21e
KTRKKLRKHPR

SEQ ID NO: 512 - protein, 11 aa, Methanopyrus kandleri
  Methanopyrus kandleri 50S ribosomal protein L21e
RTRKKLRKKPR

SEQ ID NO: 513 - protein, 11 aa, Arabidopsis thaliana
  Mouse-ear cress F-box protein At4g19940
RERRRTKRRRR

SEQ ID NO: 514 - protein, 11 aa, Pelotomaculum thermopropionicum
  Pelotomaculum thermopropionicum (strain DSM 13744 / JCM 10971 / SI) 50S ribosomal protein L18
KRRRRVRKKIR

SEQ ID NO: 515 - protein, 11 aa, Listeria welshimeri
  Listeria welshimeri (strain ATCC 35897 / DSM 20650 / SLCC5334)  Putative protein
KWKRKRKKILR

SEQ ID NO: 516 - protein, 11 aa, Murex brandaris
  Purple dye murex Sperm protamine P3
KGKKRRRRGRK

SEQ ID NO: 517 - protein, 11 aa, Orcinus orca
  Killer whale Sperm protamine-P1
RPRRRCRRRIR

SEQ ID NO: 518 - protein, 11 aa, Danio rerio
  Zebrafish UPF0384 protein CGI-117 homolog
KNKKKLKKKLR

SEQ ID NO: 519 - protein, 11 aa, Paramecium tetraurelia
  Paramecium tetraurelia Chromosome undetermined scaffold_13
KEKKRKKKRLR

SEQ ID NO: 520 - protein, 11 aa, Homo sapiens
  Human Tetratricopeptide repeat protein 26
KRKKKGRKIPK

SEQ ID NO: 521 - protein, 11 aa, Kluyveromyces lactis
  Yeast ATP-dependent RNA helicase DBP3
KSKKKLKKDKK

SEQ ID NO: 522 - protein, 11 aa, Eledone cirrhosa
  Curled octopus Cysteine-rich protamine
RSKKRCRRCRR

SEQ ID NO: 523 - protein, 11 aa, Oryza sativa
  Rice Zinc finger CCCH domain-containing protein 16
KERRRERKKER

SEQ ID NO: 524 - protein, 11 aa, Paramecium tetraurelia
  Paramecium tetraurelia Chromosome undetermined scaffold_15
KDKKKIKKKSK

SEQ ID NO: 525 - protein, 11 aa, Scyliorhinus canicula
  Spotted dogfish Spermatid-specific protein S1
RCRRKGRRISR

SEQ ID NO: 526 - protein, 11 aa, Octopus vulgaris
  Octopus Sperm protamine P1
RGRRRGRRRGR

SEQ ID NO: 527 - protein, 11 aa, Homo sapiens
  Human Transmembrane protein TTMA
KRKRKAKKRRK

SEQ ID NO: 528 - protein, 11 aa, Gnetum parvifolium
  Gnetum parvifolium 30S ribosomal protein S4
KYKKRVRRSSR

SEQ ID NO: 529 - protein, 11 aa, Paramecium tetraurelia
  Paramecium tetraurelia Chromosome undetermined scaffold_24
KHKKKDKKQKK

SEQ ID NO: 530 - protein, 11 aa, Saccharomyces cerevisiae
  Baker′s yeast Protein INO4
KRKRRSKKINK

SEQ ID NO: 531 - protein, 11 aa, Simian immunodeficiency virus
  Simian immunodeficiency virus (isolate AGM3) Protein Rev
RQRRRARRRWR

SEQ ID NO: 532 - protein, 11 aa, Simian immunodeficiency virus
  Simian immunodeficiency virus (isolate AGM155) Protein Rev
RQRRRARRRWK

SEQ ID NO: 533 - protein, 11 aa, Lagorchestes hirsutus
  Western hare-wallaby Sperm protamine-P1
RYRRRQRRSRR

SEQ ID NO: 534 - protein, 11 aa, Sorangium cellulosum
  Sorangium cellulosum (strain So ce56) 50S ribosomal protein L35
KSKKRLRRLRK

SEQ ID NO: 535 - protein, 11 aa, Human adenovirus
  Human adenovirus F serotype 40 Minor core protein
RVKKREKKEEK

SEQ ID NO: 536 - protein, 11 aa, Neurospora crassa
  Neurospora crassa ATP-dependent RNA helicase dbp-3
KERKKDKKEKK

SEQ ID NO: 537 - protein, 11 aa, Saccharomyces cerevisiae
  Baker′s yeast Myb domain-containing protein YDR026C
KTKKKLKKQKK

SEQ ID NO: 538 - protein, 11 aa, Paramecium tetraurelia
  Paramecium tetraurelia Chromosome undetermined scaffold_106
KPRKKTRKIVK

SEQ ID NO: 539 - protein, 11 aa, Mus musculus
  Mouse 39S ribosomal protein L15
RDRRRGRKCGR

SEQ ID NO: 540 - protein, 11 aa, Ashbya gossypii
  Yeast ATP-dependent RNA helicase DRS1
KSRKKTKKGRK

SEQ ID NO: 541 - protein, 11 aa, Homo sapiens
  Human 39S ribosomal protein L15
RGRRRGRKCGR

SEQ ID NO: 542 - protein, 11 aa, Pichia stipitis
  Yeast Histone H2A.Z-specific chaperone CHZ1
KDKKKHKKRRR

SEQ ID NO: 543 - protein, 11 aa, Drosophila melanogaster
  Fruit fly Polycomb protein esc
RSKRRGRRSTK

SEQ ID NO: 544 - protein, 11 aa, Drosophila virilis
  Fruit fly Polycomb protein esc
RSKRRGRRSHK

SEQ ID NO: 545 - protein, 11 aa, Aquifex aeolicus
  Aquifex aeolicus Uncharacterized protein aq_1791
RERRRFKRELR

SEQ ID NO: 546 - protein, 11 aa, Archaeoglobus fulgidus
  Archaeoglobus fulgidus Uncharacterized protein AF_2193
KKRRRIRRSQK

SEQ ID NO: 547 - protein, 11 aa, Prochlorococcus marinus
  Prochlorococcus marinus 50S ribosomal protein L20
RDRKRRKRDFR

SEQ ID NO: 548 - protein, 11 aa, Oltmannsiellopsis viridis
  Oltmannsiellopsis viridis 50S ribosomal protein L20
RDRRKMKREFR

SEQ ID NO: 549 - protein, 11 aa, Campylobacter jejuni
  Campylobacter jejuni strain 81116 / NCTC 11828) 50S ribosomal protein L20
RDRRRKKRDFR

SEQ ID NO: 550 - protein, 11 aa, Acaryochloris marina
  Acaryochloris marina (strain MBIC 11017) 50S ribosomal protein L20
RDRRKRKRDFR

SEQ ID NO: 551 - protein, 11 aa, Torque teno virus
  Torque teno virus (isolate Human/Finland/Hel32/2002) Capsid protein
RWRRRYRRWRR

SEQ ID NO: 552 - protein, 11 aa, Chloroflexus aurantiacus
  Chloroflexus aurantiacus (strain ATCC 29366 / DSM 635 / J-10-fl) 50S ribosomal protein L20
RDRRRRKRDFR

SEQ ID NO: 553 - protein, 11 aa, Bradyrhizobium japonicum
  Bradyrhizobium japonicum 50S ribosomal protein L20
RDRKRKKRTFR

SEQ ID NO: 554 - protein, 11 aa, Homo sapiens
  Human Homeobox protein HMX1
RPRRRGRRAGR

SEQ ID NO: 555 - protein, 11 aa, Xenopus laevis
  African clawed frog Arginine/serine-rich coiled-coil protein 2
KHRRKEKRSSR

SEQ ID NO: 556 - protein, 11 aa, Dictyostelium discoideum
  Slime mold Exocyst complex component 6
KEKKKEKKELK

SEQ ID NO: 557 - protein, 11 aa, Homo sapiens
  Human Zinc finger CCCH domain-containing protein 6
REKKKSKRRKR

SEQ ID NO: 558 - protein, 11 aa, Schizosaccharomyces pombe
  Fission yeast tRNA (guanine-N(1)-)- methyltransferase
REKKRLRKEER

SEQ ID NO: 559 - protein, 11 aa, Saccharomyces cerevisiae
  Baker′s yeast Uncharacterized protein YLR137W
RKKKRVRRRNK

SEQ ID NO: 560 - protein, 11 aa, Sclerotinia sclerotiorum
  White mold ATP-dependent RNA helicase dbp3
RERKRLKKAKR

SEQ ID NO: 561 - protein, 11 aa, Danio rerio
  Zebrafish Putative ATP-dependent RNA helicase DDX46
RERKRQRRSSR

SEQ ID NO: 562 - protein, 11 aa, Neurospora crassa
  Neurospora crassa Glycylpeptide Ntetradecanoyltransferase
KNKKKSKKKNK

SEQ ID NO: 563 - protein, 11 aa, Gibberella zeae
  Gibberella zeae ATP-dependent RNA helicase DBP3
KERKKAKKAKK

SEQ ID NO: 564 - protein, 11 aa, Botryotinia fuckeliana
  Noble rot fungus (strain B05.10) ATP-dependent RNA helicase dbp3
RERKRLKKEKK

SEQ ID NO: 565 - protein, 11 aa, Gallus gallus
  Chicken Bromodomain-containing protein 7
RKRKKRKKGEK

SEQ ID NO: 566 - protein, 11 aa, Bos taurus
  Bovine RNA (guanine-9-)- methyltransferase domain-containing protein 2
KQKRKEKRKRK

SEQ ID NO: 567 - protein, 11 aa, Mus musculus
  Mouse RNA (guanine-9-)- methyltransferase domain-containing protein 2
KEKRKEKRKRK

SEQ ID NO: 568 - protein, 11 aa, Debaryomyces hansenii
  Yeast Transcription elongation factor SPT6
RKRRKHKKRKR

SEQ ID NO: 569 - protein, 11 aa, Rattus norvegicus
  Rat 39S ribosomal protein L40
RLKKRIRKLER

SEQ ID NO: 570 - protein, 11 aa, Homo sapiens
  Human 39S ribosomal protein L40
RLKRKIRKLEK

SEQ ID NO: 571 - protein, 11 aa, Mus musculus
  Mouse 39S ribosomal protein L40
RLKKRIRKLEK

SEQ ID NO: 572 - protein, 11 aa, Yarrowia lipolytica
  Yarrowia lipolytica Transcription elongation factor SPT5
RQRKKPRRERR

SEQ ID NO: 573 - protein, 11 aa, Candida albicans
  Yeast Stress response protein NST1
KKRKKHKKKSK

SEQ ID NO: 574 - protein, 11 aa, Xenopus tropicalis
  Western clawed frog RNA (guanine-9-)- methyltransferase domain-containing protein 2
KQKRKEKRQKR

SEQ ID NO: 575 - protein, 11 aa, Cryptococcus neoformans
  Cryptococcus neoformans Transcription elongation factor SPT6
RRRKKERRMRR

SEQ ID NO: 576 - protein, 11 aa, Ashbya gossypii
  Yeast Transcription elongation factor SPT6
RRRRKHKRRPR

SEQ ID NO: 577 - protein, 11 aa, Candida glabrata
  Yeast Transcription elongation factor SPT6
KKKRRHKRRAR

SEQ ID NO: 578 - protein, 11 aa, Neurospora crassa
  Neurospora crassa Transcription elongation factor spt-6
RERRRRKKRRR

SEQ ID NO: 579 - protein, 11 aa, Saccharomyces cerevisiae
  Baker′s yeast Transcription elongation factor SPT6
KKRRKHKRRER

SEQ ID NO: 580 - protein, 11 aa, Emericella nidulans
  Emericella nidulans Transcription elongation factor spt6
RERRKRRREER

SEQ ID NO: 581 - protein, 11 aa, Saccharomyces cerevisiae
  Baker′s yeast rRNA-processing protein EBP2
KEKRKLKKELK

SEQ ID NO: 582 - protein, 11 aa, Alouatta seniculus
  Red howler monkey Protamine-2
RYRRRNRRGCR

SEQ ID NO: 583 - protein, 11 aa, Mus musculus
  Mouse High mobility group protein B4
KRRKRRKRDPK

SEQ ID NO: 584 - protein, 11 aa, Aspergillus fumigatus
  Aspergillus fumigatus Transcription elongation factor spt6
REKRKRRREER

SEQ ID NO: 585 - protein, 11 aa, Hylobates lar
  Gibbon Protamine-2
RQRRRHRRGCR

SEQ ID NO: 586 - protein, 11 aa, Pan troglodytes
  Chimpanzee Protamine-2
RHRRKHRRGCR

SEQ ID NO: 587 - protein, 11 aa, Gorilla gorilla
  Lowland gorilla Protamine-2
RHRRRHRKGCR

SEQ ID NO: 588 - protein, 11 aa, Bos taurus
  Bovine Prokineticin-2
RRKRKRRRKKK

SEQ ID NO: 589 - protein, 11 aa, Erythrocebus patas
  Red guenon Protamine-2
RHRRRHRRGCR

SEQ ID NO: 590 - protein, 11 aa, Callithrix jacchus
  Marmoset Protamine-2
RYRRRPRRGCR

SEQ ID NO: 591 - protein, 11 aa, Danio rerio
  Zebrafish ADP-ribosylation factor-like protein 6-interacting protein 4
REKKKEKKEKK

SEQ ID NO: 592 - protein, 11 aa, Schizosaccharomyces pombe
  Fission yeast Signal recognition particle subunit srp14
RDRKKTKKNKK

SEQ ID NO: 593 - protein, 11 aa, Mus musculus
  Mouse Uncharacterized protein C1orf115 homolog
KPKKRYRRKLK

SEQ ID NO: 594 - protein, 11 aa, Schizosaccharomyces pombe
  Fission yeast Meiotically up-regulated gene 116 protein
KTKKKRKKEKK

SEQ ID NO: 595 - protein, 11 aa, Adelaide River virus
  ARV Protein alpha-1
RKRRKKKRKGK

SEQ ID NO: 596 - protein, 11 aa, Methanocaldococcus jannaschii
  Methanocaldococcus jannaschii 50S ribosomal protein L1P
KNKRKLRKIAK

SEQ ID NO: 597 - protein, 11 aa, Paramecium tetraurelia
  Paramecium tetraurelia Chromosome undetermined scaffold_171
RRKKRERKARK

SEQ ID NO: 598 - protein, 11 aa, Homo sapiens
  Human Uncharacterized protein C1orf115
RYRRKLKKYGK

SEQ ID NO: 599 - protein, 11 aa, Homo sapiens
  Human Protein FAM133A
RKKKRKKKSCR

SEQ ID NO: 600 - protein, 11 aa, Danio rerio
  Zebrafish Arginine/serine-rich coiled-coil protein 2
RHRRKDKKTSR

SEQ ID NO: 601 - protein, 11 aa, Paramecium tetraurelia
  Paramecium tetraurelia Chromosome undetermined scaffold_169
KEKRRIKKIIR

SEQ ID NO: 602 - protein, 11 aa, Mus musculus
  Mouse Proline-rich protein 13
KTRKKMKKAHK

SEQ ID NO: 603 - protein, 11 aa, Bos taurus
  Bovine U1 small nuclear ribonucleoprotein A
RDRKREKRKPK

SEQ ID NO: 604 - protein, 11 aa, Xenopus tropicalis
  Western clawed frog Arginine/serine-rich coiled-coil protein 2
RHRRKEKRSSR

SEQ ID NO: 605 - protein, 11 aa, Methanocaldococcus jannaschii
  Methanocaldococcus jannaschii Putative RNA-binding protein MJ0652 MJ0652
KPKKKVKKDEK

SEQ ID NO: 606 - protein, 11 aa, Paramecium tetraurelia
  Paramecium tetraurelia Chromosome undetermined scaffold_178
KSKRKGKRSSR

SEQ ID NO: 607 - protein, 11 aa, Danio rerio
  Zebrafish Transcription elongation factor SPT6
KVKRRKKKYSR

SEQ ID NO: 608 - protein, 11 aa, Saccharomyces cerevisiae
  Baker′s yeast Pre-mRNA-splicing factor 8
KSRKRAKKMTK

SEQ ID NO: 609 - protein, 11 aa, Bos taurus
  Bovine Arginine/serine-rich coiled-coil protein 2
RHKRKERKSSR

SEQ ID NO: 610 - protein, 11 aa, Drosophila melanogaster
  Fruit fly CWF19-like protein 2 homolog
KERKRRKKKSK

SEQ ID NO: 611 - protein, 11 aa, Schizosaccharomyces pombe
  Fission yeast Uncharacterized protein C22F3.11c
KLRRKQKKVNK

SEQ ID NO: 612 - protein, 11 aa, Homo sapiens
  Human Zinc finger CCCH domain-containing protein 4
KEKRRSKKRRK

SEQ ID NO: 613 - protein, 11 aa, Paramecium tetraurelia
  Paramecium tetraurelia Chromosome undetermined scaffold_12
RHKRKVKRHRR

SEQ ID NO: 614 - protein, 11 aa, Streptococcus pneumoniae
  Streptococcus pneumoniae Transposase for insertion sequence IS1202
KTKKRVRKQAK

SEQ ID NO: 615 - protein, 11 aa, Dictyostelium discoideum
  Slime mold Uncharacterized protein DDB_0237901
RRKKRRRRKHR

SEQ ID NO: 616 - protein, 11 aa, Cervus albirostris
  White-lipped deer Sex-determining region Y protein
RPRRKRKRQQK

SEQ ID NO: 617 - protein, 11 aa, Bison bison
  American bison Sex-determining region Y protein
RPRRRAKRPQK

SEQ ID NO: 618 - protein, 11 aa, Balaenoptera acutorostrata
  Minke whale Sex-determining region Y protein
RPRRKAKRPQK

SEQ ID NO: 619 - protein, 11 aa, Kogia simus
  Dwarf sperm whale Sex-determining region Y protein
RPRRKPKRPQK

SEQ ID NO: 620 - protein, 11 aa, Bubalus bubalis
  Domestic water buffalo Sex-determining region Y protein
RPRRKAKRLQK

SEQ ID NO: 621 - protein, 11 aa, Balaenoptera physalus
  Finback whale Sex-determining region Y protein
RPRRKAKRSQK

SEQ ID NO: 622 - protein, 11 aa, Alces alces
  Siberian moose Sex-determining region Y protein
RPRRKTKRQQK

SEQ ID NO: 623 - protein, 11 aa, Mullus surmuletus
  Striped red mullet Protamine-like protein
KKRKRRRRKSK

SEQ ID NO: 624 - protein, 11 aa, Xenopus tropicalis
  Western clawed frog Protein SFRS12IP1
KDKRKEKRERK

SEQ ID NO: 625 - protein, 11 aa, Paramecium tetraurelia
  Paramecium tetraurelia Chromosome undetermined scaffold_122
RQRRKGKRMLR

SEQ ID NO: 626 - protein, 11 aa, Paramecium tetraurelia
  Paramecium tetraurelia Chromosome undetermined scaffold_114
KQRRKGKRMLR

SEQ ID NO: 627 - protein, 11 aa, Mus musculus
  Mouse Protein SFRS12IP1
KEKKKRKKEKR

SEQ ID NO: 628 - protein, 11 aa, Sus scrofa
  Pig Antibacterial peptide PMAP-36
RLRKKTRKRLK

SEQ ID NO: 629 - protein, 11 aa, Bos taurus
  Bovine Cathelicidin-6
RFRKKFKKLFK

SEQ ID NO: 630 - protein, 11 aa, Paramecium tetraurelia
  Paramecium tetraurelia Chromosome undetermined scaffold_106
RNKRKLKRLPR

SEQ ID NO: 631 - protein, 11 aa, Caenorhabditis elegans
  C. elegans Transcriptional regulator ATRX homolog
KSKKKSKKTKK

SEQ ID NO: 632 - protein, 11 aa, Oryza sativa
  Rice DEAD-box ATP-dependent RNA helicase 13
KKKRRSRKKRK

SEQ ID NO: 633 - protein, 11 aa, Homo sapiens
  Human Membrane protein FAM174A
RMRRRNRKTRR

SEQ ID NO: 634 - protein, 11 aa, Mus musculus
  Mouse Zinc finger protein 329
KIKKRSKRFYK

SEQ ID NO: 635 - protein, 11 aa, Homo sapiens
  Human Coiled-coil domain-containing protein 140
RKRRKERKKER

SEQ ID NO: 636 - protein, 11 aa, Mus musculus
  Mouse Surfeit locus protein 6
RERKKRKRKER

SEQ ID NO: 637 - protein, 11 aa, Homo sapiens
  Human Vacuolar protein sorting-associated protein 13C
RKRKKHKKHFK

SEQ ID NO: 638 - protein, 11 aa, Homo sapiens
  Human Transcription initiation protein SPT3 homolog
KDKKKLRRLLK

SEQ ID NO: 639 - protein, 11 aa, Homo sapiens
  Human Beta-galactoside alpha-2
RVKKRHRRQRR

SEQ ID NO: 640 - protein, 11 aa, Bos taurus
  Bovine Wiskott-Aldrich syndrome protein family member 2
KEKRKHRKEKK

SEQ ID NO: 641 - protein, 11 aa, Ajellomyces capsulata
  Darling′s disease fungus  (strain NAm1 / WU24) Protein PXR1
KARKKEKRRAR

SEQ ID NO: 642 - protein, 11 aa, Dictyostelium discoideum
  Slime mold Protein SCAR
RQRKRERREAR

SEQ ID NO: 643 - protein, 11 aa, Paramecium tetraurelia
  Paramecium tetraurelia Chromosome undetermined scaffold_18
KDKKKLKRYTK

SEQ ID NO: 644 - protein, 11 aa, Paramecium tetraurelia
  Paramecium tetraurelia Chromosome undetermined scaffold_110
KDKRREKRLLK

SEQ ID NO: 645 - protein, 11 aa, Pyrococcus horikoshii
  Pyrococcus horikoshii Transcription factor E
KSKKRSKKSKK

SEQ ID NO: 646 - protein, 11 aa, Bos taurus
  Bovine Uncharacterized protein C12orf43 homolog
KKKRKLKKKAK

SEQ ID NO: 647 - protein, 10 aa, Saccharomyces cerevisiae
  Baker′s yeast Protein PXR1
KDKKKDKKDK

SEQ ID NO: 648 - protein, 11 aa, Saccharomyces cerevisiae
  Baker′s yeast (strain YJM789) Protein PXR1
KDKKKDKKDKK

SEQ ID NO: 649 - protein, 11 aa, Methanopyrus kandleri
  Methanopyrus kandleri 50S ribosomal protein L4P
KGKRRGRRYVK

SEQ ID NO: 650 - protein, 11 aa, Rattus norvegicus
  Rat Cell growth-regulating nucleolar protein
KSRKKEKKELK

SEQ ID NO: 651 - protein, 11 aa, Mus musculus
  Mouse Cell growth-regulating nucleolar protein
KNRKKEKKELK

SEQ ID NO: 652 - protein, 11 aa, Homo sapiens
  Human Cell growth-regulating nucleolar protein
KKRKREKKELK

SEQ ID NO: 653 - protein, 11 aa, Aspergillus clavatus
  Aspergillus clavatus Protein pxr1
KERKREKRQMR

SEQ ID NO: 654 - protein, 11 aa, Paramecium tetraurelia
  Paramecium tetraurelia Chromosome undetermined scaffold_175
KTKKKYKKQMK

SEQ ID NO: 655 - protein, 11 aa, Dictyostelium discoideum
  Slime mold Cell division control protein 45 homolog
KSKKKNKKDKK

SEQ ID NO: 656 - protein, 11 aa, Paramecium tetraurelia
  Paramecium tetraurelia Chromosome undetermined scaffold_108
KHKKKEKKSKK

SEQ ID NO: 657 - protein, 11 aa, Neurospora crassa
  Neurospora crassa Protein pxr-1
RSKKKEKKDKK

SEQ ID NO: 658 - protein, 11 aa, Aedes aegypti
  Yellowfever mosquito Mediator of RNA polymerase II transcription subunit 19
KERKKRKKEKK

SEQ ID NO: 659 - protein, 11 aa, Paramecium tetraurelia
  Paramecium tetraurelia Chromosome undetermined scaffold_105
KNKKKEKKSKK

SEQ ID NO: 660 - protein, 11 aa, Paramecium tetraurelia
  Paramecium tetraurelia Chromosome undetermined scaffold_121
KHKKKEKKSNK

SEQ ID NO: 661 - protein, 11 aa, Mus musculus
  Mouse Protein FAM133B
KKRRKHKKHSK

SEQ ID NO: 662 - protein, 11 aa, Mus musculus
  Mouse Lon protease homolog
KSRRKLKRGKK

SEQ ID NO: 663 - protein, 11 aa, Mus musculus
  Mouse Peptidyl-prolyl cis-trans isomerase G
KSRKRKKKHRK

SEQ ID NO: 664 - protein, 11 aa, Gallus gallus
  Chicken Protein FAM133
KKRKKHKKHSK

SEQ ID NO: 665 - protein, 11 aa, Homo sapiens
  Human Peptidyl-prolyl cis-trans isomerase G
KSKKRKKKHRK

SEQ ID NO: 666 - protein, 11 aa, Ashbya gossypii
  Yeast Translocation protein SEC62
KQKRRKRKLEK

SEQ ID NO: 667 - protein, 11 aa, Homo sapiens
  Human Lon protease homolog
KPRRKSKRGKK

SEQ ID NO: 668 - protein, 11 aa, Bos taurus
  Bovine Lon protease homolog
KLRKKPKRGKK

SEQ ID NO: 669 - protein, 11 aa, Arabidopsis thaliana
  Mouse-ear cress Zinc finger protein CONSTANS-LIKE 3
REKRKNRKFEK

SEQ ID NO: 670 - protein, 11 aa, Arabidopsis thaliana
  Mouse-ear cress Protein TIFY 4A
REKRKDRKFSK

SEQ ID NO: 671 - protein, 11 aa, Gallus gallus
  Chicken Monocarboxylate transporter 3
KAKKKPKKGKK

SEQ ID NO: 672 - protein, 11 aa, Syntrophobacter fumaroxidans
  Syntrophobacter fumaroxidans (strain DSM 10017 / MPOB) Translation initiation factor IF-2
KAKKRRRKKVR

SEQ ID NO: 673 - protein, 11 aa, Magnaporthe grisea
  Rice blast fungus Protein PXR1
KRRKKEKKERK

SEQ ID NO: 674 - protein, 11 aa, Arabidopsis thaliana
  Mouse-ear cress CAX-interacting protein 4
RVKRKSRKEKR

SEQ ID NO: 675 - protein, 11 aa, Chaetomium globosum
  Soil fungus Protein PXR1
KDKKRDKKEKK

SEQ ID NO: 676 - protein, 11 aa, Natranaerobius thermophilus
  Natranaerobius thermophilus (strain ATCC BAA-1301 / DSM 18059 / JW/NM-WN-LF) 50S ribosomal protein L2
KTRRKAKKSDK

SEQ ID NO: 677 - protein, 11 aa, Finegoldia magna
  Finegoldia magna (strain ATCC 29328) 50S ribosomal protein L2
KTRKKNKKSNK

SEQ ID NO: 678 - protein, 11 aa, Gramella forsetii
  Gramella forsetii (strain KT0803) Translation initiation factor IF-2
RRKKRRRRISK

SEQ ID NO: 679 - protein, 11 aa, Homo sapiens
  Human Protein Wnt-16
KTKRKMRRREK

SEQ ID NO: 680 - protein, 11 aa, Enterobacteria phage T7
  Enterobacteria phage T7 Protein kinase
RFKRRNRKARK

SEQ ID NO: 681 - protein, 11 aa, Idiomarina loihiensis
  Idiomarina loihiensis Translation initiation factor IF-2
KSRKRGKKRRR

SEQ ID NO: 682 - protein, 11 aa, Bos taurus
  Bovine Transmembrane protein 198
KEKRRKKRPPR

SEQ ID NO: 683 - protein, 11 aa, Botryotinia fuckeliana
  Noble rot fungus (strain B05.10) Protein pxr1
KARKKEKKEKK

SEQ ID NO: 684 - protein, 11 aa, Paramecium tetraurelia
  Paramecium tetraurelia Chromosome undetermined scaffold_145
KSRKRGKRNLK

SEQ ID NO: 685 - protein, 11 aa, Bacillus anthracis
  Bacillus anthracis Teichoic acids export ATP-binding protein tagH
RKRKKGKKTSR

SEQ ID NO: 686 - protein, 11 aa, Saccharomyces cerevisiae
  Baker′s yeast Chromosome transmission fidelity protein 18
KDKKRKKKRSK

SEQ ID NO: 687 - protein, 11 aa, Homo sapiens
  Human Ankyrin repeat domain-containing protein 18B
KKRKKRKKLKK

SEQ ID NO: 688 - protein, 11 aa, Caenorhabditis elegans
  C. elegans Protein let-756
KKRRREKKKRR

SEQ ID NO: 689 - protein, 11 aa, Homo sapiens
  Human Leucine-rich repeat-containing protein 47
RRKRRERKQRR

SEQ ID NO: 690 - protein, 11 aa, Mus musculus
  Mouse Leucine-rich repeat-containing protein 47
RKKRRERKQHR

SEQ ID NO: 691 - protein, 11 aa, Homo sapiens
  Human Protein FAM153A
KPKKKTRKPSK

SEQ ID NO: 692 - protein, 11 aa, Xylella fastidiosa
  Xylella fastidiosa (strain Temecula1 / ATCC 700964) Bis(5′-nucleosyl)-tetraphosphatase
KAKKRHKRSPK

SEQ ID NO: 693 - protein, 11 aa, Arabidopsis thaliana
  Mouse-ear cress Zinc finger protein CONSTANS-LIKE 2
REKKKTRKFDK

SEQ ID NO: 694 - protein, 11 aa, Dictyostelium discoideum
  Slime mold Uncharacterized mitochondrial protein Mp36
KYKRRTKKKVK

SEQ ID NO: 695 - protein, 11 aa, Dictyostelium discoideum
  Slime mold Oxysterol-binding protein 7
KEKKKEKKKAK

SEQ ID NO: 696 - protein, 11 aa, Arabidopsis thaliana
  Mouse-ear cress Zinc finger protein CONSTANS-LIKE 1
REKKKMRKFEK

SEQ ID NO: 697 - protein, 11 aa, Homo sapiens
  Human G patch domain-containing protein 8
KSKKRKKRKRK

SEQ ID NO: 698 - protein, 11 aa, Arabidopsis thaliana
  Mouse-ear cress Zinc finger protein CONSTANS-LIKE 13
KEKKKSRRYEK

SEQ ID NO: 699 - protein, 11 aa, Drosophila melanogaster
  Fruit fly Vanin-like protein 2
RAKRRMKRDLK

SEQ ID NO: 700 - protein, 11 aa, Pediococcus pentosaceus
  Pediococcus pentosaceus (strain ATCC 25745 / 183-1w) Translation initiation factor IF-2
KNKKRNRKAKR

SEQ ID NO: 701 - protein, 11 aa, Rattus norvegicus
  Rat CBF1-interacting corepressor
KEKRKKRKKTK

SEQ ID NO: 702 - protein, 11 aa, Fugu rubripes
  Japanese pufferfish Homeobox protein Hox-C8a
KGKRRKRKKRK

SEQ ID NO: 703 - protein, 11 aa, Saccharomyces cerevisiae
  Baker′s yeast Protein YRO2
KLKKKHKKDKK

SEQ ID NO: 704 - protein, 11 aa, Arabidopsis thaliana
  Mouse-ear cress Zinc finger protein CONSTANS
REKRKTRKFEK

SEQ ID NO: 705 - protein, 11 aa, Deinococcus radiodurans
  Deinococcus radiodurans Fatty acid/phospholipid synthesis protein plsX
RQRRRPRRQKR

SEQ ID NO: 706 - protein, 11 aa, Dictyostelium discoideum
  Slime mold Multiple RNA-binding domaincontaining protein 1
KEKKKDKKKNK

SEQ ID NO: 707 - protein, 11 aa, Arabidopsis thaliana
  Mouse-ear cress Probable xyloglucan endotransglucosylase/hydrolase protein 30
RERRRNRRQQR

SEQ ID NO: 708 - protein, 11 aa, Neurospora crassa
  Neurospora crassa Palmitoyltransferase ERF2
RFKRRAKRQNR

SEQ ID NO: 709 - protein, 11 aa, Xenopus laevis
  African clawed frog Bone morphogenetic protein 3
KNKKKLRKGSR

SEQ ID NO: 710 - protein, 11 aa, Erwinia chrysanthemi
  Erwinia chrysanthemi Extracellular phospholipase C
RCKKRCRRITR

SEQ ID NO: 711 - protein, 11 aa, Mus musculus
  Mouse Scaffold attachment factor B2
RHRRKRKRRRK

SEQ ID NO: 712 - protein, 11 aa, Arabidopsis thaliana
  Mouse-ear cress Zinc finger protein CONSTANS-LIKE 10
KEKKKARKFDK

SEQ ID NO: 713 - protein, 11 aa, Soybean chlorotic mottle virus
  Soybean chlorotic mottle virus Capsid protein
KPKKKFRKIKK

SEQ ID NO: 714 - protein, 11 aa, Homo sapiens
  Human RING and PHD-finger domain containing protein KIAA1542
RRKRKTRRRKK

SEQ ID NO: 715 - protein, 11 aa, Paramecium tetraurelia
  Paramecium tetraurelia Chromosome undetermined scaffold_22
KQKKRIKKLEK

SEQ ID NO: 716 - protein, 11 aa, Danio rerio
  Zebrafish  La-related protein 7
KAKRKRKKKLK

SEQ ID NO: 717 - protein, 11 aa, Homo sapiens
  Human Cylicin-1
KDKKKLKKDDK

SEQ ID NO: 718 - protein, 11 aa, Homo sapiens
  Human Glycine receptor subunit alpha-2
RLRRRQKRQNK

SEQ ID NO: 719 - protein, 11 aa, Salmonella typhi
  Salmonella typhi Undecaprenyl-phosphate alpha-Nacetylglucosaminyl 1-phosphate transferase
RVKRRLRRQRK

SEQ ID NO: 720 - protein, 11 aa, Yersinia pestis
  Yersinia pestis Undecaprenyl-phosphate alpha-Nacetylglucosaminyl 1-phosphate transferase
RFKRRMRRASK

SEQ ID NO: 721 - protein, 11 aa, Mus musculus
  Mouse La-related protein 7
KVKRKHKKKHK

SEQ ID NO: 722 - protein, 11 aa, Drosophila melanogaster
  Fruit fly Transcriptional regulator ATRX homolog
KQKKKGRKRIK

SEQ ID NO: 723 - protein, 11 aa, Bos taurus
  Bovine Early growth response protein 4
KARRKGRRGGK

SEQ ID NO: 724 - protein, 11 aa, Homo sapiens
  Human Splicing factor
KDKRKGRKRSR

SEQ ID NO: 725 - protein, 11 aa, Rattus norvegicus
  Rat La-related protein 7
KGKRKHKKKHK

SEQ ID NO: 726 - protein, 11 aa, Homo sapiens
  Human La-related protein 7
KTKRKHKKKHK

SEQ ID NO: 727 - protein, 11 aa, Arabidopsis thaliana
  Mouse-ear cress Zinc finger protein CONSTANS-LIKE 14
KEKKKTRRYDK

SEQ ID NO: 728 - protein, 11 aa, Schizosaccharomyces pombe
  Fission yeast Curved DNA-binding protein
KNKKKSKKPSK

SEQ ID NO: 729 - protein, 11 aa, Saimiriine herpesvirus
  Saimiriine herpesvirus 2 (strain 11) Thymidine kinase
RVKKRNRKEEK

SEQ ID NO: 730 - protein, 11 aa, Mus musculus
  Mouse Vitronectin
KSKRRSRKRYR

SEQ ID NO: 731 - protein, 11 aa, Homo sapiens
  Human UPF0632 protein B
RFRKKRRRSQR

SEQ ID NO: 732 - protein, 11 aa, Arabidopsis thaliana
  Mouse-ear cress Phosphatidylinositol-4-phosphate 5- kinase 9
RAKRKHKRLVK

SEQ ID NO: 733 - protein, 11 aa, Oryctolagus cuniculus
  Rabbit Vitronectin
KSRRRSRKRYR

SEQ ID NO: 734 - protein, 11 aa, Caenorhabditis elegans
  C. elegans Probable splicing factor
KDRKRDKKRSR

SEQ ID NO: 735 - protein, 11 aa, Xenopus tropicalis
  Western clawed frog La-related protein 7
KVKRKRKKKHK

SEQ ID NO: 736 - protein, 11 aa, Gallus gallus
  Chicken Gametogenetin-binding protein 2
RQKRKNRRKNK

SEQ ID NO: 737 - protein, 11 aa, Methanocaldococcus jannaschii
  Methanocaldococcus jannaschii Uncharacterized NOP5 family protein MJ0694
KAKKKEKKGKK

SEQ ID NO: 738 - protein, 11 aa, Mus musculus
  Mouse RNA exonuclease 1 homolog
KVRKKDKKKEK

SEQ ID NO: 739 - protein, 11 aa, Ajellomyces capsulata
  Darling′s disease fungus WD repeat-containing protein JIP5
KKKRKKRKKGK

SEQ ID NO: 740 - protein, 11 aa, Coccidioides immitis
  Coccidioides immitis WD repeat-containing protein JIP5
KGKRKKRKRGK

SEQ ID NO: 741 - protein, 11 aa, Xenopus laevis
  African clawed frog Midnolin-A
RLRRKARRDSR

SEQ ID NO: 742 - protein, 11 aa, Arabidopsis thaliana
  Mouse-ear cress Zinc finger protein CONSTANS-LIKE 15
KEKRKTRRYDK

SEQ ID NO: 743 - protein, 11 aa, Phaeosphaeria nodorum
  Phaeosphaeria nodorum WD repeat-containing protein JIP5
KQKRKKRRKGK

SEQ ID NO: 744 - protein, 11 aa, Arabidopsis thaliana
  Mouse-ear cress F-box protein At3g19890
RQRKKRKRKSK

SEQ ID NO: 745 - protein, 11 aa, Buchnera aphidicola
  Buchnera aphidicola Arginyl-tRNA synthetase
KIKKKNRKIKK

SEQ ID NO: 746 - protein, 11 aa, Homo sapiens
  Human U4/U6.U5 tri-snRNP-associated protein 1
KTKRRVKKIRK

SEQ ID NO: 747 - protein, 11 aa, Homo sapiens
  Human Midnolin
RLRRKARRDAR

SEQ ID NO: 748 - protein, 11 aa, Coccidioides immitis
  Coccidioides immitis Chromosome segregation in meiosis protein 3
RKRKRIKKSLK

SEQ ID NO: 749 - protein, 11 aa, Neurospora crassa
  Neurospora crassa Mitochondrial Rho GTPase 1
KPRKRRRRPGR

SEQ ID NO: 750 - protein, 11 aa, Aspergillus fumigatus
  Aspergillus fumigatus Mitochondrial Rho GTPase 1
RPRKKRKRPGR

SEQ ID NO: 751 - protein, 11 aa, Aspergillus oryzae
  Aspergillus oryzae Mitochondrial Rho GTPase 1
RPRKRRKRPGR

SEQ ID NO: 752 - protein, 11 aa, Helianthus tuberosus
  Jerusalem artichoke Calnexin homolog
RWKKRNRRLKK

SEQ ID NO: 753 - protein, 11 aa, Homo sapiens
  Human Bromodomain-containing protein 1
KVRKKAKKAKK

SEQ ID NO: 754 - protein, 11 aa, Bos taurus
  Bovine Calpain-1 catalytic subunit
KHRRRERRFGR

SEQ ID NO: 755 - protein, 11 aa, Rattus norvegicus
  Rat Ankyrin repeat and zinc finger domain-containing protein 1
KRRKKRKKKER

SEQ ID NO: 756 - protein, 11 aa, Koala retrovirus
  KoRV Gag polyprotein
RERRRDRRQEK

SEQ ID NO: 757 - protein, 11 aa, Bos taurus
  Bovine Ankyrin repeat and zinc finger domain-containing protein 1
RRRKRNKRERK

SEQ ID NO: 758 - protein, 11 aa, Homo sapiens
  Human Ankyrin repeat domain-containing protein 11
KVKKKRKKETK

SEQ ID NO: 759 - protein, 11 aa, Xenopus tropicalis
  Western clawed frog Zinc finger matrin-type protein 1
KDKRRKRKHHR

SEQ ID NO: 760 - protein, 11 aa, Paramecium tetraurelia
  Paramecium tetraurelia Chromosome undetermined scaffold_171
KNRKKNKKKIR

SEQ ID NO: 761 - protein, 11 aa, Aspergillus fumigatus
  Aspergillus fumigatus Centromere/microtubule-binding protein cbf5
KRKKKEKKEKK

SEQ ID NO: 762 - protein, 11 aa, Candida albicans
  Yeast Nucleolar protein NOP58
KDKKKDKKEKK

SEQ ID NO: 763 - protein, 11 aa, Homo sapiens
  Human H/ACA ribonucleoprotein complex subunit 4
KEKKKSKKDKK

SEQ ID NO: 764 - protein, 11 aa, Paramecium tetraurelia
  Paramecium tetraurelia Chromosome undetermined scaffold_10
RKKRKERKDKK

SEQ ID NO: 765 - protein, 11 aa, Drosophila melanogaster
  Fruit fly Pre-mRNA-splicing factor Slu7
KTKKKSKKREK

SEQ ID NO: 766 - protein, 11 aa, Lactococcus lactis
  Lactococcus lactis Uncharacterized RNA methyltransferase ywfF
KGKKRGRRPQK

SEQ ID NO: 767 - protein, 11 aa, Podospora anserina
  Podospora anserina Protein SQS1
RERKKERKLAR

SEQ ID NO: 768 - protein, 11 aa, Myxococcus xanthus
  Myxococcus xanthus Transcription-repair-coupling factor
RARRRPKRSKK

SEQ ID NO: 769 - protein, 11 aa, Candida glabrata
  Candida glabrata High-osmolarity-induced transcription protein 1
RKRKKLRKINK

SEQ ID NO: 770 - protein, 11 aa, Saccharomyces cerevisiae
  Baker′s yeast Uncharacterized membrane protein YFL034W
RNRRRSKRRKK

SEQ ID NO: 771 - protein, 11 aa, Caenorhabditis elegans
  C. elegans Pre-mRNA-splicing factor SLU7
RDKKREKRERK

SEQ ID NO: 772 - protein, 11 aa, Xenopus laevis
  African clawed frog UPF0622 protein
KTKKKRRRGPR

SEQ ID NO: 773 - protein, 11 aa, Homo sapiens
  Human UPF0622 protein
KHKKKRRRGPR

SEQ ID NO: 774 - protein, 11 aa, Homo sapiens
  Human Kinesin-like protein KIF21A
KLKRKEKRKKK

SEQ ID NO: 775 - protein, 11 aa, Homo sapiens
  Human Probable global transcription activator SNF2L2
KEKKKRRRRKK

SEQ ID NO: 776 - protein, 11 aa, Potato leafroll virus
  Potato leafroll virus (strain 1) Uncharacterized protein ORF1
KQKKRGRRGGK

SEQ ID NO: 777 - protein, 11 aa, Coccidioides immitis
  Coccidioides immitis ATP-dependent rRNA helicase SPB4
KEKRRERKRAK

SEQ ID NO: 778 - protein, 11 aa, Kluyveromyces lactis
  Yeast SWR1-complex protein 3
RVKRKYRRRSK

SEQ ID NO: 779 - protein, 11 aa, Candida glabrata
  Yeast KNR4/SMI1 homolog
KNKKKNKKKNK

SEQ ID NO: 780 - protein, 11 aa, Saccharomyces cerevisiae
  Baker′s yeast Signal recognition particle subunit SRP72
KKKRKIKRLEK

SEQ ID NO: 781 - protein, 11 aa, Chaetomium globosum
  Soil fungus Topoisomerase 1-associated factor 1
RSRKRTRRKKK

SEQ ID NO: 782 - protein, 11 aa, Neurospora crassa
  Neurospora crassa Topoisomerase 1-associated factor 1
RSRKRARRKKK

SEQ ID NO: 783 - protein, 11 aa, Emericella nidulans
  Emericella nidulans Topoisomerase 1-associated factor 1
RSRRRARKRKR

SEQ ID NO: 784 - protein, 11 aa, Aspergillus clavatus
  Aspergillus clavatus Topoisomerase 1-associated factor 1
RSRRRIKRKAK

SEQ ID NO: 785 - protein, 11 aa, Aspergillus fumigatus
  Aspergillus fumigatus Topoisomerase 1-associated factor 1
RSRRRAKRKAK

SEQ ID NO: 786 - protein, 11 aa, Phaeosphaeria nodorum
  Phaeosphaeria nodorum Topoisomerase 1-associated factor 1
KSKRRARKIKK

SEQ ID NO: 787 - protein, 11 aa, Aspergillus niger
  Aspergillus niger (strain CBS 513.88 / FGSC A1513) Topoisomerase 1-associated factor 1
RSRRRARRKAK

SEQ ID NO: 788 - protein, 11 aa, Schizosaccharomyces pombe
  Fission yeast Serine/threonine-protein kinase ppk4
KRRRKGKRRKR

SEQ ID NO: 789 - protein, 11 aa, Neurospora crassa
  Neurospora crassa ATP-dependent rRNA helicase spb-4
REKKRRKREAK

SEQ ID NO: 790 - protein, 11 aa, Caenorhabditis briggsae
  Caenorhabditis briggsae Mediator of RNA polymerase II transcription subunit 13
KAKRKVKKFVR

SEQ ID NO: 791 - protein, 11 aa, Mus musculus
  Mouse Zinc finger protein 804A
RRKKKRRKLCR

SEQ ID NO: 792 - protein, 11 aa, Yarrowia lipolytica
  Yarrowia lipolytica Endopolyphosphatase
KGKKKGKKGKK

SEQ ID NO: 793 - protein, 11 aa, Schizophyllum commune
  Bracket fungus Mating-type protein A-alpha Y1
RARRKARKEKK

SEQ ID NO: 794 - protein, 11 aa, Ajellomyces capsulata
  Darling′s disease fungus ATP-dependent rRNA helicase SPB4
KERRRERKKIR

SEQ ID NO: 795 - protein, 11 aa, Schizophyllum commune
  Bracket fungus Mating-type protein A-alpha Y3
RAKRRARKEKK

SEQ ID NO: 796 - protein, 11 aa, Homo sapiens
  Human Uncharacterized protein C18orf34
KVKKKLRKKGK

SEQ ID NO: 797 - protein, 11 aa, Schizophyllum commune
  Bracket fungus Mating-type protein A-alpha Y4
RARRKERKQRK

SEQ ID NO: 798 - protein, 11 aa, Xenopus laevis
  African clawed frog Protein KIAA0649 homolog
KSKRKYKKRPK

SEQ ID NO: 799 - protein, 11 aa, Mus musculus
  Mouse ESF1 homolog
KEKKRLKKKQK

SEQ ID NO: 800 - protein, 11 aa, Gallus gallus
  Chicken Smoothened homolog
RSRKKKRRKKK

SEQ ID NO: 801 - protein, 11 aa, Homo sapiens
  Human ESF1 homolog
KEKKRLKRKQK

SEQ ID NO: 802 - protein, 11 aa, Bos taurus
  Bovine Probable ATP-dependent RNA helicase DDX27
RGKKKRKKFMK

SEQ ID NO: 803 - protein, 11 aa, Mus musculus
  Mouse Protein KIAA0649
RSKRKFKKRCR

SEQ ID NO: 804 - protein, 11 aa, Paramecium tetraurelia
  Paramecium tetraurelia Chromosome undetermined scaffold_13
KFKRKEKKKIK

SEQ ID NO: 805 - protein, 11 aa, Gibberella zeae
  Gibberella zeae ATP-dependent RNA helicase DBP4
KQKRREKREKR

SEQ ID NO: 806 - protein, 11 aa, Caenorhabditis elegans
  C. elegans Protein SWAP
RERKRHRKRSR

SEQ ID NO: 807 - protein, 11 aa, Magnaporthe grisea
  Rice blast fungus ATP-dependent RNA helicase DBP4
KQKKREKKLKR

SEQ ID NO: 808 - protein, 11 aa, Gallus gallus
  Chicken Transcription initiation factor TFIID subunit 3
KEKKKDKKEKK

SEQ ID NO: 809 - protein, 11 aa, Homo sapiens
  Human Extracellular sulfatase Sulf-2
KRKKKLRKLLK

SEQ ID NO: 810 - protein, 11 aa, Homo sapiens
  Human Protein KIAA0649
RSKRKLKKRCR

SEQ ID NO: 811 - protein, 11 aa, Yarrowia lipolytica
  Yarrowia lipolytica KNR4/SMI1 homolog
KSKKKNKKKGK

SEQ ID NO: 812 - protein, 11 aa, Kluyveromyces lactis
  Yeast Stress response protein NST1
KEKKKLKKKEK

SEQ ID NO: 813 - protein, 11 aa, Sclerotinia sclerotiorum
  White mold (strain ATCC 18683 / 1980 / Ss-1) ATP-dependent RNA helicase dbp4
KDKKREKREKR

SEQ ID NO: 814 - protein, 11 aa, Aspergillus niger
  Aspergillus niger (strain CBS 513.88 / FGSC A1513) ATP-dependent RNA helicase dbp4
KQKKREKKEKR

SEQ ID NO: 815 - protein, 11 aa, Gallus gallus
  Chicken Tyrosine-protein kinase-like 7
KKRRKAKRLKK

SEQ ID NO: 816 - protein, 11 aa, Aspergillus clavatus
  Aspergillus clavatus ATP-dependent RNA helicase dbp4
KQKRREKKEKR

SEQ ID NO: 817 - protein, 11 aa, Coturnix coturnix
  Quail Extracellular sulfatase Sulf-1
RRRKKERKGKK

SEQ ID NO: 818 - protein, 11 aa, Mus musculus
  Mouse Extracellular sulfatase Sulf-1
RRRKKERKEKK

SEQ ID NO: 819 - protein, 11 aa, Homo sapiens
  Human Extracellular sulfatase Sulf-1
RRRKKERKEKR

SEQ ID NO: 820 - protein, 11 aa, Homo sapiens
  Human AP-3 complex subunit delta-1
KDKRRKKRKEK

SEQ ID NO: 821 - protein, 11 aa, Homo sapiens
  Human Neuroblastoma breakpoint family member 8
RSKKKRRRGRK

SEQ ID NO: 822 - protein, 11 aa, Xenopus laevis
  African clawed frog JmjC domain-containing histone demethylation protein 1B
KEKKKIRRKRR

SEQ ID NO: 823 - protein, 11 aa, Homo sapiens
  Human Protocadherin-9
KKKRKKRKSPK

SEQ ID NO: 824 - protein, 11 aa, Drosophila melanogaster
  Fruit fly Extracellular sulfatase SULF-1 homolog
RQRKKERKRIK

SEQ ID NO: 825 - protein, 11 aa, Paramecium tetraurelia
  Paramecium tetraurelia Chromosome undetermined scaffold_114
KYKRRLRREIK

SEQ ID NO: 826 - protein, 11 aa, Homo sapiens
  Human Bromodomain and WD repeat containing protein 1
KRRRKRKKENK

SEQ ID NO: 827 - protein, 11 aa, Mus musculus
  Mouse Bromodomain and WD repeat-containing protein 1
KRRRKRRKESK

SEQ ID NO: 828 - protein, 11 aa, Yarrowia lipolytica
  Yarrowia lipolytica Increased rDNA silencing protein 4
KARRKVRRDRK

SEQ ID NO: 829 - protein, 11 aa, Mus musculus
  Mouse RNA polymerase-associated protein CTR9 homolog
RKKKRRRRPPK

SEQ ID NO: 830 - protein, 11 aa, Drosophila hydei
  Fruit fly Axoneme-associated protein mst101(2)
KGKKKCKKLGK

SEQ ID NO: 831 - protein, 11 aa, Drosophila melanogaster
  Fruit fly Nucampholin
KDKKKSKKATK

SEQ ID NO: 832 - protein, 11 aa, Saccharomyces cerevisiae
  Baker′s yeast Topoisomerase 1-associated factor 1
RSKKKDKRKRR

SEQ ID NO: 833 - protein, 11 aa, Heterosigma carterae
  Heterosigma carterae DNA-directed RNA polymerase subunit beta
KQKKKYKKTKK

SEQ ID NO: 834 - protein, 11 aa, Odontella sinensis
  Marine centric diatom DNA-directed RNA polymerase subunit beta
KLKKRMKRKYK

SEQ ID NO: 835 - protein, 11 aa, Paramecium tetraurelia
  Paramecium tetraurelia Chromosome undetermined scaffold_174
KYRKKIRKLQK

SEQ ID NO: 836 - protein, 11 aa, Plasmodium falciparum
  Plasmodium falciparum (isolate CDC / Honduras) DNA-directed RNA polymerase II subunit RPB1
KRRKRRRRKNK

SEQ ID NO: 837 - protein, 11 aa, Homo sapiens
  Human WD repeat-containing protein 87
KLKKKHKKKGK

SEQ ID NO: 838 - protein, 11 aa, Homo sapiens
  Human AT-rich interactive domain-containing protein 4A
RRRKRLKKKDR

SEQ ID NO: 839 - protein, 11 aa, Pinus thunbergii
  Green pine Protein ycf2
KRKRKTKRKTK

SEQ ID NO: 840 - protein, 11 aa, Homo sapiens
  Human AT-rich interactive domain-containing protein 4B
RRRKRLKKKER

SEQ ID NO: 841 - protein, 11 aa, Mus musculus
  Mouse Probable histone-lysine Nmethyltransferase NSD2
KGKRKKRRCWR

SEQ ID NO: 842 - protein, 11 aa, Drosophila melanogaster
  Fruit fly ATP-dependent helicase brm
KRKRRKRKNRK

SEQ ID NO: 843 - protein, 11 aa, Homo sapiens
  Human Methyl-CpG-binding domain protein 5
RVRKRNRKSGK

SEQ ID NO: 844 - protein, 11 aa, Homo sapiens
  Human Ankyrin repeat and KH domaincontaining protein 1
REKRKEKRKKK

SEQ ID NO: 845 - protein, 11 aa, Homo sapiens
  Human Transcriptional regulator ATRX
KGRKKIRKILK

SEQ ID NO: 846 - protein, 11 aa, Homo sapiens
  Human Ankyrin repeat domain-containing protein 17
REKRKEKRRKK

SEQ ID NO: 847 - protein, 11 aa, Caenorhabditis briggsae
  Caenorhabditis briggsae Ankyrin repeat and KH domain-containing protein CBG24701
KDKKKQKKEAK

SEQ ID NO: 848 - protein, 11 aa, Homo sapiens
  Human Down syndrome cell adhesion molecule-like protein 1
RKKRKEKRLKR

SEQ ID NO: 849 - protein, 11 aa, Gibberella zeae
  Gibberella zeae Helicase SWR1
KDKKKGKKKGK

SEQ ID NO: 850 - protein, 11 aa, Homo sapiens
  Human Dedicator of cytokinesis protein 1
KDKKKEKRNSK

SEQ ID NO: 851 - protein, 11 aa, Ustilago maydis
  Smut fungus Lysophospholipase NTE1
KIRKKRRRTRR

SEQ ID NO: 852 - protein, 11 aa, Mus musculus
  Mouse Fer-1-like protein 4
KKRKRKRRAGR

SEQ ID NO: 853 - protein, 11 aa, Homo sapiens
  Human Lupus brain antigen 1 homolog
RPRRRSRKCGK

SEQ ID NO: 854 - protein, 11 aa, Mus musculus
  Mouse Zonadhesin
RMKRKEKKLLR

SEQ ID NO: 855 - protein, 11 aa, Drosophila melanogaster
  Fruit fly Titin
KIKKKSRKIKK

SEQ ID NO: 856 - protein, 10 aa, Feline immunodeficiency virus
  FIV rev protein
RRKRRQRRRR

SEQ ID NO: 857 - protein, 9 aa, Homo sapiens
  Human Ankyrin repeat domain-containing protein 2
RKKRKQKKR

SEQ ID NO: 858 - protein, 9 aa, Drosophila mojavensis
  Fruit fly GI14201 protein
KRRKRQRRR

SEQ ID NO: 859 - protein, 10 aa, Tobacco mild green mosaic virus
  TMGMV (TMV strain U2) Movement protein
KKRKKEKKKR

SEQ ID NO: 860 - protein, 10 aa, Mus musculus
  Mouse T-cell surface antigen CD2
KRRKRNRRRR

SEQ ID NO: 861 - protein, 10 aa, Ustilago maydis
  Smut fungus Vacuolar fusion protein MON1
RKRRREKRRR

SEQ ID NO: 862 - protein, 10 aa, Methylobacterium
  Methylobacterium (strain 4-46) General secretory system II protein E domain protein
RPRRPRPDRR

SEQ ID NO: 863 - protein, 10 aa, Mus musculus
  Mouse Zinc finger protein 41
KPRKPRRPRK

SEQ ID NO: 864 - protein, 10 aa, Homo sapiens
  Human Coiled-coil and C2 domain-containing protein 2A
RPLRPRRKGR

SEQ ID NO: 865 - protein, 11 aa, Homo sapiens
  Human E3 ubiquitin-protein ligase HERC5
RSRRKSRRNGR

SEQ ID NO: 866 - protein, 11 aa, Xenopus laevis
  African clawed frog Midnolin-B
RLRRKARRDSR

SEQ ID NO: 867 - protein, 11 aa, Homo sapiens
  Human Early growth response protein 4
KARRKGRRGGK

SEQ ID NO: 868 - protein, 21 aa, Bufo gargarizans
  Asian toad Buforin II
TRSSRAGLQFPVGRVHRLLRK

SEQ ID NO: 869 - protein, 16 aa, Homo sapiens
  Human DPV3
RKKRRRESRKKRRRES

SEQ ID NO: 870 - protein, 17 aa, Homo sapiens
  Human DPV6
GRPRESGKKRKRKRLKP

SEQ ID NO: 871 - protein, 15 aa, Homo sapiens
  Human DPV7
GKRKKKGKLGKKRDP

SEQ ID NO: 872 - protein, 17 aa, Homo sapiens
  Human DPV7b
GKRKKKGKLGKKRPRSR

SEQ ID NO: 873 - protein, 18 aa, Homo sapiens
  Human DPV3/10
RKKRRRESRRARRSPRHL

SEQ ID NO: 874 - protein, 19 aa, Homo sapiens
  Human DPV10/6
SRRARRSPRESGKKRKRKR

SEQ ID NO: 875 - protein, 19 aa, Homo sapiens
  Human DPV1047
VKRGLKLRHVRPRVTRMDV

SEQ ID NO: 876 - protein, 18 aa, Homo sapiens
  Human DPV1048
VKRGLKLRHVRPRVTRDV

SEQ ID NO: 877 - protein, 14 aa, Homo sapiens
  Human DPV10
SRRARRSPRHLGSG

SEQ ID NO: 878 - protein, 16 aa, Homo sapiens
  Human DPV15
LRRERQSRLRRERQSR

SEQ ID NO: 879 - protein, 22 aa, Homo sapiens
  Human DPV15b
GAYDLRRRERQSRLRRRERQSR

SEQ ID NO: 880 - protein, 30 aa, Artificial Sequence
  GALA
WEAALAEALAEALAEHLAEALAEALEALAA

SEQ ID NO: 881 - protein, 21 aa, Homo sapiens
  Human Fibrogen beta chain
KGSWYSMRKMSMKIRPFFPQQ

SEQ ID NO: 882 - protein, 20 aa, Homo sapiens
  Human fibrinogen gamma chain precursor
KTRYYSMKKTTMKIIPFNRL

SEQ ID NO: 883 - protein, 20 aa, Homo sapiens
  Human Fibrinogen apha chain
RGADYSLRAVRMKIRPLVTQ

SEQ ID NO: 884 - protein, 24 aa, Homo sapiens
  Human hCT_(9-32)
LGTYTQDFNKFHTFPQTAIGVGAP

SEQ ID NO: 885 - protein, 12 aa, Artificial Sequence
  HN-_1
TSPLNIHNGQKL

SEQ ID NO: 886 - protein, 12 aa, Influenza virus
  Influenza virus nucleoprotein (NLS)
NSAAFEDLRVLS

SEQ ID NO: 887 - protein, 30 aa, Artificial Sequence
  KALA
WEAKLAKALAKALAKHLAKALAKALKACEA

SEQ ID NO: 888 - protein, 10 aa, Artificial Sequence
  Ku70
VPMLKPMLKE

SEQ ID NO: 889 - protein, 18 aa, Artificial Sequence
  MAP
KLALKLALKALKAALKLA

SEQ ID NO: 890 - protein, 27 aa, Artificial Sequence
  MPG
GALFLGFLGAAGSTMGAWSQPKKKRKV

SEQ ID NO: 891 - protein, 16 aa, Homo sapiens
  Human Fibroblast growth factor 4
AAVALLPAVLLALLAP

SEQ ID NO: 892 - protein, 9 aa, Homo sapiens
  Human N50 (NLS of NF-kB P50)
VQRKRQKLM

SEQ ID NO: 893 - protein, 21 aa, Artificial Sequence
  Pep-_1
KETWWETWWTEWSQPKKKRKV

SEQ ID NO: 894 - protein, 15 aa, Artificial Sequence
  Pep-_7
SDLWEMMMVSLACQY

SEQ ID NO: 895 - protein, 16 aa, Drosophila antennapedia
  Fruit fly Penetratin
RQIKIWFQNRRMKWKK

SEQ ID NO: 896 - protein, 17 aa, Drosophila antennapedia
  Fruit fly Penetratin variant
GRQIKIWFQNRRMKWKK

SEQ ID NO: 897 - protein, 7 aa, Drosophila antennapedia
  Fruit fly Short Penetratin
RRMKWKK

SEQ ID NO: 898 - protein, 17 aa, Drosophila antennapedia
  Fruit fly Penetratin 42-58
ERQIKIWFQNRRMKWKK

SEQ ID NO: 899 - protein, 7 aa, Artificial Sequence
  Poly Arginine - R7
RRRRRRR

SEQ ID NO: 900 - protein, 9 aa, Artificial Sequence
  Poly Arginine - R9
RRRRRRRRR

SEQ ID NO: 901 - protein, 16 aa, Rattus norvegicus
  Rat pISL
RVIRVWFQNKRCKDKK

SEQ ID NO: 902 - protein, 28 aa, Mus musculus
  Prion mouse PrPc1-28
MANLGYWLLALFVTMWTDVGLCKKRPKP

SEQ ID NO: 903 - protein, 18 aa, Mus musculus
  Mouse pVEC
LLIILRRRIRKQAHAHSK

SEQ ID NO: 904 - protein, 16 aa, Mus musculus
  Mouse pVEC variant
LLIILRRRIRKQAHAH

SEQ ID NO: 905 - protein, 18 aa, Artificial Sequence
  SAP
VRLPPPVRLPPPVRLPPP

SEQ ID NO: 906 - protein, 7 aa, Simian virus
  SV-40 (NLS)
PKKKRKV

SEQ ID NO: 907 - protein, 18 aa, Sus scrofa
  Porcine SynB1
RGGRLSYSRRRFSTSTGR

SEQ ID NO: 908 - protein, 10 aa, Sus scrofa
  Porcine SynB3
RRLSYSRRRF

SEQ ID NO: 909 - protein, 17 aa, Artificial Sequence
  SynB4
AWSFRVSYRGISYRRSR

SEQ ID NO: 910 - protein, 14 aa, Human immunodeficiency virus
  HIV type I Tat47-60
YGRKKRRQRRRPPQ

SEQ ID NO: 911 - protein, 11 aa, Human immunodeficiency virus
  HIV type I Tat47-57
YGRKKRRQRRR

SEQ ID NO: 912 - protein, 10 aa, Human immunodeficiency virus
  HIV type 1 Tat47-56
YGRKKRRQRR

SEQ ID NO: 913 - protein, 9 aa, Human immunodeficiency virus
  HIV type 1 Tat48-56
GRKKRRQRR

SEQ ID NO: 914 - protein, 10 aa, Human immunodeficiency virus
  HIV type 1 Tat48-57
GRKKRRQRRR

SEQ ID NO: 915 - protein, 9 aa, Human immunodeficiency virus
  HIV type 1 Tat49-57
RKKRRQRRR

SEQ ID NO: 916 - protein, 8 aa, Human immunodeficiency virus
  HIV type 1 Tat49-56
RKKRRQRR

SEQ ID NO: 917 - protein, 13 aa, Human immunodeficiency virus
  HIV type 1 Tat48-60
GRKKRRQRRRPPQ

SEQ ID NO: 918 - protein, 5 aa, Human immunodeficiency virus
  HIV type 1 Tat48-52
GRKKR

SEQ ID NO: 919 - protein, 36 aa, Human immunodeficiency virus
  HIV type 1 Tat37-72
CFITKALGISYGRKKRRQRRRPPQFSQTHQVSLSKQ

SEQ ID NO: 920 - protein, 34 aa, Human immunodeficiency virus
  HIV type 1 Tat38-72
FITKALGISYGRKKRRQRRRPQFSQTHQVSLSKQ

SEQ ID NO: 921 - protein, 13 aa, Human immunodeficiency virus
  HIV type 1 Tat47-59
YGRKKRRQRRRPP

SEQ ID NO: 922 - protein, 27 aa, Artificial Sequence
  Transportan
GWTLNSAGYLLGKINLKALAALAKKIL

SEQ ID NO: 923 - protein, 21 aa, Artificial Sequence
  Transportan 10
AGYLLGKINLKALAALAKKIL

SEQ ID NO: 924 - protein, 12 aa, Artificial Sequence
  Transportan derivative
GWTLNSAGYLLG

SEQ ID NO: 925 - protein, 14 aa, Artificial Sequence
  Transportan derivative
INLKALAALAKKIL

SEQ ID NO: 926 - protein, 34 aa, Herpes simplex virus
  Herpes simplex virus 1 VP22
DAATATRGRSAASRPTERPRAPARSASRPRRPVD

SEQ ID NO: 927 - protein, 26 aa, Artificial Sequence
  VT5
DPKGDPKGVTVTVTVTVTGKGDPKPD

SEQ ID NO: 928 - protein, 27 aa, Artificial Sequence
  Signal sequence-based peptide
GALFLGWLGAAGSTMGAWSQPKKKRKV

SEQ ID NO: 929 - protein, 18 aa, Artificial Sequence
  Amphiphilic model peptide
KLALKLALKALKAALKLA

SEQ ID NO: 930 - protein, 10 aa, Artificial Sequence
  Bacterial cell wall permeating
KFFKFFKFFK

SEQ ID NO: 931 - protein, 37 aa, Homo sapiens
  Human LL- 37
LLGDFFRKSKEKIGKEFKRIVQRIKDFLRNLVPRTES

SEQ ID NO: 932 - protein, 31 aa, Sus scrofa
  Porcine Cecropin P1
SWLSKTAKKLENSAKKRISEGIAIAIQGGPR

SEQ ID NO: 933 - protein, 30 aa, Homo sapiens
  Human  alpha defensin
ACYCRIPACIAGERRYGTCIYQGRLWAFCC

SEQ ID NO: 934 - protein, 36 aa, Homo sapiens
  Human beta defensin
DHYNCVSSGGQCLYSACPIFTKIQGTCYRGKAKCCK

SEQ ID NO: 935 - protein, 11 aa, Artificial Sequence
  Bactenecin
RKCRIWIRVCR

SEQ ID NO: 936 - protein, 42 aa, Sus scrofa
  Porcine PR- 39
RRRPRPPYLPRPRPPPFFPPRLPPRIPPGFPPRFPPRFPGKR

SEQ ID NO: 937 - protein, 13 aa, Bos taurus
  Bovine Indolicidin
ILPWKWPWWPWRR

SEQ ID NO: 938 - protein, 27 aa, Simian immunodeficiency virus
  SIV MPS
GALFLGWLGAAGSTMGAWSQPKKKRKV

SEQ ID NO: 939 - protein, 17 aa, Artificial Sequence
  pIs1
PVIRRVWFQNKRCKDKK

SEQ ID NO: 940 - protein, 11 aa, Artificial Sequence
  Protein transduction domain
YARKARRQARR

SEQ ID NO: 941 - protein, 11 aa, Artificial Sequence
  Protein transduction domain
YARAAARQARA

SEQ ID NO: 942 - protein, 11 aa, Artificial Sequence
  Protein transduction domain
YARAARRAARR

SEQ ID NO: 943 - protein, 11 aa, Artificial Sequence
  Protein transduction domain
YARAARRAARA

SEQ ID NO: 944 - protein, 11 aa, Artificial Sequence
  Protein transduction domain
YARRRRRRRRR

SEQ ID NO: 945 - protein, 11 aa, Artificial Sequence
  Protein transduction domain
YAAARRRRRRR

SEQ ID NO: 946 - protein, 11 aa, Artificial Sequence
  Protein transduction domain
AGRKKRRQRRR

SEQ ID NO: 947 - protein, 11 aa, Artificial Sequence
  Protein transduction domain
YARKARRQRRR

SEQ ID NO: 948 - protein, 11 aa, Artificial Sequence
  Protein transduction domain
YARAARRQARA

SEQ ID NO: 949 - protein, 10 aa, Artificial Sequence
  Protein transduction domain
YARAARRPRR

SEQ ID NO: 950 - protein, 11 aa, Artificial Sequence
  Protein transduction domain
YARAPRRARRR

SEQ ID NO: 951 - protein, 11 aa, Artificial Sequence
  Protein transduction domain
YARAPRRPRRR

SEQ ID NO: 952 - protein, 11 aa, Artificial Sequence
  Protein transduction domain
YARAAARPARA

SEQ ID NO: 953 - protein, 11 aa, Artificial Sequence
  Protein transduction domain
YARAPARQARA

SEQ ID NO: 954 - protein, 11 aa, Artificial Sequence
  Protein transduction domain
YARAPARPARA

SEQ ID NO: 955 - protein, 20 aa, Artificial Sequence
  Protein transduction domain
GLXRAXRXLXRSLXXXXXXX

SEQ ID NO: 956 - protein, 20 aa, Artificial Sequence
  Protein transduction domain
GLWRAWRXLXRSLWXXXXXX

SEQ ID NO: 957 - protein, 19 aa, Artificial Sequence
  Protein transduction domain
GLWRAWRXLXRSLWXXXKX

SEQ ID NO: 958 - protein, 20 aa, Artificial Sequence
  Protein transduction domain
GLWRALWRLLRSLWRLLWKA

SEQ ID NO: 959 - protein, 21 aa, Artificial Sequence
  Protein transduction domain
GLWRALWRALWRSLWKLKRKV

SEQ ID NO: 960 - protein, 20 aa, Artificial Sequence
  Protein transduction domain
GLWRALWRALRSLWKLKRKV

SEQ ID NO: 961 - protein, 20 aa, Artificial Sequence
  Protein transduction domain
GLWRALWRGLRSLWKKKRKV

SEQ ID NO: 962 - protein, 20 aa, Artificial Sequence
  Protein transduction domain
GLWRALWRLLRSLWRLLWKA

SEQ ID NO: 963 - protein, 21 aa, Artificial Sequence
  Protein transduction domain
GLWRALWRALWRSLWKLKWKV

SEQ ID NO: 964 - protein, 21 aa, Artificial Sequence
  Protein transduction domain
GLWRALWRALWRSLWKSKRKV

SEQ ID NO: 965 - protein, 21 aa, Artificial Sequence
  Protein transduction domain
GLWRALWRALWRSLWKKKRKV

SEQ ID NO: 966 - protein, 20 aa, Artificial Sequence
  Protein transduction domain
GLWRALWRLLRSLWRLLWSQ

SEQ ID NO: 967 - protein, 27 aa, Artificial Sequence
  Protein transduction domain
GLWRALWRLLRSLWRLLWSQPKKKRKV

SEQ ID NO: 968 - protein, 20 aa, Artificial Sequence
  Protein transduction domain
GLWRALWRLLRSLWRLLWKA

SEQ ID NO: 969 - protein, 16 aa, Artificial Sequence
  Protein transduction domain
GRRHHCRSKAKRSRHH

SEQ ID NO: 970 - protein, 10 aa, Artificial Sequence
  Protein transduction domain
GRRRRRRRRR

SEQ ID NO: 971 - protein, 10 aa, Artificial Sequence
  Protein transduction domain
GKKKKKKKKK

SEQ ID NO: 972 - protein, 10 aa, Artificial Sequence
  Protein transduction domain
GHHHHHHHHH

SEQ ID NO: 973 - protein, 9 aa, Artificial Sequence
  Protein transduction domain
GDPKKKRKV

SEQ ID NO: 974 - protein, 19 aa, Artificial Sequence
  Protein transduction domain
GKKTGKNRKLKSKRVKPRD

SEQ ID NO: 975 - protein, 12 aa, Artificial Sequence
  Protein transduction domain
GRKGKHKRKKLP

SEQ ID NO: 976 - protein, 18 aa, Artificial Sequence
  Protein transduction domain
GKRVAKRKLIEQNRERRR

SEQ ID NO: 977 - protein, 18 aa, Artificial Sequence
  Protein transduction domain
GRKLKKKKNEKEDKRPRT

SEQ ID NO: 978 - protein, 17 aa, Artificial Sequence
  Protein transduction domain
GKKTNLFSALIKKKKTA

SEQ ID NO: 979 - protein, 18 aa, Artificial Sequence
  Protein transduction domain
GRRERNKMAAAKCRNRRR

SEQ ID NO: 980 - protein, 18 aa, Artificial Sequence
  Protein transduction domain
GRRERNKMAAAKCRNRRR

SEQ ID NO: 981 - protein, 18 aa, Artificial Sequence
  Protein transduction domain
GKRARNTEAARRSRARKL

SEQ ID NO: 982 - protein, 14 aa, Artificial Sequence
  Protein transduction domain
GRRRRATAKYRTAH

SEQ ID NO: 983 - protein, 15 aa, Artificial Sequence
  Protein transduction domain
GKRRRRATAKYRSAH

SEQ ID NO: 984 - protein, 12 aa, Artificial Sequence
  Protein transduction domain
GRRRRKRLSHRT

SEQ ID NO: 985 - protein, 10 aa, Artificial Sequence
  Protein transduction domain
GRRRRRERNK

SEQ ID NO: 986 - protein, 16 aa, Artificial Sequence
  Protein transduction domain
GKHRHERGHHRDRRER

SEQ ID NO: 987 - protein, 19 aa, Artificial Sequence
  Protein transduction domain
TFGKKKRKLSNRESAKRSR

SEQ ID NO: 988 - protein, 16 aa, Artificial Sequence
  Protein transduction domain
SRRHHCRSKAKRSRHH

SEQ ID NO: 989 - protein, 16 aa, Artificial Sequence
  Protein transduction domain
SARHHCRSKAKRSRHH

SEQ ID NO: 990 - protein, 16 aa, Artificial Sequence
  Protein transduction domain
SRAHHCRSKAKRSRHH

SEQ ID NO: 991 - protein, 16 aa, Artificial Sequence
  Protein transduction domain
SRRAHCRSKAKRSRHH

SEQ ID NO: 992 - protein, 16 aa, Artificial Sequence
  Protein transduction domain
SRRHACRSKAKRSRHH

SEQ ID NO: 993 - protein, 16 aa, Artificial Sequence
  Protein transduction domain
SRRHHARSKAKRSRHH

SEQ ID NO: 994 - protein, 16 aa, Artificial Sequence
  Protein transduction domain
SRRHHCASKAKRSRHH

SEQ ID NO: 995 - protein, 16 aa, Artificial Sequence
  Protein transduction domain
SRRHHCRAKAKRSRHH

SEQ ID NO: 996 - protein, 16 aa, Artificial Sequence
  Protein transduction domain
SRRHHCRSAAKRSRHH

SEQ ID NO: 997 - protein, 16 aa, Artificial Sequence
  Protein transduction domain
SRRHHCRSKAARSRHH

SEQ ID NO: 998 - protein, 16 aa, Artificial Sequence
  Protein transduction domain
SRRHHCRSKAKASRHH

SEQ ID NO: 999 - protein, 16 aa, Artificial Sequence
  Protein transduction domain
SRRHHCRSKAKRARHH

SEQ ID NO: 1000 - protein, 16 aa, Artificial Sequence
  Protein transduction domain
SRRHHCRSKAKRSAHH

SEQ ID NO: 1001 - protein, 13 aa, Artificial Sequence
  Protein transduction domain
RRHHCRSKAKRSR

SEQ ID NO: 1002 - protein, 10 aa, Artificial Sequence
  Protein transduction domain
XXXXXXXXXX

SEQ ID NO: 1003 - protein, 10 aa, Artificial Sequence
  Protein transduction domain
MIIYRDLISH

SEQ ID NO: 1004 - protein, 9 aa, Artificial Sequence
  Protein transduction domain
MIIYRDLIS

SEQ ID NO: 1005 - protein, 8 aa, Artificial Sequence
  Protein transduction domain
MIIYRDLI

SEQ ID NO: 1006 - protein, 9 aa, Artificial Sequence
  Protein transduction domain
IIYRDLISH

SEQ ID NO: 1007 - DNA, 5369 bp, Artificial Sequence
  pET 28
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagtgcggccgcaagcttgtcgacggagctcgaattcggatccgcgacccatttgctgtccaccagtcatgctagccatatggctgccgcgcggcaccaggccgctgctgtgatgatgatgatgatggctgctgcccatggtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 1008 - DNA, 6145 bp, Artificial Sequence
  pET28AC8scFv
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgaaaaagacagctatcgcgattgcagtggcactggctggtttcgctaccgtggcccaggcggccgagatagtcctcacgcagtctccaggcaccctgtctttgtctccaggggaaagagccaccctctcctgcagggccagtcagagtgttagtagcgcctacttagcctggtaccagcagaaacctggccaggctcccaggctcctcatctatggtgcatccagcagggccactggcatcccagacaggttcagtggcagtgggtctgggacagacttcactctcaccatcagcagactggaacctgaagattttgcagtgtattactgtcagcagtatggtaggtcacccactttcggcggagggaccaaggtggagatcaaaggtggttcgtctagatcttcctcctctggtggcggtggctcgggcggtggtggccaggtccagctcgtccagtcaggggctgaggtgaagaagcctgggtcctcggtgaaggtctcctgcaaggcttctggaggttccttcagcagctatgctatcaactgggtgcgacaggcccctggacaagggcttgagtggatgggagggctcatgcctatctttgggacaacaaactacgcacagaagttccaggacagactcacgattaccgcggacgtatccacgagtacagcctacatgcagctgagcggcctgacatatgaagacacggccatgtattactgtgcgagagttgcctatatgttggaacctaccgtcactgcagggggtttggacgtctggggccaagggaccacggtcaccgtgagctcagcttccaccaagggcggccaggccggccagcaccatcaccatcaccatggcgcatacccgtacgacgttccggactacgcttcttaggcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 1009 - DNA, 903 bp, Artificial Sequence
  AC8scFv
atgaaaaagacagctatcgcgattgcagtggcactggctggtttcgctaccgtggcccaggcggccgagatagtcctcacgcagtctccaggcaccctgtctttgtctccaggggaaagagccaccctctcctgcagggccagtcagagtgttagtagcgcctacttagcctggtaccagcagaaacctggccaggctcccaggctcctcatctatggtgcatccagcagggccactggcatcccagacaggttcagtggcagtgggtctgggacagacttcactctcaccatcagcagactggaacctgaagattttgcagtgtattactgtcagcagtatggtaggtcacccactttcggcggagggaccaaggtggagatcaaaggtggttcgtctagatcttcctcctctggtggcggtggctcgggcggtggtggccaggtccagctcgtccagtcaggggctgaggtgaagaagcctgggtcctcggtgaaggtctcctgcaaggcttctggaggttccttcagcagctatgctatcaactgggtgcgacaggcccctggacaagggcttgagtggatgggagggctcatgcctatctttgggacaacaaactacgcacagaagttccaggacagactcacgattaccgcggacgtatccacgagtacagcctacatgcagctgagcggcctgacatatgaagacacggccatgtattactgtgcgagagttgcctatatgttggaacctaccgtcactgcagggggtttggacgtctggggccaagggaccacggtcaccgtgagctcagcttccaccaagggcggccaggccggccagcaccatcaccatcaccatggcgcatacccgtacgacgttccggactacgcttcttag

SEQ ID NO: 1010 - DNA, 936 bp, Artificial Sequence
  AC8scFvTat
atgaaaaagacagctatcgcgattgcagtggcactggctggtttcgctaccgtggcccaggcggccgagatagtcctcacgcagtctccaggcaccctgtctttgtctccaggggaaagagccaccctctcctgcagggccagtcagagtgttagtagcgcctacttagcctggtaccagcagaaacctggccaggctcccaggctcctcatctatggtgcatccagcagggccactggcatcccagacaggttcagtggcagtgggtctgggacagacttcactctcaccatcagcagactggaacctgaagattttgcagtgtattactgtcagcagtatggtaggtcacccactttcggcggagggaccaaggtggagatcaaaggtggttcgtctagatcttcctcctctggtggcggtggctcgggcggtggtggccaggtccagctcgtccagtcaggggctgaggtgaagaagcctgggtcctcggtgaaggtctcctgcaaggcttctggaggttccttcagcagctatgctatcaactgggtgcgacaggcccctggacaagggcttgagtggatgggagggctcatgcctatctttgggacaacaaactacgcacagaagttccaggacagactcacgattaccgcggacgtatccacgagtacagcctacatgcagctgagcggcctgacatatgaagacacggccatgtattactgtgcgagagttgcctatatgttggaacctaccgtcactgcagggggtttggacgtctggggccaagggaccacggtcaccgtgagctcagcttccaccaagggcggccaggccggccagcaccatcaccatcaccatggcgcatacccgtacgacgttccggactacgcttctaggggccgaaagaagcggagacagcgacgaagatag

SEQ ID NO: 1011 - DNA, 903 bp, Artificial Sequence
  AC8scFvscrambled
atgaaaaagacagctatcgcgattgcagtggcactggctggtttcgctaccgtggcccaggcggccgagatagtcctcacgcagtctccaggcaccctgtctttgtctccaggggaaagagccaccctctcctgcagggccagtcagagtgttagtagcgcctacttagcctggtaccagcagaaacctggccaggctcccaggctcctcatctatggtgcatccagcagggccactggcatcccagacaggttcagtggcagtgggtctgggacagacttcactctcaccatcagcagactggaacctgaagattttgcagtgtattactgtcagcagtatggtaggtcacccactttcggcggagggaccaaggtggagatcaaaggtggttcgtctagatcttcctcctctggtggcggtggctcgggcggtggtggccaggtccagctcgtccagtcaggggctgaggtgaagaagcctgggtcctcggtgaaggtctcctgcaaggcttctggaggttccttcagcagctatgctatcaactgggtgcgacaggcccctggacaagggcttgagtggatgggagggctcatgcctatctttgggacaacaaactacgcacagaagttccaggacagactcacgattaccgcggacgtatccacgagtacagcctacatgcagctgagcggcctgacatatgaagacacggccatgtattactgtgcgagagttgtcgccgactatttgatgggtttgggggaagcacctactaccgtctggggccaagggaccacggtcaccgtgagctcagcttccaccaagggcggccaggccggccagcaccatcaccatcaccatggcgcatacccgtacgacgttccggactacgcttcttag

SEQ ID NO: 1012 - DNA, 936 bp, Artificial Sequence
  AC8scFvscrambledTat
atgaaaaagacagctatcgcgattgcagtggcactggctggtttcgctaccgtggcccaggcggccgagatagtcctcacgcagtctccaggcaccctgtctttgtctccaggggaaagagccaccctctcctgcagggccagtcagagtgttagtagcgcctacttagcctggtaccagcagaaacctggccaggctcccaggctcctcatctatggtgcatccagcagggccactggcatcccagacaggttcagtggcagtgggtctgggacagacttcactctcaccatcagcagactggaacctgaagattttgcagtgtattactgtcagcagtatggtaggtcacccactttcggcggagggaccaaggtggagatcaaaggtggttcgtctagatcttcctcctctggtggcggtggctcgggcggtggtggccaggtccagctcgtccagtcaggggctgaggtgaagaagcctgggtcctcggtgaaggtctcctgcaaggcttctggaggttccttcagcagctatgctatcaactgggtgcgacaggcccctggacaagggcttgagtggatgggagggctcatgcctatctttgggacaacaaactacgcacagaagttccaggacagactcacgattaccgcggacgtatccacgagtacagcctacatgcagctgagcggcctgacatatgaagacacggccatgtattactgtgcgagagttgtcgccgactatttgatgggtttgggggaagcacctactaccgtctggggccaagggaccacggtcaccgtgagctcagcttccaccaagggcggccaggccggccagcaccatcaccatcaccatggcgcatacccgtacgacgttccggactacgcttctaggggccgaaagaagcggagacagcgacgaagatag

SEQ ID NO: 1013 - DNA, 127 bp, Artificial Sequence
  forward primer
gcctgacatatgaagacacggccatgtattactgtgcgagagttgtcgccgactatttgatgggtttgggggaagcacctactaccgtctggggccaagggaccacggtcaccgtgagctcagcttc

SEQ ID NO: 1014 - DNA, 127 bp, Artificial Sequence
  reverse primer
gaagctgagctcacggtgaccgtggtcccttggccccagacggtagtaggtgcttcccccaaacccatcaaatagtcggcgacaactctcgcacagtaatacatggccgtgtcttcatatgtcaggc

SEQ ID NO: 1015 - DNA, 783 bp, Artificial Sequence
  AC8FabTat Heavy chain
atggaatggagctgggtctttctcttcttcctgtcagtaactacaggtgtccactcccaggtccagctcgtccagtcaggggctgaggtgaagaagcctggatcctcggtgaaggtctcctgcaaggcttctggaggttccttcagcagctatgctatcaactgggtgcgacaggcccctggacaagggcttgagtggatgggagggctcatgcctatctttgggacaacaaactacgcacagaagttccaggacagactcacgattaccgcggacgtatccacgagtacagcctacatgcagctgagcggcctgacatatgaagacacggccatgtattactgtgcgagagttgcctatatgttggaacctaccgtcactgcagggggtttggacgtctggggccaagggaccacggtcaccgtcgcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactaggggccgaaagcggagacagcgacgaagatga

SEQ ID NO: 1016 - protein, 262 aa, Artificial Sequence
  AC8FabTat Heavy chain
MEWSWVFLFFLSVTTGVHSQVQLVQSGAEVKKPGSSVKVSCKASGGSFSSYAINWVRQAPGQGLEWMGGLMPIFGTTNYAQKFQDRLTITADVSTSTAYMQLSGLTYEDTAMYYCARVAYMLEPTVTAGGLDVWGQGTTVTVASASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTRGGRKKRRQRRR

SEQ ID NO: 1017 - DNA, 708 bp, Artificial Sequence
  AC8FabTat Light chain
atgggtgtgcccactcaggtcctggggttgctgctgctgtggcttacagatgccagatgtgagatagtcctcacgcagtctccaggcaccctgtctttgtctccaggggaaagagccaccctctcctgcagggccagtcagagtgttagtagcgcctacttagcctggtaccagcagaaacctggccaggctcccaggctcctcatctatggtgcatccagcagggccactggcatcccagacaggttcagtggcagtgggtctgggacagacttcactctcaccatcagcagactggaacctgaagattttgcagtgtattactgtcagcagtatggtaggtcacccactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcaacaccctgacgctgagcaaagcagactacgagaaacacaaagtctacgcctgcgaagtcacccatcagggcctgagatcgcccgtcacaaagagcttcaacaggggagagtgttaatga

SEQ ID NO: 1018 - protein, 234 aa, Artificial Sequence
  AC8 Fab Light chain
MGVPTQVLGLLLLWLTDARCEIVLTQSPGTLSLSPGERATLSCRASQSVSSAYLAWYQQKPGQAPRLLIYGASSRATGIPDRFSGSGSGTDFTLTISRLEPEDFAVYYCQQYGRSPTFGGGTKVEIKRTVAAPSVFIFPPSDEQLKSGTASVVCLLNNFYPREAKVQWKVDNALQSGNSQESVTEQDSKDSTYSLSNTLTLSKADYEKHKVYACEVTHQGLRSPVTKSFNRGEC

SEQ ID NO: 1019 - DNA, 939 bp, Artificial Sequence
  AC8scFvTAT1A
atgaaaaagacagctatcgcgattgcagtggcactggctggtttcgctaccgtggcccaggcggccgagatagtcctcacgcagtctccaggcaccctgtctttgtctccaggggaaagagccaccctctcctgcagggccagtcagagtgttagtagcgcctacttagcctggtaccagcagaaacctggccaggctcccaggctcctcatctatggtgcatccagcagggccactggcatcccagacaggttcagtggcagtgggtctgggacagacttcactctcaccatcagcagactggaacctgaagattttgcagtgtattactgtcagcagtatggtaggtcacccactttcggcggagggaccaaggtggagatcaaaggtggttcgtctagatcttcctcctctggtggcggtggctcgggcggtggtggccaggtccagctcgtccagtcaggggctgaggtgaagaagcctgggtcctcggtgaaggtctcctgcaaggcttctggaggttccttcagcagctatgctatcaactgggtgcgacaggcccctggacaagggcttgagtggatgggagggctcatgcctatctttgggacaacaaactacgcacagaagttccaggacagactcacgattaccgcggacgtatccacgagtacagcctacatgcagctgagcggcctgacatatgaagacacggccatgtattactgtgcgagagttgcctatatgttggaacctaccgtcactgcagggggtttggacgtctggggccaagggaccacggtcaccgtgagctcagcttccaccaagggcggccaggccggccagcaccatcaccatcaccatggcgcatacccgtacgacgttccggactacgctagcggagggcgtcgtaaacgtcgtcagcgtcgtcgtcgttaa

SEQ ID NO: 1020 - DNA, 936 bp, Artificial Sequence
  AC8scFvTAT1B
atgaaaaagacagctatcgcgattgcagtggcactggctggtttcgctaccgtggcccaggcggccgagatagtcctcacgcagtctccaggcaccctgtctttgtctccaggggaaagagccaccctctcctgcagggccagtcagagtgttagtagcgcctacttagcctggtaccagcagaaacctggccaggctcccaggctcctcatctatggtgcatccagcagggccactggcatcccagacaggttcagtggcagtgggtctgggacagacttcactctcaccatcagcagactggaacctgaagattttgcagtgtattactgtcagcagtatggtaggtcacccactttcggcggagggaccaaggtggagatcaaaggtggttcgtctagatcttcctcctctggtggcggtggctcgggcggtggtggccaggtccagctcgtccagtcaggggctgaggtgaagaagcctgggtcctcggtgaaggtctcctgcaaggcttctggaggttccttcagcagctatgctatcaactgggtgcgacaggcccctggacaagggcttgagtggatgggagggctcatgcctatctttgggacaacaaactacgcacagaagttccaggacagactcacgattaccgcggacgtatccacgagtacagcctacatgcagctgagcggcctgacatatgaagacacggccatgtattactgtgcgagagttgcctatatgttggaacctaccgtcactgcagggggtttggacgtctggggccaagggaccacggtcaccgtgagctcagcttccaccaagggcggccaggccggccagcaccatcaccatcaccatggcgcatacccgtacgacgttccggactacgctagcggagggcgtaagaaacgtaagcaaaagaaacgttaa

SEQ ID NO: 1021 - DNA, 936 bp, Artificial Sequence
  AC8scFvTAT1C
atgaaaaagacagctatcgcgattgcagtggcactggctggtttcgctaccgtggcccaggcggccgagatagtcctcacgcagtctccaggcaccctgtctttgtctccaggggaaagagccaccctctcctgcagggccagtcagagtgttagtagcgcctacttagcctggtaccagcagaaacctggccaggctcccaggctcctcatctatggtgcatccagcagggccactggcatcccagacaggttcagtggcagtgggtctgggacagacttcactctcaccatcagcagactggaacctgaagattttgcagtgtattactgtcagcagtatggtaggtcacccactttcggcggagggaccaaggtggagatcaaaggtggttcgtctagatcttcctcctctggtggcggtggctcgggcggtggtggccaggtccagctcgtccagtcaggggctgaggtgaagaagcctgggtcctcggtgaaggtctcctgcaaggcttctggaggttccttcagcagctatgctatcaactgggtgcgacaggcccctggacaagggcttgagtggatgggagggctcatgcctatctttgggacaacaaactacgcacagaagttccaggacagactcacgattaccgcggacgtatccacgagtacagcctacatgcagctgagcggcctgacatatgaagacacggccatgtattactgtgcgagagttgcctatatgttggaacctaccgtcactgcagggggtttggacgtctggggccaagggaccacggtcaccgtgagctcagcttccaccaagggcggccaggccggccagcaccatcaccatcaccatggcgcatacccgtacgacgttccggactacgctagcggagggaagcgtcgtaaacgacagcgtcgacgataa

SEQ ID NO: 1022 - DNA, 939 bp, Artificial Sequence
  AC8scFvTAT2A
atgaaaaagacagctatcgcgattgcagtggcactggctggtttcgctaccgtggcccaggcggccgagatagtcctcacgcagtctccaggcaccctgtctttgtctccaggggaaagagccaccctctcctgcagggccagtcagagtgttagtagcgcctacttagcctggtaccagcagaaacctggccaggctcccaggctcctcatctatggtgcatccagcagggccactggcatcccagacaggttcagtggcagtgggtctgggacagacttcactctcaccatcagcagactggaacctgaagattttgcagtgtattactgtcagcagtatggtaggtcacccactttcggcggagggaccaaggtggagatcaaaggtggttcgtctagatcttcctcctctggtggcggtggctcgggcggtggtggccaggtccagctcgtccagtcaggggctgaggtgaagaagcctgggtcctcggtgaaggtctcctgcaaggcttctggaggttccttcagcagctatgctatcaactgggtgcgacaggcccctggacaagggcttgagtggatgggagggctcatgcctatctttgggacaacaaactacgcacagaagttccaggacagactcacgattaccgcggacgtatccacgagtacagcctacatgcagctgagcggcctgacatatgaagacacggccatgtattactgtgcgagagttgcctatatgttggaacctaccgtcactgcagggggtttggacgtctggggccaagggaccacggtcaccgtgagctcagcttccaccaagggcggccaggccggccagcaccatcaccatcaccatggcgcatacccgtacgacgttccggactacgctagcggagggaagaagcgtaaaaaggagaagaagaagcgataa

SEQ ID NO: 1023 - DNA, 939 bp, Artificial Sequence
  AC8scFvTAT2B
atgaaaaagacagctatcgcgattgcagtggcactggctggtttcgctaccgtggcccaggcggccgagatagtcctcacgcagtctccaggcaccctgtctttgtctccaggggaaagagccaccctctcctgcagggccagtcagagtgttagtagcgcctacttagcctggtaccagcagaaacctggccaggctcccaggctcctcatctatggtgcatccagcagggccactggcatcccagacaggttcagtggcagtgggtctgggacagacttcactctcaccatcagcagactggaacctgaagattttgcagtgtattactgtcagcagtatggtaggtcacccactttcggcggagggaccaaggtggagatcaaaggtggttcgtctagatcttcctcctctggtggcggtggctcgggcggtggtggccaggtccagctcgtccagtcaggggctgaggtgaagaagcctgggtcctcggtgaaggtctcctgcaaggcttctggaggttccttcagcagctatgctatcaactgggtgcgacaggcccctggacaagggcttgagtggatgggagggctcatgcctatctttgggacaacaaactacgcacagaagttccaggacagactcacgattaccgcggacgtatccacgagtacagcctacatgcagctgagcggcctgacatatgaagacacggccatgtattactgtgcgagagttgcctatatgttggaacctaccgtcactgcagggggtttggacgtctggggccaagggaccacggtcaccgtgagctcagcttccaccaagggcggccaggccggccagcaccatcaccatcaccatggcgcatacccgtacgacgttccggactacgctagcggagggaagcgtcgaaagcgtaaccgtcgacgacgttaa

SEQ ID NO: 1024 - DNA, 939 bp, Artificial Sequence
  AC8scFvTAT2C
atgaaaaagacagctatcgcgattgcagtggcactggctggtttcgctaccgtggcccaggcggccgagatagtcctcacgcagtctccaggcaccctgtctttgtctccaggggaaagagccaccctctcctgcagggccagtcagagtgttagtagcgcctacttagcctggtaccagcagaaacctggccaggctcccaggctcctcatctatggtgcatccagcagggccactggcatcccagacaggttcagtggcagtgggtctgggacagacttcactctcaccatcagcagactggaacctgaagattttgcagtgtattactgtcagcagtatggtaggtcacccactttcggcggagggaccaaggtggagatcaaaggtggttcgtctagatcttcctcctctggtggcggtggctcgggcggtggtggccaggtccagctcgtccagtcaggggctgaggtgaagaagcctgggtcctcggtgaaggtctcctgcaaggcttctggaggttccttcagcagctatgctatcaactgggtgcgacaggcccctggacaagggcttgagtggatgggagggctcatgcctatctttgggacaacaaactacgcacagaagttccaggacagactcacgattaccgcggacgtatccacgagtacagcctacatgcagctgagcggcctgacatatgaagacacggccatgtattactgtgcgagagttgcctatatgttggaacctaccgtcactgcagggggtttggacgtctggggccaagggaccacggtcaccgtgagctcagcttccaccaagggcggccaggccggccagcaccatcaccatcaccatggcgcatacccgtacgacgttccggactacgctagcggagggcgtaagcgtcgacgtgagaaacgtcgtcgataa

SEQ ID NO: 1025 - DNA, 939 bp, Artificial Sequence
  AC8scFvTAT3A
atgaaaaagacagctatcgcgattgcagtggcactggctggtttcgctaccgtggcccaggcggccgagatagtcctcacgcagtctccaggcaccctgtctttgtctccaggggaaagagccaccctctcctgcagggccagtcagagtgttagtagcgcctacttagcctggtaccagcagaaacctggccaggctcccaggctcctcatctatggtgcatccagcagggccactggcatcccagacaggttcagtggcagtgggtctgggacagacttcactctcaccatcagcagactggaacctgaagattttgcagtgtattactgtcagcagtatggtaggtcacccactttcggcggagggaccaaggtggagatcaaaggtggttcgtctagatcttcctcctctggtggcggtggctcgggcggtggtggccaggtccagctcgtccagtcaggggctgaggtgaagaagcctgggtcctcggtgaaggtctcctgcaaggcttctggaggttccttcagcagctatgctatcaactgggtgcgacaggcccctggacaagggcttgagtggatgggagggctcatgcctatctttgggacaacaaactacgcacagaagttccaggacagactcacgattaccgcggacgtatccacgagtacagcctacatgcagctgagcggcctgacatatgaagacacggccatgtattactgtgcgagagttgcctatatgttggaacctaccgtcactgcagggggtttggacgtctggggccaagggaccacggtcaccgtgagctcagcttccaccaagggcggccaggccggccagcaccatcaccatcaccatggcgcatacccgtacgacgttccggactacgctagcggagggcgtcctcgacgacctcgtcctgaccgacgataa

SEQ ID NO: 1026 - DNA, 939 bp, Artificial Sequence
  AC8scFvTAT3B
atgaaaaagacagctatcgcgattgcagtggcactggctggtttcgctaccgtggcccaggcggccgagatagtcctcacgcagtctccaggcaccctgtctttgtctccaggggaaagagccaccctctcctgcagggccagtcagagtgttagtagcgcctacttagcctggtaccagcagaaacctggccaggctcccaggctcctcatctatggtgcatccagcagggccactggcatcccagacaggttcagtggcagtgggtctgggacagacttcactctcaccatcagcagactggaacctgaagattttgcagtgtattactgtcagcagtatggtaggtcacccactttcggcggagggaccaaggtggagatcaaaggtggttcgtctagatcttcctcctctggtggcggtggctcgggcggtggtggccaggtccagctcgtccagtcaggggctgaggtgaagaagcctgggtcctcggtgaaggtctcctgcaaggcttctggaggttccttcagcagctatgctatcaactgggtgcgacaggcccctggacaagggcttgagtggatgggagggctcatgcctatctttgggacaacaaactacgcacagaagttccaggacagactcacgattaccgcggacgtatccacgagtacagcctacatgcagctgagcggcctgacatatgaagacacggccatgtattactgtgcgagagttgcctatatgttggaacctaccgtcactgcagggggtttggacgtctggggccaagggaccacggtcaccgtgagctcagcttccaccaagggcggccaggccggccagcaccatcaccatcaccatggcgcatacccgtacgacgttccggactacgctagcggagggaagcctcgaaagcctcgacgtcctcgtaagtaa

SEQ ID NO: 1027 - DNA, 939 bp, Artificial Sequence
  AC8scFvTAT3C
atgaaaaagacagctatcgcgattgcagtggcactggctggtttcgctaccgtggcccaggcggccgagatagtcctcacgcagtctccaggcaccctgtctttgtctccaggggaaagagccaccctctcctgcagggccagtcagagtgttagtagcgcctacttagcctggtaccagcagaaacctggccaggctcccaggctcctcatctatggtgcatccagcagggccactggcatcccagacaggttcagtggcagtgggtctgggacagacttcactctcaccatcagcagactggaacctgaagattttgcagtgtattactgtcagcagtatggtaggtcacccactttcggcggagggaccaaggtggagatcaaaggtggttcgtctagatcttcctcctctggtggcggtggctcgggcggtggtggccaggtccagctcgtccagtcaggggctgaggtgaagaagcctgggtcctcggtgaaggtctcctgcaaggcttctggaggttccttcagcagctatgctatcaactgggtgcgacaggcccctggacaagggcttgagtggatgggagggctcatgcctatctttgggacaacaaactacgcacagaagttccaggacagactcacgattaccgcggacgtatccacgagtacagcctacatgcagctgagcggcctgacatatgaagacacggccatgtattactgtgcgagagttgcctatatgttggaacctaccgtcactgcagggggtttggacgtctggggccaagggaccacggtcaccgtgagctcagcttccaccaagggcggccaggccggccagcaccatcaccatcaccatggcgcatacccgtacgacgttccggactacgctagcggagggcgtcctctccgacctcgtcgtaaaggacgataa

SEQ ID NO: 1028 - DNA, 942 bp, Artificial Sequence
  AC8scFvTAT4A
atgaaaaagacagctatcgcgattgcagtggcactggctggtttcgctaccgtggcccaggcggccgagatagtcctcacgcagtctccaggcaccctgtctttgtctccaggggaaagagccaccctctcctgcagggccagtcagagtgttagtagcgcctacttagcctggtaccagcagaaacctggccaggctcccaggctcctcatctatggtgcatccagcagggccactggcatcccagacaggttcagtggcagtgggtctgggacagacttcactctcaccatcagcagactggaacctgaagattttgcagtgtattactgtcagcagtatggtaggtcacccactttcggcggagggaccaaggtggagatcaaaggtggttcgtctagatcttcctcctctggtggcggtggctcgggcggtggtggccaggtccagctcgtccagtcaggggctgaggtgaagaagcctgggtcctcggtgaaggtctcctgcaaggcttctggaggttccttcagcagctatgctatcaactgggtgcgacaggcccctggacaagggcttgagtggatgggagggctcatgcctatctttgggacaacaaactacgcacagaagttccaggacagactcacgattaccgcggacgtatccacgagtacagcctacatgcagctgagcggcctgacatatgaagacacggccatgtattactgtgcgagagttgcctatatgttggaacctaccgtcactgcagggggtttggacgtctggggccaagggaccacggtcaccgtgagctcagcttccaccaagggcggccaggccggccagcaccatcaccatcaccatggcgcatacccgtacgacgttccggactacgctagcggagggcgtagccgtcgaaagagccgacgaaacggacgttaa

SEQ ID NO: 1029 - DNA, 942 bp, Artificial Sequence
  AC8scFvTAT4B
atgaaaaagacagctatcgcgattgcagtggcactggctggtttcgctaccgtggcccaggcggccgagatagtcctcacgcagtctccaggcaccctgtctttgtctccaggggaaagagccaccctctcctgcagggccagtcagagtgttagtagcgcctacttagcctggtaccagcagaaacctggccaggctcccaggctcctcatctatggtgcatccagcagggccactggcatcccagacaggttcagtggcagtgggtctgggacagacttcactctcaccatcagcagactggaacctgaagattttgcagtgtattactgtcagcagtatggtaggtcacccactttcggcggagggaccaaggtggagatcaaaggtggttcgtctagatcttcctcctctggtggcggtggctcgggcggtggtggccaggtccagctcgtccagtcaggggctgaggtgaagaagcctgggtcctcggtgaaggtctcctgcaaggcttctggaggttccttcagcagctatgctatcaactgggtgcgacaggcccctggacaagggcttgagtggatgggagggctcatgcctatctttgggacaacaaactacgcacagaagttccaggacagactcacgattaccgcggacgtatccacgagtacagcctacatgcagctgagcggcctgacatatgaagacacggccatgtattactgtgcgagagttgcctatatgttggaacctaccgtcactgcagggggtttggacgtctggggccaagggaccacggtcaccgtgagctcagcttccaccaagggcggccaggccggccagcaccatcaccatcaccatggcgcatacccgtacgacgttccggactacgctagcggagggcgtctccgtcgtaaagcacgacgagacagccgttaa

SEQ ID NO: 1030 - DNA, 942 bp, Artificial Sequence
  AC8scFvTAT4C
atgaaaaagacagctatcgcgattgcagtggcactggctggtttcgctaccgtggcccaggcggccgagatagtcctcacgcagtctccaggcaccctgtctttgtctccaggggaaagagccaccctctcctgcagggccagtcagagtgttagtagcgcctacttagcctggtaccagcagaaacctggccaggctcccaggctcctcatctatggtgcatccagcagggccactggcatcccagacaggttcagtggcagtgggtctgggacagacttcactctcaccatcagcagactggaacctgaagattttgcagtgtattactgtcagcagtatggtaggtcacccactttcggcggagggaccaaggtggagatcaaaggtggttcgtctagatcttcctcctctggtggcggtggctcgggcggtggtggccaggtccagctcgtccagtcaggggctgaggtgaagaagcctgggtcctcggtgaaggtctcctgcaaggcttctggaggttccttcagcagctatgctatcaactgggtgcgacaggcccctggacaagggcttgagtggatgggagggctcatgcctatctttgggacaacaaactacgcacagaagttccaggacagactcacgattaccgcggacgtatccacgagtacagcctacatgcagctgagcggcctgacatatgaagacacggccatgtattactgtgcgagagttgcctatatgttggaacctaccgtcactgcagggggtttggacgtctggggccaagggaccacggtcaccgtgagctcagcttccaccaagggcggccaggccggccagcaccatcaccatcaccatggcgcatacccgtacgacgttccggactacgctagcggagggaaagcacgtcgtaagggacgacgaggaggaaaataa

SEQ ID NO: 1031 - protein, 312 aa, Artificial Sequence
  AC8scFvTAT1A
MKKTAIAIAVALAGFATVAQAAEIVLTQSPGTLSLSPGERATLSCRASQSVSSAYLAWYQQKPGQAPRLLIYGASSRATGIPDRFSGSGSGTDFTLTISRLEPEDFAVYYCQQYGRSPTFGGGTKVEIKGGSSRSSSSGGGGSGGGGQVQLVQSGAEVKKPGSSVKVSCKASGGSFSSYAINWVRQAPGQGLEWMGGLMPIFGTTNYAQKFQDRLTITADVSTSTAYMQLSGLTYEDTAMYYCARVAYMLEPTVTAGGLDVWGQGTTVTVSSASTKGGQAGQHHHHHHGAYPYDVPDYASGGRRKRRQRRRR

SEQ ID NO: 1032 - protein, 311 aa, Artificial Sequence
  AC8scFvTAT1B
MKKTAIAIAVALAGFATVAQAAEIVLTQSPGTLSLSPGERATLSCRASQSVSSAYLAWYQQKPGQAPRLLIYGASSRATGIPDRFSGSGSGTDFTLTISRLEPEDFAVYYCQQYGRSPTFGGGTKVEIKGGSSRSSSSGGGGSGGGGQVQLVQSGAEVKKPGSSVKVSCKASGGSFSSYAINWVRQAPGQGLEWMGGLMPIFGTTNYAQKFQDRLTITADVSTSTAYMQLSGLTYEDTAMYYCARVAYMLEPTVTAGGLDVWGQGTTVTVSSASTKGGQAGQHHHHHHGAYPYDVPDYASGGRKKRKQKKR

SEQ ID NO: 1033 - protein, 311 aa, Artificial Sequence
  AC8scFvTAT1C
MKKTAIAIAVALAGFATVAQAAEIVLTQSPGTLSLSPGERATLSCRASQSVSSAYLAWYQQKPGQAPRLLIYGASSRATGIPDRFSGSGSGTDFTLTISRLEPEDFAVYYCQQYGRSPTFGGGTKVEIKGGSSRSSSSGGGGSGGGGQVQLVQSGAEVKKPGSSVKVSCKASGGSFSSYAINWVRQAPGQGLEWMGGLMPIFGTTNYAQKFQDRLTITADVSTSTAYMQLSGLTYEDTAMYYCARVAYMLEPTVTAGGLDVWGQGTTVTVSSASTKGGQAGQHHHHHHGAYPYDVPDYASGGKRRKRQRRR

SEQ ID NO: 1034 - protein, 312 aa, Artificial Sequence
  AC8scFvTAT2A
MKKTAIAIAVALAGFATVAQAAEIVLTQSPGTLSLSPGERATLSCRASQSVSSAYLAWYQQKPGQAPRLLIYGASSRATGIPDRFSGSGSGTDFTLTISRLEPEDFAVYYCQQYGRSPTFGGGTKVEIKGGSSRSSSSGGGGSGGGGQVQLVQSGAEVKKPGSSVKVSCKASGGSFSSYAINWVRQAPGQGLEWMGGLMPIFGTTNYAQKFQDRLTITADVSTSTAYMQLSGLTYEDTAMYYCARVAYMLEPTVTAGGLDVWGQGTTVTVSSASTKGGQAGQHHHHHHGAYPYDVPDYASGGKKRKKEKKKR

SEQ ID NO: 1035 - protein, 312 aa, Artificial Sequence
  AC8scFvTAT2B
MKKTAIAIAVALAGFATVAQAAEIVLTQSPGTLSLSPGERATLSCRASQSVSSAYLAWYQQKPGQAPRLLIYGASSRATGIPDRFSGSGSGTDFTLTISRLEPEDFAVYYCQQYGRSPTFGGGTKVEIKGGSSRSSSSGGGGSGGGGQVQLVQSGAEVKKPGSSVKVSCKASGGSFSSYAINWVRQAPGQGLEWMGGLMPIFGTTNYAQKFQDRLTITADVSTSTAYMQLSGLTYEDTAMYYCARVAYMLEPTVTAGGLDVWGQGTTVTVSSASTKGGQAGQHHHHHHGAYPYDVPDYASGGKRRKRNPRRR

SEQ ID NO: 1036 - protein, 312 aa, Artificial Sequence
  AC8scFvTAT2C
MKKTAIAIAVALAGFATVAQAAEIVLTQSPGTLSLSPGERATLSCRASQSVSSAYLAWYQQKPGQAPRLLIYGASSRATGIPDRFSGSGSGTDFTLTISRLEPEDFAVYYCQQYGRSPTFGGGTKVEIKGGSSRSSSSGGGGSGGGGQVQLVQSGAEVKKPGSSVKVSCKASGGSFSSYAINWVRQAPGQGLEWMGGLMPIFGTTNYAQKFQDRLTITADVSTSTAYMQLSGLTYEDTAMYYCARVAYMLEPTVTAGGLDVWGQGTTVTVSSASTKGGQAGQHHHHHHGAYPYDVPDYASGGRKRRREKRRR

SEQ ID NO: 1037 - protein, 312 aa, Artificial Sequence
  AC8scFvTAT3A
MKKTAIAIAVALAGFATVAQAAEIVLTQSPGTLSLSPGERATLSCRASQSVSSAYLAWYQQKPGQAPRLLIYGASSRATGIPDRFSGSGSGTDFTLTISRLEPEDFAVYYCQQYGRSPTFGGGTKVEIKGGSSRSSSSGGGGSGGGGQVQLVQSGAEVKKPGSSVKVSCKASGGSFSSYAINWVRQAPGQGLEWMGGLMPIFGTTNYAQKFQDRLTITADVSTSTAYMQLSGLTYEDTAMYYCARVAYMLEPTVTAGGLDVWGQGTTVTVSSASTKGGQAGQHHHHHHGAYPYDVPDYASGGRPRRPRPDRR

SEQ ID NO: 1038 - protein, 312 aa, Artificial Sequence
  AC8scFvTAT3B
MKKTAIAIAVALAGFATVAQAAEIVLTQSPGTLSLSPGERATLSCRASQSVSSAYLAWYQQKPGQAPRLLIYGASSRATGIPDRFSGSGSGTDFTLTISRLEPEDFAVYYCQQYGRSPTFGGGTKVEIKGGSSRSSSSGGGGSGGGGQVQLVQSGAEVKKPGSSVKVSCKASGGSFSSYAINWVRQAPGQGLEWMGGLMPIFGTTNYAQKFQDRLTITADVSTSTAYMQLSGLTYEDTAMYYCARVAYMLEPTVTAGGLDVWGQGTTVTVSSASTKGGQAGQHHHHHHGAYPYDVPDYASGGKPRKPRRPRK

SEQ ID NO: 1039 - protein, 312 aa, Artificial Sequence
  AC8scFvTAT3C
MKKTAIAIAVALAGFATVAQAAEIVLTQSPGTLSLSPGERATLSCRASQSVSSAYLAWYQQKPGQAPRLLIYGASSRATGIPDRFSGSGSGTDFTLTISRLEPEDFAVYYCQQYGRSPTFGGGTKVEIKGGSSRSSSSGGGGSGGGGQVQLVQSGAEVKKPGSSVKVSCKASGGSFSSYAINWVRQAPGQGLEWMGGLMPIFGTTNYAQKFQDRLTITADVSTSTAYMQLSGLTYEDTAMYYCARVAYMLEPTVTAGGLDVWGQGTTVTVSSASTKGGQAGQHHHHHHGAYPYDVPDYASGGRPLRPRRKGR

SEQ ID NO: 1040 - protein, 313 aa, Artificial Sequence
  AC8scFvTAT4A
MKKTAIAIAVALAGFATVAQAAEIVLTQSPGTLSLSPGERATLSCRASQSVSSAYLAWYQQKPGQAPRLLIYGASSRATGIPDRFSGSGSGTDFTLTISRLEPEDFAVYYCQQYGRSPTFGGGTKVEIKGGSSRSSSSGGGGSGGGGQVQLVQSGAEVKKPGSSVKVSCKASGGSFSSYAINWVRQAPGQGLEWMGGLMPIFGTTNYAQKFQDRLTITADVSTSTAYMQLSGLTYEDTAMYYCARVAYMLEPTVTAGGLDVWGQGTTVTVSSASTKGGQAGQHHHHHHGAYPYDVPDYASGGRSRRKSRRNGR

SEQ ID NO: 1041 - protein, 313 aa, Artificial Sequence
  AC8scFvTAT4B
MKKTAIAIAVALAGFATVAQAAEIVLTQSPGTLSLSPGERATLSCRASQSVSSAYLAWYQQKPGQAPRLLIYGASSRATGIPDRFSGSGSGTDFTLTISRLEPEDFAVYYCQQYGRSPTFGGGTKVEIKGGSSRSSSSGGGGSGGGGQVQLVQSGAEVKKPGSSVKVSCKASGGSFSSYAINWVRQAPGQGLEWMGGLMPIFGTTNYAQKFQDRLTITADVSTSTAYMQLSGLTYEDTAMYYCARVAYMLEPTVTAGGLDVWGQGTTVTVSSASTKGGQAGQHHHHHHGAYPYDVPDYASGGRLRRKARRDSR

SEQ ID NO: 1042 - protein, 313 aa, Artificial Sequence
  AC8scFvTAT4C
MKKTAIAIAVALAGFATVAQAAEIVLTQSPGTLSLSPGERATLSCRASQSVSSAYLAWYQQKPGQAPRLLIYGASSRATGIPDRFSGSGSGTDFTLTISRLEPEDFAVYYCQQYGRSPTFGGGTKVEIKGGSSRSSSSGGGGSGGGGQVQLVQSGAEVKKPGSSVKVSCKASGGSFSSYAINWVRQAPGQGLEWMGGLMPIFGTTNYAQKFQDRLTITADVSTSTAYMQLSGLTYEDTAMYYCARVAYMLEPTVTAGGLDVWGQGTTVTVSSASTKGGQAGQHHHHHHGAYPYDVPDYASGGKARRKGRRGGK

SEQ ID NO: 1043 - protein, 9 aa, Artificial Sequence
  TAT-like transduction domain with Gln at position 6
XXXXXQXXX

SEQ ID NO: 1044 - protein, 9 aa, Artificial Sequence
  TAT-like transduction domain without Gln at position 6
XXXXXXXXX

SEQ ID NO: 1045 - protein, 10 aa, Artificial Sequence
  Prion-like transduction domain
XPXXPXXXXX

SEQ ID NO: 1046 - protein, 11 aa, Artificial Sequence
  Transduction peptide with basic charges on one face of alpha-helix
XXXXXXXXXXX

SEQ ID NO: 1047 - protein, 18 aa, Artificial Sequence
  Linker
GGSSRSSSSGGGGSGGGG

SEQ ID NO: 1048 - protein, 394 aa, Homo sapiens
  HSV1 Glycoprotein D
MGGAAARLGAVILFVVIVGLHGVRGKYALADASLKMADPNRFRGKDLPVLDPLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPKAPYTSTLLPPELSETPNATQPELAPEDPEDSALLEDPVGTVAPQIPPNWHIPSIQDAATPYHPPATPNNMGLIAGAVGGSLLAALVICGIVYWMHRRTRKAPKRIRLPHIREDDQPSSHQPLFY

SEQ ID NO: 1049 - protein, 393 aa, Homo sapiens
  HSV2 Glycoprotein D
MGRLTSGVGTAALLVVAVGLRVVCAKYALADPSLKMADPNRFRGKNLPVLDQLTDPPGVKRVYHIQPSLEDPFQPPSIPITVYYAVLERACRSVLLHAPSEAPQIVRGASDEARKHTYNLTIAWYRMGDNCAIPITVMEYTECPYNKSLGVCPIRTQPRWSYYDSFSAVSEDNLGFLMHAPAFETAGTYLRLVKINDWTEITQFILEHRARASCKYALPLRIPPAACLTSKAYQQGVTVDSIGMLPRFTPENQRTVALYSLKIAGWHGPKPPYTSTLLPPELSDTTNATQPELVPEDPEDSALLEDPAGTVSSQIPPNWHIPSIQDVAPHHAPAAPANPGLIIGALAGSTLAALVIGGIAFWVRRRRSVAPKRLRLPHIRDDDAPPSHQPLFY

SEQ ID NO: 1050 - protein, 10 aa, Artificial Sequence
  Prion PTD variant
KPSKPKTLNK

SEQ ID NO: 1051 - protein, 16 aa, Artificial Sequence
  AC8 CDRH3
VAYMLEPTVTAGGLDV

SEQ ID NO: 1052 - protein, 107 aa, Artificial Sequence
  AC8 Light Chain Variable Domain
EIVLTQSPGTLSLSPGERATLSCRASQSVSSAYLAWYQQKPGQAPRLLIYGASSRATGIPDRFSGSGSGTDFTLTISRLEPEDFAVYYCQQYGRSPTFGGGTKVEIK

SEQ ID NO: 1053 - protein, 125 aa, Artificial Sequence
  AC8 Heavy Chain Variable Domain
QVQLVQSGAEVKKPGSSVKVSCKASGGSFSSYAINWVRQAPGQGLEWMGGLMPIFGTTNYAQKFQDRLTITADVSTSTAYMQLSGLTYEDTAMYYCARVAYMLEPTVTAGGLDVWGQGTTVTVAS

SEQ ID NO: 1054 - protein, 18 aa, Artificial Sequence
  AC8 scFv Linker
GGSSRSSSSGGGGSGGGG

SEQ ID NO: 1055 - protein, 16 aa, Artificial Sequence
  AC8 CDR3
VAYMLEPTVTAGGLDV

SEQ ID NO: 1056 - protein, 250 aa, Artificial Sequence
  AC8 Fab Heavy Chain
MEWSWVFLFFLSVTTGVHSQVQLVQSGAEVKKPGSSVKVSCKASGGSFSSYAINWVRQAPGQGLEWMGGLMPIFGTTNYAQKFQDRLTITADVSTSTAYMQLSGLTYEDTAMYYCARVAYMLEPTVTAGGLDVWGQGTTVTVASASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKT